(19) United States
(12) Patent Application Publication
(10) Pub. No.: US 2024/0229073 A1
(43) Pub. Date: Jul. 11, 2024

(54) ARENAVIRUSES AS VECTORS

(71) Applicants: Hookipa Biotech GmbH, Wien (AT); Universtät Basel, Basel (CH)

(72) Inventors: Timo SCHIPPERS, Vienna (AT); Klaus ORLINGER, Vienna (AT); Daniel PINSCHEWER, Binningen (CH); Weldi Bonilla PINSCHEWER, Binningen (CH); Sarah SCHMIDT, Vienna (AT)

(21) Appl. No.: 18/558,885

(22) PCT Filed: May 12, 2022
(86) PCT No.: PCT/EP2022/062976

§ 371 Date: Nov. 3, 2023

Related U.S. Application Data
(60) Provisional application No. 63/188,317, filed on May 13, 2021.

(51) Int. Cl. C12N 15/86; A61K 39/00; A61K 39/12; C07K 14/005; C07K 14/82

(52) U.S. Cl. CPC C12N 15/86; A61K 39/12; C07K 14/005; C07K 14/82; A61K 2039/5256; C07K 2319/02; C12N 2710/20022; C12N 2760/10022; C12N 2760/10043; C12N 2800/40

(57) ABSTRACT

The present application relates to arenavirus particles containing a genome engineered such that an arenaviral open reading frame (“ORF”) is sequestered into two or more functional fragments and these fragments are expressed from two or more viral mRNA transcripts. The arenavirus particles described herein are genetically stable and provide high-level transgene expression. In certain embodiments, the arenavirus particles are tri-segmented. In particular, described herein is a nucleotide sequence comprising one or more ORFs comprising a nucleotide sequence encoding a functional fragment of arenavirus GP, NP, L or Z. Also described herein is an arenavirus particle containing a genome engineered such that an arenaviral ORF is sequestered into two or more functional fragments and these fragments are expressed from two or more viral mRNA transcripts. Also described herein is an arenavirus genomic or antigenomic segment engineered such that the transcription thereof results in one or more mRNA transcripts comprising a nucleotide sequence encoding a functional fragment of arenavirus GP, NP, L or Z. The arenavirus particles described herein may be suitable for vaccines and/or treatment of diseases and/or for the use in immunotherapies.

This application claims benefit of U.S. Provisional Patent Application No. 63/188,317, filed May 13, 2021, the disclosure of which is incorporated by reference herein in its entirety.

### REFERENCE TO SEQUENCE LISTING SUBMITTED ELECTRONICALLY

This application incorporates by reference a Sequence Listing submitted with this application in ASCII format entitled “13194-072-228_Sequence_Listing.txt” created on May 10, 2022 and having a size of 741,993 bytes.

### 1. INTRODUCTION

The present application relates to arenavirus particles engineered such that an arenaviral open reading frame (“ORF”) is separated over two or more mRNA transcripts. The arenavirus particles described herein are genetically stable and provide high-level transgene expression. In certain embodiments, the arenavirus particles are tri-segmented. In particular, described herein is a nucleotide sequence comprising one or more ORFs comprising a nucleotide sequence encoding a functional fragment of arenavirus GP, NP, L or Z. Also described herein is an arenavirus particle engineered such that an arenaviral ORF is separated over two or more mRNA transcripts. Also described herein is an arenavirus genomic or antigenomic segment engineered such that the transcription thereof results in one or more mRNA transcripts comprising a nucleotide sequence encoding a functional fragment of arenavirus GP, NP, L or Z. The arenavirus particles described herein may be suitable for vaccines and/or treatment of diseases and/or for the use in immunotherapies.

### 2. BACKGROUND

### 2.1 Replicating Viral Vector Systems

Replicating viral vector systems are optimally suited for delivering a target antigen of choice with the aim of inducing a potent immune response against it. In particular, viral vector systems are useful when aiming to elicit strong CD8+ T cell responses such as in tumor immunotherapy or with the intent of curing a persistent viral infection. To serve as a viral delivery system, wild-type viruses can be re-engineered to incorporate transgenic sequences, allowing them to induce immune responses against the respective translation production, i.e., the target of choice. Additionally, when based on viruses with a (known or insufficiently defined) disease-causing potential, re-engineering should confer the resulting vector with a defined degree of attenuation. On the other hand, immunogenicity of virally vectored delivery systems commonly depends on the vector dose administered. Accordingly, attenuation should not reduce viral growth in tissue culture systems to the point of rendering industrial production impractical. The usefulness of a replication-competent vector system for medical use therefore depends substantially on the following criteria:
1) good growth in cell culture, enabling the vector's production to high titers in industrial fermentation processes (production yields),
2) stable attenuation, i.e., the inability of the vector to revert to wild-type-like (more virulent) replication behavior in the vaccinated host (genetic stability),
3) high-level transgene expression to elicit strong immune responses against the desired target antigen(s) (transgene expression levels).

Arenaviruses such as lymphocytic choriomeningitis virus (LCMV) and Pichinde virus (PICV) have been re-engineered to serve as replicating yet attenuated delivery systems inducing immune responses against incorporated heterologous antigens (Cheng B Y, et al. (2015) J Virol 89, 7373-7384; Dhanwani R, et al. (2015) J Virol 90, 2551-2560; Emonet S F, et al. (2009) Proc Natl Acad Sci USA 106, 3473-3478; Kallert S M, et al. (2017) Nat Commun 8, 15327; Popkin D L, et al. (2011) J Virol 85, 7928-7932). The natural arenavirus genome consists of two RNA segments and contains open reading frames (ORFs) encoding for the glycoprotein and nucleoprotein, both encoded on the short (S) segment, as well as the polymerase L and matrix protein Z, which are encoded on the large (L) segment (FIG. 1A). In order to incorporate foreign transgenic sequences in addition to the four arenaviral ORFs, the S segment can be duplicated and the GP and NP ORFs of arenaviruses can be sequestered onto the resulting two S segments (S_{NP}, S_{GP}; FIGS. 1B-E; (Cheng B Y, et al. (2015) J Virol 89, 7373-7384; Emonet S F, et al. (2009) Proc Natl Acad Sci USA 106, 3473-3478; Kallert S M, et al. (2017) Nat Commun 8, 15327). The concept behind segregating NP and GP onto separate S segments is to force the virus to carry three genomic RNA segments in order to maintain its complete proteome. By consequence, there are four possibilities how to position NP, GP and two heterologous ORFs (designated as “h.p.” in FIGS. 1B-1G) on the two segments. These four possibilities here are referred to as r3LCMV, artLCMV, r3LCMVrev and artLCMVrev (FIGS. 1B-E). It has been demonstrated that r3LCMVrev and artLCMVrev grow to only very poor titers, much lower than r3LCMV or artLCMV (Cheng B Y, et al. (2015) J Virol 89, 7373-7384), and thus fail to meet the above criterion 1. Poor growth of r3LCMVrev and artLCMVrev supposedly results from NP being positioned next to the 5′ untranslated region (UTR), and thus being expressed under control of the respective (weaker) viral promoter sequences. Accordingly, the r3LCMVrev and artLCMVrev designs are not well suited for industrial production. Kallert et al. (Kallert S M, et al. (2017) Nat Commun 8, 15327) have further demonstrated that r3LCMV is genetically unstable: During infection of immunodeficient mice, r3LCMV underwent inter-segmental RNA recombination yielding a bi-segmented virus, which had reunited the NP and GP ORFs on one RNA segment. The resulting virus had lost (most of) its transgenes and no longer was attenuated (FIG. 1F), thus failed to meet the above criterion 2 owing to safety concerns. In summary, neither r3LCMV nor r3LCMVrev met all three criteria (production yields, genetic stability, transgene expression levels). In contrast artLCMV was genetically stable even after prolonged periods of replication in immunodeficient animals. The underlying mechanistic explanation was that the NP and GP ORFs in artLCMV could hypothetically be recombined on one S segment (FIG. 1G), but such recombination could only occur at the expense of losing the 5′ UTR while duplicating the 3′ UTR on both ends of the resulting S segment. The 5′ UTR and the 3′ UTR are known to form a non-covalent panhandle structure by RNA base-pairing, which allows template recognition by the viral RNA-dependent RNA polymerase (RdRp) in both a sequence- and structure-dependent manner. Accordingly, biological activity requires a precise 5′ UTR and 3′ UTR, tolerating no deviation from the natural sequence (Perez and de la Torre (2003) J Virol 77, 1184-1194). Accordingly, a hypothetical recombination product as depicted in FIG. 1G could not serve as a template for recognition by the viral RdRp and would be biologically inactive. artLCMV not only is genetically stable but also grows well in cell culture (Cheng B Y, et al. (2015) J Virol 89, 7373-7384; Kallert S M, et al. (2017) Nat Commun 8, 15327; Popkin D L, et al. (2011) J Virol 85, 7928-7932). artLCMV therefore was the only tri-segmented arenavirus design amongst the four strategies outlined in FIGS. 1B-1E, which met the specifications 1 and 2 above, i.e. good production yields and genetic stability ensuring safety.

### 2.2 Glycoprotein and Precursors Thereof

The GP open reading frame encodes three functional protein subunits, which are expressed as one ribosomal translation product. During insertion of the nascending polypeptide into the endoplasmic reticulum (ER) the signal peptide is cleaved off from the subsequent GP1 domain by the signal peptidase. The remainder GP1-GP2 sequence is membrane-inserted and it is post-translationally cleaved into GP1 and GP2 subunits by the subtilisin kexin isozyme-1 (SKI-1)/site 1 protease (SIP) but the GP1 and GP2 domains remain non-covalently associated in the mature glycoprotein complex. Especially noteworthy is the essential nature of the arenavirus GP signal peptide (SP): Unlike most signal peptides which are approximately 20 amino acids in length and are rapidly degraded after signal peptidase cleavage, the arenavirus glycoprotein SPs typically are >50 amino acids long (Eichler R, et al. (2003) FEBS Lett 538, 203-206) and serve not only to insert the GP protein into the ER but they remain associated with the GP1/GP2 complex and are incorporated into mature virions. Furthermore, the arenavirus glycoprotein SP plays an essential role in the post-translational proteolytic cleavage of GP1 from GP2 by SKI-1/SIP. When the arenavirus SP was substituted for a foreign signal peptide in transient transfection experiments, the GP1/GP2 sequence was inserted into the ER, but it failed to undergo processing by SKI-1/SIP (Agnihothram, S S, et al. (2006) J Virol 80, 5189-5198; Eichler R, et al. (2003) EMBO Rep 4, 1084-1088; Schrempf S, et al. (2007) J Virol 81, 12515-12524). GP1/GP2 cleavage by SKI-1/SIP is, however, essential for rendering the glycoprotein complex biologically active (Pinschewer, D D, et al. (2003) Proc Natl Acad Sci USA 100, 7895-7900).

### 2.3 Engineered Arenavirus Particles and Arenavirus Genomic Segments

Recently, it has been shown that an infectious arenavirus particle can be engineered to contain a genome with the ability to amplify and express its genetic material in infected cells but unable to produce further progeny in normal, not genetically engineered cells (i.e., an infectious, replication-deficient arenavirus particle) (International Publication Nos.: WO 2009/083210 A1, WO 2014/140301 A1 and WO 2015/082570 A1). It has also been shown that a modified arenavirus genomic segment can be engineered to carry a viral ORF in a position other than the wild-type position of the ORF (International Publication No.: WO 2016/075250 A1 and Publication No.: US 2017/0319673 A1). It has further been shown that a modified Pichinde virus genomic segment can be engineered to carry a viral ORF in a position other than the wild-type position of the ORF (International Publication No.: WO 2017/198726 A1 and Publication No.: US 2019/0135875 A1).

There is an urgent need for compositions, such as the engineered arenavirus particles and related compositions, to be used in vaccines and immunotherapies of various diseases.

### 3. SUMMARY OF THE INVENTION

Provided herein are nucleotide sequences, arenavirus particles, arenavirus genomic or antigenomic segments, and related compositions. Also provided herein are methods of generating an arenavirus genomic or antigenomic RNA segment, methods of generating an arenavirus particle, and methods of rescuing an arenavirus particle from cDNA or RNA.

In one aspect provided herein is an engineered arenavirus particle, wherein the genome of the arenavirus particle comprises:
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR;
b) an S segment that encodes NP under the control of an arenavirus genomic 3′ UTR and a second heterologous non-arenaviral polypeptide or no polypeptide under the control of an arenavirus genomic 5′ UTR; and
c) an L segment that encodes the L protein under the control of an arenavirus genomic 3′ UTR and the Z protein under the control of an arenavirus genomic 5′ UTR; or wherein the genome of the arenavirus particle comprises:
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR;
b) an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and
c) an L segment that encodes the L protein under the control of an arenavirus genomic 3′ UTR and the Z protein under the control of an arenavirus genomic 5′ UTR, wherein the first heterologous non-arenaviral polypeptide and the second heterologous non-arenaviral polypeptide are the same or different from each other.

In some aspects, the buffer peptide comprises or consists of a fragment of the arenavirus GP1. In some aspects, the fragment of the arenavirus GP1 is shorter than the arenavirus GP1. In some aspects, the fragment of the arenavirus GP1 comprises about, at most about, or at least about 1, 2, 3, 4, 5, 10, 15, 20, 25, 30, 35, 40, 45, 50, 55, 60, or more than 60 amino acids of the arenavirus GP1, wherein the fragment of the arenavirus GP1 is the N-terminal fragment of the arenavirus GP1. In some aspects, the buffer peptide comprises about or at most about 1%, 2%, 5%, 10%, 15%, 20%, 25%, 30% basic amino acids. In some aspects, the first 8 N-terminal amino acids of the buffer peptide comprise at most 1, 2, 3, 4, or 5 basic amino acids. In some aspects, the first 5 N-terminal amino acids of the buffer peptide comprise 1, 2, 3, 4, or 5 acidic and/or neutral and/or nonpolar aliphatic amino acids. In some aspects, the first 6 N-terminal amino acids of the buffer peptide comprise 1, 2, 3, 4, 5, or 6 acidic and/or neutral and/or nonpolar aliphatic amino acids. In some aspects, the first 7 N-terminal amino acids of the buffer peptide comprise 1, 2, 3, 4, 5, 6, or 7 acidic and/or neutral and/or nonpolar aliphatic amino acids. In some aspects, the first 8 N-terminal amino acids of the buffer peptide comprise 1, 2, 3, 4, 5, 6, 7, or 8 acidic and/or neutral and/or nonpolar aliphatic amino acids. In some aspects, the last C-terminal amino acid of the buffer peptide is a basic amino acid. In some aspects, the last 2 C-terminal amino acids of the buffer peptide comprise 1 or 2 basic amino acids. In some aspects, the last 3 C-terminal amino acids of the buffer peptide comprise 1, 2, or 3 basic amino acids. In some aspects, the last 4 C-terminal amino acids of the buffer peptide comprise 1, 2, 3, or 4 basic amino acids. In some aspects, the last 5 C-terminal amino acids of the buffer peptide comprise 1, 2, 3, 4, or 5 basic amino acids. In some aspects, the 5 amino acids C-terminal to the first 5, 6, 7, or 8 N-terminal amino acids of the buffer peptide comprises about or at most about 1, 2, or 3 basic amino acids. In some aspects, the buffer peptide does not comprise basic amino acids. In some aspects, the first 3, 4, 5, 6, 7, or 8 N-terminal amino acids of the buffer peptide do not comprise basic amino acids. In some aspects, the buffer peptide comprises about or at least about 50%, 60%, 70%, 80%, 90%, 95%, 96%, 97%, 98%, 99%, or 100% acidic amino acids and/or neutral and/or nonpolar aliphatic amino acids. In some aspects, the first 3, 4, 5, 6, 7, or 8 N-terminal amino acids of the buffer peptide comprise about or at least about 75%, 80%, 85%, 90%, 95%, 96%, 97%, 98%, 99%, or 100% acidic amino acids and/or neutral and/or nonpolar aliphatic amino acids. In some aspects, the buffer peptide is about, at least about, or at most about, 1, 2, 3, 4, 5, 10, 15, 20, 25, 30, 35, 40, 45, 50, 55, 60, 65, 70, or more than 70 amino acids long. In some aspects, any native signal peptide of the first heterologous non-arenaviral polypeptide has been truncated or deleted. In some aspects, the arenavirus GP signal peptide can be cleaved from the buffer peptide or a portion thereof by a signal peptide peptidase. In some aspects, the signal peptide peptidase cleaves immediately N-terminal to the buffer peptide. In some aspects, the signal peptide peptidase cleaves after the first N-terminal amino acid of the buffer peptide. In some aspects, the signal peptide peptidase cleaves after the second N-terminal amino acid of the buffer peptide. In some aspects, the signal peptide peptidase cleaves after the third N-terminal amino acid of the buffer peptide. In some aspects, the signal peptide peptidase cleaves after the fourth N-terminal amino acid of the buffer peptide. In some aspects, the signal peptide peptidase cleaves after the fifth N-terminal amino acid of the buffer peptide.

In some aspects, any native signal peptide of the second heterologous non-arenaviral polypeptide is retained. In some aspects, the arenavirus GP signal peptide is cleaved at exactly the position where the buffer peptide starts. In some aspects, the arenavirus GP signal peptide is cleaved at amino acid 1, 2, 3, 4, 5, 6, 7, 8, 9, 10, or more than 10 amino acids N-terminal from where the buffer peptide starts. In some aspects, the arenavirus GP signal peptide is cleaved at amino acid 1, 2, 3, 4, 5, 6, 7, 8, 9, 10, or more than 10 amino acids C-terminal from where the buffer peptide starts. In some aspects, the buffer peptide is about, at least about, or at most about, 1, 2, 3, 4, 5, 10, 15, 20, 25, 30, 35, 40, 45, 50, 55, 60, 65, 70, or more than 70 amino acids long.

In some aspects, the arenavirus particle is derived from an arenavirus of Clade A. In some aspects, the arenavirus of Clade A is Pichinde virus (PICV). In some aspects, the sequence of the arenavirus GP signal peptide, the buffer peptide, and the first heterologous non-arenaviral polypeptide is from N-terminus to C-terminus. In some aspects, the arenavirus GP signal peptide, the buffer peptide, and the first heterologous non-arenaviral polypeptide form a fusion protein. In some aspects, the heterologous non-arenaviral signal peptide is the signal peptide of vesicular stomatitis virus glycoprotein.

In some aspects, inter-segmental recombination of the two S segments, uniting two arenavirus ORFs on only one instead of two separate S segments, results in a bi-segmented arenavirus that is not viable or that in cell culture reaches peak titers that are lower than or equivalent to the tri-segmented arenavirus from which the bi-segmented arenavirus was recombined. In some aspects, the arenavirus GP1 and arenavirus GP2 comprises an amino acid sequence starting at about amino acid 59 to about 508 of SEQ ID NO:3, or a biologically active fragment thereof. In some aspects, the GP signal peptide fused to the buffer peptide comprises or consists of an amino acid sequence that is about or at least about 95%, 96%, 97%, 98%, 99%, or 100% identical to the sequence of amino acid 1 to amino acid 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:3. In some aspects, the GP signal peptide fused to the buffer peptide comprises or consists of an amino acid sequence that is the sequence of amino acid 1 to amino acid 66 of SEQ ID NO:3. In some aspects, the GP signal peptide fused to the buffer peptide comprises or consists of an amino acid sequence that is the sequence of amino acid 1 to amino acid 81 of SEQ ID NO:3. In some aspects, the GP signal peptide fused to the buffer peptide comprises or consists of an amino acid sequence that is the sequence of amino acid 1 to amino acid 113 of SEQ ID NO:3.

In some aspects, the GP signal peptide fused to the buffer peptide comprises or consists of an amino acid sequence that is about or at least about 95%, 96%, 97%, 98%, 99%, or 100% identical to the sequence of amino acid 1 to amino acid: 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:6. In some aspects, the GP signal peptide fused to the buffer peptide comprises or consists of an amino acid sequence that is the sequence of amino acid 1 to amino acid 66 of SEQ ID NO:6. In some aspects, the GP signal peptide fused to the buffer peptide comprises or consists of an amino acid sequence that is the sequence of amino acid 1 to amino acid 81 of SEQ ID NO:6. In some aspects, the GP signal peptide fused to the buffer peptide comprises or consists of an amino acid sequence that is about or at least about 95%, 96%, 97%, 98%, 99%, or 100% identical to the sequence of amino acid 1 to amino acid: 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:120. In some aspects the GP signal peptide fused to the buffer peptide comprises or consists of an amino acid sequence that is the sequence of amino acid 1 to amino acid 66 of SEQ ID NO:120. In some aspects the GP signal peptide fused to the buffer peptide comprises or consists of an amino acid sequence that is the sequence of amino acid 1 to amino acid 81 of SEQ ID NO:120.

In some aspects, the coding sequence of the first heterologous non-arenaviral polypeptide starts at about amino acid 60 or more than about 60 amino acids downstream of the methionine start codon of the GP signal peptide. In some aspects, the first heterologous non-arenaviral polypeptide, the second heterologous non-arenaviral polypeptide, or both heterologous non-arenaviral polypeptides are each an antigen derived from an infectious organism, tumor, or allergen.

In some aspects, the antigen is selected from the group consisting of viral antigens, wherein the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae; bacterial antigens, wherein the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and tumor neoantigens or neo-epitopes and tumor associated antigens; and wherein the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMIB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

In some aspects, the genome of the arenavirus particle encodes the first heterologous non-arenaviral polypeptide and/or the second heterologous non-arenaviral polypeptide; wherein the growth or infectivity of the arenavirus particle is not inferior to a second arenavirus particle; wherein the genome of the second arenavirus particle encodes the same first heterologous non-arenaviral polypeptide and/or the same second heterologous non-arenaviral polypeptide; and wherein all arenaviral GP, NP, Z and L in the second arenavirus particle are each expressed as one ORF.

In some aspects, the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In some aspects, the arenavirus particle expresses a heterologous non-arenaviral polypeptide under control of an arenavirus genomic 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus genomic 5′ UTR is administered to the subject or to a comparable subject.

In one aspect provided herein is a set of cDNAs from which the genomic segments of the engineered arenavirus particle of the disclosure can be transcribed. In one aspect provided herein is an expression vector or a set of expression vectors comprising the cDNAs. In one aspect provided herein is a host cell comprising the set of cDNAs or the set of DNA expression vectors.

Also provided herein is a method of manufacturing the engineered arenavirus particle of the disclosure, comprising maintaining the host cell under conditions suitable for virus formation and harvesting the engineered arenavirus particle. Also provided herein is a pharmaceutical composition comprising the engineered arenavirus particle of the disclosure and a pharmaceutically acceptable carrier. In some aspects, the engineered arenavirus particle of the disclosure is used for treating or preventing a disease in a human patient.

Also provided herein is a nucleotide sequence comprising an open reading frame encoding a polypeptide consisting of an amino acid sequence that is about or at least about 95%, 96%, 97%, 98%, 99%, or 100% identical to the sequence of amino acid 1 to amino acid 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than 113 of SEQ ID NO:3. In some aspects, the nucleotide sequence comprises an open reading frame encoding a polypeptide consisting of amino acid 1 to 66 of SEQ ID NO:3. In some aspects, the nucleotide sequence comprises an open reading frame encoding a polypeptide consisting of amino acid 1 to 81 of SEQ ID NO:3. In some aspects, the nucleotide sequence comprises an open reading frame encoding a polypeptide consisting of amino acid 1 to 113 of SEQ ID NO:3.

In one aspect provided herein is a method of immunizing a subject suspected of having, diagnosed with, or at risk of developing a disease or disorder, comprising: a. administering a first engineered arenavirus particle to the subject, and b. administering a second engineered arenavirus particle to the subject, wherein the first engineered arenavirus particle is derived from a first arenavirus species and the second engineered arenavirus particle is derived from a second arenavirus species.

In some aspects provided herein, the first engineered arenavirus particle and the second engineered arenavirus particle are derived from the same arenavirus species. In some aspects provided herein, the first engineered arenavirus particle and the second engineered arenavirus particle are derived from different arenavirus species. In some aspects provided herein, the same arenavirus species is PICV. In some aspects provided herein, the same arenavirus species is LCMV. In some aspects provided herein, the same arenavirus species is Tamiami virus (TAMV). In some aspects provided herein, the same arenavirus species is Tacaribe virus (TCRV). In some aspects provided herein, the first arenavirus species is LCMV and the second arenavirus species is PICV. In some aspects provided herein, the first arenavirus species is PICV and the second arenavirus species is LCMV. In some aspects provided herein, the first arenavirus species is LCMV and the second arenavirus species is TAMV. In some aspects provided herein, the first arenavirus species is TAMV and the second arenavirus species is LCMV. In some aspects provided herein, the first arenavirus species is LCMV and the second arenavirus species is TCRV. In some aspects provided herein, the first arenavirus species is TCRV and the second arenavirus species is LCMV. In some aspects provided herein, the first arenavirus species is TAMV and the second arenavirus species is PICV. In some aspects provided herein, the first arenavirus species is PICV and the second arenavirus species is TAMV. In some aspects provided herein, the first arenavirus species is TCRV and the second arenavirus species is PICV. In some aspects provided herein, the first arenavirus species is PICV and the second arenavirus species is TCRV. In some aspects provided herein, the first arenavirus species is TCRV and the second arenavirus species is TAMV. In some aspects provided herein, the first arenavirus species is TAMV and the second arenavirus species is TCRV. In some aspects provided herein, the disease or disorder is an infection. In some aspects provided herein, the disease or disorder is a cancer.

### 3.1 Definitions

As used herein and unless otherwise indicated, the term “GP” means both the arenavirus glycoprotein and any arenavirus glycoprotein precursor. In an exemplary embodiment, the arenavirus glycoprotein precursor can be post-translationally cleaved into a signal peptide, GP1 and GP2. In certain embodiments, the arenavirus glycoprotein or any arenavirus glycoprotein precursor may be wild-type. In other embodiments, the arenavirus glycoprotein or any arenavirus glycoprotein precursor may be recombinant. GP is further described in Section 5.1.

As used herein and unless otherwise indicated, the term “NP” means both the arenavirus nucleoprotein and any arenavirus nucleoprotein precursor. In certain embodiments, the arenavirus nucleoprotein or any arenavirus nucleoprotein precursor may be wild-type. In other embodiments, the arenavirus nucleoprotein or any arenavirus nucleoprotein precursor may be recombinant. NP is further described in Section 5.1.

As used herein and unless otherwise indicated, the term “Z” means both the arenavirus Z protein and any arenavirus Z protein precursor. In certain embodiments, the arenavirus Z protein or any arenavirus Z protein precursor may be wild-type. In other embodiments, the arenavirus Z protein or any arenavirus Z protein precursor may be recombinant. Z is further described in Section 5.1.

As used herein and unless otherwise indicated, the term “L” means both the arenavirus L protein and any arenavirus L protein precursor. In certain embodiments, the arenavirus L protein or any arenavirus L protein precursor may be wild-type. In other embodiments, the arenavirus L protein or any arenavirus L protein precursor may be recombinant. L is further described in Section 5.1.

As used herein and unless otherwise indicated, the term “functional fragment” means a fragment of a polypeptide. The functional fragment of a polypeptide as described herein is not the full-length polypeptide. In certain embodiments, the functional fragment may possess one or more functions that is known to a person of ordinary skills in the art. In certain embodiments, the functional fragment may possess the biological functions of the polypeptide from which the functional fragment is derived. In an exemplary embodiment, the functional fragment may be a signal peptide that possesses a function of mediating the insertion of glycoprotein precursor into the membrane of the endoplasmic reticulum (ER). In further embodiments, besides the function of mediating the insertion of glycoprotein precursor into the membrane of the ER, the signal peptide may possess other functions, for example mediating cleavage of the polypeptide and/or acting as a trans-acting maturation factor. In other embodiments, the functional fragment may not possess any function that is known to a person of ordinary skills in the art. Functional fragment is further described in Section 5.2.

As used herein and unless otherwise indicated, the term “heterologous non-arenaviral polypeptide” means a polypeptide that is not of arenavirus origin. In certain embodiments, the heterologous non-arenaviral polypeptide is a reporter protein (see Section 5.3.1). In other embodiments, the heterologous non-arenaviral polypeptide is a signal peptide (see Section 5.3.2). In other embodiments, the heterologous non-arenaviral polypeptide is an antigen (see Section 5.3.3)

As used herein and unless otherwise indicated, the term “buffer peptide” refers to an amino acid sequence following the GP signal peptide as encoded by a viral genomic segment described herein. A buffer peptide can be 1 amino acid or more than one amino acid long. A buffer peptide can be between about 1 to about 5 amino acids, about 1 to about 10 amino acids, about 1 to about 20 amino acids, about 1 to about 30 amino acids, about 1 to about 40 amino acids, about 1 to about 50 amino acids, about 1 to about 60 amino acids, about 1 to about 70 amino acids, about 1 to about 80 amino acids, about 1 to about 90 amino acids, about 1 to about 100 amino acids, about 1 to about 110 amino acids, about 1 to about 120 amino acids, about 1 to about 130 amino acids, about 1 to about 140 amino acids, about 1 to about 150 amino acids, about 1 to about 160 amino acids, about 1 to about 170 amino acids, about 1 to about 180 amino acids, about 1 to about 190 amino acids, about 1 to about 200 amino acids, or about 1 to more than about 200 amino acids long. In some embodiments, a buffer peptide is about, at least about, or at most about 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, or more than about 200 amino acids long. Without being bound by theory, a buffer peptide can increase the cleavage efficiency by a peptidase at the cleavage site following the GP signal peptide. Buffer peptides are further described in Section 5.4. Specifically, the amino acid composition and examples are described in Sections 5.4 and 6.

The term “about” indicates and encompasses an indicated value and a range above and below that value. In certain embodiments, the term “about” indicates the designated value±1000, ±500, or ±100. In certain embodiments, the term “about” indicates the designated value ±one standard deviation of that value.

As used herein, the singular forms “a,” “an,” and “the” ˜ include the plural referents unless the context clearly indicates otherwise.

### 3.2 Conventions and Abbreviations

| Abbreviation | Convention |
|---|---|
| art | Artificial |
| CAT | Chloramphenicol acetyltransferase |
| CMI | cell-mediated immunity |
| CD8 | Cluster of differentiation 8 |
| CD4 | Cluster of differentiation 4 |
| ER | Endoplasmic reticulum |
| GFP | Green fluorescent protein |
| GP | Glycoprotein and precursors thereof |
| GP1 | Glycoprotein-1 subunit |
| GP2 | Glycoprotein-2 subunit |
| h.p. | heterologous non-arenaviral polypeptide |
| IGR | Intergenic region |
| JUNV | Junin virus |
| LCMV | Lymphocytic choriomeningitis virus |
| L | L protein and precursors thereof |
| L protein | RNA-dependent RNA polymerase |
| L segment | Long segment |
| MHC | Major Histocompatibility Complex |
| Z | Matrix protein Z and precursors thereof |
| Z protein | Matrix protein Z |
| nat | Natural |
| NP | Nucleoprotein and precursors thereof |
| ORF | Open reading frame |
| PICV | Pichinde virus |
| RFP | Red fluorescent protein |
| r2JUNV | Recombinant bi-segmented JUNV |
| r3JUNV | Recombinant tri-segmented JUNV |
| r2LCMV | Recombinant bi-segmented LCMV |
| r3LCMV | Recombinant tri-segmented LCMV |
| S segment | Short segment |
| S1P | Site 1 protease |
| SKI-1 | Subtilisin kexin isozyme-1 |
| SP | Signal peptide |
| TOM | Tomato (red fluorescent protein) |
| UTR | Untranslated region |
| VSV | Vesicular Stomatitis Virus |
| wt | Wild-type |
| ALLV | Allpahuayo virus |
| AMAV | Amapari virus |
| MOBV | Mobala virus |
| MOPV | Mopeia virus |
| FLEV | Flexal virus |
| GTOV | Guanarito virus |
| LATV | Latino virus |
| MACV | Machupo virus |
| BCNV | Bear Canyon virus |
| PARV | Parana virus |
| PIRV | Pirital virus |
| SABV | Sabiá virus |
| TAMV | Tamiami virus |
| TCRV | Tacaribe virus |
| WWAV | Whitewater Arroyo virus |
| IPPV | Ippy virus |
| ALXV | Alxa virus |
| BCNV | Bear Canyon virus |
| SBAV | Sabia virus |
| CHAPV | Chapare virus |
| LIJV | Lìjiāng virus |
| CUPXV | Cupixi virus |
| GAIV | Gairo virus |
| LORV | Loei River virus |
| LUJV | Lujo virus |
| LUAV | Luna virus |
| LULV | Luli virus |
| LNKV | Lunk virus |
| MRLV | Mariental virus |
| MRWV | Merino Walk virus |
| MORV | Morogoro virus |
| OKAV | Okahandja virus |
| PRAV | Paraná virus |
| APOV | Aporé virus |
| RYKV | Ryukyu virus |
| SOLV | Solwezi virus |
| SOUV | souris virus |
| TMMV | Tamiami virus |
| WENV | Wēnzhōu virus |
| BBRTV | Big Brushy Tank virus |
| CTNV | Catarina virus |
| SKTV | Skinner Tank virus |
| TTCV | Tonto Creek virus |
| XAPV | Xapuri virus |
| SSP | Stable signal peptide |

### 4. BRIEF DESCRIPTION OF THE FIGURES

FIGS. 1A-1G: Published tri-segmented arenavirus vector genome design strategies, and molecular mechanism underlying r3LCMV phenotypic reversion and genetic stability of artLCMV. FIGS. 1A-1E show the schematic of the RNA segments forming the genomes of wild type LCMV (FIG. 1A), r3LCMV (FIG. 1), artLCMV (FIG. 1C), r3LCMVrev (FIG. 1D) and artLCMVrev (FIG. 1E). Segment denominations are boxed and indicated in bold. UTR: untranslated region; IGR: intergenic region; ORFs are indicated by arrows. GPC: full-length glycoprotein ORF (including the natural signal peptide); NP: nucleoprotein; L: RNA-dependent RNA polymerase L; Z: Matrix protein; h.p.: heterologous non-arenaviral polypeptide. FIG. 1F shows a non-homologous inter-segmental RNA recombination event re-uniting NP and GPC on one RNA segment (Kallert S M, et al. (2017) Nat Commun 8, 15327). This process underlies the spontaneous reversion of r3LCMV to a bi-segmented viral genome with wildtype-like virulence. FIG. 1G shows a hypothetical recombination event in artLCMV reuniting GPC and NP on one single RNA segment, which is devoid of a 5′ UTR and thus lacks a functional viral promoter (Kallert S M, et al. (2017) Nat Commun 8, 15327).

FIG. 2: The output of the online tool PrediSi (Prediction of SIgnalpeptides) website (from the Technical University of Braunschweig) analyzing the PICV glycoprotein sequence is shown.

FIGS. 3A-3C: Schematic of the genomic RNA segments of PICV wildtype, PICV-Split58-(TOM/GFP) and PICV-Split59-(TOM/GFP). The length of the SSP and of the GP1/2 is indicated by the amino acids as counted in the full-length PICV-GP ORF.

FIG. 4: Schematic of the expression cassettes contained in the bacterial plasmids pol-I-L, pol-I-S1, pol-I-S2, pC-L and pC-NP, which were used to generate tri-segmented arenavirus vectors. Pol-I-P: murine polymerase I promoter; G: non-template G residue in the 5′ end of arenavirus genomes; 5′UTR: genomic 5′ untranslated region; IGR: intergenic region; 3′UTR: genomic 3′ untranslated region; pol-I-T: murine polymerase I terminator; S1 and S2: genetically engineered S segments as designed for each trisegmented vector; Act-P: Chicken beta-actin promoter; CMV-Enh: cytomegalovirus enhancer; intron: artificially introduced intron; poly-A: polyadenylation signal.

FIGS. 5A-5C: BHK-21 cells stably expressing the LCMV glycoprotein (BHK-23 cells) were transfected with the plasmids as schematically depicted in FIG. 4 and expressing viral genomic segments as described in FIGS. 3B-3C. FIG. 5A: Fluorescence microscopy images of cells transfected to rescue PICV-Split59-(TOM/GFP) imaged on day 6 after transfection. FIG. 5B: Vector titers on day 7 after transfection were determined on 3T3 and 293T-GP cells as indicated. FIG. 5C: Fresh BHK-21 cells were infected with the indicated vectors at multiplicity of infection (MOI) of 0.01 and replication-competent vector titers were determined on 3T3 cells at the indicated time points. Symbols represent the mean of three independent cell culture wells.

FIGS. 6A-6D: Schematic of the genomic RNA segments of PICV-Split58-(TOM/E7E6), PICV-Split59-(TOM/E7E6) and of artPICV-(TOM/E7E6) (FIGS. 6A-6C). FIG. 6D: BHK-23 cells were transfected with the plasmids as schematically depicted in FIG. 4 and expressing viral genomic segments as described FIGS. 6A-6C, respectively. Vector titers on day 9 after transfection were determined on 3T3 and 293T-GP cells as indicated.

FIGS. 7A-7C: FIG. 7A shows the schematic of the (glycoprotein-expressing) S1 segments of PICV-Split58-(TOM/GFP), PICV-Split59-(TOM/GFP), PICV-Split58-(TOM/E7E6) and PICV-Split59-(TOM/E7E6) in comparison to the PICV wildtype S segment. FIG. 7B shows the alignment of the N-terminus of PICV-GP with the SSP-GFP and SSP-E7E6 fusion proteins as follows: SSP(Aa1-58)-GFP expressed by PICV-Split58-(TOM/GFP), SSP(Aa1-59)-GFP expressed by PICV-Split59-(TOM/GFP), SSP(Aa1-58)-E7E6 expressed by PICV-Split58-(TOM/E7E6), and SSP(Aa1-59)-E7E6 expressed by PICV-Split59-(TOM/E7E6). FIG. 7C shows the enlargement of the sequence alignment from Aa59 Aa69. Letters indicate single-letter amino acid codes.

FIGS. 8A-8H: BHK-21 cells stably expressing the LCMV glycoprotein (BHK-23 cells) were transfected with the plasmids as schematically depicted in FIG. 4 and expressing viral genomic segments as described in each respective figure panel. Vector titers on day 7 and on day 9 after transfection were determined on 3T3 and 293T-GP cells as indicated.

FIGS. 9A-9F: BHK-21 cells were infected with the indicated viruses (vector genomes schematically depicted in FIG. 8A-H) at MOI of 0.001 (FIGS. 9A-9C) or at MOI of 0.01 (FIGS. 9D-9F). Supernatant was collected at the indicated time points and replication-competent vector titers were determined on 3T3 cells. Symbols represent the mean of two independent cell culture wells.

FIGS: 10A-1OD: BHK-21 cells were infected with the indicated viruses (vector genomes schematically depicted in FIG. 8A-H) at MOI of 0.001 (FIGS. 10A-10C) or at MOI of 0.01 (FIGS. 10D-10F). Supernatant was collected at the indicated time points and replication-competent vector titers were determined on 3T3 cells. Symbols represent the mean of two independent cell culture wells. The same results as displayed in FIG. 9A-F are shown, but grouped differently.

FIGS. 11A-11F: C57BL/6 mice were intravenously infected with 10E5 FFU of the vectors schematically displayed in FIGS. 11A-11C. Blood was collected on day eight (8) and the frequency of E749-57-specific (RAHYNIVTF) CD8 T cells was determined by H-2D^{b} dextramer staining (FIG. 11D). PICV nucleoprotein-specific CD8 T cells binding to H-2K^{b}dextramers loaded with the NP38-45 epitope (SALDFHKV) were also determined (FIG. 11E). The values plotted in FIGS. 11D and 11E were used to calculate E7 epitope dominance by dividing for each individual animal the frequency of E749-57-specific CD8+ T cells by the frequency of NP38-45-specific CD8+ T cells. The differences between the groups were determined by one-way ANOVA followed by Bonferroni's post-test using GraphPad Prism software.

FIG. 12: Schematic of the LCMV wildtype virus genome in comparison to the genome of LCMV-Split58 vectors expressing two heterologous non-arenaviral polypeptides. The length of the SSP and of the GP1/2 is indicated by the amino acids as counted in the full-length LCMV-GP ORF.

FIGS. 13A-13C: BHK-21 cells stably expressing the LCMV glycoprotein (BHK-23 cells) were transfected with the plasmids as schematically depicted in FIG. 4 and expressing viral genomic segments as described in FIG. 12 encoding full length TRP2 as a heterologous non-arenaviral polypeptide on both S segments. Subsequently, vector supernatants were titrated to determine infectious titer on 293T-GP cells (FFU) or replication competent virus titer on adherent HEK293 cells (RCV FFU). To generate vector stock material, HEK293 suspension cells were infected at MOI of 0.01 and supernatants harvested four days later. FIG. 13A: Vector titers of 12 (LCMV-Split58-(TRP2/TRP2)—bars with “*” symbol) and 6 (LCMV-Split58-(TRP2/TRP2_delSP)—bars with “#” symbol) individual rescue cultures, respectively, were determined on day 6 post transfection. Shown are arithmetic means (+/−STD deviation). FIG. 13B: Vector titers of 6 (LCMV-Split58-(TRP2/TRP2)—bars with “*” symbol) and 3 (LCMV-Split58-(TRP2/TRP2_delSP)—bars with “#” symbol) individual vector stocks (p1), respectively, were determined on day 4 post infection at MOI 0.01. FIG. 13C: Genetic stability of the TRP2 transgene was determined by RT-PCR of stock material (p1) and subsequent passage (p2), targeting either the S1-encoded (TRP2-GP; TRP2_delSP-GP) or the S2-encoded (TRP-NP) transgenes, respectively. Shown are exemplary replicates (labelled “1”, “2”, “3”, next to marker lanes “M”) for each vector. Expected PCR amplicon size 1939 bp for full-length TRP2 and 1870 bp for TRP2_delSP. Stars indicate PCR amplicons of shorter than expected size, indicating deletions in the respective transgene.

FIGS. 14A-14B: Sequence analysis of the TRP2 transgene region revealed various nucleotide exchanges within or in close proximity to the TRP2 signal peptide. Stock material (p1) of two LCMV-Split58-(TRP2/TRP2) vectors encoding the full-length TRP2 transgene on both S segments was used for Sanger consensus sequence analysis. Prior to sequencing, transgene integrity of both vectors was confirmed by a transgene-spanning RT-PCR amplification and amplicon size analysis by gel electrophoresis. FIG. 14A: Schematic of detected mutations within the N-terminal region of the TRP2 transgene. FIG. 14B: results of SignalP for cleavage probability of mutated TRP2 versions relative to the original TRP2 sequence.

FIGS. 15A-15B: Deletion of internal signal peptides of PSA and GP70 increases vector rescue and stock titers. LCMV-Split58 vectors encoding full length PSA or GP70 (i.e., LCMV-Split58-(PSA/PSA), LCMV-Split58-(GP70/GP70)) as well as vectors encoding full length PSA or GP70 on their S2 segments and PSA and GP70 without the respective native signal peptide (_delSP) on their S1 segments (i.e., LCMV-Split58-(PSA/PSA_delSP), LCMV-Split58-(GP70/GP70_delSP)), were generated by transfection of BHK-21 cells stably expressing the LCMV glycoprotein (BHK-23 cells) with the plasmids as shown in FIG. 4. Subsequently, vector supernatants were titrated to determine infectious titer on 293TVL cells (FFU) or replication competent virus titer on adherent HEK293 (RCV FFU). To generate vector stock material, HEK293 suspension cells were infected at MOI of 0.01. FIG. 15A: Vector titers of 3 individual rescues, respectively, were determined on day 6 post transfection. Shown are arithmetic means (+/−STD deviation). FIG. 15B: Vector titers of 3 individual vector stocks (p1), respectively, were determined on day 4 post infection at MOI of 0.01.

FIG. 16: PICV-Split59-vectors exhibit a very low rescue efficiency. BHK-21 cells stably expressing the LCMV glycoprotein (BHK-23 cells) were transfected with the plasmids as schematically depicted in FIG. 4 and expressing viral genomic segments encoding E7E6 and/or Trp2 and/or Trp2delSP on the S2 and S1 segments (indicated in the table as “S2-encoded transgene”/“S1-encoded transgene”). Rescue efficiencies of the individual vectors with the indicated transgene payload were calculated by dividing the number of RCV titer-positive rescues by the number of total rescues (=technical repeats) performed. Data from several independent experiments were combined.

FIGS. 17A-17B: An arenavirus signal peptide of 59 amino acids in length (SSP_59Aa) yields replication-competent PICV-Split vectors. RCV titer-positive rescue culture supernatants from the experiment reported in FIG. 16 were titrated to determine replication competent virus titer on adherent HEK293 cells (RCV FFU). To generate vector stock material (p1), HEK293 suspension cells were infected at MOI of 0.001 and supernatants were harvested three days later. FIG. 17A: Vector titers of 4 (artPICV, PICV-Split58) or 3 (PICV-Split59) and 2 (PICV-Split59-TRP2_delSP) individual rescues, respectively, were determined on day 7 post transfection. Shown are arithmetic means (+/−STD deviation). FIG. 17B: Vector titers of individual vector stocks (p 1), were determined on day 3 post infection at MOI 0.001. Shown are arithmetic means (+/−STD deviation). “N.A.”: not assessed. “Below LOD”: below level of detection.

FIGS. 18A-18F: Genome organization of Split-A, Split-B, Split-C, Split-D, Split-E and Split-F vector genomes and their X and Y variants. FIGS. 18A-4F shows the schematic description of the genome organizations of Split-A (FIG. 18A), Split-B (FIG. 18B), Split-C (FIG. 18C), Split-D (FIG. 18D), Split-E (FIG. 18E) and Split-F (FIG. 18F) vector genomes, each expressing two heterologous non-arenaviral polypeptides (“h.p.”), which may be the same or different from each other. X and Y variants (Split-Ax, Split-Ay, Split-Bx, Split-By etc.) carrying a transgene at only one of two possible sites are described for each one of the genome organizations displayed in FIGS. 18A-18F. FIGS. 18A-18F are provided for illustration purposes of Split-A, Split-B, Split-C, Split-D, Split-E and Split-F vector genomes and do not show a buffer peptide. However, the same genomic organizations as shown in this figure can be used including a buffer peptide as described herein.

FIGS. 19A-19D: BHK-21 cells stably expressing the LCMV glycoprotein (BHK-23 cells) were transfected with the plasmids as schematically depicted in FIG. 4 and expressing viral genomic segments as described in each respective figure panel (FIGS. 19A-19D). Vector titers on day 6 after transfection were determined on 3T3 and 293T-GP cells as indicated.

FIGS. 20A-20E: BHK-21 cells were infected with the vectors PICV-Split59-(TOM/Trp2), PICV-Split66-(TOM/Trp2), PICV-Split81-(TOM/Trp2) and PICV-Split113-(TOM/Trp2) consisting of the genome segments as schematically depicted in FIGS. 20A-20D. Samples were collected at the indicated time points and replication-competent infectious vector titers (RCV-FFU) were determined in a focus formation assay on 3T3 cells (FIG. 20E). Symbols represent the mean+/−SD of three cell culture wells.

FIGS. 21A-21C: FIG. 21A shows the schematic of the (glycoprotein-expressing) S1 segments of PICV-Split58-(TOM/GFP), PICV-Split59-(TOM/GFP), PICV-Split58-(TOM/E7E6), PICV-Split59-(TOM/E7E6) and PICV-Split59-(TOM/Trp2) in comparison to the PICV wildtype S segment. FIG. 21B shows the alignment of the N-terminus of PICV-GP with the SSP-GFP, SSP-E7E6 and SSP-Trp2-delSP fusion proteins as follows: SSP(Aa1-58)-GFP expressed by PICV-Split58-(TOM/GFP), SSP(Aa1-59)-GFP expressed by PICV-Split59-(TOM/GFP), SSP(Aa1-58)-E7E6 expressed by PICV-Split58-(TOM/E7E6), SSP(Aa1-59)-E7E6 expressed by PICV-Split59-(TOM/E7E6) and SSP(Aa1-59)-Trp2delSP expressed by PICV-Split59-(TOM/Trp2). FIG. 21C shows the enlargement of the sequence alignment from Aa59-Aa69. Letters indicate single-letter amino acid codes. Various constructs differ substantially in the amino acid sequence that follows C-terminally after the signal peptide—GP1 cleavage site (indicated by an arrow), resulting in differential biochemical properties including charge of the protein sequence.

FIGS. 22A-22B: FIG. 22A shows a schematic of the genomic segments of PICV-Split66-(TOM/E7E6) and variants thereof with their S1 segment differing in amino acid positions 60 and 61 of the SSP-E7E6 gene. WT denotes the amino acid sequence of the wildtype N-terminus of PICV-GP (methionine at positions 60 and 61), whereas the DD-, EE-, GG-, LL-, HH- and KK-variants denote variants encoding for the corresponding amino acids (single letter code) instead of methionine at positions 60 and 61. FIG. 22B shows the infectious replication-competent titer (RCV-FFU) of these various viruses at 24 hours after infection of BHK-21 cells at multiplicity of infection (MOI) of 0.01. RCV-FFU were determined by immunofocus assay on 3T3 cells. Symbols represent individual cell culture wells, the mean is indicated by a horizontal line.

FIGS. 23A-23D: Schematics of the genomes of Tacaribe virus- (TCRV-) based TCRV-Split58-(TOM/E7E6), TCRV-Split66-(TOM/E7E6), and TCRV-Split81-(TOM/E7E6) vectors are shown in FIGS. 23A-23C. BHK-21 cells were infected with either of these vectors at MOI=0.01 and infectious replication-competent titers in the supernatant were determined over time by immunofocus assay on adherent 293T cells (FIG. 23D). Symbols represent the mean+/−SD of two cell culture wells.

FIGS. 23E-23H: Schematics of the genomes of Tamiami virus- (TAMV-) based TAMV-Split58-(GFP/GFP), TAMV-Split66-(GFP/GFP) and TAMV-Split81-(GFP/GFP) vectors are shown in FIGS. 23E-23G. BHK-21 cells were infected with either of these vectors at MOI=0.001 and infectious replication-competent titers in the supernatant were determined over time by immunofocus assay on VERO cells (FIG. 23H). Symbols represent the mean+/−SD of two cell culture wells.

FIGS. 24A-24C: Schematics of the genomic organization of LCMV-Split-(GFP/E7E6), artLCMV-(GFP/E7E6), PICV-Split66-(TOM/E7E6) and artPICV-(TOM/E7E6) are shown in FIG. 24A. C57BL/6 mice were immunized intravenously (i.v.) with 10e5 RCV-FFU of the aforementioned vectors in heterologous prime boost combinations as indicated in the chart shown in FIGS. 24B-24C. MHC class I dextramers were used to enumerate over time in peripheral blood of the animals HPV E6-specific CD8 T cells recognizing the E648-57 epitope (EVYDFAFRDL) in the context of H-2Kb (FIG. 24B) and HPV E7-specific CD8 T cells recognizing the E749-57 epitope (RAHYNIVTF) in the context of H-2db (FIG. 24C). Specific CD8 T cell frequencies in peripheral blood were determined after gating on CD8+B220− lymphocytes. Symbols represent the mean+/−SD of three mice per group. Two-way ANOVA was performed to compare the different immunization regimens. **: p<0.01.

FIGS. 25A-25C: Schematics of the genomic organization of LCMV-Split-(GFP/E7E6), artLCMV-(GFP/E7E6), PICV-Split66-(TOM/E7E6), PICV-Split81-(TOM/E7E6) and artPICV-(TOM/E7E6) are shown in FIG. 25A. C57BL/6 mice were immunized intravenously (i.v.) with 10e5 RCV-FFU of the aforementioned vectors in heterologous prime boost combinations as indicated in the chart shown in FIGS. 25B-25C. MHC class I dextramers were used to enumerate over time in peripheral blood of the animals HPV E7-specific CD8 T cells recognizing the E749-57 epitope (RAHYNIVTF) in the context of H-2db (FIG. 25B). Symbols in FIG. 25B represent the mean+/−SD of four mice per group. On day 148 of the experiment the animals were sacrified and spleen cell suspensions were prepared to determine the frequencies of E6- and E7-specific CD8 T cells. Intracellular cytokine assays were performed after restimulation with overlapping peptide sets spanning the respective proteins. The frequency of IFN-g-, TNF-a- and IL-2-secreting cells amongst CD8+B220− lymphocytes is shown. Symbols in FIG. 25C represent individual mice, means are shown as bars. Statistical comparisons were performed by one-way ANOVA with Dunnett's post-test. **: p<0.01; *: p<0.05.

FIGS. 26A-26C: Proteins extracted from purified artPICV virions were digested with chymotrypsin for mass spectrometry-based peptide identification. FIG. 26A shows selected peptide search engine results (PULSAR search engine, Biognosys AG) for semi-specific chymotryptic peptides F.LILAGRSC[Carbamidomethyl]DG.M (left) and L.ILAGRSC[Carbamidomethyl]DG.M (right). PEP.Q values are indicated in FIG. 26A. FIG. 26B displays MS2 extracted ion chromatograms of both, peptides F.LILAGRSC[Carbamidomethyl]DG.M (left, “peptide 1”) and L.ILAGRSC[Carbamidomethyl]DG.M (right, “peptide 2”) determined by directDIA analysis (SpectroNaut, Biognosys AG). Chymotrypsin cleaves after large hydrophobic amino acids (tyrosine, tryptophane and phenylalanine; single letter codes Y, W, F, respectively) and after leucine (single letter codes L) but virtually never after glycine (single letter code G). Accordingly, the N-terminus of the identified peptides is readily explained by chymotrypsin cleavage after F and L, respectively, whereas the same cannot apply for their C-terminus consisting of a G residue. The C-terminal G59 residue of the two independently identified peptides 1 and 2 corresponds, therefore, to the naturally processed C-terminus of the PICV glycoprotein signal peptide (FIG. 26C).

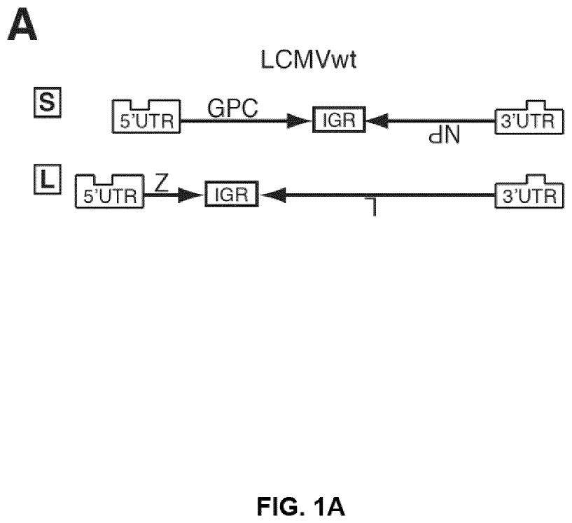

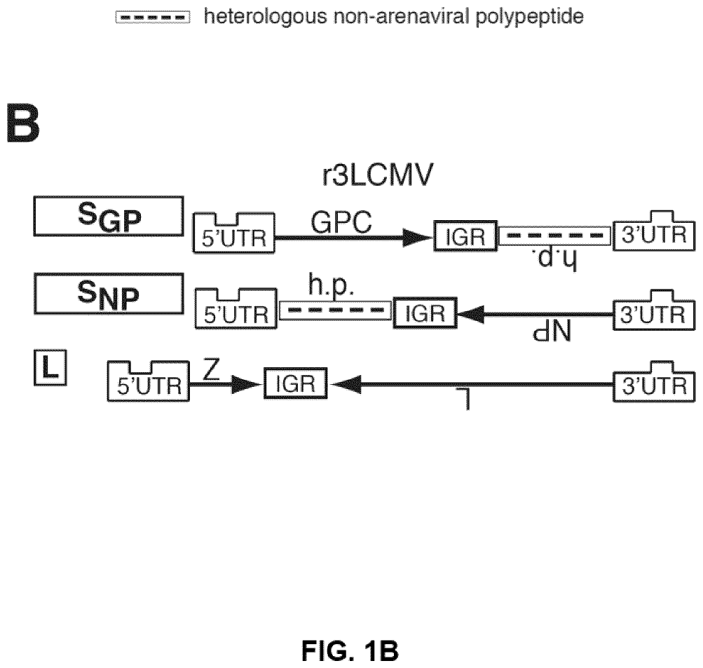

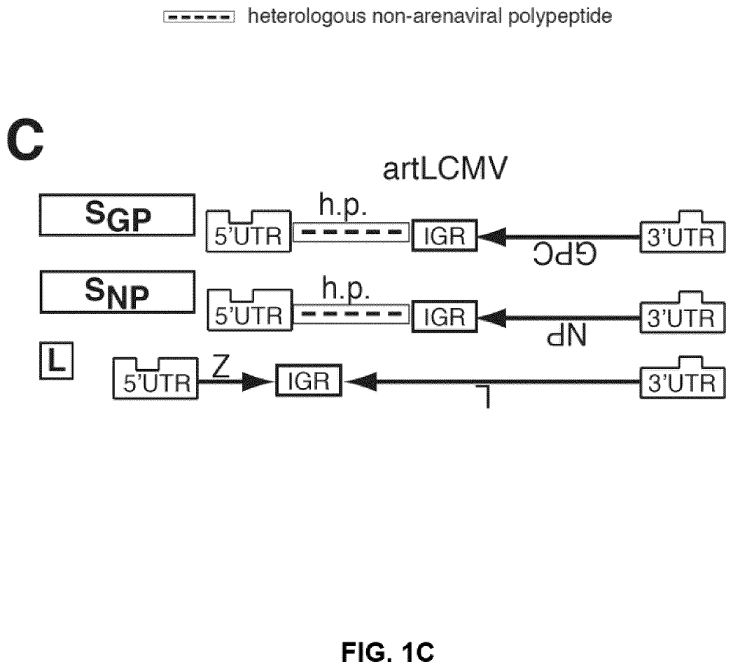

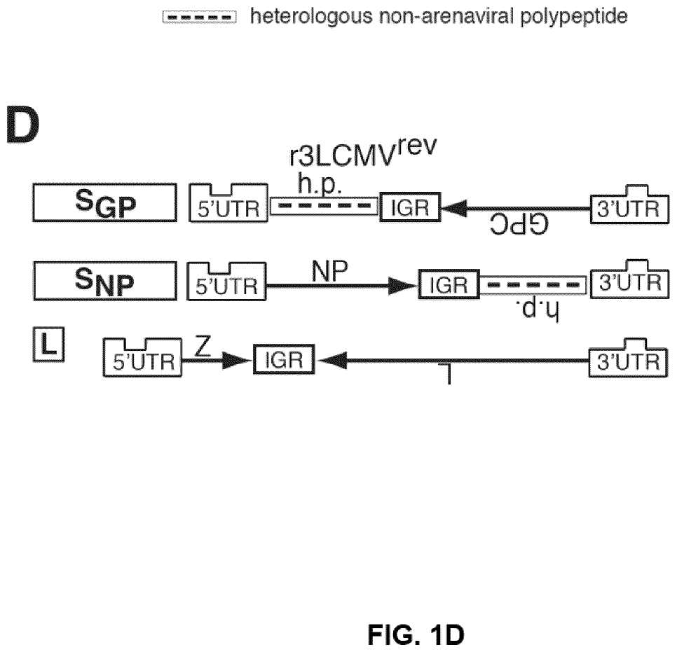

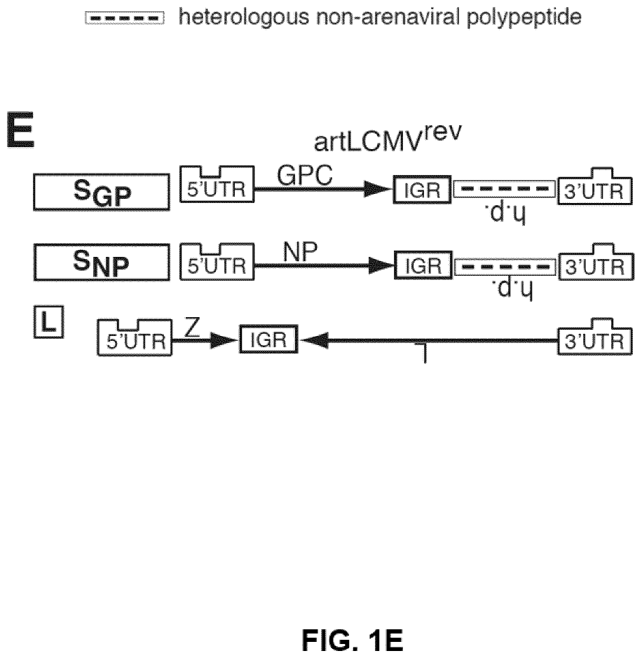

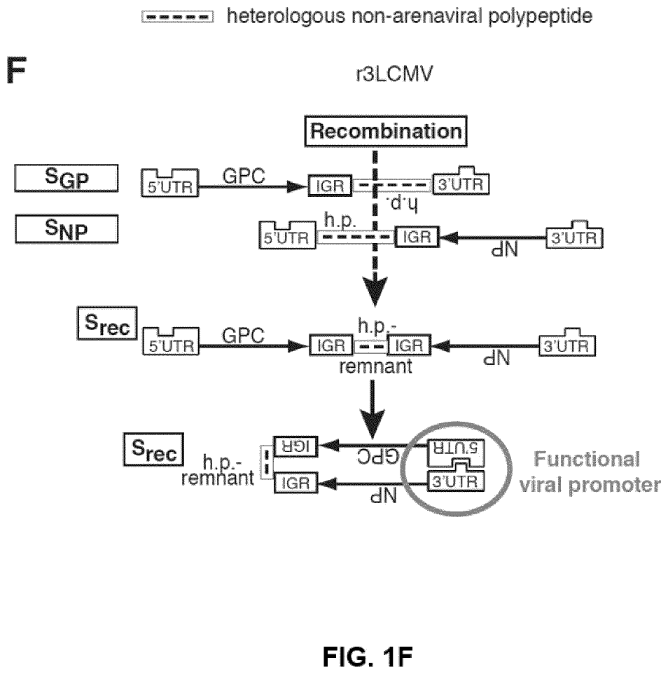

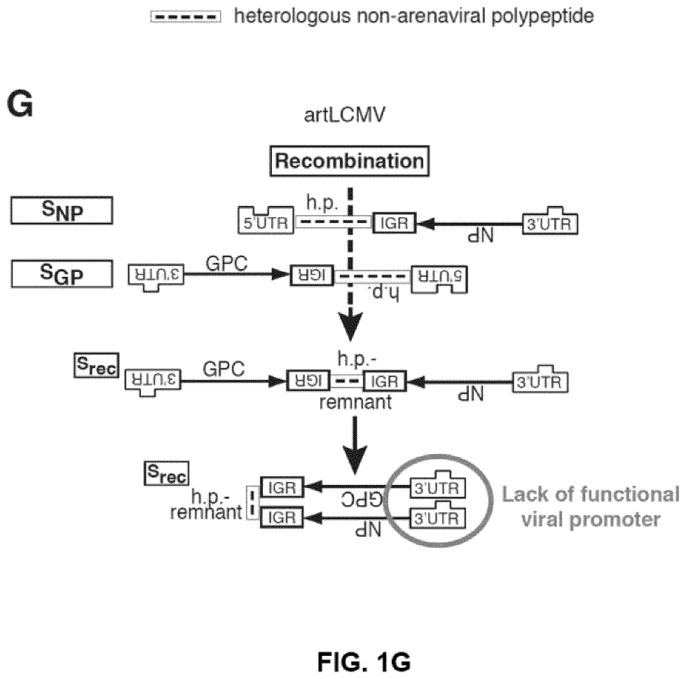

### 5. DETAILED DESCRIPTION OF THE INVENTION

Provided herein are arenavirus particles that are genetically stable and provide a high-level transgene expression. In certain embodiments, the arenavirus particles are tri-segmented. Also provided herein are nucleotide sequences and arenavirus genomic or antigenomic segments related to such arenavirus particles. In certain embodiments, provided herein is a nucleotide sequence (see Section 5.5) comprising one or more ORFs comprising a nucleotide sequence encoding a functional fragment (see Section 5.2) of arenavirus GP, NP, L or Z, and a buffer peptide (see Section 5.1) and/or a heterologous non-arenaviral polypeptide (see Section 5.3). In certain embodiments, provided herein is an arenavirus particle engineered such that an arenaviral ORF is separated over two or more mRNA transcripts (see Section 5.6). In certain embodiments, provided herein is an arenavirus genomic or antigenomic segment engineered such that the transcription thereof results in one or more mRNA transcripts comprising a nucleotide sequence encoding a functional fragment of arenavirus GP (such as the GP signal peptide and a buffer peptide as described in Section 5.4), NP, L or Z (see Section 5.7). In certain embodiments, the arenavirus particles provided herein are genetically stable, i.e. do not revert to wild-type-like (more virulent) replication behavior in the host (genetic stability) (see Section 5.14). In certain embodiments, the arenavirus particles provided herein may show high-level transgene expression to elicit strong immune responses against the desired target antigen(s) (transgene expression levels) (see Section 5.14). In certain embodiments, the arenavirus particles provided herein may show good growth in cell culture, enabling the arenavirus particle's production to high titers in industrial fermentation processes (production yields) (see Section 5.14). In certain embodiments, the arenavirus particles provided herein may be suitable for vaccines, treatment of diseases, and/or for the use in immunotherapies (see Sections 5.12 and 5.13).

Arenaviruses for use with the methods and compositions provided herein can be Old World viruses such as, for example, Lassa virus, Lymphocytic choriomeningitis virus (LCMV), Mobala virus, Mopeia virus, or Ippy virus, or New World viruses such as, for example, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Allpahuayo virus (ALLV), or Whitewater Arroyo virus. Arenaviruses for use with the methods and compositions provided herein can be, for example, arenaviruses, mammarenaviruses, Old World mammarenaviruses, New World mammarenaviruses, New World mammarenaviruses of Clade A, New World mammarenaviruses of Clade B, New World mammarenaviruses of Clade C, or New World mammarenaviruses of Clade D. Arenaviruses for use with the methods and compositions provided herein can be a mammarenavirus including, but not limited to, Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Apore virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus. In certain embodiments, the arenavirus for use with the methods and compositions provided herein is an arenavirus of Clade A. In certain embodiments, the arenavirus for use with the methods and compositions provided herein is Pichinde virus. In certain embodiments, an arenavirus as described herein wherein the GP signal peptide is fused to a buffer peptide (i) grows to higher titers, and/or (ii) reaches peak titers earlier, and/or (iii) exhibits a higher rescue efficiency (i.e., higher percentage of RCV titer-positive rescues of total rescues), as compared to a control arenavirus that has the same genomic organization except it lacks a buffer peptide. In certain embodiments, any native signal peptide of a heterologous non-arenaviral polypeptide has been removed.

### 5.1 Polypeptide Selected from the Group Consisting of Arenavirus GP, NP, Z and L

In certain embodiments, provided herein is a nucleotide sequence comprising one or more ORFs comprising a nucleotide sequence encoding a functional fragment of a polypeptide. In certain embodiments, the functional fragment is fused to a buffer peptide (as described in Section 5.4). In more specific embodiments, following translation of the functional fragment and the buffer peptide, a peptidase can cleave the functional fragment/buffer peptide fusion close to or at the fusion site. In certain embodiments, provided herein is an arenavirus genomic or antigenomic segment engineered such that transcription thereof results in one or more mRNA transcripts comprising a nucleotide sequence encoding a functional fragment of a polypeptide. In certain embodiments, the polypeptide as described herein is selected from the group consisting of arenavirus GP, NP, Z, and L, namely from the group consisting of arenavirus glycoprotein, nucleoprotein, Z protein, L protein, glycoprotein precursor, nucleoprotein precursor, Z protein precursor, and L protein precursor. In certain embodiments, the arenavirus GP, NP, Z, and L are wild-type. In other embodiments, the arenavirus GP, NP, Z, and L are recombinant. In certain embodiments, the arenavirus GP, NP, Z, and L are mutated. In certain embodiments, the arenavirus GP, NP, Z, and L are derived from an attenuated virus.

In certain embodiments, the polypeptide described herein is selected from the group consisting of wild-type arenavirus glycoprotein, nucleoprotein, Z protein, L protein, glycoprotein precursor, nucleoprotein precursor, Z protein precursor, and L protein precursor. In certain embodiments, the polypeptide described herein is a wild-type arenavirus glycoprotein precursor. In certain embodiments, the wild-type arenavirus glycoprotein precursor can be processed into a GP signal peptide, a GP1 and a GP2.

In certain embodiments, the polypeptide described herein is selected from the group consisting of recombinant arenavirus glycoprotein, nucleoprotein, Z protein, L protein, glycoprotein precursor, nucleoprotein precursor, Z protein precursor, and L protein precursor. In certain embodiments, the polypeptide described herein is a recombinant arenavirus glycoprotein precursor. In certain embodiments, the recombinant arenavirus glycoprotein precursor can be processed into a GP signal peptide, a GP1 and a GP2, one or more of which are recombinant. In some embodiments, a GP signal peptide (or functional fragment thereof or polypeptide comprising the GP signal peptide or functional fragment thereof) of the disclosure is fused to a buffer peptide (as described in Section 5.4). In more specific embodiments, following translation of the GP signal peptide (or functional fragment thereof or polypeptide comprising the GP signal peptide or functional fragment thereof) and the buffer peptide, a peptidase can cleave the GP signal peptide (or functional fragment thereof or polypeptide comprising the GP signal peptide or functional fragment thereof)/buffer peptide fusion close to (eg, −5, −4, −3, −2, −1, +1, +2, +3, +4, or +5 amino acid position from the fusion site) or at the fusion site.

In certain embodiments, the polypeptide described herein is selected from the group consisting of mutated arenavirus glycoprotein, nucleoprotein, Z protein, L protein, glycoprotein precursor, nucleoprotein precursor, Z protein precursor, and L protein precursor. In certain embodiments, the polypeptide described herein is a mutated arenavirus glycoprotein precursor. In certain embodiments, the mutated arenavirus glycoprotein precursor can be processed into a GP signal peptide, a GP1 and a GP2, one or more of which are mutated.

In certain embodiments, the polypeptide described herein is selected from the group consisting of arenavirus glycoprotein, nucleoprotein, Z protein, L protein, glycoprotein precursor, nucleoprotein precursor, Z protein precursor, and L protein precursor that are derived from an attenuated virus. In certain embodiments, the polypeptide described herein is an arenavirus glycoprotein precursor derived from an attenuated virus. In certain embodiments, the arenavirus glycoprotein precursor can be processed into a GP signal peptide, a GP1 and a GP2, one or more of which are derived from an attenuated virus.

In certain embodiments, the polypeptide described herein is selected from the group consisting of GP, NP, Z and L of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the polypeptide described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO: 107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the polypeptide described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO: 107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the polypeptide described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the polypeptide described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the polypeptide described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

In certain embodiments, the polypeptide as described herein is arenavirus GP, namely arenavirus glycoprotein or any glycoprotein precursor. In certain embodiments, the polypeptide described herein is a wild-type arenavirus glycoprotein or any wild-type glycoprotein precursor. In other embodiments, the polypeptide described herein is a recombinant arenavirus glycoprotein or any recombinant glycoprotein precursor. In certain embodiments, the arenavirus glycoprotein precursor can be processed into a GP signal peptide, a GP1 and a GP2. In certain embodiments, the arenavirus GP is arenavirus glycoprotein or any glycoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus GP as described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP as described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the GP as described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP as described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP as described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO: 134. In certain embodiments, the arenavirus GP as described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP as described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP as described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO: 134.

In certain embodiments, the polypeptide as described herein is arenavirus NP, namely arenavirus nucleoprotein or any nucleoprotein precursor. In certain embodiments, the polypeptide described herein is a wild-type arenavirus nucleoprotein or any wild-type nucleoprotein precursor. In other embodiments, the polypeptide described herein is a recombinant arenavirus nucleoprotein or any recombinant nucleoprotein precursor. In certain embodiments, the arenavirus NP is arenavirus nucleoprotein or any nucleoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus NP as described herein comprises an amino acid sequence that is at least 80%, 85%, 90%, 95%, 96%, 97%, 98%, 99% or 100% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 80% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 85% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 90% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 95% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 99% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 100% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 80% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 85% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 95% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO:138.

In certain embodiments, the polypeptide as described herein is arenavirus Z, namely arenavirus Z protein or any Z protein precursor. In certain embodiments, the polypeptide described herein is a wild-type arenavirus Z protein or any wild-type Z protein precursor. In other embodiments, the polypeptide described herein is a recombinant arenavirus Z protein or any recombinant Z protein precursor. In certain embodiments, the arenavirus Z is arenavirus Z protein or any Z protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus Z as described herein comprises an amino acid sequence that is at least 80%, 85%, 90%, 95%, 96%, 97%, 98%, 99% or 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO: 118, SEQ ID NO:125, SEQ ID NO:132, or SEQ ID NO: 139. In certain embodiments, the arenavirus Z as described herein comprises an amino acid sequence that is at least 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO: 132, or SEQ ID NO:139. In certain embodiments, the arenavirus Z as described herein comprises an amino acid sequence that is at least 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO: 139. In certain embodiments, the arenavirus Z as described herein comprises an amino acid sequence that is 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO: 139. In certain embodiments, the arenavirus Z as described herein comprises an amino acid sequence that is 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139.

In certain embodiments, the polypeptide as described herein is arenavirus L, namely arenavirus L protein or any L protein precursor. In certain embodiments, the polypeptide described herein is a wild-type arenavirus L protein or any wild-type L protein precursor. In other embodiments, the polypeptide described herein is a recombinant arenavirus L protein or any recombinant L protein precursor. In certain embodiments, the arenavirus L is arenavirus L protein or any L protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus L as described herein comprises an amino acid sequence that is at least 80%, 85%, 90%, 95%, 96%, 97%, 98%, 99% or 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is at least 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenavirus L as described herein comprises an amino acid sequence that is at least 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenavirus L as described herein comprises an amino acid sequence that is at least 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO: 140. In certain embodiments, the arenavirus L as described herein comprises an amino acid sequence that is 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO:119, SEQ ID NO:126, SEQ ID NO:133, or SEQ ID NO:140. In certain embodiments, the arenavirus L as described herein comprises an amino acid sequence that is 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO: 140. In certain embodiments, the arenavirus L as described herein comprises an amino acid sequence that is 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140.

### 5.2 Functional Fragment Provided Herein

The functional fragment to be used in the compositions and methods described herein is a fragment of a polypeptide. The functional fragment of a polypeptide as described herein is not the full-length polypeptide. In certain embodiments, the functional fragment is fused to a buffer peptide (as described in Section 5.4). In more specific embodiments, following translation of the functional fragment and the buffer peptide, a peptidase can cleave the functional fragment/buffer peptide fusion close to or at the fusion site. In some embodiments, the functional fragment is a GP signal peptide (or functional fragment thereof or polypeptide comprising the GP signal peptide or functional fragment thereof) and the GP signal peptide (or functional fragment thereof or polypeptide comprising the GP signal peptide or functional fragment thereof) is fused to a buffer peptide (as described in Section 5.4). In more specific embodiments, following translation of the GP signal peptide (or functional fragment thereof or polypeptide comprising the GP signal peptide or functional fragment thereof) and the buffer peptide, a peptidase can cleave the GP signal peptide (or functional fragment thereof or polypeptide comprising the GP signal peptide or functional fragment thereof)/buffer peptide fusion close to (eg, −5, −4, −3, −2, −1, +1, +2, +3, +4, or +5 amino acid position from the fusion site) or at the fusion site.

In certain embodiments, the functional fragment is a fragment of an arenavirus glycoprotein precursor. In certain embodiments, the functional fragment is an arenavirus GP signal peptide fused to a buffer peptide (as described in Section 5.4). In certain embodiments, the arenavirus GP signal peptide fused to a buffer peptide as described herein comprises an amino acid sequence that is at least 80%, 85%, 90%, 95%, 96%, 97%, 98%, 99% or 100% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 80% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 85% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 90% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 95% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP signal peptide fused to a buffer peptide as described herein comprises an amino acid sequence that is at least 96% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 97% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is at least 98% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, or SEQ ID NO:134. In certain embodiments, the arenavirus GP signal peptide fused to a buffer peptide as described herein comprises an amino acid sequence that is at least 99% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP signal peptide fused to a buffer peptide as described herein comprises an amino acid sequence that is 100% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO: 127, or SEQ ID NO:134. In some embodiments, the portion of the amino acid sequence is any portion of a sequence and can range in the number of amino acids. In some embodiments, the portion of the amino acid sequence is from about amino acid 1 to about amino acid 5, from about amino acid 1 to about amino acid 10, from about amino acid 1 to about amino acid 15, from about amino acid 1 to about amino acid 20, from about amino acid 1 to about amino acid 25, from about amino acid 1 to about amino acid 30, from about amino acid 1 to about amino acid 35, from about amino acid 1 to about amino acid 40, from about amino acid 1 to about amino acid 45, from about amino acid 1 to about amino acid 50, from about amino acid 1 to about amino acid 51, from about amino acid 1 to about amino acid 52, from about amino acid 1 to about amino acid 53, from about amino acid 1 to about amino acid 54, from about amino acid 1 to about amino acid 55, from about amino acid 1 to about amino acid 56, from about amino acid 1 to about amino acid 57, from about amino acid 1 to about amino acid 58, from about amino acid 1 to about amino acid 59, from about amino acid 1 to about amino acid 60, from about amino acid 1 to about amino acid 61, from about amino acid 1 to about amino acid 62, from about amino acid 1 to about amino acid 63, from about amino acid 1 to about amino acid 64, from about amino acid 1 to about amino acid 65, from about amino acid 1 to about amino acid 66, from about amino acid 1 to about amino acid 67, from about amino acid 1 to about amino acid 68, from about amino acid 1 to about amino acid 69, from about amino acid 1 to about amino acid 70, from about amino acid 1 to about amino acid 71, from about amino acid 1 to about amino acid 72, from about amino acid 1 to about amino acid 73, from about amino acid 1 to about amino acid 74, from about amino acid 1 to about amino acid 75, from about amino acid 1 to about amino acid 76, from about amino acid 1 to about amino acid 77, from about amino acid 1 to about amino acid 78, from about amino acid 1 to about amino acid 79, from about amino acid 1 to about amino acid 80, from about amino acid 1 to about amino acid 81, from about amino acid 1 to about amino acid 82, from about amino acid 1 to about amino acid 83, from about amino acid 1 to about amino acid 84, from about amino acid 1 to about amino acid 85, from about amino acid 1 to about amino acid 86, from about amino acid 1 to about amino acid 87, from about amino acid 1 to about amino acid 88, from about amino acid 1 to about amino acid 89, from about amino acid 1 to about amino acid 90, from about amino acid 1 to about amino acid 91, from about amino acid 1 to about amino acid 92, from about amino acid 1 to about amino acid 93, from about amino acid 1 to about amino acid 94, from about amino acid 1 to about amino acid 95, from about amino acid 1 to about amino acid 96, from about amino acid 1 to about amino acid 97, from about amino acid 1 to about amino acid 98, from about amino acid 1 to about amino acid 99, from about amino acid 1 to about amino acid 100, from about amino acid 1 to about amino acid 101, from about amino acid 1 to about amino acid 102, from about amino acid 1 to about amino acid 103, from about amino acid 1 to about amino acid 104, from about amino acid 1 to about amino acid 105, from about amino acid 1 to about amino acid 106, from about amino acid 1 to about amino acid 107, from about amino acid 1 to about amino acid 108, from about amino acid 1 to about amino acid 109, from about amino acid 1 to about amino acid 110, from about amino acid 1 to about amino acid 111, from about amino acid 1 to about amino acid 112, from about amino acid 1 to about amino acid 113, or from about amino acid 1 to about more than amino acid 114 (e.g., 115, 120, 125, 130, 135, 140, 145, 150, 200, 250, 300, etc.) of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134, or an arenavirus glycoprotein precursor sequence. In some embodiments, the portion of the amino acid sequence includes or is from about amino acid 1 to about amino acid 66 of SEQ ID NO:3. In some embodiments, the portion of the amino acid sequence includes or is from about amino acid 1 to about amino acid 81 of SEQ ID NO:3. In some embodiments, the portion of the amino acid sequence includes or is from about amino acid 1 to about amino acid 113 of SEQ ID NO:3.

In certain embodiments, the functional fragment may possess one or more functions that is known to a person of ordinary skills in the art. In certain embodiments, the functional fragment may possess the biological functions of the polypeptide from which the functional fragment is derived, for example the biological functions required for growth of the arenavirus particle. In certain embodiments, the functional fragment can support the biological functions of the polypeptide from which it is derived as a separated fragment independent of the context of the full-length polypeptide. In an exemplary embodiment, the functional fragment may be a signal peptide that possesses a function of mediating the insertion of glycoprotein precursor into the membrane of the endoplasmic reticulum (ER). In further embodiments, besides mediating the insertion of glycoprotein precursor into the membrane of the ER, the signal peptide may possess other functions, for example mediating cleavage of the polypeptide and/or acting as a trans-acting maturation factor. In other embodiments, the functional fragment may not possess any function that is known to a person of ordinary skills in the art.

In certain embodiments, the functional fragment may be 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, or 300 amino acids in length. In certain embodiments, the functional fragment is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In certain embodiments, the functional fragment does not exceed 2500 amino acids in length.

In certain embodiments, the functional fragment may consist of 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, or 300 amino-acid residues. In certain embodiments, the functional fragment may consist of 58 amino-acid residues. In certain embodiments, the functional fragment may consist of 193 amino-acid residues. In certain embodiments, the functional fragment may consist of 200 amino-acid residues. In certain embodiments, the functional fragment may consist of 201 amino-acid residues. In certain embodiments, the functional fragment may consist of 207 amino-acid residues. In certain embodiments, the functional fragment may consist of 215 amino-acid residues. In certain embodiments, the functional fragment may consist of 225 amino-acid residues. In certain embodiments, the functional fragment may consist of 232 amino-acid residues. In certain embodiments, the functional fragment may consist of 233 amino-acid residues. In certain embodiments, the functional fragment may consist of 234 amino-acid residues. In certain embodiments, the functional fragment may consist of 235 amino-acid residues. In certain embodiments, the functional fragment may consist of 15 amino-acid residues. In certain embodiments, the functional fragment may consist of 20 amino-acid residues. In certain embodiments, the functional fragment may consist of 1 to 15 amino-acid residues. In certain embodiments, the functional fragment may consist of 2 to 20 amino-acid residues.

In certain embodiments, the functional fragment is a fragment of arenavirus GP, NP, Z, or L, namely a fragment of arenavirus glycoprotein, nucleoprotein, Z protein, L protein, glycoprotein precursor, nucleoprotein precursor, Z protein precursor, or L protein precursor. In certain embodiments, the functional fragment of arenavirus GP, NP, Z, or L is wild-type. In other embodiments, the functional fragment of arenavirus GP, NP, Z, and L is recombinant. In certain embodiments, the functional fragment of arenavirus GP, NP, Z, or L is mutated. In certain embodiments, the functional fragment of arenavirus GP, NP, Z, or L is derived from an attenuated virus.

In certain embodiments, the functional fragment is a fragment of wild-type arenavirus glycoprotein, nucleoprotein, Z protein, or L protein. In certain embodiments, the functional fragment is a fragment of wild-type arenavirus glycoprotein. In certain embodiments, the functional fragment is a fragment of wild-type arenavirus nucleoprotein. In certain embodiments, the functional fragment is a fragment of wild-type arenavirus Z protein. In certain embodiments, the functional fragment is a fragment of wild-type arenavirus L protein.

In certain embodiments, the functional fragment is a fragment of wild-type arenavirus glycoprotein precursor, nucleoprotein precursor, Z protein precursor, or L protein precursor. In certain embodiments, the functional fragment is a fragment of wild-type arenavirus glycoprotein precursor. In certain embodiments, the wild-type arenavirus glycoprotein precursor can be processed into a GP signal peptide, a GP1 and a GP2. In certain embodiments, the functional fragment is a fragment of wild-type arenavirus nucleoprotein precursor. In certain embodiments, the functional fragment is a fragment of wild-type arenavirus Z protein precursor. In certain embodiments, the functional fragment is a fragment of wild-type arenavirus L protein precursor.

In certain embodiments, the functional fragment is a fragment of recombinant arenavirus glycoprotein, nucleoprotein, Z protein or L protein. In certain embodiments, the functional fragment is a fragment of recombinant arenavirus glycoprotein. In certain embodiments, the functional fragment is a fragment of recombinant arenavirus nucleoprotein. In certain embodiments, the functional fragment is a fragment of recombinant arenavirus Z protein. In certain embodiments, the functional fragment is a fragment of recombinant arenavirus L protein.

In certain embodiments, the functional fragment is recombinant arenavirus glycoprotein precursor, nucleoprotein precursor, Z protein precursor or L protein precursor. In certain embodiments, the functional fragment is a fragment of recombinant arenavirus glycoprotein precursor. In certain embodiments, the arenavirus glycoprotein precursor can be processed into a GP signal peptide, a GP1 and a GP2, one or more of which may be recombinant. In certain embodiments, the functional fragment is a fragment of recombinant arenavirus nucleoprotein precursor. In certain embodiments, the functional fragment is a fragment of recombinant arenavirus Z protein precursor. In certain embodiments, the functional fragment is a fragment of recombinant arenavirus L protein precursor.

In certain embodiments, the functional fragment is a fragment of mutated arenavirus glycoprotein, nucleoprotein, Z protein or L protein. In certain embodiments, the functional fragment is a fragment of mutated arenavirus glycoprotein. In certain embodiments, the functional fragment is a fragment of mutated arenavirus nucleoprotein. In certain embodiments, the functional fragment is a fragment of mutated arenavirus Z protein. In certain embodiments, the functional fragment is a fragment of mutated arenavirus L protein.

In certain embodiments, the functional fragment is mutated arenavirus glycoprotein precursor, nucleoprotein precursor, Z protein precursor or L protein precursor. In certain embodiments, the functional fragment is a fragment of mutated arenavirus glycoprotein precursor. In certain embodiments, the arenavirus glycoprotein precursor can be processed into a GP signal peptide, a GP1 and a GP2, one or more of which may be mutated. In certain embodiments, the functional fragment is a fragment of mutated arenavirus nucleoprotein precursor. In certain embodiments, the functional fragment is a fragment of mutated arenavirus Z protein precursor. In certain embodiments, the functional fragment is a fragment of mutated arenavirus L protein precursor.

In certain embodiments, the functional fragment is a fragment of arenavirus glycoprotein, nucleoprotein, Z protein or L protein that is derived from an attenuated virus. In certain embodiments, the functional fragment is a fragment of arenavirus glycoprotein derived from an attenuated virus. In certain embodiments, the functional fragment is a fragment of arenavirus nucleoprotein derived from an attenuated virus. In certain embodiments, the functional fragment is a fragment of arenavirus Z protein derived from an attenuated virus. In certain embodiments, the functional fragment is a fragment of arenavirus L protein derived from an attenuated virus.

In certain embodiments, the functional fragment is arenavirus glycoprotein precursor, nucleoprotein precursor, Z protein precursor or L protein precursor that is derived from an attenuated virus. In certain embodiments, the functional fragment is a fragment of arenavirus glycoprotein precursor derived from an attenuated virus. In certain embodiments, the arenavirus glycoprotein precursor can be processed into a GP signal peptide, a GP1 and a GP2, one or more of which may be derived from an attenuated virus. In certain embodiments, the functional fragment is a fragment of arenavirus nucleoprotein precursor derived from an attenuated virus. In certain embodiments, the functional fragment is a fragment of arenavirus Z protein precursor derived from an attenuated virus. In certain embodiments, the functional fragment is a fragment of arenavirus L protein precursor derived from an attenuated virus.

In certain embodiments, the functional fragment is a fragment of arenavirus glycoprotein precursor. In certain embodiments, the functional fragment is arenavirus GP signal peptide. In certain embodiments, the arenavirus GP signal peptide as described herein comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 80% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 85% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 90% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 95% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 96% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 97% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 98% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is at least 99% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO:163. In certain embodiments, the arenavirus GP signal peptide as described herein comprises an amino acid sequence that is 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 80% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 85% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 90% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 95% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 96% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 97% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 98% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163. In certain embodiments, the arenavirus GP signal peptide as described herein comprises an amino acid sequence that is 99% identical to the amino acid sequence of SEQ ID NO:25, SEQ ID NO:26, SEQ ID NO:27, SEQ ID NO:28, SEQ ID NO:29, SEQ ID NO:30, SEQ ID NO:53, SEQ ID NO:60, SEQ ID NO:67, SEQ ID NO:74, SEQ ID NO:85, SEQ ID NO:92, SEQ ID NO:99, SEQ ID NO:114, SEQ ID NO:121, SEQ ID NO:128, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163.

In certain embodiments, the functional fragment is GP1. In certain embodiments, the GP1 as described herein comprises an amino acid sequence that is at least 80%, 85%, 90%, 95%, 96%, 97%, 98%, 99% or 100% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 80% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 85% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 90% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 95% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 96% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 97% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 98% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is at least 99% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 100% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 80% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO: 100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 85% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 90% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 95% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 96% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO: 165. In certain embodiments, the arenavirus GP1 as described herein comprises an amino acid sequence that is 97% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO: 100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 98% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In certain embodiments, the arenavirus GP1 as described herein comprises an amino acid sequence that is 99% identical to the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165.

In certain embodiments, the functional fragment is GP2. In certain embodiments, the GP2 as described herein comprises an amino acid sequence that is at least 80%, 85%, 90%, 95%, 96%, 97%, 98%, 99% or 100% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 80% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 85% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 90% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO: 166. In certain embodiments, the arenavirus GP2 as described herein comprises an amino acid sequence that is at least 95% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 96% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 97% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 98% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is at least 99% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 100% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 80% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 85% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 90% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 95% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 96% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 97% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 98% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166. In certain embodiments, the arenavirus GP2 as described herein comprises an amino acid sequence that is 99% identical to the amino acid sequence of SEQ ID NO:37, SEQ ID NO:38, SEQ ID NO:39, SEQ ID NO:40, SEQ ID NO:41, SEQ ID NO:42, SEQ ID NO:55, SEQ ID NO:62, SEQ ID NO:69, SEQ ID NO:76, SEQ ID NO:87, SEQ ID NO:94, SEQ ID NO:101, SEQ ID NO:116, SEQ ID NO:123, SEQ ID NO:130, SEQ ID NO:137, SEQ ID NO:149, SEQ ID NO:154, SEQ ID NO:161, or SEQ ID NO:166.

In certain embodiments, the functional fragment may consist of more than one of a GP signal peptide and a buffer peptide, GP1 and GP2. In some embodiments, an arenavirus GP1 and/or arenavirus GP2 comprises an amino acid sequence starting at about amino acid 59 to about 508 of SEQ ID NO:3, or a biologically active fragment thereof. In some embodiments, an arenavirus GP1 and/or arenavirus GP2 comprises an amino acid sequence starting at about amino acid 60 (or after 60) to about 508 of SEQ ID NO:3, or a biologically active fragment thereof. In some embodiments, an arenavirus GP1 and/or arenavirus GP2 comprises an amino acid sequence starting at about amino acid 58 to about 508 of SEQ ID NO:3, or a biologically active fragment thereof. In some embodiments, an arenavirus GP1 and/or arenavirus GP2 comprises an amino acid sequence starting at about amino acid 59 to about the end of any one of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134, or a biologically active fragment thereof. In some embodiments, an arenavirus GP1 and/or arenavirus GP2 comprises an amino acid sequence starting at a position after amino acid 59 of any one of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In some embodiments, an arenavirus GP1 and/or arenavirus GP2 comprises an amino acid sequence starting at a position before amino acid 59 of any one of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the functional fragment may consist of GP signal peptide, a buffer peptide, and GP1. In certain embodiments, the functional fragment may consist of GP signal peptide, a buffer peptide, and GP2. In certain embodiments, the functional fragment may consist of GP1 and GP2. In certain embodiments, the functional fragment is not the full-length glycoprotein precursor.

In certain embodiments, the functional fragment is a fragment of an arenavirus GP signal peptide and a buffer peptide. In certain embodiments, the arenavirus GP signal peptide is wild-type. In other embodiments, the arenavirus GP signal peptide is recombinant. In certain embodiments, the arenavirus GP signal peptide is selected from the group consisting of GP signal peptide of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the functional fragment is selected from the group consisting of the n region, hydrophobic region 1 (h-1 region) and hydrophobic region 2 (h-2 region) of the arenavirus GP signal peptide (fused to a buffer peptide as described herein). In certain embodiments, the functional fragment is the n region of the arenavirus GP signal peptide and a buffer peptide. In certain embodiments, the functional fragment is the h-1 region of the arenavirus GP signal peptide and a buffer peptide. In certain embodiments, the functional fragment is the h-2 region of the arenavirus GP signal peptide and a buffer peptide.

In certain embodiments, the functional fragment as described herein is a fragment of a polypeptide that comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO: 107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO: 126, SEQ ID NO: 133, or SEQ ID NO:140. In certain embodiments, the functional fragment as described herein is a fragment of a polypeptide that comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO: 107, SEQ ID NO:111, SEQ ID NO: 118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO: 140. In certain embodiments, the functional fragment as described herein is a fragment of a polypeptide that comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the functional fragment as described herein is a fragment of a polypeptide that comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein is a fragment of a polypeptide that comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO:112, SEQ ID NO:119, SEQ ID NO:126, SEQ ID NO:133, or SEQ ID NO: 140.

In certain embodiments, the functional fragment as described herein comprises an amino acid that is at least 80% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 85% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 90% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 95% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 96% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 97% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 98% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is at least 99% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 100% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 80% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 85% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 90% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 95% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 96% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 97% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 98% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the functional fragment as described herein comprises an amino acid that is 99% identical to the amino acid sequence of a fragment of a polypeptide comprising the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

### 5.3 Heterologous Non-Arenaviral Polypeptide

As described herein, in certain embodiments, the heterologous non-arenaviral polypeptide may be not of arenavirus origin. In some embodiments, any native signal peptide of a heterologous non-arenaviral polypeptide (e.g., a first heterologous non-arenaviral polypeptide) is truncated or deleted. In some embodiments, any native signal peptide of a heterologous non-arenaviral polypeptide (e.g., a second heterologous non-arenaviral polypeptide) is retained. In some embodiments, a heterologous non-arenaviral polypeptide (e.g., a first heterologous non-arenaviral polypeptide) is fused to a buffer peptide, which in turn is fused to the GP signal peptide (or a functional fragment thereof or a polypeptide comprising such a functional fragment or the GP signal peptide). In some embodiments, a heterologous non-arenaviral polypeptide (e.g., a first heterologous non-arenaviral polypeptide) is fused to the C-terminus of a buffer peptide.

In certain embodiments, the nucleotide sequence provided herein (see Section 5.5) comprises a first open reading frame (ORF) that comprises a nucleotide sequence encoding a functional fragment of a first polypeptide selected from the group consisting of arenavirus GP, NP, Z and L, and a second ORF that comprises a nucleotide sequence encoding the heterologous non-arenaviral polypeptide provided herein. In certain embodiments, the nucleotide sequence provided herein comprises an open reading frame (ORF), and the ORF comprises a nucleotide sequence encoding a functional fragment of a first polypeptide selected from the group consisting of arenavirus GP, NP, Z and L that are not from Lassa virus, and a heterologous non-arenaviral polypeptide. In certain embodiments, the arenavirus particle described herein comprises a genome encoding a heterologous polypeptide of arenavirus origin, but derived from a type of arenavirus that is different from the type of arenavirus from which the arenavirus particle comprises GP, NP, Z as well as L. In an exemplary embodiment, the heterologous polypeptide is from Lassa virus, whereas the arenavirus particle comprises the GP, NP, Z as well as L of LCMV or Pichinde virus.

In certain embodiments, the arenavirus particle provided herein is engineered such that an arenaviral ORF is separated over two or more mRNA transcripts (see Section 5.6). In certain embodiments, one of the mRNA transcripts further comprises a nucleotide sequence encoding a heterologous non-arenaviral polypeptide. In certain embodiments, one of the mRNA transcripts further comprises a nucleotide sequence encoding heterologous polypeptide of arenavirus origin, but derived from a type of arenavirus that is different from the type of arenavirus from which the arenavirus particle comprises GP, NP, Z as well as L. In an exemplary embodiment, the heterologous polypeptide is from Lassa virus, whereas the arenavirus particle comprises the GP, NP, Z as well as L of LCMV or Pichinde virus.

In certain embodiments, the arenavirus genomic or antigenomic segment provided herein (see Section 5.7) is engineered such that the viral transcription thereof results in a first mRNA transcript comprising a nucleotide sequence encoding a functional fragment of a first polypeptide selected from the group consisting of arenavirus GP, NP, Z and L and a second mRNA transcript comprising a nucleotide sequence encoding a heterologous non-arenaviral polypeptide. In certain embodiments, the arenavirus genomic or antigenomic segment provided herein is engineered such that the viral transcription thereof results in an mRNA transcript encoding a functional fragment of a first polypeptide selected from the group consisting of arenavirus GP, NP, Z and L, and a heterologous non-arenaviral polypeptide or a second polypeptide. In certain embodiments, the arenavirus genomic or antigenomic segment provided herein is engineered such that the viral transcription thereof results in an mRNA transcript encoding a functional fragment of a first polypeptide selected from the group consisting of arenavirus GP, NP, Z and L, and a heterologous polypeptide of arenavirus origin, but derived from a type of arenavirus that is different from the type of arenavirus from which the arenavirus genome comprises GP, NP, Z as well as L. In an exemplary embodiment, the heterologous polypeptide is from Lassa virus, whereas the arenavirus genome comprises the GP, NP, Z as well as L of LCMV or Pichinde virus.

In certain embodiments, the heterologous non-arenaviral polypeptide is about, at most about, or at least about 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, 300, 350, 400, 450, 500, 550, 600, 650, 700, 750, 800, 850, 900, 950, 1000, or more than 1000 amino acids in length. In certain embodiments, the heterologous non-arenaviral polypeptide is about, at least about, or at most about 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 300 to 500 amino acids in length, 350 to 600 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In certain embodiments, the heterologous non-arenaviral polypeptide does not exceed 500 amino acids in length. In certain embodiments, the heterologous non-arenaviral polypeptide does not exceed 750 amino acids in length. In certain embodiments, the heterologous non-arenaviral polypeptide does not exceed 1000 amino acids in length. In certain embodiments, the heterologous non-arenaviral polypeptide does not exceed 2500 amino acids in length.

In certain embodiments, the heterologous non-arenaviral polypeptide consists of about, at least about, or at most about 5, 8, 10, 12, 15, 18, 20, 22, 25, 28, 30, 32, 35, 38, 40, 42, 45, 48, 50, 52, 55, 58, 60, 65, 70, 75, 80, 85, 90, 95, 100, 150, 200, 250, 300, 350, 400, 450, 500, 550, 600, 650, 700, 750, 800, 850, 900, 950, or 1000 amino-acid residues. In certain embodiments, the heterologous non-arenaviral polypeptide may consist of about, at least about, or at most about 58 amino-acid residues. In certain embodiments, the heterologous non-arenaviral polypeptide may consist of about, at least about, or at most about 193 amino-acid residues. In certain embodiments, the heterologous non-arenaviral polypeptide may consist of about, at least about, or at most about 200 amino-acid residues. In certain embodiments, the heterologous non-arenaviral polypeptide may consist of about, at least about, or at most about 480 amino-acid residues. In certain embodiments, the heterologous non-arenaviral polypeptide may consist of about, at least about, or at most about 450 amino-acid residues. In certain embodiments, the heterologous non-arenaviral polypeptide may consist of about, at least about, or at most about 500 amino-acid residues. In certain embodiments, the heterologous non-arenaviral polypeptide may consist of about, at least about, or at most about 350 amino-acid residues. In certain embodiments, the heterologous non-arenaviral polypeptide may consist of about, at least about, or at most about 400 amino-acid residues. In certain embodiments, the heterologous non-arenaviral polypeptide may consist of about, at least about, or at most about 550 amino-acid residues. In certain embodiments, the heterologous non-arenaviral polypeptide may consist of about, at least about, or at most about 600 amino-acid residues. In certain embodiments, the heterologous non-arenaviral polypeptide may consist of about, at least about, or at most about 15 amino-acid residues. In certain embodiments, the heterologous non-arenaviral polypeptide may consist of about, at least about, or at most about 20 amino-acid residues. In certain embodiments, the heterologous non-arenaviral polypeptide may consist of about, at least about, or at most about 1 to 20 amino-acid residues. In certain embodiments, the heterologous non-arenaviral polypeptide may consist of about, at least about, or at most about 200 to 600 amino-acid residues. In certain embodiments, the coding sequence of the heterologous non-arenaviral polypeptide (e.g., the first heterologous non-arenaviral polypeptide) starts at about amino acid 60 or more than about 60 amino acids downstream of the methionine start codon of the GP signal peptide.

In certain embodiments, the heterologous non-arenaviral polypeptide is a reporter protein (see Section 5.3.1). In certain embodiments, the heterologous non-arenaviral polypeptide is a heterologous non-arenaviral signal peptide (see Section 5.3.2). In certain embodiments, the heterologous non-arenaviral polypeptide is an antigen (see Section 5.3.3).

### 5.3.1 Reporter Protein

In certain embodiments, the heterologous non-arenaviral polypeptide described herein is a reporter protein or a fluorescent protein. In certain embodiments, the reporter protein is a fluorescent protein. In certain embodiments, the reporter protein is green fluorescent protein (GFP). GFP emits bright green light when exposed to UV or blue like. In other embodiments, the reporter protein is a red fluorescent protein (RFP). In an exemplary embodiments, the report protein is TOM.

Reporter protein and genes would be readily recognized by one of skill in the art. Non-limiting examples of reporter proteins include various enzymes, such as, but not to 0-galactosidase, chloramphenicol acetyltransferase, neomycin phosphotransferase, luciferase or RFP.

In certain embodiments, the reporter protein is capable of expression at the same time as the antigen described herein. Ideally, expression is visible in normal light or other wavelengths of light. In certain embodiments, the intensity of the effect created by the reporter protein can be used to directly measure and monitor the arenavirus particle or tri-segmented arenavirus particle.

### 5.3.2 Heterologous Non-Arenaviral Signal Peptide

In certain embodiments, the heterologous non-arenaviral polypeptide described herein is a heterologous non-arenaviral signal peptide. In certain embodiments, the heterologous non-arenaviral signal peptide is a signal peptide of a glycoprotein. In certain embodiments, the heterologous non-arenaviral signal peptide is from a virus family selected from the group consisting of adenoviridae (e.g., mastadenovirus and aviadenovirus), herpesviridae (e.g., herpes simplex virus 1, herpes simplex virus 2, herpes simplex virus 5, herpes simplex virus 6, Epstein-Barr virus, HHV6-HHV8, cytomegalovirus, and varicella zoster virus), leviviridae (e.g., levivirus, enterobacteria phase MS2, allolevirus), orthomyxoviridae (e.g., influenzavirus A, influenzavirus B, influenzavirus C), parvoviridae (e.g., Parvovirus B19), filoviridae (e.g., Ebola virus, Marburg virus), hantaviridae, poxviridae (e.g., chordopoxviridae, parapoxvirus, avipoxvirus, capripoxvirus, leporiipoxvirus, suipoxvirus, molluscipoxvirus, and entomopoxyirinae), polyomaviridae, papillomaviridae (e.g., human papillomavirus), paramyxoviridae (e.g., paramyxovirus, parainfluenza virus 1, mobillivirus (e.g., measles virus), rubulavirus (e.g., mumps virus)), pneumoviridae (e.g., pneumovirus, human respiratory syncytial virus and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus (HIV) 1 and HIV-2 (e.g., HIV gp160)), spumavirus), flaviviridae (e.g., hepatitis C virus, dengue virus, West Nile virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, mobala virus, mopeia virus, amapari virus, flexal virus, guanarito virus, junin virus, latino virus, machupo virus, oliveros virus, parana virus, pichinde virus, pirital virus, sabia virus, tacaribe virus, tamiami virus, bear canyon virus, whitewater arroyo virus, allpahuayo virus (ALLV), and lassa virus), and coronaviridae (e.g., coronavirus and torovirus). In certain embodiments, the heterologous non-arenaviral signal peptide is the signal peptide of the vesicular stomatitis virus serotype Indiana glycoprotein.

### 5.3.3 Antigen

In certain embodiments, the heterologous non-arenaviral polypeptide is an antigen. In certain embodiments, the antigen is derived from an infectious organism, tumor, or allergen. In one embodiment, the antigen is of an infectious pathogen or associated with any disease that is capable of eliciting an immune response. In certain embodiments, the heterologous non-arenaviral polypeptide is an antigen derived from a virus, a bacterium, a fungus, a parasite, or can be expressed in a tumor or tumor associated disease (i.e., cancer), an autoimmune disease, a degenerative disease, an inherited disease, substance dependency, obesity, or an allergic disease.

In certain embodiments, the heterologous non-arenaviral polypeptide is a viral antigen. Non-limiting examples of viral antigens include antigens from adenoviridae (e.g., mastadenovirus and aviadenovirus), herpesviridae (e.g., herpes simplex virus 1, herpes simplex virus 2, herpes simplex virus 5, herpes simplex virus 6, Epstein-Barr virus, HHV6-HHV8, cytomegalovirus, and varicella zoster virus), leviviridae (e.g., levivirus, enterobacteria phase MS2, allolevirus), orthomyxoviridae (e.g., influenzavirus A, influenzavirus B, influenzavirus C), parvoviridae (e.g., Parvovirus B19), filoviridae (e.g., Ebola virus, Marburg virus), hantaviridae, poxviridae (e.g., chordopoxviridae, parapoxvirus, avipoxvirus, capripoxvirus, leporiipoxvirus, suipoxvirus, molluscipoxvirus, and entomopoxyirinae), polyomaviridae, papillomaviridae (e.g., human papillomavirus), paramyxoviridae (e.g., paramyxovirus, parainfluenza virus 1, mobillivirus (e.g., measles virus), rubulavirus (e.g., mumps virus)), pneumoviridae (e.g., pneumovirus, human respiratory syncytial virus and metapneumovirus (e.g., avian pneumovirus and human metapneumovirus)), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus (HIV) 1 and HIV-2 (e.g., HIV gp160)), spumavirus), flaviviridae (e.g., hepatitis C virus, dengue virus, West Nile virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, mobala virus, mopeia virus, amapari virus, flexal virus, guanarito virus, junin virus, latino virus, machupo virus, oliveros virus, parana virus, pichinde virus, pirital virus, sabia virus, tacaribe virus, tamiami virus, bear canyon virus, whitewater arroyo virus, allpahuayo virus (ALLV), and lassa virus), and coronaviridae (e.g., coronavirus and torovirus). In certain embodiments, the arenavirus particle described herein comprises a genome encoding an antigen from a mammarenavirus (e.g., Allpahuayo virus, Alxa virus, Junin virus, Bear Canyon virus, Sabia virus, Pichinde virus, Chapare virus, Lijiang virus, Cupixi virus, Flexal virus, Gairo virus, Guanarito virus, Ippy virus, Lassa virus, Latino virus, Loei River virus, Lujo virus, Luna virus, Luli virus, Lunk virus, lymphocytic choriomeningitis virus, Machupo virus, Mariental virus, Merino Walk virus, Mobala virus, Mopeia virus, Morogoro virus, Okahandja virus, Oliveros virus, Parana virus, Pirital virus, Apore virus, Ryukyu virus, Amapari virus, Solwezi virus, souris virus, Tacaribe virus, Tamiami virus, Wenzhou virus, Whitewater Arroyo virus, Big Brushy Tank virus, Catarina virus, Skinner Tank virus, Tonto Creek virus, or Xapuri virus), wherein the mammarenavirus is different from the species of arenavirus from which the arenavirus particle comprises GP, NP, Z as well as L. In a specific embodiment the viral antigen, is HIV gp120, gp41, HIV Nef, RSV F glycoprotein, RSV G glycoprotein, HTLV tax, herpes simplex virus glycoprotein (e.g., gB, gC, gD, and gE) or hepatitis B surface antigen, hepatitis C virus E protein or coronavirus spike protein. In one embodiment, the viral antigen is not an HIV antigen.

In other embodiments, the heterologous non-arenaviral polypeptide is a bacterial antigen (e.g., bacterial coat protein). Non-limiting examples of bacterial antigens include antigens from a bacteria family selected from the group consisting of the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Envinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcus (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, Yersinia family, Bacillus antracis and Vampirovibrio family.

In other embodiments, the heterologous non-arenaviral polypeptide is a parasitic antigen (e.g., a protozoan antigen). Non-limiting examples of parasite antigens include antigens from a parasite such as an amoeba, a malarial parasite, Plasmodium, Trypanosoma cruzi.

In yet other embodiments, the heterologous non-arenaviral polypeptide is a fungal antigen. Non-limiting examples of fungal antigens include antigens from fungus of Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kern, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neoforms, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

In some embodiments, the heterologous non-arenaviral polypeptide is a tumor neoantigen. A “neoantigen,” used herein, means an antigen that arises by mutation in a tumor cell and such an antigen is not generally expressed in normal cells or tissue. Without being bound by theory, because healthy tissues generally do not possess these antigens, neoantigens represent a preferred target. Additionally, without being bound by theory, in the context of the present invention, since the T cells that recognize the neoantigen may not have undergone negative thymic selection or functionally impacted by peripheral tolerance mechanisms, such cells can have high functional avidity to the antigen and mount a strong immune response against tumors, while lacking the risk to induce destruction of normal tissue and autoimmune damage. In certain embodiments, the neoantigen is an MHC class I-restricted neoantigen. In certain embodiments, the neoantigen is an MHC class II-restricted neoantigen. In certain embodiments, a mutation in a tumor cell of the patient results in a novel protein that produces the neoantigen. In certain embodiments, the heterologous non-arenaviral polypeptide is a tumor neo-epitope. In certain embodiments, the neo-epitope is an MHC class I-restricted neo-epitope. In certain embodiments, the neo-epitope is an MHC class II-restricted neo-epitope.

In some embodiments, the heterologous non-arenaviral polypeptide is a tumor antigen or tumor associated antigen. In some embodiments, the tumor antigen or tumor associated antigen includes antigens from tumor associated diseases including acute lymphoblastic leukemia, acute myeloid leukemia, adrenocortical carcinoma, childhood adrenocortical carcinoma, AIDS-Related Cancers, Kaposi Sarcoma, anal cancer, appendix cancer, astrocytomas, atypical teratoid/rhabdoid tumor, basal-cell carcinoma, bile duct cancer, extrahepatic (see cholangiocarcinoma), bladder cancer, bone osteosarcoma/malignant fibrous histiocytoma, brainstem glioma, brain cancer, brain tumor, cerebellar astrocytoma, cerebral astrocytoma/malignant glioma brain tumor, ependymoma, medulloblastoma, supratentorial primitive neuroectodermal tumors, visual pathway and hypothalamic glioma, breast cancer, bronchial adenomas/carcinoids, burkitt's lymphoma, carcinoid tumor, carcinoid gastrointestinal tumor, carcinoma of unknown primary, central nervous system lymphoma, primary, cerebellar astrocytoma, cerebral astrocytoma/malignant glioma, cervical cancer, childhood cancers, chronic bronchitis, chronic lymphocytic leukemia, chronic myelogenous leukemia, chronic myeloproliferative disorders, colon cancer, cutaneous T-cell lymphoma, desmoplastic small round cell tumor, emphysema, endometrial cancer, ependymoma, esophageal cancer, ewing's sarcoma in the Ewing family of tumors, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor, germ cell tumor: extracranial, extragonadal, or ovarian gestational trophoblastic tumor, glioma of the brain stem, glioma, childhood cerebral astrocytoma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma, intraocular melanoma, islet cell carcinoma (endocrine pancreas), kaposi sarcoma, kidney cancer (renal cell cancer), laryngeal cancer, acute lymphoblastic lymphoma, acute lymphocytic leukemia, acute myelogenous leukemia, chronic lymphocytic leukemia, chronic myeloid leukemia, lip and oral cavity cancer, liposarcoma, liver cancer (primary), lung cancer, non-small cell, small cell, AIDS-related lymphoma, Burkitt lymphoma, cutaneous T-cell lymphoma, hodgkin lymphoma, non-hodgkin lymphoma, lymphoma, primary central nervous system, macroglobulinemia, Waldenstrom, male breast cancer, malignant fibrous histiocytoma of bone/osteosarcoma, medulloblastoma, melanoma, intraocular (eye), merkel cell cancer, mesothelioma, adult malignant, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia, chronic, myeloid leukemia, adult acute, myeloid leukemia, childhood acute, myeloma, multiple (cancer of the bone-marrow), myeloproliferative disorders, chronic, nasal cavity and paranasal sinus cancer, nasopharyngeal carcinoma, neuroblastoma, non-small cell lung cancer, oligodendroglioma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, islet cell, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma, childhood, salivary gland cancer, sarcoma, Ewing family of tumors, Kaposi sarcoma, soft tissue sarcoma, uterine sarcoma, sezary syndrome, skin cancer (non-melanoma), skin cancer (melanoma), merkel cell skin carcinoma, small cell lung cancer, small intestine cancer, soft tissue sarcoma, squamous cell carcinoma—see skin cancer (non-melanoma), squamous neck cancer with occult primary, metastatic, stomach cancer, supratentorial primitive neuroectodermal tumor, T-Cell lymphoma, cutaneous—see Mycosis Fungoides and Sezary syndrome, testicular cancer, throat cancer, thymoma and thymic carcinoma, thyroid cancer, childhood transitional cell cancer of the renal pelvis and ureter, gestational trophoblastic tumor, unknown primary site, carcinoma of, adult unknown primary site, cancer of childhood, ureter and renal pelvis, transitional cell cancer, rethral cancer, uterine cancer, endometrial uterine sarcoma, bronchial tumor, central nervous system embryonal tumor; childhood chordoma, colorectal cancer, craniopharyngioma, ependymoblastoma, langerhans cell histiocytosis, acute lymphoblastic leukemia, acute myeloid leukemia (adult/childhood), small cell lung cancer, medulloepithelioma, oral cavity cancer, papillomatosis, pineal parenchymal tumors of intermediate differentiation, pituary tumor, respiratory tract carcinoma involving the NUT gene on chromosome 15, spinal cord tumor, thymoma, thyroid cancer, vaginal cancer; vulvar cancer, and Wilms tumor.

Non-limiting examples of tumor or tumor associated antigens include Adipophilin, AIM-2, ALDH1A1, BCLX (L), BING-4, CALCA, CD45, CPSF, cyclin D1, DKK1, ENAH (hMena), EpCAM, EphA3, EZH2, FGF5, glypican-3, G250/MN/CAIX, HER-2/neu, IDO1, IGF2B3, IL13Ralpha2, Intestinal carboxyl esterase, alpha-fetoprotein, Kallikrein 4, KIF20A, Lengsin, M-CSF, MCSP, mdm-2, Meloe, MMP-2, MMP-7, MUC1, MUC5AC, p53, PAX5, PBF, PRAME, PSMA, RAGE-1, RGS5, RhoC, RNF43, RU2AS, secernin 1, SOX10, STEAPI, survivinn, Telomerase, VEGF, or WT1, EGF-R, CEA, CD52, gp 100 protein, MELANA/MART1, NY-ESO-1, p53, MAGE1, MAGE3 and CDK4, alpha-actinin-4, ARTC1, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML1 fusion protein, FLT3-ITD, FN1, GPNMB, LDLR-fucosyltransferaseAS fusion protein, NFYC, OGT, OS-9, pml-RARalpha fusion protein, PRDX5, PTPRK, K-ras, N-ras, RBAF600, SIRT2, SNRPD1, SYT-SSX1 or -SSX2 fusion protein, TGF-betaRII, Triosephosphate isomerase, Lengsin, M-CSF, MCSP, or mdm-2.

In some embodiments, the heterologous non-arenaviral polypeptide is a respiratory pathogen antigen. In a specific embodiment, the respiratory pathogen is a virus such as RSV, coronavirus, human metapneumovirus, parainfluenza virus, hendra virus, nipah virus, adenovirus, rhinovirus, or PRRSV. Non-limiting examples of respiratory viral antigens include Respiratory Syncytial virus F, G and M2 proteins, Coronavirus (SARS, HuCoV) spike proteins (S), human metapneumovirus fusion proteins, Parainfluenza virus fusion and hemagglutinin proteins (F, HN), Hendra virus (HeV) and Nipah virus (NiV) attachment glycoproteins (G and F), Adenovirus capsid proteins, Rhinovirus proteins, and PRRSV wild type or modified GP5 and M proteins.

In a specific embodiment, the respiratory pathogen is a bacteria such as Bacillus anthracis, Mycobacterium tuberculosis, Bordetella pertussis, Streptococcus pneumoniae, Yersinia pestis, Staphylococcus aureus, Francisella tularensis, Legionella pneumophila, Chlamydia pneumoniae, Pseudomonas aeruginosa, Neisseria meningitides, and Haemophilus influenzae. Non-limiting examples of respiratory bacterial antigens include Bacillus anthracis Protective antigen PA, Mycobacterium tuberculosis mycobacterial antigen 85A and heat shock protein (Hsp65), Bordetella pertussis pertussis toxoid (PT) and filamentous hemagglutinin (FHA), Streptococcus pneumoniae sortase A and surface adhesin A (PsaA), Yersinia pestis F1 and V subunits, and proteins from Staphylococcus aureus, Francisella tularensis, Legionella pneumophila, Chlamydia pneumoniae, Pseudomonas aeruginosa, Neisseria meningitides, and Haemophilus influenzae.

In some embodiments, the heterologous non-arenaviral polypeptide is a T-cell epitope. In other embodiments, the heterologous ORF encodes a cytokine or growth factor.

In other embodiments, the heterologous non-arenaviral polypeptide is an antigen expressed in an autoimmune disease. In more specific embodiments, the autoimmune disease can be type I diabetes, multiple sclerosis, rheumatoid arthritis, lupus erythmatosus, and psoriasis. Non-limiting examples of autoimmune disease antigens include Ro60, dsDNA, or RNP.

In other embodiments, the heterologous non-arenaviral polypeptide is an antigen expressed in an allergic disease. In more specific embodiments, the allergic disease can include but is not limited to seasonal and perennial rhinoconjunctivitis, asthma, and eczema. Non-limiting examples of allergy antigens include Bet v 1 and Fel d 1.

### 5.4 Buffer Peptide

In certain embodiments, provided herein is a buffer peptide. In some embodiments, the buffer peptide is on the C-terminus or is fused to the C-terminus of an arenavirus GP signal peptide. In some embodiments, the buffer peptide is fused to the N-terminus of a heterologous non-arenaviral polypeptide. In some embodiments, the arenavirus does not comprise a buffer peptide. In some embodiments, the GP signal peptide (or a functional fragment thereof or a polypeptide comprising such a functional fragment or the GP signal peptide) is fused to a buffer peptide, which in turn is fused to a heterologous non-arenaviral polypeptide. In some embodiments, following translation of the GP signal peptide (or functional fragment thereof or polypeptide comprising the GP signal peptide or functional fragment thereof) and the buffer peptide (and, optionally, the heterologous non-arenaviral polypeptide), a peptidase can cleave the GP signal peptide (or functional fragment thereof or polypeptide comprising the GP signal peptide or functional fragment thereof)/buffer peptide fusion close to (eg, −5, −4, −3, −2, −1, +1, +2, +3, +4, or +5 amino acid position from the fusion site) or at the fusion site. In some embodiments, the sequence of an arenavirus GP signal peptide, a buffer peptide, and a heterologous non-arenaviral polypeptide is from N-terminus to C-terminus. In some embodiments, a GP signal peptide is fused to a buffer peptide. In some embodiments, a buffer peptide is fused to a heterologous non-arenaviral polypeptide. In some embodiments, a GP signal peptide, a buffer peptide, and a heterologous non-arenaviral polypeptide generate a fusion protein. In some embodiments, a GP signal peptide, a buffer peptide, and a heterologous non-arenaviral polypeptide are fused in this order from N-terminus to C-terminus. In some embodiments, a buffer peptide is about, at least about, or at most about: 1, 2, 3, 4, 5, 6,7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, or more than about 200 amino acids long.

Without being bound by theory, the presence of a buffer peptide following the GP signal peptide (or functional fragment thereof or polypeptide comprising such a functional fragment or the GP signal peptide) enhances the cleavage of the GP signal peptide from the remainder of the fusion peptide. In a specific embodiment, growth of a trisegmented virus as described herein with a buffer peptide as described herein (or test virus) is compared to the growth of a control virus, which has the same genomic organization as the test virus except that the buffer peptide is absent from the control virus. In a specific embodiment, growth of a trisegmented virus as described herein with a buffer peptide as described herein (or test virus) is compared to the growth of a control virus, which has the same genomic organization as the test virus except that the buffer peptide is a wild type peptide sequence. In certain embodiments, the test virus grows to a titre about or at least about 2-fold, 3-fold, 4-fold, 5-fold, 6-fold, 7-fold, 8-fold, 9-fold, 10-fold, 15-fold, 20-fold, or about or at least about 25-fold higher than the control virus using the same culture conditions for test and control virus. In certain embodiments, the test virus reaches peak titers 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, 60 hours, 72 hours, 84 hours, or 96 hours earlier than the control virus using the same culture conditions for test and control virus. In certain embodiments, the test virus exhibits about or at least about 2-fold, 3-fold, 4-fold, 5-fold, 6-fold, 7-fold, 8-fold, 9-fold, 10-fold, or higher than 10-fold rescue efficiency (i.e., higher percentage of RCV titer-positive rescues of total rescues) than the control virus using the same vector rescue conditions for test and control virus. In some embodiments, the control virus is an arenavirus that does not comprise a buffer peptide. In some embodiments, the control virus is identical to an arenavirus of the present disclosure except that the control virus does not include a buffer peptide. In some embodiments, the control virus is a wild-type arenavirus.

In some embodiments, a buffer peptide comprises or consists of a fragment of an arenavirus GP1. In some embodiments, the fragment is shorter than the sequence of an arenavirus GP1. In some embodiments, the fragment of an arenavirus GP1 comprises about, at most about, or at least about 1, 2, 3, 4, 5, 10, 15, 20, 25, 30, 35, 40, 45, 50, 55, 60, or more than 60 amino acids of an arenavirus GP1. In some embodiments, a fragment of an arenavirus GP1 is an N-terminal fragment of an arenavirus GP1. In some embodiments, a fragment of an arenavirus GP1 is a C-terminal fragment of an arenavirus GP1. In some embodiments, a buffer peptide comprises or consists of an amino acid sequence that is about or at least about 80%, 85%, 90%, 95%, 96%, 97%, 98%, 99% or 100% identical to a portion of the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO:115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. In some embodiments, a buffer peptide comprises a basic and/or neutral and/or nonpolar aliphatic and/or nonpolar aromatic and/or polar (e.g., neutral, positively charged or negatively charged) and/or acidic amino acid corresponding to one or more of positions 1, 2, 3, 4, 5, 6, and/or 7 of the amino acid sequence of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO: 100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or SEQ ID NO:165. For example, in some embodiments, the methionines at positions 2 and 3 of SEQ ID NO: 33 are mutated to a nonpolar aliphatic amino acid (e.g., glycine, alanine, proline, valine, leucine, methionine, or isoleucine), a basic amino acid (e.g., arginine, lysine, or histidine), an acidic amino acid (e.g., aspartic acid or glutamic acid), a nonpolar aromatic amino acid (e.g., phenylalanine, tyrosine, or tryptophan) and/or a polar amino acid (e.g., serine, threonine, cysteine, asparagine, glutamine, lysine, arginine, histidine, aspartic acid, or glutamic acid). In some embodiments, the methionines at positions 2 and 3 of SEQ ID NO: 33 are both mutated to leucine. In some embodiments, the methionines at positions 2 and 3 of SEQ ID NO: 33 are both mutated to aspartic acid. In some embodiments, the methionines at positions 2 and 3 of SEQ ID NO: 33 are both mutated to glycine. In some embodiments, the methionines at positions 2 and 3 of SEQ ID NO: 33 are both mutated to glutamic acid. In some embodiments, the methionines at positions 2 and 3 of SEQ ID NO: 33 are both mutated to histidine. In some embodiments, the methionines at positions 2 and 3 of SEQ ID NO: 33 are both mutated to lysine. In some embodiments, the portion of the amino acid sequence is any portion of a sequence and can range in the number of amino acids. In some embodiments, the portion of the amino acid sequence is from about amino acid 1 to about amino acid 5, from about amino acid 1 to about amino acid 10, from about amino acid 1 to about amino acid 15, from about amino acid 1 to about amino acid 20, from about amino acid 1 to about amino acid 25, from about amino acid 1 to about amino acid 30, from about amino acid 1 to about amino acid 35, from about amino acid 1 to about amino acid 40, from about amino acid 1 to about amino acid 45, from about amino acid 1 to about amino acid 50, from about amino acid 1 to about amino acid 51, from about amino acid 1 to about amino acid 52, from about amino acid 1 to about amino acid 53, from about amino acid 1 to about amino acid 54, from about amino acid 1 to about amino acid 55, from about amino acid 1 to about amino acid 56, from about amino acid 1 to about amino acid 57, from about amino acid 1 to about amino acid 58, from about amino acid 1 to about amino acid 59, from about amino acid 1 to about amino acid 60, from about amino acid 1 to about amino acid 61, from about amino acid 1 to about amino acid 62, from about amino acid 1 to about amino acid 63, from about amino acid 1 to about amino acid 64, from about amino acid 1 to about amino acid 65, from about amino acid 1 to about amino acid 66, from about amino acid 1 to about amino acid 67, from about amino acid 1 to about amino acid 68, from about amino acid 1 to about amino acid 69, from about amino acid 1 to about amino acid 70, from about amino acid 1 to about amino acid 71, from about amino acid 1 to about amino acid 72, from about amino acid 1 to about amino acid 73, from about amino acid 1 to about amino acid 74, from about amino acid 1 to about amino acid 75, from about amino acid 1 to about amino acid 76, from about amino acid 1 to about amino acid 77, from about amino acid 1 to about amino acid 78, from about amino acid 1 to about amino acid 79, from about amino acid 1 to about amino acid 80, from about amino acid 1 to about amino acid 81, from about amino acid 1 to about amino acid 82, from about amino acid 1 to about amino acid 83, from about amino acid 1 to about amino acid 84, from about amino acid 1 to about amino acid 85, from about amino acid 1 to about amino acid 86, from about amino acid 1 to about amino acid 87, from about amino acid 1 to about amino acid 88, from about amino acid 1 to about amino acid 89, from about amino acid 1 to about amino acid 90, from about amino acid 1 to about amino acid 91, from about amino acid 1 to about amino acid 92, from about amino acid 1 to about amino acid 93, from about amino acid 1 to about amino acid 94, from about amino acid 1 to about amino acid 95, from about amino acid 1 to about amino acid 96, from about amino acid 1 to about amino acid 97, from about amino acid 1 to about amino acid 98, from about amino acid 1 to about amino acid 99, from about amino acid 1 to about amino acid 100, from about amino acid 1 to about amino acid 101, from about amino acid 1 to about amino acid 102, from about amino acid 1 to about amino acid 103, from about amino acid 1 to about amino acid 104, from about amino acid 1 to about amino acid 105, from about amino acid 1 to about amino acid 106, from about amino acid 1 to about amino acid 107, from about amino acid 1 to about amino acid 108, from about amino acid 1 to about amino acid 109, from about amino acid 1 to about amino acid 110, from about amino acid 1 to about amino acid 111, from about amino acid 1 to about amino acid 112, from about amino acid 1 to about amino acid 113, or from about amino acid 1 to about or more than about amino acid 114 (e.g., 115, 120, 125, 130, 135, 140, 145, 150, 200, 250, 300, etc.) of SEQ ID NO:31, SEQ ID NO:32, SEQ ID NO:33, SEQ ID NO:142, SEQ ID NO:34, SEQ ID NO:35, SEQ ID NO:36, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, or of an arenavirus GP1 sequence.

In some embodiments, a buffer peptide comprises about 1%, 2%, 5%, 10%, 15%, 20%, 25%, 30% basic amino acids. In some embodiments, a buffer peptide comprises at most about 1%, 2%, 5%, 10%, 15%, 20%, 25%, 30% basic amino acids. In some embodiments, a buffer peptide comprises at least about 1%, 2%, 5%, 10%, 15%, 20%, 25%, 30% basic amino acids. In some embodiments, the first 3 N-terminal amino acids of the buffer peptide include about, or at most about 1, 2, or 3 basic amino acids. In some embodiments, the first 3 N-terminal amino acids of the buffer peptide include about, at least about, or at most about 1 or 2 basic amino acids. In some embodiments, the first 5 N-terminal amino acids of the buffer peptide include about, or at most about 1, 2, 3, 4, or 5 basic amino acids. In some embodiments, the first 5 N-terminal amino acids of the buffer peptide include about, at least about, or at most about 1, 2, or 3 basic amino acids. In some embodiments, the first 8 N-terminal amino acids of the buffer peptide include about, or at most about 1, 2, 3, 4, 5, 6, 7, or 8 basic amino acids. In some embodiments, the first 8 N-terminal amino acids of the buffer peptide include about, at least about, or at most about 1, 2, 3, 4, or 5 basic amino acids. In some embodiments, the first 10 N-terminal amino acids of the buffer peptide include about, or at most about 1, 2, 3, 4, 5, 6, 7, 8, 9, or 10 basic amino acids. In some embodiments, the first 10 N-terminal amino acids of the buffer peptide include about, at least about, or at most about 1, 2, 3, 4, 5, 6, 7, or 8 basic amino acids. In some embodiments, the first 20 N-terminal amino acids of the buffer peptide include about, or at most about 1, 2, 3, 4, 5, 6, 7, 8, 9, 10, 11, 12, 13, 14, 15, 16, 17, 18, 19, or 20 basic amino acids. In some embodiments, the first 20 N-terminal amino acids of the buffer peptide include about, at least about, or at most about 1, 2, 3, 4, 5, 6, 7, 8, 9, 10, 11, 12, 13, 14, 15, 16, 17, or 18 basic amino acids.

In some embodiments, the first 3 N-terminal amino acids of the buffer peptide comprise about or at least about 1, 2, or 3 acidic and/or neutral and/or nonpolar aliphatic amino acids. In some embodiments, the first 4 N-terminal amino acids of the buffer peptide comprise about or at least about 1, 2, 3, or 4 acidic and/or neutral and/or nonpolar aliphatic amino acids. In some embodiments, the first 5 N-terminal amino acids of the buffer peptide comprise about or at least about 1, 2, 3, 4, or 5 acidic and/or neutral and/or nonpolar aliphatic amino acids. In some embodiments, the first 6 N-terminal amino acids of the buffer peptide comprise about or at least about 1, 2, 3, 4, 5, or 6 acidic and/or neutral and/or nonpolar aliphatic amino acids. In some embodiments, the first 7 N-terminal amino acids of the buffer peptide comprise about or at least about 1, 2, 3, 4, 5, 6, or 7 acidic and/or neutral and/or nonpolar aliphatic amino acids. In some embodiments, the first 8 N-terminal amino acids of the buffer peptide comprise about or at least about 1, 2, 3, 4, 5, 6, 7, or 8 acidic and/or neutral and/or nonpolar aliphatic amino acids. In some embodiments, the first 9 N-terminal amino acids of the buffer peptide comprise about or at least about 1, 2, 3, 4, 5, 6, 7, 8, or 9 acidic and/or neutral and/or nonpolar aliphatic amino acids. In some embodiments, the first 10 N-terminal amino acids of the buffer peptide comprise about or at least about 1, 2, 3, 4, 5, 6, 7, 8, 9, or 10 acidic and/or neutral and/or nonpolar aliphatic amino acids. In some embodiments, the first 20 N-terminal amino acids of the buffer peptide comprise about or at least about 1, 2, 3, 4, 5, 6, 7, 8, 9, 10, 11, 12, 13, 14, 15, 16, 17, 18, 19, or 20 acidic and/or neutral and/or nonpolar aliphatic amino acids.

In some embodiments, the last C-terminal amino acid of the buffer peptide is a basic amino acid. In some embodiments, the last 2 C-terminal amino acids of the buffer peptide comprise 1 or 2 basic amino acids. In some embodiments, the last 3 C-terminal amino acids of the buffer peptide comprise 1, 2, or 3 basic amino acids. In some embodiments, the last 4 C-terminal amino acids of the buffer peptide comprise 1, 2, 3, or 4 basic amino acids. In some embodiments, the last 5 C-terminal amino acids of the buffer peptide comprise 1, 2, 3, 4, or 5 basic amino acids. In some embodiments, the last 6 C-terminal amino acids of the buffer peptide comprise 1, 2, 3, 4, 5, or 6 basic amino acids. In some embodiments, the last 7 C-terminal amino acids of the buffer peptide comprise 1, 2, 3, 4, 5, 6, or 7 basic amino acids. In some embodiments, the last 8 C-terminal amino acids of the buffer peptide comprise 1, 2, 3, 4, 5, 6, 7, or 8 basic amino acids. In some embodiments, the last 9 C-terminal amino acids of the buffer peptide comprise 1, 2, 3, 4, 5, 6, 7, 8, or 9 basic amino acids. In some embodiments, the last 10 C-terminal amino acids of the buffer peptide comprise 1, 2, 3, 4, 5, 6, 7, 8, 9, or 10 basic amino acids. In some embodiments, the 5 amino acids C-terminal to the first 5, 6, 7, or 8 N-terminal amino acids of the buffer peptide comprise about or at most about 1, 2, or 3 basic amino acids. In some embodiments, the 3 C-terminal amino acids to the first 3 to 10 N-terminal amino acids of the buffer peptide comprise about or at most about 1, 2, or 3 basic amino acids. In some embodiments, the buffer peptide does not comprise a basic amino acid. In some embodiments, the buffer peptide is devoid of basic amino acids. In some embodiments, the first 2 N-terminal amino acids of the buffer peptide do not comprise basic amino acids. In some embodiments, the first 3 N-terminal amino acids of the buffer peptide do not comprise basic amino acids. In some embodiments, the first 4 N-terminal amino acids of the buffer peptide do not comprise basic amino acids. In some embodiments, the first 5 N-terminal amino acids of the buffer peptide do not comprise basic amino acids. In some embodiments, the first 6 N-terminal amino acids of the buffer peptide do not comprise basic amino acids. In some embodiments, the first 7 N-terminal amino acids of the buffer peptide do not comprise basic amino acids. In some embodiments, the first 8 N-terminal amino acids of the buffer peptide do not comprise basic amino acids. In some embodiments, the first 9 N-terminal amino acids of the buffer peptide do not comprise basic amino acids. In some embodiments, the first 10 N-terminal amino acids of the buffer peptide do not comprise basic amino acids.

In some embodiments, the buffer peptide comprises about, at most about, or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the buffer peptide comprises about, at most about, or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids. In some embodiments, the buffer peptide comprises about, at most about, or at least about 20%, 25%, 30%, 35%, 40%, 45%, 50%, 55% 60%, 65%, 70%, 75%, 80%, 85%, 90%, 95%, 96%, 97%, 98%, 99%, or 100% neutral amino acids. In some embodiments, the buffer peptide comprises about, at most about, or at least about 20%, 25%, 30%, 35%, 40%, 45%, 50%, 55% 60%, 65%, 70%, 75%, 80%, 85%, 90%, 95%, 96%, 97%, 98%, 99%, or 100% nonpolar aliphatic amino acids.

In some embodiments, the first 2 N-terminal amino acids of the buffer peptide comprise about or at least about 50% or 100% acidic amino acids and/or neutral and/or nonpolar aliphatic amino acids. In some embodiments, the first 3 N-terminal amino acids of the buffer peptide comprise about or at least about 20%, 25%, 30%, 35%, 40%, 45%, 50%, 55% 60%, 65%, 70%, 75%, 80%, 8% 5, ⁹ 0, 95% 96%, 97%, 98%, 99%, or 100% acidic amino acids and/or neutral and/or nonpolar aliphatic amino acids. In some embodiments, the first 4 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 5 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 6 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 7 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 8 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 9 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids. In some embodiments, the first 10 N-terminal amino acids of the buffer peptide comprise about or at least about 20%, 25%, 30%, 35%, 40%, 45%, 50%, 55% 60%, 65%, 70%, 75%, 80%, 85%, 90%, 95%, 96%, 97%, 98%, 99%, or 100% acidic amino acids and/or neutral and/or nonpolar aliphatic amino acids.

In some embodiments, an arenavirus GP signal peptide can be cleaved from a buffer peptide or a portion thereof by a signal peptide peptidase. In some embodiments, a signal peptide peptidase cleaves immediately N-terminal to a buffer peptide (e.g., cleaves an an arenavirus GP signal peptide from a buffer peptide immediately N-terminal to a buffer peptide). In some embodiments, a signal peptide peptidase cleaves after the first N-terminal amino acid of the buffer peptide. In some embodiments, a signal peptide peptidase cleaves after the second N-terminal amino acid of the buffer peptide. In some embodiments, a signal peptide peptidase cleaves after the third N-terminal amino acid of the buffer peptide. In some embodiments, a signal peptide peptidase cleaves after the fourth N-terminal amino acid of the buffer peptide. In some embodiments, a signal peptide peptidase cleaves after the fifth N-terminal amino acid of the buffer peptide. In some embodiments, a signal peptide peptidase cleaves after the sixth N-terminal amino acid of the buffer peptide. In some embodiments, a signal peptide peptidase cleaves after the seventh N-terminal amino acid of the buffer peptide. In some embodiments, a signal peptide peptidase cleaves after the 8 N-terminal amino acid of the buffer peptide. In some embodiments, a signal peptide peptidase cleaves after the 9 N-terminal amino acid of the buffer peptide. In some embodiments, a signal peptide peptidase cleaves after the 10 N-terminal amino acid of the buffer peptide (or more than after the 10 N-terminal amino acid).

In some embodiments, an arenavirus GP signal peptide is cleaved at exactly the position where a buffer peptide starts. In some embodiments, an arenavirus GP signal peptide is cleaved at amino acid 1, 2, 3, 4, 5, 6, 7, 8, 9, 10, or more than 10 amino acids N-terminal from where a buffer peptide starts. In some embodiments, an arenavirus GP signal peptide is cleaved at amino acid 1, 2, 3, 4, 5, 6, 7, 8, 9, 10, or more than 10 amino acids C-terminal from where a buffer peptide starts.

In some embodiments, a buffer peptide starts at about or at least about amino acid 54, 55, 56, 57, 58, 59, 60, 61, or 62 of an amino acid sequence of a glycoprotein precursor. In some embodiments, a buffer peptide starts at about amino acid 58 of an amino acid sequence of a glycoprotein precursor. In some embodiments, a buffer peptide starts at about amino acid 59 of an amino acid sequence of a glycoprotein precursor. In some embodiments, a buffer peptide starts at about amino acid 60 of an amino acid sequence of a glycoprotein precursor. In some embodiments, a buffer peptide starts at about amino acid 61 of an amino acid sequence of a glycoprotein precursor. In some embodiments, a buffer peptide starts at about or at least about amino acid 54, 55, 56, 57, 58, 59, 60, 61, or 62 of an amino acid sequence of any one of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, and/or SEQ ID NO: 134. In some embodiments, one, two, or more than two, or at least one amino acid at position 54, 55, 56, 57, 58, 59, 60, 61, 62, 63, 64, 65, 66, 67, and/or 68 of an amino acid sequence of any one of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, and/or SEQ ID NO:134 comprises or is mutated to a basic amino acid, neutral amino acid, nonpolar aliphatic amino acid, nonpolar aromatic amino acid, polar (e.g., neutral, positively charged or negatively charged) amino acid, acidic amino acid, or a combination thereof. In some embodiments, the amino acid at position 60 of an amino acid sequence of a glycoprotein precursor comprises or is mutated to a nonpolar aliphatic amino acid (e.g., glycine, alanine, proline, valine, leucine, methionine, or isoleucine), a basic amino acid (e.g., arginine, lysine, or histidine), an acidic amino acid (e.g., aspartic acid or glutamic acid), a nonpolar aromatic amino acid (e.g., phenylalanine, tyrosine, or tryptophan) and/or a polar amino acid (e.g., serine, threonine, cysteine, asparagine, glutamine, lysine, arginine, histidine, aspartic acid, or glutamic acid). In some embodiments, the amino acid at position 61 of an amino acid sequence of a glycoprotein precursor comprises or is mutated to a nonpolar aliphatic amino acid (e.g., glycine, alanine, proline, valine, leucine, methionine, or isoleucine), a basic amino acid (e.g., arginine, lysine, or histidine), an acidic amino acid (e.g., aspartic acid or glutamic acid), a nonpolar aromatic amino acid (e.g., phenylalanine, tyrosine, or tryptophan) and/or a polar amino acid (e.g., serine, threonine, cysteine, asparagine, glutamine, lysine, arginine, histidine, aspartic acid, or glutamic acid). In some embodiments, the amino acid at position 60 of an amino acid sequence of a glycoprotein precursor comprises or is mutated to a basic amino acid. In some embodiments, the amino acid at position 61 of an amino acid sequence of a glycoprotein precursor comprises or is mutated to a basic amino acid. In some embodiments, the amino acid at positions 60 and 61 of an amino acid sequence of a glycoprotein precursor (e.g., SEQ ID NO: 3) comprise or are mutated to a basic amino acid (e.g., arginine, histidine, and/or lysine). In some embodiments, the amino acid at position 60 of an amino acid sequence of a glycoprotein precursor comprises or is mutated to a nonpolar aliphatic amino acid (e.g., glycine, alanine, proline, valine, leucine, isoleucine, or methionine). In some embodiments, the amino acid at position 61 of an amino acid sequence of a glycoprotein precursor comprises or is mutated to a nonpolar aliphatic amino acid (e.g., glycine, alanine, proline, valine, leucine, isoleucine, or methionine). In some embodiments, the amino acids at positions 60 and 61 of an amino acid sequence of a glycoprotein precursor (e.g., SEQ ID NO: 3) comprise or are mutated to a nonpolar aliphatic amino acid (e.g., glycine, alanine, proline, valine, leucine, isoleucine, and/or methionine). In some embodiments, the amino acid at position 60 of an amino acid sequence of a glycoprotein precursor comprises or is mutated to a polar amino acid. In some embodiments, the amino acid at position 61 of an amino acid sequence of a glycoprotein precursor comprises or is mutated to a polar amino acid. In some embodiments, the amino acids at positions 60 and 61 of an amino acid sequence of a glycoprotein precursor (e.g., SEQ ID NO: 3) comprise or are mutated to a polar amino acid. In some embodiments, the amino acid at position 60 of an amino acid sequence of a glycoprotein precursor comprises or is mutated to an acidic amino acid. In some embodiments, the amino acid at position 61 of an amino acid sequence of a glycoprotein precursor comprises or is mutated to an acidic amino acid. In some embodiments, the amino acids at positions 60 and 61 of an amino acid sequence of a glycoprotein precursor (e.g., SEQ ID NO: 3) comprise or are mutated to an acidic amino acid. In some embodiments, the amino acid at position 60 of an amino acid sequence of a glycoprotein precursor comprises or is mutated to a nonpolar aromatic amino acid. In some embodiments, the amino acid at position 61 of an amino acid sequence of a glycoprotein precursor comprises or is mutated to a nonpolar aromatic amino acid. In some embodiments, the amino acids at positions 60 and 61 of an amino acid sequence of a glycoprotein precursor (e.g., SEQ ID NO: 3) comprise or are mutated to a nonpolar aromatic amino acid. In some embodiments, the methionines at positions 60 and 61 of an amino acid sequence of a glycoprotein precursor (e.g., SEQ ID NO: 3) are both mutated to leucine. In some embodiments, the methionines at positions 60 and 61 of an amino acid sequence of a glycoprotein precursor (e.g., SEQ ID NO: 3) are both mutated to aspartic acid. In some embodiments, the methionines at positions 60 and 61 of an amino acid sequence of a glycoprotein precursor (e.g., SEQ ID NO: 3) are both mutated to glycine. In some embodiments, the methionines at positions 60 and 61 of an amino acid sequence of a glycoprotein precursor (e.g., SEQ ID NO: 3) are both mutated to glutamic acid. In some embodiments, the methionines at positions 60 and 61 of an amino acid sequence of a glycoprotein precursor (e.g., SEQ ID NO: 3) are both mutated to histidine. In some embodiments, the methionines at positions 60 and 61 of an amino acid sequence of a glycoprotein precursor (e.g., SEQ ID NO: 3) are both mutated to lysine.

### 5.5 Nucleotide Sequence Provided Herein

In one aspect, provided herein are nucleotide sequences. In certain embodiments, provided herein is a nucleotide sequence comprising a first open reading frame (ORF) and a second ORF, wherein one of the two ORFs is in sense orientation and the other ORF is in antisense orientation;

wherein the first ORF comprises a nucleotide sequence encoding a functional fragment of a first polypeptide fused to a buffer peptide (as described above; see Section 5.4), and the first ORF does not encode the full-length first polypeptide;
wherein the second ORF comprises a nucleotide sequence encoding:
a) a second polypeptide; or
b) a functional fragment of the first polypeptide, and the second ORF does not encode the full-length first polypeptide; or
c) a functional fragment of a second polypeptide, and the second ORF does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first ORF and the second ORF are separated by an arenavirus intergenic region (IGR) and each ORF is under control of an arenavirus 3′ untranslated region (UTR) or an arenavirus 5′ UTR.

In certain embodiments, the first ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second ORF further comprises a nucleotide sequence encoding a second heterologous non-arenaviral polypeptide or a third polypeptide; wherein the third polypeptide is different from the first polypeptide and second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the first ORF is part of a transcription unit that further comprises another ORF, wherein the other ORF and the first ORF are separated by an internal ribosome entry site (IRES). In certain embodiments, the second ORF is part of a transcription unit that further comprises another ORF, wherein the other ORF and the second ORF are separated by an IRES. In certain embodiments, the first ORF and/or the second ORF comprises a nucleotide sequence encoding an arenavirus GP signal peptide and a buffer peptide or a functional fragment of the GP signal peptide or a polypeptide comprising such a functional fragment fused to a buffer peptide and the other ORF comprises a nucleotide sequence encoding a heterologous non-arenaviral polypeptide or an arenavirus NP, Z, or L.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is a nucleotide sequence comprising an open reading frame (ORF), wherein the ORF comprises a nucleotide sequence encoding
a) a functional fragment of a first polypeptide, and
b) a heterologous non-arenaviral polypeptide or a second polypeptide;
   wherein the ORF does not encode the full-length first polypeptide; and wherein the first and second polypeptides are different from each other and selected from the group consisting of arenavirus GP, NP, Z and L that are not from Lassa virus. In certain embodiments, the first and second polypeptides are selected from the group consisting of arenavirus GP, NP, Z and L of LCMV, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the ORF is a first ORF and the nucleotide sequence further comprises a second ORF. In certain embodiments, the second ORF comprises a nucleotide sequence encoding a third polypeptide, a functional fragment of the first polypeptide, a functional fragment of a third polypeptide, or a second heterologous non-arenaviral polypeptide; wherein the third polypeptide is different from the first polypeptide and the second polypeptide and selected from the group consisting of arenavirus GP, NP, Z and L; wherein one of the two ORFs is in sense orientation and the other ORF is in antisense orientation; wherein the second ORF does not encode the full-length first polypeptide; wherein the second ORF does not encode the full-length third polypeptide; and wherein the heterologous non-arenaviral polypeptide and the second heterologous non-arenaviral polypeptide are the same or different from each other. In certain embodiments, the second ORF comprises a nucleotide sequence encoding a functional fragment of a first polypeptide, and wherein the functional fragment encoded by the first ORF is different from the functional fragment encoded by the second ORE.

In certain embodiments, the nucleotide sequence does not further comprise a second ORF.

In certain embodiments, the nucleotide sequence is an arenavirus genomic or antigenomic S segment. In certain embodiments, the nucleotide sequence is an arenavirus genomic or antigenomic L segment.

In certain embodiments, the first ORF is under control of an arenavirus 3′ UTR, and the second ORF is under control of an arenavirus 5′ UTR. In certain embodiments, the first ORF is under control of an arenavirus 5′ UTR, and the second ORF is under control of an arenavirus 3′ UTR.

In certain embodiments, the first ORF comprises a nucleotide sequence encoding arenavirus GP signal peptide and a buffer peptide, either alone or fused to a heterologous non-arenaviral polypeptide. In certain embodiments, the second ORF comprises a nucleotide sequence encoding a heterologous non-arenaviral signal peptide and the arenavirus GP1 and GP2. In certain embodiments, the second ORF comprises a nucleotide sequence encoding a heterologous non-arenaviral polypeptide; wherein the heterologous non-arenaviral polypeptide encoded by the first ORF and the heterologous non-arenaviral polypeptide encoded by the second ORF are the same or different from each other. In certain embodiments, the first ORF is under control of an arenavirus 3′ UTR and the second ORF is under control of an arenavirus 5′ UTR.

In certain embodiments, the first ORF comprises a nucleotide sequence encoding a heterologous non-arenaviral signal peptide and the arenavirus GP1 and GP2. In certain embodiments, the second ORF comprises a nucleotide sequence encoding NP. In certain embodiments, the first ORF is under control of an arenavirus 5′ UTR and the second ORF is under control of an arenavirus 3′ UTR.

In certain embodiments, provided herein is a nucleotide sequence comprising an open reading frame (ORF) that encodes a polypeptide comprising or consisting of an amino acid sequence that is at least 80%, 85%, 90%, 95%, 96%, 97%, 98%, 99% or 100% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) that encodes a polypeptide comprising or consisting of an amino acid sequence that is at least 80% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) that encodes a polypeptide comprising or consisting of an amino acid sequence that is at least 85% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) that encodes a polypeptide comprising or consisting of an amino acid sequence that is at least 90% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is at least 95% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is at least 96% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is at least 97% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is at least 98% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is at least 99% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the nucleotide sequence comprising an open reading frame (ORF) encodes a polypeptide comprising or consisting of an amino acid sequence that is 100% identical to a portion of the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In some embodiments, the portion of the amino acid sequence is any portion of a sequence and can range in the number of amino acids. In some embodiments, the portion of the amino acid sequence is from about amino acid 1 to about amino acid 5, from about amino acid 1 to about amino acid 10, from about amino acid 1 to about amino acid 15, from about amino acid 1 to about amino acid 20, from about amino acid 1 to about amino acid 25, from about amino acid 1 to about amino acid 30, from about amino acid 1 to about amino acid 35, from about amino acid 1 to about amino acid 40, from about amino acid 1 to about amino acid 45, from about amino acid 1 to about amino acid 50, from about amino acid 1 to about amino acid 51, from about amino acid 1 to about amino acid 52, from about amino acid 1 to about amino acid 53, from about amino acid 1 to about amino acid 54, from about amino acid 1 to about amino acid 55, from about amino acid 1 to about amino acid 56, from about amino acid 1 to about amino acid 57, from about amino acid 1 to about amino acid 58, from about amino acid 1 to about amino acid 59, from about amino acid 1 to about amino acid 60, from about amino acid 1 to about amino acid 61, from about amino acid 1 to about amino acid 62, from about amino acid 1 to about amino acid 63, from about amino acid 1 to about amino acid 64, from about amino acid 1 to about amino acid 65, from about amino acid 1 to about amino acid 66, from about amino acid 1 to about amino acid 67, from about amino acid 1 to about amino acid 68, from about amino acid 1 to about amino acid 69, from about amino acid 1 to about amino acid 70, from about amino acid 1 to about amino acid 71, from about amino acid 1 to about amino acid 72, from about amino acid 1 to about amino acid 73, from about amino acid 1 to about amino acid 74, from about amino acid 1 to about amino acid 75, from about amino acid 1 to about amino acid 76, from about amino acid 1 to about amino acid 77, from about amino acid 1 to about amino acid 78, from about amino acid 1 to about amino acid 79, from about amino acid 1 to about amino acid 80, from about amino acid 1 to about amino acid 81, from about amino acid 1 to about amino acid 82, from about amino acid 1 to about amino acid 83, from about amino acid 1 to about amino acid 84, from about amino acid 1 to about amino acid 85, from about amino acid 1 to about amino acid 86, from about amino acid 1 to about amino acid 87, from about amino acid 1 to about amino acid 88, from about amino acid 1 to about amino acid 89, from about amino acid 1 to about amino acid 90, from about amino acid 1 to about amino acid 91, from about amino acid 1 to about amino acid 92, from about amino acid 1 to about amino acid 93, from about amino acid 1 to about amino acid 94, from about amino acid 1 to about amino acid 95, from about amino acid 1 to about amino acid 96, from about amino acid 1 to about amino acid 97, from about amino acid 1 to about amino acid 98, from about amino acid 1 to about amino acid 99, from about amino acid 1 to about amino acid 100, from about amino acid 1 to about amino acid 101, from about amino acid 1 to about amino acid 102, from about amino acid 1 to about amino acid 103, from about amino acid 1 to about amino acid 104, from about amino acid 1 to about amino acid 105, from about amino acid 1 to about amino acid 106, from about amino acid 1 to about amino acid 107, from about amino acid 1 to about amino acid 108, from about amino acid 1 to about amino acid 109, from about amino acid 1 to about amino acid 110, from about amino acid 1 to about amino acid 111, from about amino acid 1 to about amino acid 112, from about amino acid 1 to about amino acid 113, or from about amino acid 1 to about more than amino acid 114 (e.g., 115, 120, 125, 130, 135, 140, 145, 150, 200, 250, 300, etc.) of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, or an arenavirus glycoprotein precursor sequence. In some embodiments, the portion of the amino acid sequence includes or is from about amino acid 1 to about amino acid 66 of SEQ ID NO:3. In some embodiments, the portion of the amino acid sequence includes or is from about amino acid 1 to about amino acid 81 of SEQ ID NO:3. In some embodiments, the portion of the amino acid sequence includes or is from about amino acid 1 to about amino acid 113 of SEQ ID NO:3.

In certain embodiments, the first polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140; and the second polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140; and wherein the third polypeptide comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the first polypeptide comprises an amino acid sequence identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, SEQ ID NO:166, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, SEQ ID NO:165, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163.

In certain embodiments, the functional fragment encoded by the first ORF or the second ORF is an arenavirus GP signal peptide and a buffer peptide, or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of
(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMIB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

In certain embodiments, the heterologous non-arenaviral polypeptide or the second heterologous non-arenaviral polypeptide is expressed under control of an arenavirus 3′ UTR; and the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the combined expression level of both the heterologous non-arenaviral polypeptides is at least about 1%, 2%, 3%, 4%, 5%, 6%, 7%, 8%, 9%, 10%, 15%, 20%, 30%, 40%, 5%, ⁶50%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of both the heterologous non-arenaviral polypeptides expressed under control of arenavirus 5′ UTRs.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the heterologous non-arenaviral polypeptide is expressed under control of an arenavirus 5′ UTR and the second heterologous non-arenaviral polypeptide is expressed under control of an arenavirus 3′ UTR. In certain embodiments, the combined expression level of both the heterologous non-arenaviral polypeptide and the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR and the same second heterologous non-arenaviral polypeptide is expressed under control of an arenavirus 5′ UTR).

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR and the same second heterologous non-arenaviral polypeptide is expressed under control of an arenavirus 5′ UTR). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, one heterologous non-arenaviral polypeptide is expressed under control of an arenavirus 5′ UTR and the other heterologous non-arenaviral polypeptide is expressed under control of an arenavirus 3′ UTR. In certain embodiments, the heterologous non-arenaviral polypeptide is expressed under control of an arenavirus 5′ UTR and the second heterologous non-arenaviral polypeptide is expressed under control of an arenavirus 3′ UTR. In certain embodiments, the heterologous non-arenaviral polypeptide is expressed under control of an arenavirus 5′ UTR in one S segment and the second heterologous non-arenaviral polypeptide is expressed under control of an arenavirus 3′ UTR in another S segment. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR in one S segment and the same second heterologous non-arenaviral polypeptide is expressed under control of an arenavirus 5′ UTR in another S segment). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, the nucleotide sequence provided herein comprises a first open reading frame (ORF) and a second ORF, and one of the two ORFs is in sense orientation and the other ORF is in antisense orientation. In certain embodiments, the first ORF is in sense orientation and the second ORF is in antisense orientation. In other embodiments, the first ORF is in antisense orientation and the second ORF is in sense orientation. In certain embodiments, the first ORF and/or the second ORF is part of a transcription unit that further comprises another ORF, wherein the other ORF and each of the first ORF and/or the second ORF are separated by an internal ribosome entry site (IRES).

In certain embodiment, the functional fragment is encoded alone by the first ORF or the second ORF. In certain embodiments, the functional fragment is fused to a heterologous non-arenaviral polypeptide or a polypeptide selected from an arenavirus GP, NP, Z and L. In certain embodiment, the functional fragment is under control of an arenavirus 3′ UTR. In other embodiments, the functional fragment is under control of an arenavirus 5′ UTR. In certain embodiment, the functional fragment encoded by the first ORF is different from the functional fragment encoded by the second ORF.

In certain embodiments, the functional fragment encoded by the first ORF or the second ORF is arenavirus GP signal peptide and a buffer peptide. In certain embodiments, the arenavirus GP signal peptide and the buffer peptide are not fused to an arenavirus GP, NP, Z or L.

In certain embodiments, the nucleotide provided herein is an mRNA. In certain embodiments, the nucleotide provided herein is an mRNA comprising an internal ribosome entry site (IRES). In other embodiments, the nucleotide provided herein is a DNA. In certain embodiments, the nucleotide provided herein can be translated into a product, for example a polypeptide. In certain embodiments, the nucleotide provided herein is a DNA that can be transcribed to produce an arenavirus genomic or antigenomic RNA segment. In certain embodiments, the nucleotide sequence as described herein can be part of a DNA expression vector.

Non-limiting examples of the polypeptides encoded by the first and second ORFs are illustration in Table 1.

TABLE 1
Non-limiting examples of the polypeptides
encoded by the first and second ORFs.

| | 1^{st} ORF | | |
|---|---|---|---|
| | Functional | | |
| | fragment | | Functional |
| | of a first | | fragment |
| | polypeptide | | of a first |
| | and a heterologous | | polypeptide |
| | non-arenaviral | | and a third |
| | polypeptide | | polypeptide |
| | (fused | Functional | (fused |
| | together or | fragment | together or |
| | separated | of a first | separated |
| 2^{nd} ORF | by an IRES) | polypeptide | by an IRES) |
| Functional fragment | Exemplary | Exemplary | Exemplary |
| of a first polypeptide | combination | combination | combination |
| and a heterologous | | | |
| non-arenaviral | | | |
| polypeptide | | | |
| (fused together or | | | |
| separated by an | | | |
| IRES) | | | |
| Functional fragment | Exemplary | Exemplary | Exemplary |
| of a first polypeptide | combination | combination | combination |
| Functional fragment | Exemplary | Exemplary | Exemplary |
| of a first polypeptide | combination | combination | combination |
| and a third | | | |
| polypeptide | | | |
| (fused together or | | | |
| separated by an | | | |
| IRES) | | | |
| A heterologous non- | Exemplary | Exemplary | Exemplary |
| arenaviral | combination | combination | combination |
| polypeptide | | | |
| A second | Exemplary | Exemplary | Exemplary |
| polypeptide | combination | combination | combination |
| Functional fragment | Exemplary | Exemplary | Exemplary |
| of a second | combination | combination | combination |
| polypeptide and a | | | |
| heterologous non- | | | |
| arenaviral | | | |
| polypeptide | | | |
| (fused together or | | | |
| separated by an | | | |
| IRES) | | | |
| Functional fragment | Exemplary | Exemplary | Exemplary |
| of a second | combination | combination | combination |
| polypeptide | | | |
| Functional fragment | Exemplary | Exemplary | Exemplary |
| of a second | combination | combination | combination |
| polypeptide and a | | | |
| third polypeptide | | | |
| (fused together or | | | |
| separated by an | | | |
| IRES) | | | |

In certain embodiments, the functional fragment is selected from the group consisting of arenavirus GP signal peptide, a buffer peptide, arenavirus GP1, and arenavirus GP2. Non-limiting examples of the polypeptides encoded by the first and second ORFs related to functional fragments of GP are illustrated in Table 2.

TABLE 2
Non-limiting examples of the polypeptides encoded by the first
and second ORFs related to functional fragments of GP.

| First ORF | Second ORF |
|---|---|
| GP signal peptide, a buffer peptide, and a | A heterologous non-arenaviral signal |
| heterologous non-arenaviral polypeptide | peptide, arenavirus GP1, and arenavirus |
| | GP2 |
| GP signal peptide, a buffer peptide, and a | A heterologous non-arenaviral polypeptide |
| heterologous non-arenaviral polypeptide | |
| GP signal peptide, a buffer peptide, and a | NP |
| heterologous non-arenaviral polypeptide | |
| GP signal peptide, a buffer peptide, and a | Z |
| heterologous non-arenaviral polypeptide | |
| GP signal peptide, a buffer peptide, and a | L |
| heterologous non-arenaviral polypeptide | |
| GP signal peptide, a buffer peptide, and a | No second ORF |
| heterologous non-arenaviral polypeptide | |
| GP signal peptide and a buffer peptide | A heterologous non-arenaviral signal |
| | peptide, arenavirus GP1, and arenavirus |
| | GP2 |
| GP signal peptide and a buffer peptide | A heterologous non-arenaviral polypeptide |
| GP signal peptide and a buffer peptide | NP |
| GP signal peptide and a buffer peptide | Z |
| GP signal peptide and a buffer peptide | L |
| GP signal peptide and a buffer peptide | No second ORF |
| GP signal peptide, a buffer peptide, and NP | A heterologous non-arenaviral signal |
| | peptide, arenavirus GP1, and arenavirus |
| | GP2 |
| GP signal peptide, a buffer peptide, and NP | A heterologous non-arenaviral polypeptide |
| GP signal peptide, a buffer peptide, and NP | Z |
| GP signal peptide, a buffer peptide, and NP | L |
| GP signal peptide, a buffer peptide, and NP | No second ORF |
| GP signal peptide, a buffer peptide, and Z | A heterologous non-arenaviral signal |
| | peptide, arenavirus GP1, and arenavirus |
| | GP2 |
| GP signal peptide, a buffer peptide, and Z | A heterologous non-arenaviral polypeptide |
| GP signal peptide, a buffer peptide, and Z | NP |
| GP signal peptide, a buffer peptide, and Z | L |
| GP signal peptide, a buffer peptide, and Z | No second ORF |
| GP signal peptide, a buffer peptide, and L | A heterologous non-arenaviral signal |
| | peptide, arenavirus GP1, and arenavirus |
| | GP2 |
| GP signal peptide, a buffer peptide, and L | A heterologous non-arenaviral polypeptide |
| GP signal peptide, a buffer peptide, and L | NP |
| GP signal peptide, a buffer peptide, and L | Z |
| GP signal peptide, a buffer peptide, and L | No second ORF |
| GP1, GP2 and a heterologous non- | GP signal peptide, a buffer peptide, and a |
| arenaviral signal peptide | heterologous non-arenaviral polypeptide |
| GP1, GP2 and a heterologous non- | GP signal peptide and a buffer peptide |
| arenaviral signal peptide | |
| GP1, GP2 and a heterologous non- | A heterologous non-arenaviral polypeptide |
| arenaviral signal peptide | |
| GP1, GP2 and a heterologous non- | NP |
| arenaviral signal peptide | |
| GP1, GP2 and a heterologous non- | Z |
| arenaviral signal peptide | |
| GP1, GP2 and a heterologous non- | L |
| arenaviral signal peptide | |
| GP1, GP2 and a heterologous non- | No second ORF |
| arenaviral signal peptide | |

### 5.5.1 Arenavirus Genomic or Antigenomic Segment Related to the Nucleotide Sequence Provided Herein

In certain embodiments, the nucleotide sequence provided herein is an arenavirus genomic or antigenomic segment. In certain embodiments, the nucleotide sequence is an arenavirus genomic or antigenomic S segment. In certain embodiments, the nucleotide sequence is an arenavirus genomic or antigenomic L segment. In certain embodiments, the nucleotide sequence provided herein can be derived from an arenavirus genomic or antigenomic segment.

In certain embodiments, the first ORF and the second ORF of the nucleotide sequence as described herein are in an S segment or derived from an S segment and separated by an arenavirus intergenic region (IGR). In certain embodiments, the first ORF and the second ORF of the nucleotide sequence as described herein are in an L segment or derived from an L segment and separated by an arenavirus intergenic region (IGR). In certain embodiments, the first ORF in an S segment is under control of an arenavirus 3′ untranslated region (UTR) whereas the second ORF in the S segment is under control of an arenavirus 5′ UTR. In certain embodiments, the first ORF in an L segment is under control of an arenavirus 3′ untranslated region (UTR) whereas the second ORF in the L segment is under control of an arenavirus 5′ UTR. In certain embodiments, the first ORF in an S segment is under control of an arenavirus 5′ UTR whereas the second ORF in the S segment is under control of an arenavirus 3′ UTR. In certain embodiments, the first ORF in an L segment is under control of an arenavirus 5′ UTR whereas the second ORF in the L segment is under control of an arenavirus 3′ UTR.

More details on the arenavirus genomic or antigenomic segment provided herein are described in Section 5.7.

### 5.5.2 Arenavirus Particles Comprising the Nucleotide Sequence Provided Herein

In certain embodiments, provided herein is an arenavirus particle containing a genome comprising the nucleotide sequence provided herein. In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide, alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the genome of the arenavirus particle consists of
a. an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR;
b. an S segment that encodes NP under the control of an arenavirus genomic 3′ UTR and a second heterologous non-arenaviral polypeptide or no polypeptide under the control of an arenavirus genomic 5′ UTR; and
c. an L segment that encodes the L protein under the control of an arenavirus genomic 3′ UTR and the Z protein under the control of an arenavirus genomic 5′ UTR, wherein the first heterologous non-arenaviral polypeptide and the second heterologous non-arenaviral polypeptide are the same or different from each other.

In certain embodiments, the genome of the arenavirus particle consists of
a. an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR;
b. an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and
c. an L segment that encodes the L protein under the control of an arenavirus genomic 3′ UTR and the Z protein under the control of an arenavirus genomic 5′ UTR, wherein the first heterologous non-arenaviral polypeptide and the second heterologous non-arenaviral polypeptide are the same or different from each other.

In some embodiments, the GP signal peptide (or functional fragment thereof or polypeptide comprising the GP signal peptide or functional fragment thereof) is cleaved from the buffer peptide. In some embodiments, a peptidase can cleave the GP signal peptide (or functional fragment thereof or polypeptide comprising the GP signal peptide or functional fragment thereof) from the buffer peptide (eg, −5, −4, −3, −2, −1, +1, +2, +3, +4, or +5 amino acid position from the fusion site, or at the fusion site). In some embodiments, an arenavirus particle as described herein wherein the GP signal peptide is fused to a buffer peptide (i) grows to higher titers (e.g., by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, or higher than about 100%), and/or (ii) reaches peak titers earlier (e.g., by about or at least about 1 hr, 5 hr, 10 hr, 12 hr, 14 hr, 20 hr, 24 hrs, 1 day, 2 days, 3 days, 4 days, 5 days, 6 days, 7 days, 1.5 weeks, 2 weeks, 2.5 weeks, 3 weeks, 3.5 weeks, 4 weeks, or more than about 4 weeks), and/or (iii) exhibits a higher rescue efficiency (i.e., higher percentage of RCV titer-positive rescues of total rescues) (e.g., higher by about or at least about 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, or higher than about 100%), as compared to a control arenavirus particle that has the same genomic organization except it lacks a buffer peptide. In some embodiments, any native signal peptide of a heterologous non-arenaviral polypeptide (e.g., a first heterologous non-arenaviral polypeptide) has been removed.

In certain embodiments, the arenavirus particle is derived from an arenavirus of the New World Clade A. In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF. In certain embodiments, the growth or infectivity of the arenavirus particle described herein is not inferior to a second arenavirus particle. In some embodiments, the genome of the second arenavirus particle does not encode a buffer peptide.

In certain embodiments, the arenavirus particle is infectious and replication competent. In certain embodiments, the arenavirus particle is attenuated as compared to its parental virus. In certain embodiments, the arenavirus particle is infectious but unable to produce further infectious progeny in non-complementing cells.

In certain embodiments, the titer of the arenavirus particle of the disclosure is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%1, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, provided herein is an arenavirus particle comprising the nucleotide sequence provided herein. In certain embodiments, the arenavirus particle described herein is genetically stable. In certain embodiments, the arenavirus particle described herein provides high-level transgene expression. In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments. More details on the arenavirus genomic or antigenomic segment provided herein are described in Section 5.7.

### 5.5.3 Compositions and Methods Related to the Nucleotide Sequence Provided Herein

In certain embodiments, provided herein is a translation product of the nucleotide sequence provided herein.

In certain embodiments, the nucleotide sequence is a DNA sequence, which can be transcribed into an arenavirus genomic or antigenomic segment. In certain embodiments, provided herein is a method of producing an arenavirus genomic or antigenomic RNA segment, wherein the method comprises transcribing the DNA sequence provided herein.

In certain embodiments, provided herein is a method of generating an arenavirus particle, wherein the method comprises:
a) transfecting into a host cell one or more DNA sequences provided herein or one or more RNA sequences each transcribed in vitro from the DNA sequence provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more DNA sequences are transcribed using a bidirectional promoter. In certain embodiments, the one or more DNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the nucleotide sequence provided herein.

In certain embodiments, provided herein is a method of rescuing an arenavirus particle using the nucleotide sequence provided herein.

In certain embodiments, provided herein is a host cell comprising the nucleotide sequence provided herein, the translation product provided herein, the arenavirus particle provided herein, or the DNA expression vector provided herein.

In certain embodiments, provided herein is a vaccine comprising the nucleotide sequence provided herein, the translation product provided herein, the arenavirus particle provided herein, and a pharmaceutically acceptable carrier.

In certain embodiments, provided herein is a pharmaceutical composition comprising the nucleotide sequence provided herein, the translation product provided herein, the arenavirus particle provided herein, and a pharmaceutically acceptable carrier.

### 5.6 Arenavirus Particle Provided Herein

In another aspect, provided herein are arenavirus particles. In certain embodiments, provided herein is an arenavirus particle engineered such that an arenaviral ORF is separated over two or more mRNA transcripts.

In certain embodiments, at least one of the mRNA transcripts comprises an internal ribosome entry site (IRES).

In certain embodiments, the mRNA transcripts can be transcribed from the arenavirus genomic or antigenomic segment. In certain embodiments, the arenavirus genomic or antigenomic segment is an S segment. In certain embodiments, the arenavirus genomic or antigenomic segment is an L segment.

In certain embodiments, the two or more mRNA transcripts are under control of an arenavirus 3′ UTR or an arenavirus 5′ UTR.

In certain embodiments, the arenaviral ORF encodes a polypeptide comprising an amino acid sequence that is at least 80%, 85%, 90%, 95%, 96%, 97%, 98%, 99% and 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is identical to SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the arenaviral ORF encodes arenavirus GP signal peptide and a buffer peptide, arenavirus GP1 and GP2 and the arenavirus GP signal peptide and a buffer peptide or a functional fragment thereof is expressed from a first mRNA transcript (e.g., viral mRNA transcript) and arenavirus GP1 and GP2 are expressed from a second mRNA transcript (e.g., viral mRNA transcript). In some embodiments, the GP signal peptide (or functional fragment thereof or polypeptide comprising the GP signal peptide or functional fragment thereof) is cleaved from the buffer peptide. In some embodiments, a peptidase can cleave the GP signal peptide (or functional fragment thereof or polypeptide comprising the GP signal peptide or functional fragment thereof) from the buffer peptide (eg, −5, −4, −3, −2, −1, +1, +2, +3, +4, or +5 amino acid position from the fusion site, or at the fusion site). In some embodiments, an arenavirus as described herein wherein the GP signal peptide is fused to a buffer peptide (i) grows to higher titers, and/or (ii) reaches peak titers earlier, and/or (iii) exhibits a higher rescue efficiency (i.e., higher percentage of RCV titer-positive rescues of total rescues), as compared to a control arenavirus that has the same genomic organization except it lacks a buffer peptide.

In certain embodiments, the first mRNA transcript is under control of an arenavirus 3′ UTR.

In certain embodiments, the second mRNA transcript further encodes a heterologous non-arenaviral signal peptide.

In certain embodiments, the heterologous non-arenaviral signal peptide is the signal peptide of the vesicular stomatitis virus serotype Indiana glycoprotein.

In certain embodiments, the first mRNA transcript further comprises a nucleotide sequence encoding a heterologous non-arenaviral polypeptide or arenavirus GP, NP, Z and L.

In certain embodiments, the heterologous non-arenaviral polypeptide is an antigen derived from an infectious organism, tumor, or allergen. In certain embodiments, the antigen is selected from the group consisting of
(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPDl, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMIB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

In certain embodiments, the expression level of the heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express a heterologous non-arenaviral polypeptide after an arenavirus particle of the present disclosure (e.g., an arenavirus expressing a heterologous non-arenaviral polypeptide under control of an arenavirus 3′ UTR) is introduced to (or infects) the cells (e.g., to a population of cells). In certain embodiments, the proportion of cells that express a heterologous non-arenaviral polypeptide after an arenavirus particle of the present disclosure (e.g., expressing a heterologous non-arenaviral polypeptide under control of an arenavirus 3′ UTR) is introduced to (or infects) a population of cells is higher compared to the proportion of cells that express the heterologous non-arenaviral polypeptide after an arenavirus particle expressing the same heterologous non-arenaviral polypeptide expressed under control of an arenavirus 5′ UTR is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express a heterologous non-arenaviral polypeptide after an arenavirus particle of the present disclosure (e.g., expressing a heterologous non-arenaviral polypeptide under control of an arenavirus 3′ UTR) is introduced to (or infects) a population of cells as compared to the proportion of cells that express the heterologous non-arenaviral polypeptides after an arenavirus particle expressing the same heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the expression level of a heterologous non-arenaviral polypeptide expressed under control of an arenavirus 3′ UTR and/or the expression level of a second heterologous non-arenaviral polypeptide expressed under control of an arenavirus 3′ UTR is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR and/or higher than the expression level of the same second heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express the heterologous non-arenaviral polypeptide under control of an arenavirus 3′ UTR and/or express the second heterologous non-arenaviral polypeptide under control of an arenavirus 3′ UTR after an arenavirus particle of the present disclosure (e.g., an arenavirus expressing the heterologous non-arenaviral polypeptide under control of an arenavirus 3′ UTR and/or an arenavirus expressing the second heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide and/or express the second heterologous non-arenaviral polypeptide after an arenavirus particle of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells is higher compared to the proportion of cells that express the heterologous non-arenaviral polypeptide and/or express the second heterologous non-arenaviral polypeptide after an arenavirus particle expressing the same heterologous non-arenaviral polypeptide under control of an arenavirus 5′ UTR and/or expressing the same second heterologous non-arenaviral polypeptide under control of an arenavirus 5′ UTR is introduced to (or infects) a population of cells. In certain embodiments, at least about 1%, 2%, 3%, 4%, 5%, 6%, 7%, 8%, 9%, 10%1, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide and/or express the second heterologous non-arenaviral polypeptide after an arenavirus particle of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells as compared to the proportion (or amount) of cells that express the heterologous non-arenaviral polypeptides and/or express the second heterologous non-arenaviral polypeptides after an arenavirus particle expressing the same heterologous non-arenaviral polypeptide under control of an arenavirus 5′ UTR and/or expressing the same second heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle expresses two heterologous non-arenaviral polypeptides. In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment. In certain embodiments, the expression of a first of the two heterologous non-arenaviral polypeptides is under control of an arenavirus 5′ UTR in a first S segment and the expression of a second of the two heterologous non-arenaviral polypeptides is under control of an arenavirus 3′ UTR in a second S segment. In certain embodiments, the combined expression level of the two heterologous non-arenaviral polypeptides is at least about, or about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptide expressed under control of arenavirus 5′ UTRs. In certain embodiments, the expression of a first of the same two heterologous non-arenaviral polypeptides is under control of an arenavirus 5′ UTR in a first S segment and the expression of a second of the same two heterologous non-arenaviral polypeptides is under control of an arenavirus 5′ UTR in a second S segment.

In certain embodiments, one heterologous non-arenaviral polypeptide is expressed under control of an arenavirus 3′ UTR and the other heterologous non-arenaviral polypeptide is expressed under control of an arenavirus 5′ UTR. In certain embodiments, the heterologous non-arenaviral polypeptide is expressed under control of an arenavirus 3′ UTR and the second heterologous non-arenaviral polypeptide is expressed under control of an arenavirus 5′ UTR. In certain embodiments, the heterologous non-arenaviral polypeptide is expressed under control of an arenavirus 3′ UTR in one S segment and the second heterologous non-arenaviral polypeptide is expressed under control of an arenavirus 5′ UTR in another S segment. In certain embodiments, the heterologous non-arenaviral polypeptide is expressed under control of an arenavirus 5′ UTR in one S segment and the second heterologous non-arenaviral polypeptide is expressed under control of an arenavirus 3′ UTR in another S segment. In certain embodiments, the proportion of cells that express both the heterologous non-arenaviral polypeptide and the second heterologous non-arenaviral polypeptide (e.g., cells that co-express the heterologous non-arenaviral polypeptide and the second heterologous non-arenaviral polypeptide) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the proportion of cells that express both the same heterologous non-arenaviral polypeptide and the same second heterologous non-arenaviral polypeptide expressed under control of arenavirus 5′ UTRs (i.e., the same heterologous non-arenaviral polypeptide is expressed under control of an arenavirus 5′ UTR in one S segment and the same second heterologous non-arenaviral polypeptide is expressed under control of an arenavirus 5′ UTR in another S segment). In some embodiments, the proportion of cells is determined after a population of cells are infected with an arenavirus of the present disclosure (e.g., one heterologous non-arenaviral polypeptide is expressed under control of an arenavirus 3′ UTR and the other heterologous non-arenaviral polypeptide is expressed under control of an arenavirus 5′ UTR). In some embodiments, the proportion of cells is determined after a population of cells are infected with another arenavirus (e.g., both heterologous non-arenaviral polypeptides expressed under control of an arenavirus 5′ UTR).

In certain embodiments, at least about, or about, 5%, 10%, 15%, 20%, 25%, 30%, 35%, 40%, 45%, 50%, 55%, 60%, 65%, 70%, 75%, 80%, 8% 5, ⁹ 0, 95% 99%, or 100% of cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion of cells that express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a population of cells. In certain embodiments, at least about 1%, 2%, 3%, 4%, 5%, 6%, 7%, 8%, 9%0, 10%, 15%, 20%, 25%, 30%, 35%, 40%, 45%, 50%, 55%, 60%, 65%, 70%, 75%, 80%, 85%, 90%, 95%, 99%, or 100% more cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion (or amount) of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF. In some embodiments, the genome of the second arenavirus particle does not encode a buffer peptide.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

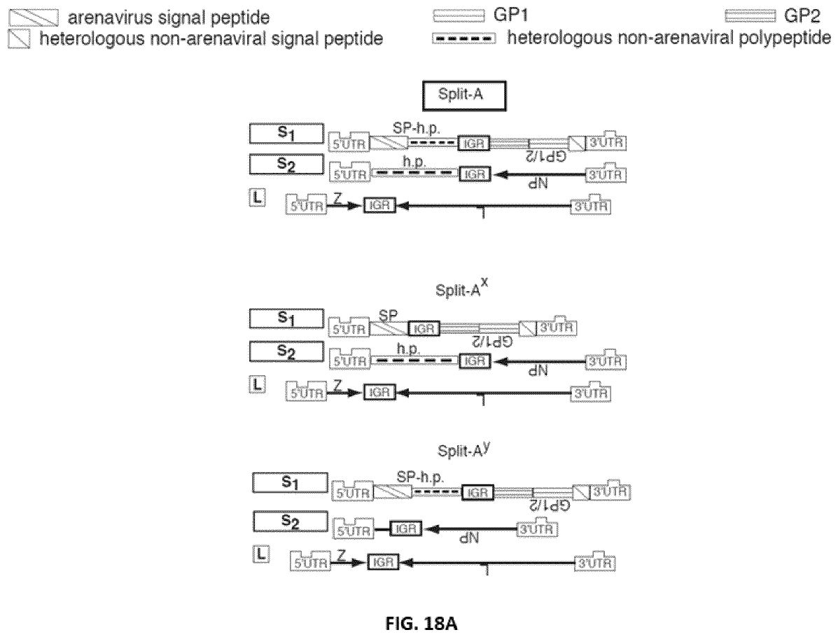

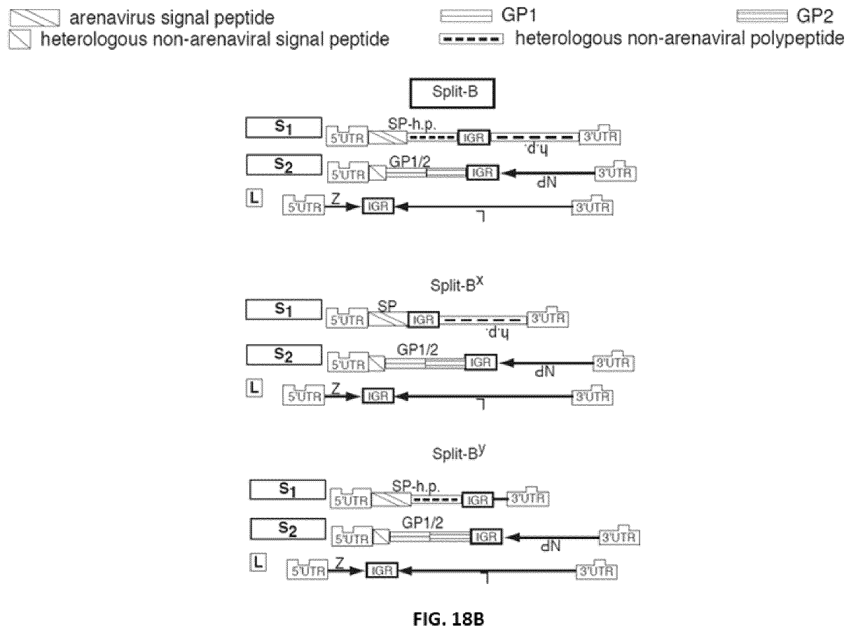

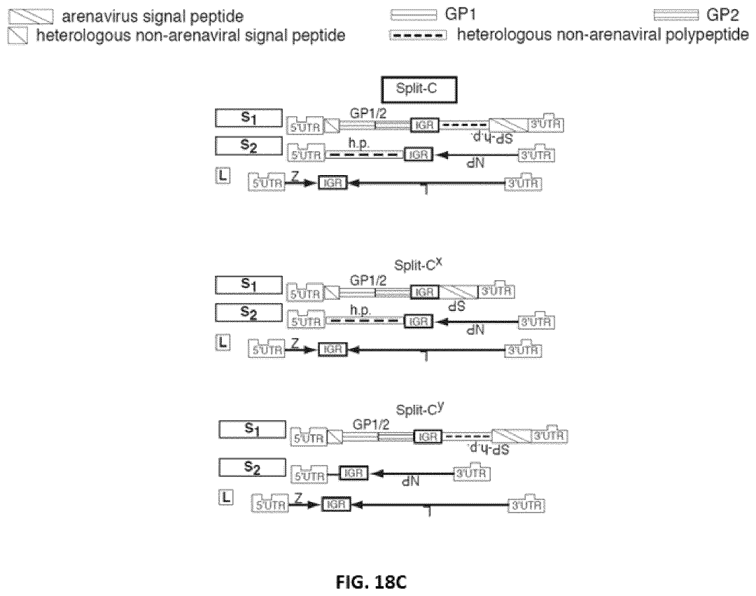

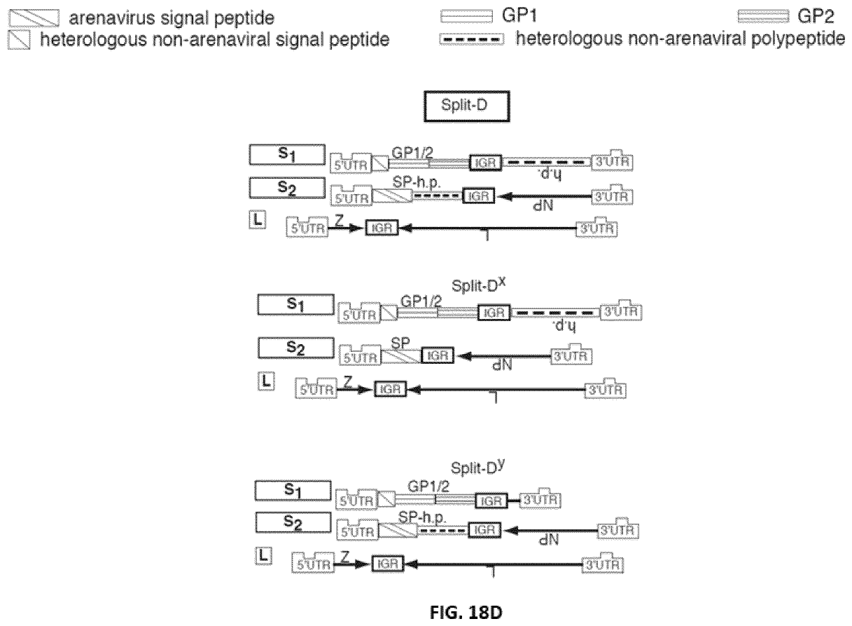

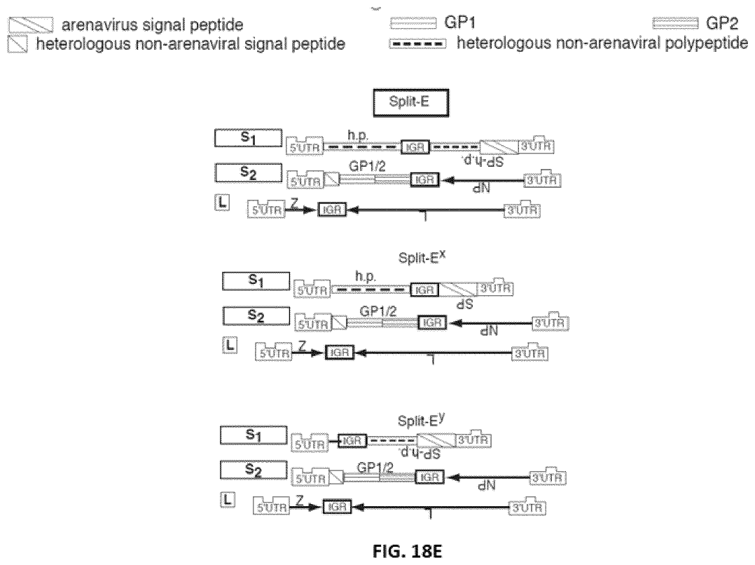

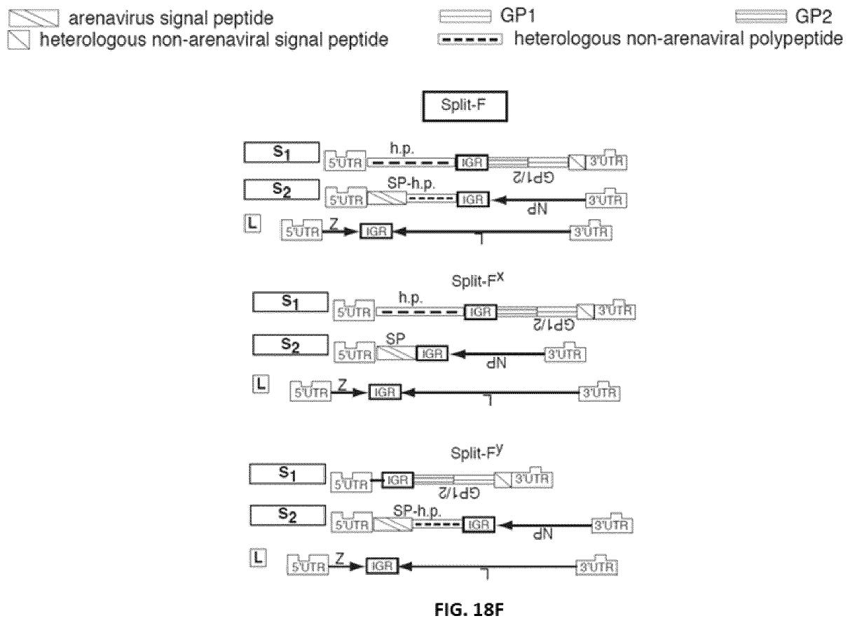

In certain embodiments, the arenavirus particle comprises a genome organization as outlined in FIG. 18C (with a buffer peptide). In certain embodiments, the arenavirus particle comprises a genome organization as outlined in FIG. 18E (with a buffer peptide). FIGS. 18C and 18E show a general genomic organization, and while the buffer peptide is not present in FIGS. 18C and 18E, a buffer peptide can be present following the GP signal peptide.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and wherein the two heterologous non-arenaviral polypeptides are the same or different from each other.

In certain embodiments, the genome of the arenavirus particle consists of
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR;
b) an S segment that encodes NP under the control of an arenavirus genomic 3′ UTR and a second heterologous non-arenaviral polypeptide or no polypeptide under the control of an arenavirus genomic 5′ UTR; and
c) an L segment that encodes the L protein under the control of an arenavirus genomic 3′ UTR and the Z protein under the control of an arenavirus genomic 5′ UTR; wherein the first heterologous non-arenaviral polypeptide and the second heterologous non-arenaviral polypeptide are the same or different from each other.

In certain embodiments, the genome of the arenavirus particle consists of
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR;
b) an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and
c) an L segment that encodes the L protein under the control of an arenavirus genomic 3′ UTR and the Z protein under the control of an arenavirus genomic 5′ UTR; wherein the first heterologous non-arenaviral polypeptide and the second heterologous non-arenaviral polypeptide are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle of the disclosure is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, provided herein is an arenavirus particle engineered such that an arenaviral ORF is separated over two or more mRNA transcripts. In certain embodiments, provided herein is an arenavirus particle comprising the nucleotide sequence provided herein (see Section 5.5). In certain embodiments, provided herein is an arenavirus particle comprising the arenavirus genomic or antigenomic segment provided herein (see Section 5.7). The arenavirus particles described herein are genetically stable and provide high-level transgene expression.

In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments. In certain embodiments, the tri-segmented arenavirus particle comprises arenaviral ORFs encoding arenavirus GP, NP, Z and L. In certain embodiments, the tri-segmented arenavirus particle expresses one or more heterologous non-arenaviral polypeptide. In certain embodiments, the tri-segmented arenavirus particle described herein may consist of six positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus S segment 5′ UTR; position 4 is under the control of an arenavirus S segment 3′ UTR; position 5 is under the control of an arenavirus L segment 5′ UTR; position 6 is under the control of an arenavirus L segment 3′ UTR. In certain embodiments, positions 1-6 may encode the same polypeptide in two or more positions. In other embodiments, positions 1-6 may each encodes a different polypeptide. In certain embodiments, the tri-segmented arenavirus particle comprising positions 1-6 in the arenavirus genomic or antigenomic segments is replication competent. In other embodiments, the tri-segmented arenavirus particle comprising positions 1-6 in the arenavirus genomic or antigenomic segments is replication-defective.

In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the genome of a bi-segmented arenavirus particle may consist of four positions in the arenavirus genomic or antigenomic segments, for example, position 1 is under the control of an arenavirus S segment genomic 5′ UTR; position 2 is under the control of an arenavirus S segment genomic 3′ UTR; position 3 is under the control of an arenavirus L segment genomic 5′ UTR; position 4 is under the control of an arenavirus L segment genomic 3′ UTR. In certain embodiments, positions 1-4 may encode the same polypeptide in two or more positions. In other embodiments, positions 1-4 may each encode a different polypeptide. In certain embodiments, the bi-segmented arenavirus particle comprising positions 1-4 in the arenavirus genomic or antigenomic segments is replication competent. In other embodiments, the bi-segmented arenavirus particle comprising positions 1-4 in the arenavirus genomic or antigenomic segments is replication-defective. In some embodiments, the bi-segmented arenavirus depends on trans-complementation by a genetically engineered helper cell in order to form infectious progeny particles.

In certain embodiments, the arenavirus particle is derived from a Lymphocytic choriomeningitis virus (LCMV). In certain embodiments, the LCMV is a MP strain, an Armstrong strain, or an Armstrong Clone 13 strain. In other embodiments, the arenavirus particle is derived from a Lassa virus. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the growth or infectivity of the arenavirus particle comprising the nucleotide sequence described herein is not inferior to a second arenavirus particle carrying one or more of the same heterologous non-arenaviral polypeptides, and all arenaviral GP, NP, Z and L in the second arenavirus particle are each expressed as one ORF. In certain embodiments, the growth or infectivity of the arenavirus particle comprising the nucleotide sequence described herein is not inferior to a second arenavirus particle, wherein the second arenavirus particle does not comprise a nucleotide sequence of a buffer peptide.

### 5.6.1 Arenaviral Open Reading Frame

In certain embodiments, the arenaviral ORF encodes a polypeptide selected from the group consisting of arenavirus GP, NP, Z, and L (see Section 5.1).

In certain embodiments, the arenaviral ORF encodes a polypeptide selected from the group consisting of arenavirus GP, NP, Z, and L, namely from the group consisting of arenavirus glycoprotein, nucleoprotein, Z protein, L protein, glycoprotein precursor, nucleoprotein precursor, Z protein precursor, and L protein precursor. In certain embodiments, the arenavirus GP, NP, Z, and L are wild-type. In other embodiments, the arenavirus GP, NP, Z, and L are recombinant. In certain embodiments, the arenavirus GP, NP, Z, and L are mutated. In certain embodiments, the arenavirus GP, NP, Z, and L are derived from an attenuated virus.

In certain embodiments, the arenaviral ORF encodes a polypeptide selected from the group consisting of wild-type arenavirus glycoprotein, nucleoprotein, Z protein, L protein, glycoprotein precursor, nucleoprotein precursor, Z protein precursor, and L protein precursor. In certain embodiments, the arenaviral ORF encodes a wild-type arenavirus glycoprotein precursor. In certain embodiments, the wild-type arenavirus glycoprotein precursor can be processed into a GP signal peptide, a GP1 and a GP2.

In certain embodiments, the arenaviral ORF encodes a polypeptide selected from the group consisting of recombinant arenavirus glycoprotein, nucleoprotein, Z protein, L protein, glycoprotein precursor, nucleoprotein precursor, Z protein precursor, and L protein precursor. In certain embodiments, the arenaviral ORF encodes a recombinant arenavirus glycoprotein precursor. In certain embodiments, the recombinant arenavirus glycoprotein precursor can be processed into a GP signal peptide, a GP1 and a GP2, one or more of which are recombinant.

In certain embodiments, the arenaviral ORF encodes a polypeptide selected from the group consisting of mutated arenavirus glycoprotein, nucleoprotein, Z protein, L protein, glycoprotein precursor, nucleoprotein precursor, Z protein precursor, and L protein precursor. In certain embodiments, the arenaviral ORF encodes a mutated arenavirus glycoprotein precursor. In certain embodiments, the mutated arenavirus glycoprotein precursor can be processed into a GP signal peptide, a GP1 and a GP2, one or more of which are mutated.

In certain embodiments, the arenaviral ORF encodes a polypeptide selected from the group consisting of arenavirus glycoprotein, nucleoprotein, Z protein, L protein, glycoprotein precursor, nucleoprotein precursor, Z protein precursor, and L protein precursor that are derived from an attenuated virus. In certain embodiments, the arenaviral ORF encodes arenavirus glycoprotein precursor derived from an attenuated virus. In certain embodiments, the arenavirus glycoprotein precursor derived from an attenuated virus can be processed into a GP signal peptide, a GP1 and a GP2, one or more of which are derived from an attenuated virus.

In certain embodiments, the arenaviral ORF encodes a polypeptide selected from the group consisting of GP, NP, Z and L of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, the arenaviral ORF encodes a polypeptide comprising an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO:126, SEQ ID NO:133, or SEQ ID NO: 140. In certain embodiments, the arenaviral ORF encodes a polypeptide comprising an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO:126, SEQ ID NO:133, or SEQ ID NO: 140. In certain embodiments, the arenaviral ORF encodes a polypeptide comprising an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO: 109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenaviral ORF encodes a polypeptide comprising an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenaviral ORF encodes a polypeptide comprising an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenaviral ORF encodes a polypeptide comprising an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenaviral ORF encodes a polypeptide comprising an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO: 112, SEQ ID NO: 119, SEQ ID NO:126, SEQ ID NO:133, or SEQ ID NO: 140. In certain embodiments, the arenaviral ORF encodes a polypeptide comprising an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO: 108, SEQ ID NO:112, SEQ ID NO:119, SEQ ID NO:126, SEQ ID NO:133, or SEQ ID NO:140.

In certain embodiments, the arenaviral ORF encodes arenavirus GP, namely arenavirus glycoprotein or any glycoprotein precursor. In certain embodiments, the arenaviral ORF encodes a wild-type arenavirus glycoprotein or any wild-type glycoprotein precursor. In other embodiments, the arenaviral ORF encodes a recombinant arenavirus glycoprotein or any recombinant glycoprotein precursor. In certain embodiments, the arenavirus glycoprotein precursor can be processed into a GP signal peptide, a GP1 and a GP2. In certain embodiments, the arenavirus GP is arenavirus glycoprotein or any glycoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus GP as described herein comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is at least 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is at least 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO: 134. In certain embodiments, the arenavirus GP as described herein comprises an amino acid sequence that is at least 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is at least 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the GP as described herein comprises an amino acid sequence that is 80% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 85% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 90% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 95% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 96% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 97% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134. In certain embodiments, the arenavirus GP as described herein comprises an amino acid sequence that is 98% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO: 113, SEQ ID NO:120, SEQ ID NO:127, or SEQ ID NO:134. In certain embodiments, the arenavirus GP as described herein comprises an amino acid sequence that is 99% identical to the amino acid sequence of SEQ ID NO:1, SEQ ID NO:2, SEQ ID NO:3, SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO: 105, SEQ ID NO:109, SEQ ID NO:113, SEQ ID NO:120, SEQ ID NO:127, or SEQ ID NO: 134.

In certain embodiments, the arenaviral ORF encodes arenavirus NP, namely arenavirus nucleoprotein or any nucleoprotein precursor. In certain embodiments, the arenaviral ORF encodes a wild-type arenavirus nucleoprotein or any wild-type nucleoprotein precursor. In other embodiments, the arenaviral ORF encodes a recombinant arenavirus nucleoprotein or any recombinant nucleoprotein precursor. In certain embodiments, the arenavirus NP is arenavirus nucleoprotein or any nucleoprotein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus NP as described herein comprises an amino acid sequence that is at least 80%, 85%, 90%, 95%, 96%, 97%, 98%, 99% or 100% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 80% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 85% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 90% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 95% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is at least 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is at least 99% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 100% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 80% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 85% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 90% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 95% identical to the amino acid sequence of SEQ ID NO:7, SEQ ID NO:8, SEQ ID NO:9, SEQ ID NO:10, SEQ ID NO: 11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 96% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 97% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO:138. In certain embodiments, the arenavirus NP as described herein comprises an amino acid sequence that is 98% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO:117, SEQ ID NO:124, SEQ ID NO:131, or SEQ ID NO:138. In certain embodiments, the arenavirus NP as described herein comprises an amino acid sequence that is 99% identical to the amino acid sequence of SEQ ID NO:7, SEQ ID NO:8, SEQ ID NO:9, SEQ ID NO:10, SEQ ID NO:11, SEQ ID NO:12, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO: 106, SEQ ID NO:110, SEQ ID NO: 117, SEQ ID NO:124, SEQ ID NO:131, or SEQ ID NO: 138.

In certain embodiments, the arenaviral ORF encodes arenavirus Z, namely arenavirus Z protein or any Z protein precursor. In certain embodiments, the polypeptide described herein is a wild-type arenavirus Z protein or any wild-type Z protein precursor. In certain embodiments, the arenaviral ORF encodes a recombinant arenavirus Z protein or any recombinant Z protein precursor. In certain embodiments, the arenavirus Z is arenavirus Z protein or any Z protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus Z as described herein comprises an amino acid sequence that is at least 80%, 85%, 90%, 95%, 96%, 97%, 98%, 99% or 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO: 139. In certain embodiments, the arenavirus Z as described herein comprises an amino acid sequence that is at least 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is at least 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is at least 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO: 103, SEQ ID NO:107, SEQ ID NO:111, SEQ ID NO: 118, SEQ ID NO:125, SEQ ID NO:132, or SEQ ID NO:139. In certain embodiments, the arenavirus Z as described herein comprises an amino acid sequence that is 100% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO: 107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 80% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 85% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 90% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 95% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO: 139. In certain embodiments, the arenavirus Z as described herein comprises an amino acid sequence that is 96% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 97% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 98% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139. In certain embodiments, the arenavirus Z as described herein comprises an amino acid sequence that is 99% identical to the amino acid sequence of SEQ ID NO:13, SEQ ID NO:14, SEQ ID NO:15, SEQ ID NO:16, SEQ ID NO:17, SEQ ID NO:18, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, or SEQ ID NO:139.

In certain embodiments, the arenaviral ORF encodes arenavirus L, namely arenavirus L protein or any L protein precursor. In certain embodiments, the arenaviral ORF encodes a wild-type arenavirus L protein or any wild-type L protein precursor. In certain embodiments, the arenaviral ORF encodes a recombinant arenavirus L protein or any recombinant L protein precursor. In certain embodiments, the arenavirus L is arenavirus L protein or any L protein precursor of LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus. In certain embodiments, the arenavirus L as described herein comprises an amino acid sequence that is at least 80%, 85%, 90%, 95%, 96%, 97%, 98%, 99% or 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenavirus L as described herein comprises an amino acid sequence that is at least 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenavirus L as described herein comprises an amino acid sequence that is at least 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is at least 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO: 119, SEQ ID NO:126, SEQ ID NO:133, or SEQ ID NO:140. In certain embodiments, the arenavirus L as described herein comprises an amino acid sequence that is 100% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO: 140. In certain embodiments, the arenavirus L as described herein comprises an amino acid sequence that is 80% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 85% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 90% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 95% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO: 140. In certain embodiments, the arenavirus L as described herein comprises an amino acid sequence that is 96% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 97% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140. In certain embodiments, the arenavirus L as described herein comprises an amino acid sequence that is 98% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO: 112, SEQ ID NO: 119, SEQ ID NO:126, SEQ ID NO:133, or SEQ ID NO:140. In certain embodiments, the arenavirus L as described herein comprises an amino acid sequence that is 99% identical to the amino acid sequence of SEQ ID NO:19, SEQ ID NO:20, SEQ ID NO:21, SEQ ID NO:22, SEQ ID NO:23, SEQ ID NO:24, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

### 5.6.2 Arenavirus Genomic or Antigenomic Segments of the Arenavirus Particle Provided Herein

In certain embodiments, the arenavirus particle is bi-segmented, namely consists of two arenavirus genomic or antigenomic segments. In certain embodiments, the two arenavirus genomic or antigenomic segments are one S segment and one L segment. In certain embodiments, the arenavirus particle is tri-segmented, namely consists of three arenavirus genomic or antigenomic segments. In certain embodiments, the three arenavirus genomic or antigenomic segments are two S segments and one L segment. In other embodiments, the three arenavirus genomic or antigenomic segments are one S segment and two L segments.

In certain embodiments, the genome of the arenavirus particle may consist of a) an S segment that encodes a heterologous non-arenaviral signal peptide, arenavirus GP1 and arenavirus GP2 under the control of 3′ UTR, and arenavirus GP signal peptide, a buffer peptide, and a heterologous non-arenaviral polypeptide or arenavirus GP signal peptide and a buffer peptide alone under the control of 5′ UTR; b) an S segment that encodes NP under the control of 3′ UTR, and another heterologous non-arenaviral polypeptide or no polypeptide under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may comprise arenavirus genomic or antigenomic segments depicted in FIG. 18A. FIG. 18A shows a general genomic organization, and while the buffer peptide is not present in FIG. 18A, a buffer peptide can be present following the GP signal peptide.

In certain embodiments, the genome of the arenavirus particle may consist of a) an S segment that encodes a heterologous non-arenaviral polypeptide or no polypeptide under the control of 3′ UTR, and arenavirus GP signal peptide, a buffer peptide, and a heterologous non-arenaviral polypeptide or arenavirus GP signal peptide and a buffer peptide alone under the control of 5′ UTR; b) an S segment that encodes NP under the control of 3′ UTR, and a heterologous non-arenaviral signal peptide, arenavirus GP1 and arenavirus GP2 under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may comprise arenavirus genomic or antigenomic segments depicted in FIG. 18B. FIG. 18B shows a general genomic organization and while the buffer peptide is not present in FIG. 18B, a buffer peptide can be present following the GP signal peptide. In certain embodiments, the genome of the arenavirus particle may consist of a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR; b) an S segment that encodes NP under the control of an arenavirus genomic 3′ UTR and a second heterologous non-arenaviral polypeptide or no polypeptide under the control of an arenavirus genomic 5′ UTR; and c) an L segment that encodes the L protein under the control of an arenavirus genomic 3′ UTR and the Z protein under the control of an arenavirus genomic 5′ UTR. In some embodiments, the first heterologous non-arenaviral polypeptide and the second heterologous non-arenaviral polypeptide are the same. In some embodiments, the first heterologous non-arenaviral polypeptide and the second heterologous non-arenaviral polypeptide are different from each other.

In certain embodiments, the genome of the arenavirus particle may consist of a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR; b) an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and c) an L segment that encodes the L protein under the control of an arenavirus genomic 3′ UTR and the Z protein under the control of an arenavirus genomic 5′ UTR. In some embodiments, the first heterologous non-arenaviral polypeptide and the second heterologous non-arenaviral polypeptide are the same. In some embodiments, the first heterologous non-arenaviral polypeptide and the second heterologous non-arenaviral polypeptide are different from each other.

In some embodiments, the GP signal peptide (or functional fragment thereof or polypeptide comprising the GP signal peptide or functional fragment thereof) is cleaved from the buffer peptide. In some embodiments, a peptidase can cleave the GP signal peptide (or functional fragment thereof or polypeptide comprising the GP signal peptide or functional fragment thereof) from the buffer peptide (eg, −5, −4, −3, −2, −1, +1, +2, +3, +4, or +5 amino acid position from the fusion site, or at the fusion site). In some embodiments, an arenavirus particle as described herein wherein the GP signal peptide is fused to a buffer peptide (i) grows to higher titers, and/or (ii) reaches peak titers earlier, and/or (iii) exhibits a higher rescue efficiency (i.e., higher percentage of RCV titer-positive rescues of total rescues), as compared to a control arenavirus particle that has the same genomic organization except it lacks a buffer peptide.

In certain embodiments, the genome of the arenavirus particle may consist of a) an S segment that encodes arenavirus GP signal peptide, a buffer peptide, and a heterologous non-arenaviral polypeptide or an arenavirus GP signal peptide and a buffer peptide alone under the control of genomic 3′ UTR, and a heterologous non-arenaviral signal peptide, arenavirus GP1 and arenavirus GP2 under the control of genomic 5′ UTR; b) an S segment that encodes NP under the control of genomic 3′ UTR, and another heterologous non-arenaviral polypeptide or no polypeptide under the control of genomic 5′ UTR; and c) an L segment that encodes L protein under the control of 3′ UTR, and Z protein under the control of genomic 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 18C. FIG. 18C shows a general genomic organization and while the buffer peptide is not present in FIG. 18C, a buffer peptide can be present following the GP signal peptide.

In certain embodiments, the genome of the arenavirus particle may consist of a) an S segment that encodes a heterologous non-arenaviral polypeptide or no polypeptide under the control of 3′ UTR, and a heterologous non-arenaviral signal peptide, arenavirus GP1 and arenavirus GP2 under the control of 5′ UTR; b) an S segment that encodes NP under the control of 3′ UTR, and arenavirus GP signal peptide, a buffer peptide, and a heterologous non-arenaviral polypeptide or arenavirus GP signal peptide and a buffer peptide alone under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 18D. FIG. 18D shows a general genomic organization and while the buffer peptide is not present in FIG. 18D, a buffer peptide can be present following the GP signal peptide.

In certain embodiments, the genome of the arenavirus particle may consist of a) an S segment that encodes arenavirus GP signal peptide, a buffer peptide, and a heterologous non-arenaviral polypeptide or arenavirus GP signal peptide and a buffer peptide alone under the control of 3′ UTR, and another heterologous non-arenaviral polypeptide or no polypeptide under the control of 5′ UTR; b) an S segment that encodes NP under the control of 3′ UTR, and a heterologous non-arenaviral signal peptide, arenavirus GP1 and arenavirus GP2 under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 18E. FIG. 18E shows a general genomic organization and while the buffer peptide is not present in FIG. 18E, a buffer peptide can be present following the GP signal peptide.

In certain embodiments, the genome of the arenavirus particle may consist of a) an S segment that encodes a heterologous non-arenaviral signal peptide, arenavirus GP1 and arenavirus GP2 under the control of 3′ UTR, and a heterologous non-arenaviral polypeptide or no polypeptide under the control of 5′ UTR; b) an S segment that encodes NP under the control of 3′ UTR, and arenavirus GP signal peptide, a buffer peptide, and a heterologous non-arenaviral polypeptide or arenavirus GP signal peptide and a buffer peptide alone under the control of 5′ UTR; and c) an L segment that encodes L protein under the control of 3′ UTR, and Z protein under the control of 5′ UTR. In certain embodiments, the two heterologous non-arenaviral polypeptides are the same. In other embodiments, the two heterologous non-arenaviral polypeptides are different from each other. In certain embodiments, the genome of the arenavirus particle may consist of arenavirus genomic or antigenomic segments depicted in FIG. 18F. FIG. 18F shows a general genomic organization and while the buffer peptide is not present in FIG. 18F, a buffer peptide can be present following the GP signal peptide.

In certain embodiments, the arenavirus particle comprising the nucleotide sequence described herein is infectious and replication competent. In certain embodiments, the arenavirus particle comprising the nucleotide sequence described herein is attenuated as compared to its parental virus. In certain embodiments, the arenavirus particle comprising the nucleotide sequence described herein is infectious but unable to produce further infectious progeny in non-complementing cells.

In certain embodiments, the genome of a bi-segmented arenavirus particle may consist of four positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus L segment 5′ UTR; position 4 is under the control of an arenavirus L segment 3′ UTR. In certain embodiments, positions 1-4 may encode the same polypeptide in two or more positions. In other embodiments, positions 1-4 may each encode a different polypeptide. In certain embodiments, the bi-segmented arenavirus particle comprising positions 1-4 in the arenavirus genomic or antigenomic segments is replication competent. In other embodiments, the bi-segmented arenavirus particle comprising positions 1-4 in the arenavirus genomic or antigenomic segments is replication-defective. In some embodiments, the bi-segmented arenavirus depends on trans-complementation by a genetically engineered helper cell in order to form infectious progeny particles.

In certain embodiments, the genome of a tri-segmented arenavirus particle may consist of six positions in the arenavirus genomic or antigenomic segments, for example, position 1 is under the control of an arenavirus S segment 5′ UTR; position 2 is under the control of an arenavirus S segment 3′ UTR; position 3 is under the control of an arenavirus S segment 5′ UTR; position 4 under the control of an arenavirus S segment 3′ UTR; position 5 is under the control of an arenavirus L segment 5′ UTR; position 6 is under the control of an arenavirus L segment 3′ UTR. In certain embodiments, positions 1-6 may encode the same polypeptide in two or more positions. In other embodiments, positions 1-6 may each encode a different polypeptide. In certain embodiments, the tri-segmented arenavirus particle comprising positions 1-6 in the arenavirus genomic or antigenomic segments is replication competent. In other embodiments, the tri-segmented arenavirus particle comprising positions 1-6 in the arenavirus genomic or antigenomic segments is replication-defective.

In certain embodiments, position 1 encodes a heterologous non-arenaviral signal peptide, arenavirus GP1 and GP2. In certain embodiments, position 1 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 1 encodes a heterologous non-arenaviral polypeptide. In certain embodiments, position 1 encodes NP. In certain embodiments, position 1 encodes Z. In certain embodiments, position 1 encodes L. In certain embodiments, position 1 does not encode a polypeptide.

In certain embodiments, position 2 encodes a heterologous non-arenaviral signal peptide, arenavirus GP1 and GP2. In certain embodiments, position 2 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 2 encodes a heterologous non-arenaviral polypeptide. In certain embodiments, position 2 encodes NP. In certain embodiments, position 2 encodes Z. In certain embodiments, position 2 encodes L. In certain embodiments, position 2 does not encode a polypeptide.

In certain embodiments, position 3 encodes a heterologous non-arenaviral signal peptide, arenavirus GP1 and GP2. In certain embodiments, position 3 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 3 encodes a heterologous non-arenaviral polypeptide. In certain embodiments, position 3 encodes NP. In certain embodiments, position 3 encodes Z. In certain embodiments, position 3 encodes L. In certain embodiments, position 3 does not encode a polypeptide.

In certain embodiments, position 4 encodes a heterologous non-arenaviral signal peptide, arenavirus GP1 and GP2. In certain embodiments, position 4 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 4 encodes a heterologous non-arenaviral polypeptide. In certain embodiments, position 4 encodes NP. In certain embodiments, position 4 encodes Z. In certain embodiments, position 4 encodes L. In certain embodiments, position 4 does not encode a polypeptide.

In certain embodiments, position 5 encodes a heterologous non-arenaviral signal peptide, arenavirus GP1 and GP2. In certain embodiments, position 5 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 5 encodes a heterologous non-arenaviral polypeptide. In certain embodiments, position 5 encodes NP. In certain embodiments, position 5 encodes Z. In certain embodiments, position 5 encodes L. In certain embodiments, position 5 does not encode a polypeptide.

In certain embodiments, position 6 encodes a heterologous non-arenaviral signal peptide, arenavirus GP1 and GP2. In certain embodiments, position 6 encodes 1) arenavirus GP signal peptide, a buffer peptide, and a heterologous non-arenaviral polypeptide; 2) arenavirus GP signal peptide and a buffer peptide alone; or 3) arenavirus GP signal peptide, a buffer peptide, and NP, Z or L. In certain embodiments, position 6 encodes a heterologous non-arenaviral polypeptide. In certain embodiments, position 6 encodes NP. In certain embodiments, position 6 encodes Z. In certain embodiments, position 6 encodes L. In certain embodiments, position 6 does not encode a polypeptide.

In certain embodiments, the arenavirus particle is derived from a Lymphocytic choriomeningitis virus (LCMV). In certain embodiments, the LCMV is a MP strain, an Armstrong strain, or an Armstrong Clone 13 strain. In other embodiments, the arenavirus particle is derived from a Lassa virus. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from a Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the growth or infectivity of the arenavirus particle described herein is not inferior to a second arenavirus particle carrying one or more of the same heterologous non-arenaviral polypeptides, and all arenaviral GP, NP, Z and L in the second arenavirus particle are each expressed as one ORF. In certain embodiments, the growth or infectivity of the arenavirus particle described herein is not inferior to a second arenavirus particle. In some embodiments, the second arenavirus particle does not comprise a buffer peptide.

In certain embodiments, the arenavirus particle comprising the nucleotide sequence described herein is infectious and replication competent. In certain embodiments, the arenavirus particle comprising the nucleotide sequence described herein is attenuated as compared to its parental virus. In certain embodiments, the arenavirus particle comprising the nucleotide sequence described herein is infectious but unable to produce further infectious progeny in non-complementing cells.

### 5.6.3 Compositions and Methods Related to the Nucleotide Sequence Provided Herein

In certain embodiments, provided herein is a translation product of the mRNA transcripts of the genome of the arenavirus particle provided herein.

In certain embodiments, provided herein is a cDNA of the mRNA transcript of the genome of the arenavirus particle provided herein, wherein the cDNA can be transcribed into an arenavirus genomic or antigenomic segment.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA of the mRNA transcript of the genome of the arenavirus particle provided herein or one or more RNA sequences each transcribed in vitro from the cDNA of the mRNA transcript of the genome of the arenavirus particle provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising the DNA sequence encoding the mRNA transcript of the genome of the arenavirus particle provided herein.

In certain embodiments, provided herein is a method of rescuing an arenavirus particle using the mRNA transcript of the genome of the arenavirus particle provided herein or the cDNA sequence thereof.

In certain embodiments, provided herein is a host cell comprising the arenavirus particle provided herein, the translation product provided herein, the cDNA provided herein, or the DNA expression vector provided herein.

In certain embodiments, provided herein is a vaccine comprising the arenavirus particle provided herein, the translation product provided herein, the cDNA provided herein, or the DNA expression vector provided herein, and a pharmaceutically acceptable carrier.

In certain embodiments, provided herein is a pharmaceutical composition comprising the arenavirus particle provided herein, the translation product provided herein, the cDNA provided herein, or the DNA expression vector provided herein, and a pharmaceutically acceptable carrier.

### 5.7 Arenavirus Genomic or Antigenomic Segments Provided Herein

In another aspect, provided herein are arenavirus genomic or antigenomic segments. In certain embodiments, provided herein is an arenavirus genomic or antigenomic segment engineered such that the viral transcription thereof results in a first mRNA transcript and a second mRNA transcript,

wherein the first mRNA transcript comprises a nucleotide sequence encoding a functional fragment of a first polypeptide, and the first mRNA transcript does not encode the full-length first polypeptide;
wherein the second mRNA transcript comprises a nucleotide sequence encoding:
a) a second polypeptide; or
b) a functional fragment of the first polypeptide, and the second mRNA transcript does not encode the full-length first polypeptide; or
c) a functional fragment of a second polypeptide, and the second mRNA transcript does not encode the full-length second polypeptide; or
d) a heterologous non-arenaviral polypeptide; and
   wherein the first and second polypeptides are different from each other and selected from the group consisting of arenavirus GP, NP, Z and L.

In certain embodiments, the first mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the second mRNA transcript further comprises a nucleotide sequence encoding a second heterologous non-arenaviral polypeptide or a third polypeptide, wherein the third polypeptide is different from the first polypeptide and the second polypeptide and is selected from the group consisting of arenavirus GP, NP, Z and L; and wherein the heterologous non-arenaviral polypeptide and the second heterologous non-arenaviral polypeptide are the same or different from each other.

In certain embodiments, the arenavirus GP, NP, Z and L are from LCMV, Lassa virus, Pichinde virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus (ALLV), Alxa virus, Chapare virus, Lijiang virus, Cupixi virus, Gairo virus, Loei River virus, Lujo virus, Luna virus, Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, Xapuri virus, or Junin virus.

In certain embodiments, provided herein is an arenavirus genomic or antigenomic segment engineered such that the viral transcription thereof results in an mRNA transcript encoding:
a) a functional fragment of a first polypeptide, and
b) a heterologous non-arenaviral polypeptide or a second polypeptide;
   wherein the mRNA transcript does not encode the full-length first polypeptide; and wherein the first and second polypeptides are different from each other and selected from the group consisting of arenavirus GP, NP, Z and L.

In certain embodiments, the mRNA transcript is a first mRNA transcript and the viral transcription of the arenavirus genomic or antigenomic segment further results in a second mRNA transcript. In certain embodiments, the second mRNA transcript comprises a nucleotide sequence encoding a third polypeptide; a functional fragment of a third polypeptide; a functional fragment of the first polypeptide; or a second heterologous non-arenaviral polypeptide; wherein the third polypeptide is different from the first and the second polypeptide and selected from the group consisting of arenavirus GP, NP, Z and L, wherein the second mRNA transcript does not encode the full-length first polypeptide; wherein the second mRNA transcript does not encode the full-length third polypeptide; and wherein the heterologous non-arenaviral polypeptide and the second heterologous non-arenaviral polypeptide are the same or different from each other.

In certain embodiments, the second mRNA transcript comprises a nucleotide sequence encoding a functional fragment of a first polypeptide, and the functional fragment encoded by the first mRNA transcript is different from the functional fragment encoded by the second mRNA transcript.

In certain embodiments, the viral transcription of the arenavirus genomic or antigenomic segment does not further result in a second mRNA transcript.

In certain embodiments, the mRNA transcript comprises an internal ribosome entry site (IRES).

In certain embodiments, the arenavirus genomic or antigenomic segment is an S segment. In certain embodiments, the arenavirus genomic or antigenomic segment is an L segment.

In certain embodiments, the functional fragment is under control of an arenavirus 3′ UTR. In certain embodiments, the functional fragment is under control of an arenavirus 5′ UTR.

In certain embodiments, the first, second and third polypeptides each comprises an amino acid sequence that is at least 80%, 85%, 90%, 95%, 96%, 97%, 98%, 99% or 100% identical to the amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, or SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, SEQ ID NO:134, SEQ ID NO:49, SEQ ID NO:56, SEQ ID NO:63, SEQ ID NO:70, SEQ ID NO:77, SEQ ID NO:81, SEQ ID NO:88, SEQ ID NO:95, SEQ ID NO:102, SEQ ID NO:106, SEQ ID NO:110, SEQ ID NO: 117, SEQ ID NO:124, SEQ ID NO:131, SEQ ID NO:138, SEQ ID NO:50, SEQ ID NO:57, SEQ ID NO:64, SEQ ID NO:71, SEQ ID NO:78, SEQ ID NO:82, SEQ ID NO:89, SEQ ID NO:96, SEQ ID NO:103, SEQ ID NO:107, SEQ ID NO:111, SEQ ID NO:118, SEQ ID NO:125, SEQ ID NO:132, SEQ ID NO:139, SEQ ID NO:51, SEQ ID NO:58, SEQ ID NO:65, SEQ ID NO:72, SEQ ID NO:79, SEQ ID NO:83, SEQ ID NO:90, SEQ ID NO:97, SEQ ID NO:104, SEQ ID NO:108, SEQ ID NO:112, SEQ ID NO:119, SEQ ID NO:126, SEQ ID NO:133, or SEQ ID NO:140.

In certain embodiments, the first polypeptide comprises an amino acid sequence identical to SEQ ID NO:1, SEQ ID NO:2, SEQ ID NO:3, or SEQ ID NO:4, SEQ ID NO:5, SEQ ID NO:6, SEQ ID NO:48, SEQ ID NO:52, SEQ ID NO:59, SEQ ID NO:66, SEQ ID NO:73, SEQ ID NO:80, SEQ ID NO:84, SEQ ID NO:91, SEQ ID NO:98, SEQ ID NO:105, SEQ ID NO:109, SEQ ID NO:113, SEQ ID NO:120, SEQ ID NO:127, or SEQ ID NO:134.

In certain embodiments, the functional fragment of the first polypeptide comprises an amino acid sequence that is at least 80%, 85%, 90%, 95%, 96%, 97%, 98%, 99% or 100% identical to the amino acid sequence of SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:142, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:53, SEQ ID NO:60, SEQ ID NO:67, SEQ ID NO:74, SEQ ID NO:85, SEQ ID NO:92, SEQ ID NO:99, SEQ ID NO: 114, SEQ ID NO:121, SEQ ID NO:128, SEQ ID NO:135, SEQ ID NO:54, SEQ ID NO:61, SEQ ID NO:68, SEQ ID NO:75, SEQ ID NO:86, SEQ ID NO:93, SEQ ID NO:100, SEQ ID NO: 115, SEQ ID NO:122, SEQ ID NO:129, SEQ ID NO:136, SEQ ID NO:55, SEQ ID NO:62, SEQ ID NO:69, SEQ ID NO:76, SEQ ID NO:87, SEQ ID NO:94, SEQ ID NO:101, SEQ ID NO: 116, SEQ ID NO:123, SEQ ID NO:130, SEQ ID NO:137, SEQ ID NO:149, SEQ ID NO:154, SEQ ID NO:161, SEQ ID NO:166, SEQ ID NO:136, SEQ ID NO:143, SEQ ID NO:144, SEQ ID NO: 147, SEQ ID NO:148, SEQ ID NO:152, SEQ ID NO:153, SEQ ID NO:158, SEQ ID NO:159, SEQ ID NO:160, SEQ ID NO:164, SEQ ID NO:165, SEQ ID NO:135, SEQ ID NO:141, SEQ ID NO:142, SEQ ID NO:145, SEQ ID NO:146, SEQ ID NO:150, SEQ ID NO:151, SEQ ID NO:155, SEQ ID NO:156, SEQ ID NO:157, SEQ ID NO:162, or SEQ ID NO: 163.

In certain embodiments, the functional fragment is an arenavirus GP signal peptide or a functional fragment thereof. In certain embodiments, the functional fragment is an arenavirus GP signal peptide and a buffer peptide, or a functional fragment thereof.

In certain embodiments, the heterologous non-arenaviral polypeptide, the second heterologous non-arenaviral polypeptide, or both heterologous non-arenaviral polypeptides are each an antigen derived from an infectious organism, tumor, or allergen. In certain embodiments, the antigen is selected from the group consisting of
(a) viral antigens, and the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
(b) bacterial antigens, and the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
(c) tumor neoantigens or neo-epitopes and tumor associated antigens; and the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE Al, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMIB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

In certain embodiments, the first mRNA transcript comprises a nucleotide sequence encoding arenavirus GP signal peptide and a buffer peptide, either alone or fused to a heterologous non-arenaviral polypeptide. In certain embodiments, the second mRNA transcript comprises a nucleotide sequence encoding a heterologous non-arenaviral signal peptide and the arenavirus GP1 and GP2. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding arenavirus GP signal peptide and a buffer peptide, either alone or fused to a heterologous non-arenaviral polypeptide. In certain embodiments, the second mRNA transcript comprises a nucleotide sequence encoding a heterologous non-arenaviral polypeptide; wherein the heterologous non-arenaviral polypeptide encoded by the first mRNA transcript and the heterologous non-arenaviral polypeptide encoded by the second mRNA transcript are the same or different from each other. In certain embodiments, the first mRNA transcript is under control of an arenavirus 3′ UTR and the second mRNA transcript is under control of an arenavirus 5′ UTR.

In certain embodiments, the first mRNA transcript comprises a nucleotide sequence encoding a heterologous non-arenaviral signal peptide and the arenavirus GP1 and GP2. In certain embodiments, the second mRNA transcript comprises a nucleotide sequence encoding NP. In certain embodiments, the first mRNA transcript is under control of an arenavirus 5′ UTR and the second mRNA transcript is under control of an arenavirus 3′ UTR.

In certain embodiments, the heterologous non-arenaviral polypeptide is expressed under control of an arenavirus 3′ UTR or the second heterologous non-arenaviral polypeptide is expressed under control of an arenavirus 3′ UTR; and the expression level of the heterologous non-arenaviral polypeptide or the expression level of the second heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR or higher than the expression level of the same second heterologous non-arenaviral polypeptide expressed under control of the respective arenavirus 5′ UTR.

In certain embodiments, the expression of one heterologous non-arenaviral polypeptide is under control of an arenavirus 3′ UTR and the expression of the other heterologous non-arenaviral polypeptide is under control of an arenavirus 5′ UTR. In certain embodiments, the expression of one heterologous non-arenaviral polypeptide is under control of an arenavirus 3′ UTR in one S segment and the expression of the other heterologous non-arenaviral polypeptide is under control of an arenavirus 5′ UTR in the other S segment. In certain embodiments, cells that are infected with the arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) express (co-express) both heterologous non-arenaviral polypeptides. In certain embodiments, the combined expression level of the two heterologous non-arenaviral polypeptides (e.g., cells expressing both heterologous non-arenaviral polypeptides) is at least about 1%, 2%, 3%, 4%, 5%, 6%, 7%, 8%, 9%, 10%, 15%, 20%, 30%, 40%5, 0%, 60%, 70%, 80%, 90%1, 00%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide is under control of an arenavirus 5′ UTR of one S segment and expression of the other heterologous non-arenaviral polypeptide is under control of an arenavirus 5′ UTR of the other S segment). In certain embodiments, the combined expression level of the two heterologous non-arenaviral polypeptides (e.g., cells expressing both heterologous non-arenaviral polypeptides) is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment). In certain embodiments, the combined expression level of the two heterologous non-arenaviral polypeptides (e.g., cells expressing both heterologous non-arenaviral polypeptides) is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment).

In certain embodiments, provided herein is an arenavirus genomic or antigenomic segment. In certain embodiments, the arenavirus genomic or antigenomic segment provided herein is the nucleotide sequence provided herein (see Section 5.5). In certain embodiments, the nucleotide sequence provided herein (see Section 5.5) is derived from the arenavirus genomic or antigenomic segment described in this Section. In certain embodiments, the transcription of the first ORF (see Section 5.5) may result in the first mRNA transcript described in this Section, whereas the transcription of the second ORF (see Section 5.5) may result in the second mRNA transcript described in the Section. In certain embodiments, the arenavirus particles provided herein (see Section 5.6) comprise the arenavirus genomic or antigenomic segment described in this section.

In certain embodiments, the arenavirus genomic or antigenomic segment is an S segment. In certain embodiments, the arenavirus genomic or antigenomic segment is an L segment. In certain embodiments, the functional fragment encoded by the arenavirus genomic or antigenomic segment is under control of a 3′ UTR. In other embodiments, the functional fragment encoded by the arenavirus genomic or antigenomic segment is under control of a 5′ UTR.

In certain embodiments, the first mRNA transcript comprises a nucleotide sequence encoding arenavirus GP signal peptide and a buffer peptide, either alone or fused to a heterologous non-arenaviral polypeptide, and the second mRNA transcript comprises a nucleotide sequence encoding a heterologous non-arenaviral signal peptide, arenavirus GP1 and arenavirus GP2; and the first mRNA transcript is under control of 5′ UTR and the second mRNA transcript is under control of 3′ UTR. In certain embodiments, the arenavirus genomic or antigenomic segment provided herein is one of the S1 segments depicted in FIG. 18A. FIG. 18A shows a general genomic organization and while the buffer peptide is not present in FIG. 18A, a buffer peptide can be present following the GP signal peptide.

In certain embodiments, the first mRNA transcript comprises a nucleotide sequence encoding arenavirus GP signal peptide and a buffer peptide, either alone or fused to a heterologous non-arenaviral polypeptide, and the second mRNA transcript comprises a nucleotide sequence encoding a heterologous non-arenaviral polypeptide; and the first mRNA transcript is under control of 5′ UTR and the second mRNA transcript is under control of 3′ UTR. In certain embodiments, the first mRNA transcript comprises a nucleotide sequence encoding arenavirus GP signal peptide and a buffer peptide, either alone or fused to a heterologous non-arenaviral polypeptide, and there is no second mRNA transcript; and the first mRNA transcript is under control of 5′ UTR. In certain embodiments, the arenavirus genomic or antigenomic segment provided herein is one of the S1 segments depicted in FIG. 18B. FIG. 18B shows a general genomic organization and while the buffer peptide is not present in FIG. 18B, a buffer peptide can be present following the GP signal peptide.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding NP; and the first mRNA transcript is under control of 5′ UTR and the second mRNA transcript is under control of 3′ UTR. In certain embodiments, the arenavirus genomic or antigenomic segment provided herein is one of the S2 segments depicted in FIG. 18B or FIG. 18E. FIGS. 18A and 18E show a general genomic organization and while the buffer peptide is not present in FIGS. 18A and 18E, a buffer peptide can be present following the GP signal peptide.

In certain embodiments, the first mRNA transcript comprises a nucleotide sequence encoding arenavirus GP signal peptide and a buffer peptide, either alone or fused to a heterologous non-arenaviral polypeptide, and the second mRNA transcript comprises a nucleotide sequence encoding a heterologous non-arenaviral signal peptide, arenavirus GP1 and arenavirus GP2; and the first mRNA transcript is under control of 3′ UTR and the second mRNA transcript is under control of 5′ UTR. In certain embodiments, the arenavirus genomic or antigenomic segment provided herein is one of the S1 segments depicted in FIG. 18C. FIG. 18C shows a general genomic organization and while the buffer peptide is not present in FIG. 18C, a buffer peptide can be present following the GP signal peptide.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding a heterologous non-arenaviral polypeptide; and the first mRNA transcript is under control of 5′ UTR and the second mRNA transcript is under control of 3′ UTR. In certain embodiments, the first mRNA transcript comprises a nucleotide sequence encoding a heterologous non-arenaviral signal peptide, arenavirus GP1, and arenavirus GP2, and there is no second mRNA transcript; and the first mRNA transcript is under control of 5′ UTR. In certain embodiments, the arenavirus genomic or antigenomic segment provided herein is one of the S1 segments depicted in FIG. 18D. FIG. 18D shows a general genomic organization and while the buffer peptide is not present in FIG. 18D, a buffer peptide can be present following the GP signal peptide.

In certain embodiments, the first mRNA transcript comprises a nucleotide sequence encoding arenavirus GP signal peptide and a buffer peptide, either alone or fused to a heterologous non-arenaviral polypeptide, and the second mRNA transcript comprises a nucleotide sequence encoding NP; and the first mRNA transcript is under control of 5′ UTR and the second mRNA transcript is under control of 3′ UTR. In certain embodiments, the arenavirus genomic or antigenomic segment provided herein is one of the S2 segments depicted in FIG. 18D or FIG. 18F. FIG. 18D or 18F shows a general genomic organization and while the buffer peptide is not present in FIG. 18D or 18F, a buffer peptide can be present following the GP signal peptide.

In certain embodiments, the first mRNA transcript comprises a nucleotide sequence encoding arenavirus GP signal peptide and a buffer peptide, either alone or fused to a heterologous non-arenaviral polypeptide, and the second mRNA transcript comprises a nucleotide sequence encoding another heterologous non-arenaviral polypeptide; and the two heterologous non-arenaviral polypeptides are the same or different from each other; and the first mRNA transcript is under control of 3′ UTR and the second mRNA transcript is under control of 5′ UTR. In certain embodiments, the first mRNA transcript comprises a nucleotide sequence encoding arenavirus GP signal peptide and a buffer peptide fused to a heterologous non-arenaviral polypeptide, and there is no second mRNA transcript; and the first mRNA transcript is under control of 3′ UTR. In certain embodiments, the arenavirus genomic or antigenomic segment provided herein is one of the S1 segments depicted in FIG. 18E. FIG. 18E shows a general genomic organization and while the buffer peptide is not present in FIG. 18E, a buffer peptide can be present following the GP signal peptide.

In certain embodiments, the first mRNA transcript comprises a nucleotide sequence encoding a heterologous non-arenaviral signal peptide, arenavirus GP1, and arenavirus GP2, and the second mRNA transcript comprises a nucleotide sequence encoding a heterologous non-arenaviral polypeptide; and the first mRNA transcript is under control of 3′ UTR and the second mRNA transcript is under control of 5′ UTR. In certain embodiments, the first mRNA transcript comprises a nucleotide sequence encoding a heterologous non-arenaviral signal peptide, arenavirus GP1, and arenavirus GP2, and there is no second mRNA transcript; and the first mRNA transcript is under control of 3′ UTR. In certain embodiments, the arenavirus genomic or antigenomic segment provided herein is one of the S1 segments depicted in FIG. 18F. FIG. 18F shows a general genomic organization and while the buffer peptide is not present in FIG. 18F, a buffer peptide can be present following the GP signal peptide.

Non-limiting examples of the polypeptides encoded by the first and second mRNA transcripts are illustrated in Table 3.

TABLE 3
Non-limiting examples of the polypeptides encoded
by the first and second mRNA transcripts.

| | 1^{st} mRNA transcript | | |
|---|---|---|---|
| | Functional | | |
| | fragment | | Functional |
| | of a first | | fragment |
| | polypeptide | | of a first |
| | and a heterologous | | polypeptide |
| | non-arenaviral | | and a third |
| | polypeptide | | polypeptide |
| | (fused | Functional | (fused |
| | together or | fragment | together or |
| 2^{nd} mRNA | separated | of a first | separated |
| transcript | by an IRES) | polypeptide | by an IRES) |
| Functional fragment | Exemplary | Exemplary | Exemplary |
| of a first polypeptide | combination | combination | combination |
| and a heterologous | | | |
| non-arenaviral | | | |
| polypeptide | | | |
| (fused together or | | | |
| separated by an | | | |
| IRES) | | | |
| Functional fragment | Exemplary | Exemplary | Exemplary |
| of a first polypeptide | combination | combination | combination |
| Functional fragment | Exemplary | Exemplary | Exemplary |
| of a first polypeptide | combination | combination | combination |
| and a third | | | |
| polypeptide | | | |
| (fused together or | | | |
| separated by an | | | |
| IRES) | | | |
| A heterologous non- | Exemplary | Exemplary | Exemplary |
| arenaviral | combination | combination | combination |
| polypeptide | | | |
| A second | Exemplary | Exemplary | Exemplary |
| polypeptide | combination | combination | combination |
| Functional fragment | Exemplary | Exemplary | Exemplary |
| of a second | combination | combination | combination |
| polypeptide and a | | | |
| heterologous non- | | | |
| arenaviral | | | |
| polypeptide | | | |
| (fused together or | | | |
| separated by an | | | |
| IRES) | | | |
| Functional fragment | Exemplary | Exemplary | Exemplary |
| of a second | combination | combination | combination |
| polypeptide | | | |
| Functional fragment | Exemplary | Exemplary | Exemplary |
| of a second | combination | combination | combination |
| polypeptide and a | | | |
| third polypeptide | | | |
| (fused together or | | | |
| separated by an | | | |
| IRES) | | | |

In certain embodiments, the functional fragment is selected from the group consisting of arenavirus GP signal peptide (and a buffer peptide), arenavirus GP1 and arenavirus GP2. In certain embodiments, non-limiting examples of the polypeptides encoded by the first and second mRNA transcripts related to functional fragments of GP are illustrated in Table 4.

TABLE 4
Non-limiting examples of the polypeptides encoded by the first and
second mRNA transcripts related to functional fragments of GP.

| First mRNA transcript | Second mRNA transcript |
|---|---|
| GP signal peptide, a buffer peptide, and a | A heterologous non-arenaviral signal |
| heterologous non-arenaviral polypeptide | peptide, arenavirus GP1, and arenavirus |
| | GP2 |
| GP signal peptide, a buffer peptide, and a | A heterologous non-arenaviral polypeptide |
| heterologous non-arenaviral polypeptide | |
| GP signal peptide, a buffer peptide, and a | NP |
| heterologous non-arenaviral polypeptide | |
| GP signal peptide, a buffer peptide, and a | Z |
| heterologous non-arenaviral polypeptide | |
| GP signal peptide, a buffer peptide, and a | L |
| heterologous non-arenaviral polypeptide | |
| GP signal peptide, a buffer peptide, and a | No second mRNA transcript |
| heterologous non-arenaviral polypeptide | |
| GP signal peptide and a buffer peptide | A heterologous non-arenaviral signal |
| | peptide, arenavirus GP1, and arenavirus |
| | GP2 |
| GP signal peptide and a buffer peptide | A heterologous non-arenaviral polypeptide |
| GP signal peptide and a buffer peptide | NP |
| GP signal peptide and a buffer peptide | Z |
| GP signal peptide and a buffer peptide | L |
| GP signal peptide and a buffer peptide | No second mRNA transcript |
| GP signal peptide, a buffer peptide, and NP | A heterologous non-arenaviral signal |
| | peptide, arenavirus GP1, and arenavirus |
| | GP2 |
| GP signal peptide, a buffer peptide, and NP | A heterologous non-arenaviral polypeptide |
| GP signal peptide, a buffer peptide, and NP | Z |
| GP signal peptide, a buffer peptide, and NP | L |
| GP signal peptide, a buffer peptide, and NP | No second mRNA transcript |
| GP signal peptide, a buffer peptide, and Z | A heterologous non-arenaviral signal |
| | peptide, arenavirus GP1, and arenavirus |
| | GP2 |
| GP signal peptide, a buffer peptide, and Z | A heterologous non-arenaviral polypeptide |
| GP signal peptide, a buffer peptide, and Z | NP |
| GP signal peptide, a buffer peptide, and Z | L |
| GP signal peptide, a buffer peptide, and Z | No second mRNA transcript |
| GP signal peptide, a buffer peptide, and L | A heterologous non-arenaviral signal |
| | peptide, arenavirus GP1, and arenavirus |
| | GP2 |
| GP signal peptide, a buffer peptide, and L | A heterologous non-arenaviral polypeptide |
| GP signal peptide, a buffer peptide, and L | NP |
| GP signal peptide, a buffer peptide, and L | Z |
| GP signal peptide, a buffer peptide, and L | No second mRNA transcript |
| GP1, GP2 and a heterologous non- | GP signal peptide, a buffer peptide, and a |
| arenaviral signal peptide | heterologous non-arenaviral polypeptide |
| GP1, GP2 and a heterologous non- | GP signal peptide and a buffer peptide |
| arenaviral signal peptide | |
| GP1, GP2 and a heterologous non- | A heterologous non-arenaviral polypeptide |
| arenaviral signal peptide | |
| GP1, GP2 and a heterologous non- | NP |
| arenaviral signal peptide | |
| GP1, GP2 and a heterologous non- | Z |
| arenaviral signal peptide | |
| GP1, GP2 and a heterologous non- | L |
| arenaviral signal peptide | |
| GP1, GP2 and a heterologous non- | No second mRNA transcript |
| arenaviral signal peptide | |

### 5.7.1 Arenavirus Particle Comprising the Arenavirus Genomic or Antigenomic Segment Provided Herein

In certain embodiments, provided herein is an arenavirus particle comprising the arenavirus genomic or antigenomic segment provided herein.

In certain embodiments, the genome of the arenavirus particle consists of an S segment and an L segment.

In certain embodiments, the arenavirus particle is tri-segmented. In certain embodiments, the tri-segmented arenavirus particle comprises two S segments and an L segment. In certain embodiments, the tri-segmented arenavirus particle comprises an S segment and two L segments.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide alone under the control of an arenavirus 3′ UTR and a heterologous non-arenaviral signal peptide, arenavirus GP1 and arenavirus GP2 under the control of an arenavirus 5′ UTR;
b) an S segment that encodes NP under the control of an arenavirus 3′ UTR and another heterologous non-arenaviral polypeptide or no polypeptide under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes arenavirus GP signal peptide, a buffer peptide, and a heterologous non-arenaviral polypeptide or arenavirus GP signal peptide and a buffer peptide alone under the control of an arenavirus 3′ UTR and another heterologous non-arenaviral polypeptide or no polypeptide under the control of an arenavirus 5′ UTR;
b) an S segment that encodes NP under the control of an arenavirus 3′ UTR and a heterologous non-arenaviral signal peptide, arenavirus GP1 and arenavirus GP2 under the control of an arenavirus 5′ UTR; and
c) an L segment that encodes L protein under the control of an arenavirus 3′ UTR and Z protein under the control of an arenavirus 5′ UTR; and
   wherein the two heterologous non-arenaviral polypeptides are the same or different from each other.

In certain embodiments, the genome of the arenavirus particle consists of
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR;
b) an S segment that encodes NP under the control of an arenavirus genomic 3′ UTR and a second heterologous non-arenaviral polypeptide or no polypeptide under the control of an arenavirus genomic 5′ UTR; and
c) an L segment that encodes the L protein under the control of an arenavirus genomic 3′ UTR and the Z protein under the control of an arenavirus genomic 5′ UTR; wherein the first heterologous non-arenaviral polypeptide and the second heterologous non-arenaviral polypeptide are the same or different from each other.

In certain embodiments, the genome of the arenavirus particle consists of
a) an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR;
b) an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and
c) an L segment that encodes the L protein under the control of an arenavirus genomic 3′ UTR and the Z protein under the control of an arenavirus genomic 5′ UTR; wherein the first heterologous non-arenaviral polypeptide and the second heterologous non-arenaviral polypeptide are the same or different from each other.

In certain embodiments, the arenavirus particle is derived from a Lassa virus. In certain embodiments, the arenavirus particle is derived from a Lymphocytic choriomeningitis virus (LCMV). In certain embodiments, the LCMV is a MP strain, a WE strain, an Armstrong strain, or an Armstrong Clone 13 strain. In certain embodiments, the arenavirus particle is derived from a New World arenavirus of Clade A. In certain embodiments, the arenavirus particle is derived from a Pichinde virus (PICV). In certain embodiments, the arenavirus particle is derived from a Junin virus vaccine Candid #1, or a Junin virus vaccine XJ Clone 3 strain. In certain embodiments, the arenavirus particle is derived from an Oliveros virus. In certain embodiments, the arenavirus particle is derived from a Tamiami virus. In certain embodiments, the arenavirus particle is derived from a Mobala virus. In certain embodiments, the arenavirus particle is derived from a Mopeia virus. In certain embodiments, the arenavirus particle is derived from an Ippy virus. In certain embodiments, the arenavirus particle is derived from an Amapari virus. In certain embodiments, the arenavirus particle is derived from a Flexal virus. In certain embodiments, the arenavirus particle is derived from a Guanarito virus. In certain embodiments, the arenavirus particle is derived from a Latino virus. In certain embodiments, the arenavirus particle is derived from a Machupo virus. In certain embodiments, the arenavirus particle is derived from a Parana virus. In certain embodiments, the arenavirus particle is derived from a Pirital virus. In certain embodiments, the arenavirus particle is derived from a Sabia virus. In certain embodiments, the arenavirus particle is derived from a Tacaribe virus. In certain embodiments, the arenavirus particle is derived from a Bear Canyon virus. In certain embodiments, the arenavirus particle is derived from a Whitewater Arroyo virus. In certain embodiments, the arenavirus particle is derived from an Allpahuayo virus (ALLV). In certain embodiments, the arenavirus particle is derived from an Alxa virus. In certain embodiments, the arenavirus particle is derived from a Chapare virus. In certain embodiments, the arenavirus particle is derived from a Lijiang virus. In certain embodiments, the arenavirus particle is derived from a Cupixi virus. In certain embodiments, the arenavirus particle is derived from a Gairo virus. In certain embodiments, the arenavirus particle is derived from a Loei River virus. In certain embodiments, the arenavirus particle is derived from a Lujo virus. In certain embodiments, the arenavirus particle is derived from a Luna virus. In certain embodiments, the arenavirus particle is derived from a Luli virus. In certain embodiments, the arenavirus particle is derived from a Lunk virus. In certain embodiments, the arenavirus particle is derived from a Mariental virus. In certain embodiments, the arenavirus particle is derived from a Merino Walk virus. In certain embodiments, the arenavirus particle is derived from a Morogoro virus. In certain embodiments, the arenavirus particle is derived from an Okahandja virus. In certain embodiments, the arenavirus particle is derived from an Apore virus. In certain embodiments, the arenavirus particle is derived from a Ryukyu virus. In certain embodiments, the arenavirus particle is derived from a Solwezi virus. In certain embodiments, the arenavirus particle is derived from a souris virus. In certain embodiments, the arenavirus particle is derived from a Wenzhou virus. In certain embodiments, the arenavirus particle is derived from a Big Brushy Tank virus. In certain embodiments, the arenavirus particle is derived from a Catarina virus. In certain embodiments, the arenavirus particle is derived from a Skinner Tank virus. In certain embodiments, the arenavirus particle is derived from a Tonto Creek virus. In certain embodiments, the arenavirus particle is derived from a Xapuri virus.

In certain embodiments, the genome of the arenavirus particle encodes the heterologous non-arenaviral polypeptide and/or the second heterologous non-arenaviral polypeptide, and the growth or infectivity of the arenavirus particle is not inferior to a second arenavirus particle, wherein the genome of the second arenavirus particle encodes the same heterologous non-arenaviral polypeptide and/or the same second heterologous non-arenaviral polypeptide; and wherein all arenaviral GP, NP, Z and L in the second arenavirus particle are each expressed as one ORF. In certain embodiments, the growth or infectivity of the arenavirus particle described herein is not inferior to a second arenavirus particle. In some embodiments, the genome of the second arenavirus particle does not encode a buffer peptide.

In certain embodiments, the arenavirus particle is infectious and replication competent. In certain embodiments, the arenavirus particle is attenuated as compared to its parental wild-type virus. In certain embodiments, the arenavirus particle is infectious but unable to produce further infectious progeny in non-complementing cells.

In certain embodiments, the titer of the arenavirus particle of the disclosure is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

In certain embodiments, the arenavirus particle of the disclosure expresses a heterologous non-arenaviral polypeptide under control of an arenavirus 3′ UTR; wherein the arenavirus particle induces at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide than another arenavirus particle expressing the same heterologous non-arenaviral polypeptide under control of an arenavirus 5′ UTR. In some embodiments, the another arenavirus particle does not comprise a buffer peptide. In some embodiments, an immune response (e.g., a higher immune response) is obtained after an arenavirus particle of the present disclosure (e.g., arenavirus particle expressing a heterologous non-arenaviral polypeptide under control of an arenavirus 3′ UTR) is administered to a subject. In some embodiments, an immune response is obtained after an arenavirus particle expressing the heterologous non-arenaviral polypeptide under control of an arenavirus 5′ UTR is administered to a subject or to a comparable subject. In some embodiments, an immune response obtained after an arenavirus particle of the present disclosure is administered to a subject is compared to an immune response obtained after another arenavirus particle (e.g., arenavirus particle expressing the heterologous non-arenaviral polypeptide under control of an arenavirus 5′ UTR) is administered to a subject or to a comparable subject. In some embodiments, the subject and/or the comparable subject is a subject in need of treatment. In some embodiments, the subject and/or the comparable subject is a subject with a disease and/or with symptoms of a disease. In some embodiments, the comparable subject is a healthy subject. In some embodiments, the comparable subject is a subject without the disease. In some embodiments, the comparable subject is a subject not in need of treatment.

In certain embodiments, at least about, or about, 5%, 10%, 15%, 20%, 25%, 30%, 35%, 40%, 4%5% 50%, 55%, 60%, 65%, 70%, 75%, 80%, 85%, 90%, 95%, 99%, or 100% of cells express the two heterologous non-arenaviral polypeptides after an arenavirus particle of the present disclosure (e.g., expressing one heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptides after an arenavirus particle of the present disclosure (e.g., expressing one heterologous non-arenaviral polypeptide under control of an arenavirus 3′ UTR in one S segment) is introduced to (or infects) a population of cells is higher as compared to the proportion of cells that express the heterologous non-arenaviral polypeptides after an arenavirus particle expressing the same heterologous non-arenaviral polypeptides under control of arenavirus 5′ UTR is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptides after an arenavirus particle of the present disclosure (e.g., expressing one heterologous non-arenaviral polypeptide under control of an arenavirus 3′ UTR in one S segment) is introduced to (or infects) a population of cells as compared to the proportion of cells that express the heterologous non-arenaviral polypeptides after an arenavirus particle expressing the same heterologous non-arenaviral polypeptides under control of arenavirus 5′ UTR is introduced to (or infects) a comparable population of cells.

In certain embodiments, at least about, or about, 5%, 10%, 15%, 20%, 25%, 30%, 35%, 40%, 4%5% 50%, 55%, 60%, 6% 5, ⁷⁰, 75%, 80%, 85%, 90%, 95%, 99%, or 100% of cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion of cells that express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a population of cells. In certain embodiments, at least about 1%, 2%, 3%, 4%, 5%, 6%, 7%, 8%, 9%0, 10%, 15%, 20%, 25%, 30%, 35%, 40%, 45%, 50%, 55%, 60%, 65%, 70%, 75%, 80%, 85%, 90%, 95%, 99%, or 100% more cells express (e.g., co-express) the two heterologous non-arenaviral polypeptides after an arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion of cells that express (e.g., co-express) the two heterologous non-arenaviral polypeptides after an arenavirus particle expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells.

### 5.7.2 Compositions and Methods Related to the Nucleotide Sequence Provided Herein

In certain embodiments, provided herein is a translation product of the arenavirus genomic or antigenomic segment provided herein.

In certain embodiments, provided herein is a cDNA of the arenavirus genomic or antigenomic segment provided herein.

In certain embodiments, provided herein is a method of producing an arenavirus genomic or antigenomic segment, wherein the method comprises transcribing the cDNA provided herein.

In certain embodiments, provided herein is a method of generating an arenavirus particle, wherein the method comprises:
a) transfecting into a host cell one or more cDNA sequences of the arenavirus genomic or antigenomic segment provided herein or one or more RNA sequences each transcribed in vitro from the cDNA sequence of the arenavirus genomic or antigenomic segment provided herein;
b) transfecting into the host cell nucleotide sequences encoding arenavirus trans-acting factors;
c) maintaining the host cell under conditions suitable for virus formation; and
d) harvesting the arenavirus particle.

In certain embodiments, the one or more cDNA sequences are transcribed using a bidirectional promoter. In certain embodiments, the one or more cDNA sequences are transcribed under the control of a promoter selected from the group consisting of:
a) a RNA polymerase I promoter;
b) a RNA polymerase II promoter; and
c) a T7 promoter.

In certain embodiments, provided herein is a DNA expression vector comprising a DNA sequence encoding the arenavirus genomic or antigenomic segment provided herein.

In certain embodiments, provided herein is a method of rescuing an arenavirus particle using the arenavirus genomic or antigenomic segment provided herein or a DNA sequence encoding the arenavirus genomic or antigenomic segment.

In certain embodiments, provided herein is a host cell comprising the arenavirus genomic or antigenomic segment provided herein, the translation product provided herein, the arenavirus particle provided herein, or the DNA expression vector provided herein.

In certain embodiments, provided herein is a vaccine comprising the arenavirus genomic or antigenomic segment provided herein, the translation product provided herein, the arenavirus particle provided herein, or the DNA expression vector provided herein, and a pharmaceutically acceptable carrier.

In certain embodiments, provided herein is a pharmaceutical composition comprising the arenavirus genomic or antigenomic segment provided herein, the translation product provided herein, the arenavirus particle provided herein, or the DNA expression vector provided herein, and a pharmaceutically acceptable carrier.

### 5.8 Translation Product Provided Herein

In certain embodiments, provided herein is a translation product of the nucleotide sequence provided herein (see Section 5.5). In certain embodiments, provided herein is a translation product of the mRNA transcript of the genome of the arenavirus particle provided herein (see Section 5.6). In certain embodiments, provided herein is a translation product of the arenavirus genomic or antigenomic segment provided herein (see Section 5.7).

In certain embodiments, the translation product is a chimeric protein. In certain embodiments, the translation product comprises a functional fragment (see Section 5.2) of a polypeptide provided herein (see Section 5.1) fused to a heterologous non-arenaviral polypeptide (see Section 5.3). In certain embodiments, the translation product comprises a functional fragment (see Section 5.2) of a polypeptide provided herein (see Section 5.1) fused to another polypeptide provided herein (see Section 5.1). In certain embodiments, the translation product comprises a functional fragment (see Section 5.2) of a polypeptide provided herein (see Section 5.1) fused to another functional fragment of the same polypeptide or another polypeptide provided herein (see Section 5.1). In certain embodiments, the translation product comprises a functional fragment (see Section 5.2) of a polypeptide provided herein (see Section 5.1). In certain embodiments, the translation product comprises a heterologous non-arenaviral polypeptide (see Section 5.3). In certain embodiments, the translation product comprises a polypeptide provided herein (see Section 5.1).

In certain embodiments, the translation product provided herein is a translation product of any one of the first or the second ORFs listed in Table 1. In certain embodiments, the translation product provided herein is a translation product of any one of the first or the second ORFs listed in Table 2. In certain embodiments, the translation product provided herein is a translation product of any one of the first or the second mRNA transcripts listed in Table 3. In certain embodiments, the translation product provided herein is a translation product of any one of the first or the second mRNA transcripts listed in Table 4.

In certain embodiments, the translation product provided herein is a translation product of any one of the arenavirus genomic or antigenomic segments depicted in any one of FIGS. 18A-18F.

### 5.9 Vector Systems And Cell Lines

In certain embodiments, provided herein are DNAs comprising or consisting of the nucleotide sequence as described in Section 5.5. In certain embodiments, provided herein is an arenavirus genomic or antigenomic segment as described in Section 5.7. In certain embodiments, provided herein is an arenavirus genomic or antigenomic segment in the arenavirus particle as described in Section 5.6. In certain embodiments, provided herein is a DNA sequence encoding the mRNA transcript in the arenavirus particle as described in Section 5.6. In certain embodiments, the DNA is a cDNA.

In one embodiment, provided herein is a DNA expression vector comprising the nucleotide sequence as described in Section 5.5. In one embodiment, provided herein is a DNA expression vector comprising a cDNA encoding an arenavirus genomic or antigenomic segment as described in Section 5.7. In one embodiment, provided herein is a DNA expression vector comprising a cDNA encoding the arenavirus genomic or antigenomic segment in the arenavirus particle as described in Section 5.6. In certain embodiments, provided herein is a DNA expression vector comprising a cDNA of the mRNA transcript in the arenavirus particle as described in Section 5.6.

In one embodiment, provided herein is a DNA expression vector system that encodes the bi-segmented or tri-segmented arenavirus particle as described herein. Specifically, provided herein is a DNA expression vector system wherein one or more vectors encode two or three arenavirus genomic or antigenomic segments, namely, one L segment and one S section of a bi-segmented arenavirus as described herein, or one L segment and two S segments or two L segments and one S segment of a tri-segmented arenavirus particle described herein. Such a vector system can encode one or more separate DNA molecules.

In another embodiment, provided herein is a DNA or cDNA of the arenavirus S segment(s) as described in Section 5.7, and is part of or incorporated into a DNA expression system. In other embodiments, a DNA or cDNA of the arenavirus L segment(s) as described in Section 5.7 is part of or incorporated into a DNA expression system.

In certain embodiments, the DNA provided herein can be derived from a particular strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In specific embodiments, the cDNA is derived from LCMV Clone 13. In other specific embodiments, the cDNA is derived from LCMV MP strain.

In certain embodiments, the DNA or cDNA provided herein can be derived from a particular strain of Lassa virus. In certain embodiments, the DNA or cDNA provided herein can be derived from a particular strain of Pichinde virus. In certain embodiments, the DNA or cDNA provided herein can be derived from a particular strain of Junin virus. In certain embodiments, the DNA or cDNA provided herein can be derived from a particular strain of Oliveros virus. In certain embodiments, the DNA or cDNA provided herein can be derived from a particular strain of Tamiami virus. In certain embodiments, the DNA or cDNA provided herein can be derived from a particular strain of Mobala virus. In certain embodiments, the DNA or cDNA provided herein can be derived from a particular strain of Mopeia virus. In certain embodiments, the DNA or cDNA provided herein can be derived from a particular strain of Ippy virus. In certain embodiments, the DNA or cDNA provided herein can be derived from a particular strain of Amapari virus. In certain embodiments, the DNA or cDNA provided herein can be derived from a particular strain of Flexal virus. In certain embodiments, the DNA or cDNA provided herein can be derived from a particular strain of Guanarito virus. In certain embodiments, the DNA or cDNA provided herein can be derived from a particular strain of Latino virus. In certain embodiments, the cDNA provided herein can be derived from a particular strain of Machupo virus. In certain embodiments, the DNA or cDNA provided herein can be derived from a particular strain of Parana virus. In certain embodiments, the DNA or cDNA provided herein can be derived from a particular strain of Pirital virus. In certain embodiments, the DNA or cDNA provided herein can be derived from a particular strain of Sabia virus. In certain embodiments, the DNA or cDNA provided herein can be derived from a particular strain of Tacaribe virus. In certain embodiments, the DNA or cDNA provided herein can be derived from a particular strain of Bear Canyon virus. In certain embodiments, the DNA or cDNA provided herein can be derived from a particular strain of Whitewater Arroyo virus. In certain embodiments, the DNA or cDNA provided herein can be derived from a particular strain of Allpahuayo virus (ALLV). In certain embodiments, the DNA or cDNA provided herein can be derived from a particular strain of Alxa virus. In certain embodiments, the DNA or cDNA provided herein can be derived from a particular strain of Chapare virus. In certain embodiments, the DNA or cDNA provided herein can be derived from a particular strain of Lijiang virus. In certain embodiments, the DNA or cDNA provided herein can be derived from a particular strain of Cupixi virus. In certain embodiments, the DNA or cDNA provided herein can be derived from a particular strain of Gairo virus. In certain embodiments, the DNA or cDNA provided herein can be derived from a particular strain of Loei River virus. In certain embodiments, the DNA or cDNA provided herein can be derived from a particular strain of Lujo virus. In certain embodiments, the DNA or cDNA provided herein can be derived from a particular strain of Luna virus. In certain embodiments, the DNA or cDNA provided herein can be derived from a particular strain of Luli virus. In certain embodiments, the DNA or cDNA provided herein can be derived from a particular strain of Lunk virus. In certain embodiments, the DNA or cDNA provided herein can be derived from a particular strain of Mariental virus. In certain embodiments, the DNA or cDNA provided herein can be derived from a particular strain of Merino Walk virus. In certain embodiments, the DNA or cDNA provided herein can be derived from a particular strain of Morogoro virus. In certain embodiments, the DNA or cDNA provided herein can be derived from a particular strain of Okahandja virus. In certain embodiments, the DNA or cDNA provided herein can be derived from a particular strain of Apore virus. In certain embodiments, the DNA or cDNA provided herein can be derived from a particular strain of Ryukyu virus. In certain embodiments, the DNA or cDNA provided herein can be derived from a particular strain of Solwezi virus. In certain embodiments, the DNA or cDNA provided herein can be derived from a particular strain of souris virus. In certain embodiments, the DNA or cDNA provided herein can be derived from a particular strain of Wenzhou virus. In certain embodiments, the DNA or cDNA provided herein can be derived from a particular strain of Big Brushy Tank virus. In certain embodiments, the DNA or cDNA provided herein can be derived from a particular strain of Catarina virus. In certain embodiments, the DNA or cDNA provided herein can be derived from a particular strain of Skinner Tank virus. In certain embodiments, the DNA or cDNA provided herein can be derived from a particular strain of Tonto Creek virus. In certain embodiments, the DNA or cDNA provided herein can be derived from a particular strain of Xapuri virus.

In certain embodiments, the vector generated to encode an arenavirus particle as described herein may be based on a specific strain of LCMV. Strains of LCMV include Clone 13, MP strain, Arm CA 1371, Arm E-250, WE, UBC, Traub, Pasteur, 810885, CH-5692, Marseille #12, HP65-2009, 200501927, 810362, 811316, 810316, 810366, 20112714, Douglas, GR01, SN05, CABN and their derivatives. In certain embodiments, an arenavirus particle or a tri-segmented arenavirus particle as described herein may be based on LCMV Clone 13. In other embodiments, the vector generated to encode an arenavirus particle or a tri-segmented arenavirus particle as described herein may be based on LCMV MP strain.

In certain embodiments, the vector generated to encode an arenavirus particle as described herein may be based on a specific strain of Lassa virus. In certain embodiments, the vector generated to encode an arenavirus particle as described herein may be based on a specific strain of Pichinde virus. In certain embodiments, the vector generated to encode an arenavirus particle as described herein may be based on a specific strain of Junin virus. In certain embodiments, the vector generated to encode an arenavirus particle as described herein may be based on a specific strain of Oliveros virus. In certain embodiments, the vector generated to encode an arenavirus particle as described herein may be based on a specific strain of Tamiami virus. In certain embodiments, the vector generated to encode an arenavirus particle as described herein may be based on a specific strain of Mobala virus. In certain embodiments, the vector generated to encode an arenavirus particle as described herein may be based on a specific strain of Mopeia virus. In certain embodiments, the vector generated to encode an arenavirus particle as described herein may be based on a specific strain of Ippy virus. In certain embodiments, the vector generated to encode an arenavirus particle as described herein may be based on a specific strain of Amapari virus. In certain embodiments, the vector generated to encode an arenavirus particle as described herein may be based on a specific strain of Flexal virus. In certain embodiments, the vector generated to encode an arenavirus particle as described herein may be based on a specific strain of Guanarito virus. In certain embodiments, the vector generated to encode an arenavirus particle as described herein may be based on a specific strain of Latino virus. In certain embodiments, the vector generated to encode an arenavirus particle as described herein may be based on a specific strain of Machupo virus. In certain embodiments, the vector generated to encode an arenavirus particle as described herein may be based on a specific strain of Parana virus. In certain embodiments, the vector generated to encode an arenavirus particle as described herein may be based on a specific strain of Pirital virus. In certain embodiments, the vector generated to encode an arenavirus particle as described herein may be based on a specific strain of Sabia virus. In certain embodiments, the vector generated to encode an arenavirus particle as described herein may be based on a specific strain of Tacaribe virus. In certain embodiments, the vector generated to encode an arenavirus particle as described herein may be based on a specific strain of Bear Canyon virus. In certain embodiments, the vector generated to encode an arenavirus particle as described herein may be based on a specific strain of Whitewater Arroyo virus. In certain embodiments, the vector generated to encode an arenavirus particle as described herein may be based on a specific strain of Allpahuayo virus (ALLV). In certain embodiments, the vector generated to encode an arenavirus particle as described herein may be based on a specific strain of Alxa virus. In certain embodiments, the vector generated to encode an arenavirus particle as described herein may be based on a specific strain of Chapare virus. In certain embodiments, the vector generated to encode an arenavirus particle as described herein may be based on a specific strain of Lijiang virus. In certain embodiments, the vector generated to encode an arenavirus particle as described herein may be based on a specific strain of Cupixi virus. In certain embodiments, the vector generated to encode an arenavirus particle as described herein may be based on a specific strain of Gairo virus. In certain embodiments, the vector generated to encode an arenavirus particle as described herein may be based on a specific strain of Loei River virus. In certain embodiments, the vector generated to encode an arenavirus particle as described herein may be based on a specific strain of Lujo virus. In certain embodiments, the vector generated to encode an arenavirus particle as described herein may be based on a specific strain of Luna virus. In certain embodiments, the vector generated to encode an arenavirus particle as described herein may be based on a specific strain of Luli virus. In certain embodiments, the vector generated to encode an arenavirus particle as described herein may be based on a specific strain of Lunk virus. In certain embodiments, the vector generated to encode an arenavirus particle as described herein may be based on a specific strain of Mariental virus. In certain embodiments, the vector generated to encode an arenavirus particle as described herein may be based on a specific strain of Merino Walk virus. In certain embodiments, the vector generated to encode an arenavirus particle as described herein may be based on a specific strain of Morogoro virus. In certain embodiments, the vector generated to encode an arenavirus particle as described herein may be based on a specific strain of Okahandja virus. In certain embodiments, the vector generated to encode an arenavirus particle as described herein may be based on a specific strain of Apore virus. In certain embodiments, the vector generated to encode an arenavirus particle as described herein may be based on a specific strain of Ryukyu virus. In certain embodiments, the vector generated to encode an arenavirus particle as described herein may be based on a specific strain of Solwezi virus. In certain embodiments, the vector generated to encode an arenavirus particle as described herein may be based on a specific strain of souris virus. In certain embodiments, the vector generated to encode an arenavirus particle as described herein may be based on a specific strain of Wenzhou virus. In certain embodiments, the vector generated to encode an arenavirus particle as described herein may be based on a specific strain of Big Brushy Tank virus. In certain embodiments, the vector generated to encode an arenavirus particle as described herein may be based on a specific strain of Catarina virus. In certain embodiments, the vector generated to encode an arenavirus particle as described herein may be based on a specific strain of Skinner Tank virus. In certain embodiments, the vector generated to encode an arenavirus particle as described herein may be based on a specific strain of Tonto Creek virus. In certain embodiments, the vector generated to encode an arenavirus particle as described herein may be based on a specific strain of Xapuri virus,

In another embodiment, provided herein is a cell, wherein the cell comprises a DNA or a vector system described above in this section. Cell lines derived from such cells, cultures comprising such cells, methods of culturing such cells are also provided herein. In certain embodiments, provided herein is a cell, wherein the cell comprises a cDNA of the tri-segmented arenavirus particle. In some embodiments, the cell comprises the S segment(s) and/or the L segment(s).

### 5.10 Generation of the Arenavirus Particles

Generally, arenavirus particles can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the arenavirus particles provided herein, these techniques can be applied as described below.

In certain embodiments, the genome of the viruses may comprise the nucleotide sequence described in Section 5.5. The genome of the viruses can be modified as described in Section 5.7. The generation of an arenavirus particle as described in Section 5.6 or an arenavirus particle comprising a genomic or antigenomic segment as described in Section 5.7 can be recombinantly produced by any reverse genetic techniques known to one skilled in the art.

In certain embodiments, an arenavirus particle as described in Section 5.6 or an arenavirus particle comprising a genomic or antigenomic segment as described in Section 5.7 can be tri-segmented. A tri-segmented arenavirus particle can be recombinantly produced by reverse genetic techniques known in the art, for example as described in International Publication No.: WO 2016/075250 A1.

### 5.10.1 Infectious and Replication Competent Arenavirus Particle

In certain embodiments, the method of generating a bi-segmented arenavirus particle comprises (i) transfecting into a host cell the cDNA or RNA sequences each transcribed in vitro from DNA sequences of a first and a second arenavirus genomic or antigenomic segments; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the cDNA is comprised in a plasmid.

In certain embodiments, when the arenavirus particle is tri-segmented, the method of generating the arenavirus particle comprises (i) transfecting into a host cell the cDNAs or RNA sequences each transcribed in vitro from DNA sequences of the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle.

Once generated from cDNA, arenavirus particles (i.e., infectious and replication competent) can be propagated. In certain embodiments, the arenavirus particle can be propagated in any host cell that allows the virus to grow to titers that permit the uses of the virus as described herein. In one embodiment, the host cell allows the arenavirus particle to grow to titers comparable to those determined for the corresponding tri-segmented artLCMV particle expressing the same heterologous non-arenaviral polypeptide(s).

In certain embodiments, the arenavirus particle may be propagated in host cells. Specific examples of host cells that can be used include BHK-21, HEK 293, VERO or other. In a specific embodiment, the arenavirus particle may be propagated in a cell line.

In certain embodiments, the host cells are kept in culture and are transfected with one or more plasmid(s). The plasmid(s) express or encode the arenavirus genomic or antigenomic segment(s) to be generated under control of one or more expression cassettes suitable for expression in mammalian cells, e.g., consisting of a polymerase I promoter and terminator.

In specific embodiments, the host cells are kept in culture and are transfected with one or more plasmid(s). The plasmid(s) express or encode the viral gene(s) to be generated under control of one or more expression cassettes suitable for expression in mammalian cells, e.g., consisting of a polymerase I promoter and terminator.

Plasmids that can be used for the generation of the arenavirus particle can include: i) a plasmid encoding the S genomic segment e.g., pol-I S, ii) a plasmid encoding the L genomic segment e.g., pol-I L. In certain embodiments, the plasmid encoding an arenavirus polymerase that directs intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, when the arenaviral particle is tri-segmented, plasmids that can be used for generating the tri-segmented arenavirus comprising one L segment and two S segments can include: i) two plasmids each encoding the S genome segment e.g., pol-I S, ii) a plasmid encoding the L genome segment e.g., pol-I L. Plasmids needed for the tri-segmented arenavirus comprising two L segments and one S segment are: i) two plasmids each encoding the L genome segment e.g., pol-L, ii) a plasmid encoding the S genome segment e.g., pol-I S.

In certain embodiments, when the arenaviral particle is tri-segmented, plasmids encoding an arenavirus polymerase that direct intracellular synthesis of the viral L and S segments can be incorporated into the transfection mixture. For example, a plasmid encoding the L protein and a plasmid encoding NP (pC-L and pC-NP, respectively). The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, the arenavirus genomic or antigenomic segments are under the control of a promoter. Typically, RNA polymerase I-driven expression cassettes, RNA polymerase II-driven cassettes or T7 bacteriophage RNA polymerase driven cassettes can be used. In certain embodiments, the plasmid(s) encoding the arenavirus genomic or antigenomic segments can be the same, i.e., the genome sequence and trans-acting factors can be transcribed by a promoter from one plasmid. Specific examples of promoters include an RNA polymerase I promoter, an RNA polymerase II promoter, an RNA polymerase III promoter, a T7 promoter, an SP6 promoter or a T3 promoter.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

To obtain a cell line stably expressing a desired protein, for example an arenaviral structural protein, transfection of a host cell with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

For recovering the arenavirus particle described herein, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (arenavirus vector preparation) is harvested, aliquoted and stored at 4° C., −20° C., or −80° C., depending on how long the arenavirus vector should be stored prior use. The arenavirus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous non-arenaviral polypeptide, wherein a plasmid encoding the genomic segment is modified to incorporate a nucleotide encoding a heterologous non-arenaviral polypeptide. The nucleotide encoding a heterologous non-arenaviral polypeptide can be incorporated into the plasmid using restriction enzymes and ligases by molecular cloning techniques as known to those skilled in the art.

### 5.10.2 Infectious, Replication-Defective Arenavirus Particle

Infectious, replication-defective arenavirus particles can be rescued as described above. However, once generated from DNA, the infectious, replication-deficient arenaviruses provided herein can be propagated in complementing cells. Complementing cells are cells that provide the functionality that has been eliminated from the replication-deficient arenavirus by modification of its genome (e.g., if the ORF encoding the GP protein is deleted or functionally inactivated, a complementing cell does provide the GP protein).

Owing to the removal or functional inactivation of one or more of the ORFs in arenavirus vectors (here deletion of the glycoprotein, GP, will be taken as an example), arenavirus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express or encode the viral gene(s) deleted in the arenavirus vector to be generated under control of one or more expression cassettes suitable for expression in mammalian cells, e.g., a mammalian polymerase II promoter such as the EF1alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids can be of two types: i) two plasmids, referred to as TF-plasmids for expressing intracellularly in C-cells the minimal trans-acting factors of the arenavirus, is derived from e.g., NP and L proteins of LCMV in the present example; and ii) plasmids, referred to as GS-plasmids, for expressing intracellularly in C-cells the arenavirus vector genome segments, e.g., the segments with designed modifications. TF-plasmids express the NP and L proteins of the respective arenavirus vector under control of an expression cassette suitable for protein expression in mammalian cells, typically e.g., a mammalian polymerase II promoter such as the CMV or EF1alpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and trans-acting factors can be transcribed by T7, polI and polII promoters from one plasmid.

For recovering of the arenavirus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and trans-acting factors can be transcribed by T7, polI and polII promoters from one plasmid. For this one can exploit any of the commonly used strategies such as calcium-phosphate, liposome-based protocols or electroporation.

3-5 days later: The culture supernatant (arenavirus vector preparation) is harvested, aliquoted and stored at 4° C., −20° C. or −80° C. depending on how long the arenavirus vector should be stored prior to use. Then the arenavirus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient arenavirus expressing an antigen. When used for expression of an antigen in cultured cells, the following two procedures can be used:
i) The cell type of interest is infected with the arenavirus vector preparation described herein at a multiplicity of infection (MOI) of one or more, e.g., two, three or four, resulting in production of the antigen in all cells already shortly after infection.
ii) Alternatively, a lower MOI can be used and individual cell clones can be selected for their level of virally driven antigen expression. Subsequently individual clones can be expanded infinitely owing to the non-cytolytic nature of arenavirus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of antigen using infectious, replication-deficient arenaviruses as vectors may be considered.

### 5.11 Methods of Using the Compositions Provided Herein—Generation and Rescue of Arenavirus Particles

In certain embodiments, the nucleotide sequence as provided herein (see Section 5.5) can be used to generate and/or rescue arenavirus particles. In certain embodiments, the arenavirus particles as provided herein (see Section 5.6) can be used to generate and/or rescue arenavirus particles. In certain embodiments, the arenavirus genomic or antigenomic segment as provided herein (see Section 5.7) can be used to generate and/or rescue arenavirus particles.

Generally, arenavirus particles can be recombinantly produced by standard reverse genetic techniques as described for LCMV (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein).

In certain embodiments, an arenavirus particle generated or rescued can be bi-segmented. In other embodiments, an arenavirus particle generated or rescued can be tri-segmented. A tri-segmented arenavirus particle can be recombinantly produced by reverse genetic techniques known in the art, for example as described in International Publication No.: WO 2016/075250 A1.

### 5.11.1 Infectious and Replication Competent Arenavirus Particle

In certain embodiments, the method of generating a bi-segmented arenavirus particle comprises (i) transfecting into a host cell one or more DNA sequences as described in Section 5.5, or one or more RNA sequences as described in Section 5.5, or one or more RNA sequences each transcribed in vitro from the DNA sequence as described in Section 5.5; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell one or more DNA sequences as described in Section 5.5, one or more RNA sequences as described in Section 5.5, or one or more RNA sequences each transcribed in vitro from the DNA sequence as described in Section 5.5, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, the method of generating a bi-segmented arenavirus particle comprises (i) transfecting into a host cell one or more cDNA sequences of the mRNA transcripts of the genome of the arenavirus particle as described in Section 5.6 or one or more RNA sequences each transcribed in vitro from the cDNA of the mRNA transcript of the genome of the arenavirus particle as described in Section 5.6; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell one or more cDNA sequences of the mRNA transcripts of the genome of the arenavirus particle as described in Section 5.6 or one or more RNA sequences each transcribed in vitro from the cDNA of the mRNA transcript of the genome of the arenavirus particle as described in Section 5.6, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, the method of generating a bi-segmented arenavirus particle comprises (i) transfecting into a host cell the cDNA sequences of the arenavirus genomic or antigenomic segment as described in Section 5.7 or one or more RNA sequences each transcribed in vitro from the cDNA sequence of the arenavirus genomic or antigenomic segment as described in Section 5.7; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

In certain embodiments, when the arenavirus particle is tri-segmented, the method of generating the arenavirus particle comprises (i) transfecting into a host cell the cDNA sequences of the arenavirus genomic or antigenomic segment as described in Section 5.7 or one or more RNA sequences each transcribed in vitro from the cDNA sequence of the arenavirus genomic or antigenomic segment as described in Section 5.7, wherein the above nucleotide sequences encode the one L segment and two S segments or two L segments and one S segment; (ii) transfecting into the host cell nucleotide sequences driving intracellular expression of arenaviral trans-acting factors; (iii) maintaining the host cell under conditions suitable for virus formation; and (iv) harvesting the arenavirus particle. In certain more specific embodiments, the DNA is comprised in a plasmid.

More details on generation of infectious and replication competent arenavirus particles are described in Section 5.10.1.

### 5.11.2 Infectious, Replication-Defective Arenavirus Particle

Infectious, replication-defective arenavirus particles can be rescued as described above. More details on generation of infectious, replication-defective arenavirus particles are described in Section 5.10.2.

### 5.12 Methods of Using the Compositions Provided Herein—Vaccine

The present application furthermore relates to vaccines, immunogenic compositions (e.g., vaccine formulations), and pharmaceutical compositions comprising a nucleotide sequence as described in Section 5.5 and related expression products, arenavirus particles and arenavirus genomic or antigenomic segments. The present application also relates to vaccines, immunogenic compositions (e.g., vaccine formulations), and pharmaceutical compositions comprising an arenavirus particle as described in Section 5.6. The present application further relates to vaccines, immunogenic compositions (e.g., vaccine formulations), and pharmaceutical compositions comprising an arenavirus genomic or antigenomic segment as described in Section 5.7 and related arenavirus particles. The present application further relates to vaccines, immunogenic compositions (e.g., vaccine formulations), and pharmaceutical compositions comprising a translation product as described in Section 5.8. The present application further relates to vaccines, immunogenic compositions (e.g., vaccine formulations), and pharmaceutical compositions comprising a cDNA, DNA sequence and/or DNA expression vector related to the nucleotide sequences as described in Section 5.5, the arenavirus particle as described in Section 5.6, and/or the arenavirus genomic or antigenomic segments as described in Section 5.7, for example a DNA sequence encoding the arenavirus genomic or antigenomic segment as described in Section 5.7. Such vaccines, immunogenic compositions and pharmaceutical compositions can be formulated according to standard procedures in the art.

It will be readily apparent to one of ordinary skill in the relevant arts that suitable modifications and adaptations to the methods and applications described herein can be obvious and can be made without departing from the scope or any embodiment thereof.

In certain embodiments, provided herein are compositions comprising the nucleotide sequences as described in Section 5.5. In another embodiment, provided herein are compositions comprising the arenavirus particle as described in Section 5.6. In another embodiment, provided herein are compositions comprising the arenavirus genomic or antigenomic segments as described in Section 5.7. In another embodiment, provided herein are compositions comprising the translation product as described in Section 5.8. In another embodiment, provided herein are compositions comprising a cDNA, DNA sequence and/or DNA expression vectors related to the nucleotide sequences as described in Section 5.5, the arenavirus particle as described in Section 5.6, and/or the arenavirus genomic or antigenomic segments as described in Section 5.7, for example a DNA sequence encoding the arenavirus genomic or antigenomic segment as described in Section 5.7. Such compositions can be used in methods of treatment and prevention of disease. In a specific embodiment, the compositions described herein are used in the treatment of subjects infected with, or susceptible to, an infection. In other embodiments, the compositions described herein are used in the treatment of subjects susceptible to or exhibiting symptoms characteristic of cancer or tumorigenesis or are diagnosed with cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention or treatment of infection or cancer of subjects (e.g., human subjects). In other embodiments, the vaccine, immunogenic composition or pharmaceutical composition are suitable for veterinary and/or human administration.

In certain embodiments, provided herein are immunogenic compositions comprising an arenavirus vector as described herein. In some embodiments, a subject is immunized or treated with one or more arenavirus vector of the disclosure. In some embodiments, a subject is primed with one arenavirus of the disclosure (e.g., LCMV, Lassa virus, PICV, Junin virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus, Alxa virus, Chapare virus, Lijiang virus, Cupixi virus, Gairo virus, Loei River virus, Lujo virus, Luna virus, a Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, and/or Xapuri virus derived arenavirus) and boosted with a different arenavirus of the disclosure (e.g., LCMV, Lassa virus, PICV, Junin virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus, Alxa virus, Chapare virus, Lijiang virus, Cupixi virus, Gairo virus, Loei River virus, Lujo virus, Luna virus, a Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, and/or Xapuri virus derived arenavirus). In some embodiments, a subject is primed with a first arenavirus of the disclosure (e.g., PICV, LCMV, TAMV, TCRV, or any arenavirus species of the disclosure) and boosted with a second arenavirus of the disclosure (e.g., PICV, LCMV, TAMV, TCRV, or any arenavirus species of the disclosure). In some embodiments, a subject is immunized twice with arenavirus vector(s) of the disclosure (e.g., split based vectors). In some embodiments, a subject is immunized twice with the same arenavirus vector(s) of the disclosure (e.g., split based vectors or “art” based vectors of the disclosure). In some embodiments, a subject is immunized twice with different arenavirus vectors of the disclosure (e.g., split based vectors of the disclosure (e.g., a split based vector comprising a buffer peptide and/or a split based vector that does not comprise a buffer peptide, and/or an “art” based vector of the disclosure). In some embodiments, a subject is immunized twice with different arenavirus vectors of the disclosure (e.g., two or more different split based vectors comprising a buffer peptide). In some embodiments, a subject is immunized twice with different arenavirus vectors of the disclosure (e.g., a split based vector comprising a buffer peptide and a split based vector that does not comprise a buffer peptide). In some embodiments, a subject is immunized twice with different arenavirus vectors of the disclosure (e.g., a split based vector comprising a buffer peptide and an “art” based vector of the disclosure). In some embodiments, a subject is immunized twice with different arenavirus vectors of the disclosure (e.g., a split based vector that does not comprise a buffer peptide and an “art” based vector of the disclosure). In some embodiments, disclosed herein is a method of immunizing a subject suspected of having, diagnosed with, or at risk of developing a disease or disorder (e.g., an infection and/or a cancer). In some embodiments, the method comprises administering a first engineered arenavirus particle to the subject; and administering a second engineered arenavirus particle to the subject. In some embodiments, the first engineered arenavirus particle is derived from a first arenavirus species or clade. In some embodiments, the second engineered arenavirus particle is derived from a second arenavirus species or clade. In some embodiments, the first and the second arenavirus species or clade are the same (e.g., PICV, LCMV, TAMV, TCRV, or any arenavirus species of the disclosure). In some embodiments, the first and the second arenavirus particles are derived from different arenavirus species or clades (e.g., from PICV, LCMV, TAMV, TCRV, or any combination of arenavirus species of the disclosure). In some embodiments, the first engineered arenavirus particle and the second engineered arenavirus particle are the same. In some embodiments, the first engineered arenavirus particle and the second engineered arenavirus particle are different. In some embodiments, the first engineered arenavirus particle is derived from a first arenavirus species. In some embodiments, the second engineered arenavirus particle is derived from a second arenavirus species. In some embodiments, the first arenavirus species is LCMV and the second arenavirus species is PICV. In some embodiments, the first arenavirus species is PICV and the second arenavirus species is LCMV. In some embodiments, the first arenavirus species is LCMV and the second arenavirus species is TAMV. In some embodiments, the first arenavirus species is TAMV and the second arenavirus species is LCMV. In some embodiments, the first arenavirus species is LCMV and the second arenavirus species is TCRV. In some embodiments, the first arenavirus species is TCRV and the second arenavirus species is LCMV. In some embodiments, the first arenavirus species is TAMV and the second arenavirus species is PICV. In some embodiments, the first arenavirus species is PICV and the second arenavirus species is TAMV. In some embodiments, the first arenavirus species is TCRV and the second arenavirus species is PICV. In some embodiments, the first arenavirus species is PICV and the second arenavirus species is TCRV. In some embodiments, the first arenavirus species is TCRV and the second arenavirus species is TAMV. In some embodiments, the first arenavirus species is TAMV and the second arenavirus species is TCRV. In some embodiments, the first arenavirus species is any arenavirus species of the disclosure (e.g., LCMV, Lassa virus, PICV, Junin virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus, Alxa virus, Chapare virus, Lijiang virus, Cupixi virus, Gairo virus, Loei River virus, Lujo virus, Luna virus, a Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, and/or Xapuri virus). In some embodiments, the second arenavirus species is any arenavirus species of the disclosure (e.g., LCMV, Lassa virus, PICV, Junin virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus, Alxa virus, Chapare virus, Lijiang virus, Cupixi virus, Gairo virus, Loei River virus, Lujo virus, Luna virus, a Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, and/or Xapuri virus). In some embodiments, the third and/or any subsequent arenavirus species is any arenavirus species of the disclosure (e.g., LCMV, Lassa virus, PICV, Junin virus, Oliveros virus, Tamiami virus, Mobala virus, Mopeia virus, Ippy virus, Amapari virus, Flexal virus, Guanarito virus, Latino virus, Machupo virus, Parana virus, Pirital virus, Sabia virus, Tacaribe virus, Bear Canyon virus, Whitewater Arroyo virus, Allpahuayo virus, Alxa virus, Chapare virus, Lijiang virus, Cupixi virus, Gairo virus, Loei River virus, Lujo virus, Luna virus, a Luli virus, Lunk virus, Mariental virus, Merino Walk virus, Morogoro virus, Okahandja virus, Apore virus, Ryukyu virus, Solwezi virus, souris virus, Wenzhou virus, Big Brushy Tank virus, Catarina virus, Skinner Tank virus, Tonto Creek virus, and/or Xapuri virus). In some embodiments, a composition is administered to the patient as a single dose followed by a second dose three to six weeks later. In some embodiments, a booster inoculation may be administered to the subject at six to twelve months intervals following a second inoculation. In some embodiments, a booster inoculation may utilize a different arenavirus or composition thereof. In some embodiments, the administration of a composition may be repeated and separated by at least 1 day, 2 days, 3 days, 4 days, 5 days, 10 days, 15 days, 30 days, 45 days, 2 months, 75 days, 3 months, or at least 6 months. In some embodiments, the first engineered arenavirus particle of the disclosure is administered prior to the second, or the third, or any subsequent engineered arenavirus particle (e.g., prior to by about, at least by about, or at most by about: 30 minutes, 1 hr, 2 hrs, 3 hrs, 6 hrs, 10 hrs, 12 hrs, 16 hrs, 20 hrs, 24 hrs, 1 day, 2 days, 3 days, 4 days, 5 days, 6 days, 7 days, 1 week, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, 8 weeks, 8.5 weeks, 9 weeks, 9.5 weeks, 10 weeks, 10.5 weeks, 11 weeks, 11.5 weeks, 12 weeks, 12.5 weeks, 13 weeks, 13.5 weeks, 14 weeks, 14.5 weeks, 15 weeks, 15.5 weeks, 16 weeks, 16.5 weeks, 17 weeks, 17.5 weeks, 18 weeks, 18.5 weeks, 19 weeks, 19.5 weeks, 20.5 weeks, 21 weeks, 21.5 weeks, 22 weeks, 22.5 weeks, 23 weeks, 23.5 weeks, 24 weeks, 24.5 weeks, 25 weeks, 25.5 weeks, 26 weeks, 26.5 weeks, 27 weeks, 27.5 weeks, 28 weeks, 28.5 weeks, 29 weeks, 29.5 weeks, 30 weeks 31.5 weeks, 32 weeks, or more than 32 weeks, 1 month, 3 months, 6 months, 9 months, 12 months, 15 months, 18 months, 20 months, 24 months, or more than 24 months). In some embodiments, the first engineered arenavirus particle of the disclosure is administered on the same day as the second engineered arenavirus particle. In some embodiments, the first engineered arenavirus particle of the disclosure is administered after the second, or the third, or any subsequent engineered arenavirus particle (e.g., after by about, at least by about, or at most by about: 30 minutes, 1 hr, 2 hrs, 3 hrs, 6 hrs, 10 hrs, 12 hrs, 16 hrs, 20 hrs, 24 hrs, 1 day, 2 days, 3 days, 4 days, 5 days, 6 days, 7 days, 1 week, 1.5 weeks, 2 weeks, 2.5 weeks, 3 weeks, 3.5 weeks, 4 weeks, 4.5 weeks, 5 weeks, 5.5 weeks, 6 weeks, 6.5 weeks, 7 weeks, 7.5 weeks, 8 weeks, 8.5 weeks, 9 weeks, 9.5 weeks, 10 weeks, 10.5 weeks, 11 weeks, 11.5 weeks, 12 weeks, 12.5 weeks, 13 weeks, 13.5 weeks, 14 weeks, 14.5 weeks, 15 weeks, 15.5 weeks, 16 weeks, 16.5 weeks, 17 weeks, 17.5 weeks, 18 weeks, 18.5 weeks, 19 weeks, 19.5 weeks, 20.5 weeks, 21 weeks, 21.5 weeks, 22 weeks, 22.5 weeks, 23 weeks, 23.5 weeks, 24 weeks, 24.5 weeks, 25 weeks, 25.5 weeks, 26 weeks, 26.5 weeks, 27 weeks, 27.5 weeks, 28 weeks, 28.5 weeks, 29 weeks, 29.5 weeks, 30 weeks 31.5 weeks, 32 weeks, or more than 32 weeks, 1 month, 3 months, 6 months, 9 months, 12 months, 15 months, 18 months, 20 months, 24 months, or more than 24 months). In some embodiments, the method further comprises administering a third engineered arenavirus particle of the disclosure. In some embodiments, the third engineered arenavirus particle of the disclosure is derived from the same arenavirus species or serotypes as the first and/or as the second engineered arenavirus particle. In some embodiments, the third engineered arenavirus particle of the disclosure is derived from a different arenavirus species or serotypes as the first and/or as the second engineered arenavirus particle. In some embodiments, the method further comprises administering several engineered arenavirus particle of the disclosure (e.g., one additional, two, three, four, five, six, seven, or more than seven engineered arenavirus particle of the disclosure). In some embodiments, the several engineered arenavirus particle of the disclosure are derived from the same arenavirus species or clades. In some embodiments, the several engineered arenavirus particle of the disclosure are derived from different arenavirus species or clades. In some embodiments, some of the several engineered arenavirus particle of the disclosure are derived from different arenavirus species or clades and some from the same arenavirus species or clades.

In some embodiments, an engineered arenavirus particle of the disclosure or an arenavirus vector of the disclosure is disclosed in Section 5.6. In some embodiments, the genome of an engineered arenavirus particle of the disclosure (e.g., first, second, third, and/or subsequent engineered arenavirus particle of the disclosure) consists of or comprises: a) an S segment that encodes arenavirus GP signal peptide and a heterologous non-arenaviral polypeptide or arenavirus GP signal peptide alone under the control of an arenavirus 3′ UTR and a heterologous non-arenaviral signal peptide, arenavirus GP1 and arenavirus GP2 under the control of an arenavirus 5′ UTR; b) an S segment that encodes NP under the control of an arenavirus 3′ UTR and another heterologous non-arenaviral polypeptide or no polypeptide under the control of an arenavirus 5′ UTR; and c) an L segment that encodes L protein under the control of an arenavirus 3′ UTR and Z protein under the control of an arenavirus 5′ UTR. In some embodiments, the genome of an engineered arenavirus particle of the disclosure (e.g., first, second, third, or several of the engineered arenavirus particle of the disclosure) consists of or comprises: a) an S segment that encodes arenavirus GP signal peptide and a heterologous non-arenaviral polypeptide or arenavirus GP signal peptide alone under the control of an arenavirus 3′ UTR and another heterologous non-arenaviral polypeptide or no polypeptide under the control of an arenavirus 5′ UTR; b) an S segment that encodes NP under the control of an arenavirus 3′ UTR and a heterologous non-arenaviral signal peptide, arenavirus GP1 and arenavirus GP2 under the control of an arenavirus 5′ UTR; and c) an L segment that encodes L protein under the control of an arenavirus 3′ UTR and Z protein under the control of an arenavirus 5′ UTR. In some embodiments, the two heterologous non-arenaviral polypeptides are the same or different from each other. In some embodiments, an arenavirus, an engineered arenavirus particle, or arenavirus vector of the disclosure is an arenavirus, arenavirus particle, or vector disclosed in WO2021/089853, the content of which is incorporated herein in its entirety.

In some embodiments, the genome of an engineered arenavirus particle of the disclosure is referred to herein as “art” (e.g., “artLCMV” or “artPICV”). In some embodiments, the genome of an engineered arenavirus particle of the disclosure (referred to herein as “art”) (e.g., first, second, third, and/or subsequent engineered arenavirus particle of the disclosure) consists of or comprises: (i) an S segment, wherein the ORF encoding the nucleoprotein is under control of an arenavirus 5′ untranslated region; (ii) an S segment, wherein the ORF encoding the matrix protein Z (“Z protein”) is under control of an arenavirus 5′ UTR; (iii) an S segment, wherein the ORF encoding the RNA dependent RNA polymerase L (“L protein”) is under control of an arenavirus 5′ UTR; (iv) an S segment, wherein the ORF encoding the viral glycoprotein is under control of an arenavirus 3′ UTR; (v) an S segment, wherein the ORF encoding the L protein is under control of an arenavirus 3′ UTR; (vi) an S segment, wherein the ORF encoding the Z protein is under control of an arenavirus 3′ UTR; (vii) an L segment, wherein the ORF encoding the GP is under control of an arenavirus 5′ UTR; (viii) an L segment, wherein the ORF encoding the NP is under control of an arenavirus 5′ UTR; (ix) an L segment, wherein the ORF encoding the L protein is under control of an arenavirus 5′ UTR; (x) an L segment, wherein the ORF encoding the GP is under control of an arenavirus 3′ UTR; (xi) an L segment, wherein the ORF encoding the NP is under control of an arenavirus 3′ UTR; (xii) an L segment, wherein the ORF encoding the Z protein is under control of an arenavirus 3′ UTR. In some embodiments, an arenavirus, an engineered arenavirus particle, or arenavirus vector of the disclosure is an arenavirus, arenavirus particle, or vector disclosed in WO2016/075250, the content of which is incorporated herein in its entirety.

In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an arenavirus particle or tri-segmented arenavirus particle and, most importantly, the gene products it vectorises, but when the compound is administered alone does not generate an immune response to the arenavirus particle or tri-segmented arenavirus particle and the gene products vectorised by the latter. In some embodiments, the adjuvant generates an immune response to the arenavirus particle or tri-segmented arenavirus particle and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition provided herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design. The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the bi-segmented arenaviruses particle or tri-segmented arenavirus particle described herein alone or together with a pharmaceutically acceptable carrier. Suspensions or dispersions of the arenavirus particle or tri-segmented arenavirus particle, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 103 to about 10¹¹ focus forming units of the bi-segmented arenavirus particle or tri-segmented arenavirus particle.

In one embodiment, administration of the pharmaceutical composition is parenteral administration. Parenteral administration can be intravenous or subcutaneous administration. Accordingly, unit dose forms for parenteral administration are, for example, ampoules or vials, e.g., vials containing from about 10³ to 10¹⁰ focus forming units or 10⁵ to 10¹⁵ physical particles of the arenavirus particle or tri-segmented arenavirus particle.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, intra-tumoral, and via scarification (scratching through the top layers of skin, e.g., using a bifurcated needle). Specifically, subcutaneous or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

The dosage of the active ingredient depends upon the type of vaccination or immunotherapy and upon the subject, and their age, weight, individual condition, the individual pharmacokinetic data, and the mode of administration. In certain embodiments, an in vitro assay is employed to help identify optimal dosage ranges. Effective doses may be extrapolated from dose response curves derived from in vitro or animal model test systems.

In certain embodiments, the vaccine, immunogenic composition, or pharmaceutical composition comprising an arenavirus particle or the tri-segmented arenavirus particle can be used as a live vaccination or immunotherapy. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed. In another embodiment, a live arenavirus is formulated in a dose that is of a volume selected from the group consisting of 0.2, 0.5, 1, 2, 5, 7, 10, 15, 20, 25, 30, 35, 40, 45, and 50 mL. In certain embodiments, the dose contains 10^{5.5}-10^{9.5} infectious focus forming units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus.

In certain embodiments, for administration to children, two doses of a bi-segmented arenavirus particle or a tri-segmented arenavirus particle described herein or a composition thereof, given at least one month apart, are administered to a child. In specific embodiments for administration to adults, a single dose of the arenavirus particle or tri-segmented arenavirus particle described herein or a composition thereof is given. In another embodiment, two doses of a bi-segmented arenavirus particle or a tri-segmented arenavirus particle described herein or a composition thereof, given at least one month apart, are administered to an adult. In another embodiment, a young child (six months to nine years old) may be administered a bi-segmented arenavirus particle or a tri-segmented arenavirus particle described herein or a composition thereof for the first time in two doses given one month apart. In a particular embodiment, a child who received only one dose in their first year of vaccination or immunotherapy should receive two doses in the following year. In some embodiments, two doses administered 4 weeks apart are preferred for children 2-8 years of age who are administered an immunogenic composition described herein, for the first time. In certain embodiments, for children 6-35 months of age, a half dose (0.25 ml) may be preferred, in contrast to 0.5 ml which may be preferred for subjects over three years of age.

In certain embodiments, the compositions can be administered to the patient in a single dosage comprising a therapeutically effective amount of the arenavirus particle or the tri-segmented arenavirus particle. In some embodiments, the bi-segmented arenavirus particle or tri-segmented arenavirus particle can be administered to the patient in a single dose comprising a therapeutically effective amount of a bi-segmented arenavirus particle or tri-segmented arenavirus particle and, one or more pharmaceutical compositions, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided herein, are processes and to the use the arenavirus particle or the tri-segmented arenavirus particle for the manufacture of vaccines in the form of pharmaceutical preparations, which comprise the arenavirus particle or tri-segmented arenavirus particle as an active ingredient. The pharmaceutical compositions of the present application are prepared in a manner known per se, for example by means of conventional mixing and/or dispersing processes.

### 5.13 Compositions, Administration, And Dosage

The present application furthermore relates to vaccines, immunogenic compositions (e.g., vaccine formulations), and pharmaceutical compositions comprising an arenavirus particle or a tri-segmented arenavirus particle as described herein. Such vaccines, immunogenic compositions and pharmaceutical compositions can be formulated according to standard procedures in the art.

It will be readily apparent to one of ordinary skill in the relevant arts that suitable modifications and adaptations to the methods and applications described herein can be obvious and can be made without departing from the scope of the scope or any embodiment thereof.

In another embodiment, provided herein are compositions comprising an arenavirus particle or a tri-segmented arenavirus particle described herein. Such compositions can be used in methods of treatment and prevention of disease. In a specific embodiment, the compositions described herein are used in the treatment of subjects infected with and infectious agent, or susceptible to an infection. In other embodiments, the compositions described herein are used in the treatment of subjects susceptible to or exhibiting symptoms characteristic of cancer or tumorigenesis or are diagnosed with cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention of infection or cancer of subjects (e.g., human subjects). In other embodiments, the vaccine, immunogenic composition or pharmaceutical composition are suitable for veterinary and/or human administration.

In certain embodiments, provided herein are immunogenic compositions comprising an arenavirus vector as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a arenavirus particle or tri-segmented arenavirus particle and, most importantly, the gene products it vectorises, but when the compound is administered alone does not generate an immune response to the arenavirus particle or tri-segmented arenavirus particle and the gene products vectorised by the latter. In some embodiments, the adjuvant generates an immune response to the arenavirus particle or tri-segmented arenavirus particle and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition provided herein comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), ASO3 (GlaxoSmithKline), ASO4 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design: The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the arenaviruses particle or tri-segmented arenavirus particle described herein alone or together with a pharmaceutically acceptable carrier. Suspensions or dispersions of the arenavirus particle or tri-segmented arenavirus particle, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 103 to about 10¹¹ focus forming units of the arenavirus particle or tri-segmented arenavirus particle.

In one embodiment, administration of the pharmaceutical composition is parenteral administration. Parenteral administration can be intravenous or subcutaneous administration. Accordingly, unit dose forms for parenteral administration are, for example, ampoules or vials, e.g., vials containing from about 10′ to 10¹⁰ focus forming units or 105 to 10¹⁵ physical particles of the arenavirus particle or tri-segmented arenavirus particle.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

The dosage of the active ingredient depends upon the type of vaccination or immunotherapy and upon the subject, and their age, weight, individual condition, the individual pharmacokinetic data, and the mode of administration. In certain embodiments, an in vitro assay is employed to help identify optimal dosage ranges. Effective doses may be extrapolated from dose response curves derived from in vitro or animal model test systems.

In certain embodiments, the vaccine, immunogenic composition, or pharmaceutical composition comprising an arenavirus particle or the tri-segmented arenavirus particle can be used as a live vaccination or immunotherapy. Exemplary doses for a live arenavirus particle may vary from 10-100, or more, PFU of live virus per dose. In some embodiments, suitable dosages of an arenavirus particle or the tri-segmented arenavirus particle are 10², 5×10², 10³, 5×10³, 10⁴, 5×10⁴, 10⁵, 5×10⁵, 10⁶, 5×10⁶, 10⁷, 5×10⁷, 10, 5×10⁸, 1×10⁹, 5×10⁹, 1×10¹⁰, 5×10¹⁰, 1×10¹¹, 5×10¹¹ or 10¹² pfu, and can be administered to a subject once, twice, three or more times with intervals as often as needed. In another embodiment, a live arenavirus is formulated such that a 0.2-mL dose contains 10^{6.5}-10^{7.5} fluorescent focal units of live arenavirus particle. In another embodiment, an inactivated vaccine is formulated such that it contains about 15 μg to about 100 μg, about 15 μg to about 75 μg, about 15 μg to about 50 μg, or about 15 μg to about 30 μg of an arenavirus.

In certain embodiments, for administration to children, two doses of an arenavirus particle or a tri-segmented arenavirus particle described herein or a composition thereof, given at least one month apart, are administered to a child. In specific embodiments for administration to adults, a single dose of the arenavirus particle or tri-segmented arenavirus particle described herein or a composition thereof is given. In another embodiment, two doses of an arenavirus particle or a tri-segmented arenavirus particle described herein or a composition thereof, given at least one month apart, are administered to an adult. In another embodiment, a young child (six months to nine years old) may be administered an arenavirus particle or a tri-segmented arenavirus particle described herein or a composition thereof for the first time in two doses given one month apart. In a particular embodiment, a child who received only one dose in their first year of vaccination or immunotherapy should receive two doses in the following year. In some embodiments, two doses administered 4 weeks apart are preferred for children 2-8 years of age who are administered an immunogenic composition described herein, for the first time. In certain embodiments, for children 6-35 months of age, a half dose (0.25 ml) may be preferred, in contrast to 0.5 ml which may be preferred for subjects over three years of age.

In certain embodiments, the compositions can be administered to the patient in a single dosage comprising a therapeutically effective amount of the arenavirus particle or the tri-segmented arenavirus particle. In some embodiments, the arenavirus particle or tri-segmented arenavirus particle can be administered to the patient in a single dose comprising a therapeutically effective amount of an arenavirus particle or tri-segmented arenavirus particle and, one or more pharmaceutical compositions, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different arenavirus or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided herein, are processes and to the use the arenavirus particle or the tri-segmented arenavirus particle for the manufacture of vaccines in the form of pharmaceutical preparations, which comprise the arenavirus particle or tri-segmented arenavirus particle as an active ingredient. The pharmaceutical compositions of the present application are prepared in a manner known per se, for example by means of conventional mixing and/or dispersing processes.

### 5.14 Assays

In certain embodiments, an arenavirus particle described herein comprises the nucleotide sequence provided herein (see Section 5.5.2). In certain embodiments, an arenavirus particle described herein is engineered such that an arenaviral ORF is separated over two or more mRNA transcripts (see Section 5.6). In certain embodiments, an arenavirus particle described herein is derived from an arenavirus genomic or antigenomic segment provided herein (see Section 5.7). In certain embodiments, an arenavirus particle described herein comprises an arenavirus genomic or antigenomic segment provided herein (see Section 5.7).

In certain embodiments, the arenavirus particle provided herein is genetically stable, i.e. the inability of the arenavirus particle to revert to wild-type-like (more virulent) replication behavior in the host (genetic stability). In certain embodiments, the genetic stability may be demonstrated using assays described in this Section and Section 6.

In certain embodiments, the arenavirus particle provided herein shows high-level transgene expression to elicit strong immune responses against the desired target antigen(s) (transgene expression levels). In certain embodiments, the high-level transgene expression may be demonstrated using assays described in this Section and Section 6.

In certain embodiments, the arenavirus particle expresses a heterologous non-arenaviral polypeptide. In certain embodiments, the heterologous non-arenaviral polypeptide is under control of an arenavirus 3′ UTR. In certain embodiments, the expression level of the heterologous non-arenaviral polypeptide is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, the expression level of the heterologous non-arenaviral polypeptide is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, the expression level of the heterologous non-arenaviral polypeptide is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the expression level of the same heterologous non-arenaviral polypeptide expressed under control of the respective arenavirus 5′ UTR. In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express the heterologous non-arenaviral polypeptide after an arenavirus of the present disclosure (e.g., expressing a heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide after an arenavirus of the present disclosure (e.g., expressing a heterologous non-arenaviral polypeptide under control of an arenavirus 3′ UTR) is introduced to (or infects) a population of cells is higher as compared to the proportion of cells that express the heterologous non-arenaviral polypeptide after an arenavirus expressing the same heterologous non-arenaviral polypeptide under control of an arenavirus 5′ UTR is introduced to (or infects) a population of cells. In certain embodiments, at least about, or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide after an arenavirus of the present disclosure (e.g., expressing a heterologous non-arenaviral polypeptide under control of an arenavirus 3′ UTR) is introduced to (or infects) a population of cells as compared to the proportion (or amount) of cells that express the heterologous non-arenaviral polypeptides after an arenavirus expressing the same heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, at least about, or about, 5%, 10%, 15%, 20%, 25%, 30%, 35%, 40%, 4%5% 50%, 55%, ⁶⁰, 65%, 70%, 75%, ⁸⁰, 85%, 90%, 95%, 99%, or 100% of cells express a heterologous non-arenaviral polypeptide and/or express a second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) the cells (e.g., to a population of cells). In certain embodiments, the proportion of cells that express the heterologous non-arenaviral polypeptide and/or express the second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells is higher as compared to the proportion of cells that express the heterologous non-arenaviral polypeptide or the second heterologous non-arenaviral polypeptide after an arenavirus expressing the same heterologous non-arenaviral polypeptide under control of an arenavirus 5′ UTR and/or expressing the same second heterologous non-arenaviral polypeptide under control of an arenavirus 5′ UTR is introduced to (or infects) a population of cells. In certain embodiments, at least about, or about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express the heterologous non-arenaviral polypeptide and/or express the second heterologous non-arenaviral polypeptide after an arenavirus of the present disclosure (e.g., expressing the heterologous non-arenaviral polypeptide under control of an arenavirus 3′ UTR and/or expressing the second heterologous non-arenaviral polypeptide under control of an arenavirus 3′ UTR) is introduced to (or infects) a population of cells as compared to the proportion (or amount) of cells that express the heterologous non-arenaviral polypeptides and/or express the second heterologous non-arenaviral polypeptides after an arenavirus expressing the same heterologous non-arenaviral polypeptide under control of an arenavirus 5′ UTR and/or expressing the same second heterologous non-arenaviral polypeptide under control of an arenavirus 5′ UTR is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle expresses two heterologous non-arenaviral polypeptides. In certain embodiments, expression of one heterologous non-arenaviral polypeptide is under control of an arenavirus 3′ UTR and expression of the other heterologous non-arenaviral polypeptide is under control of an arenavirus 5′ UTR. In certain embodiments, the expression of a first of the two heterologous non-arenaviral polypeptides is under control of an arenavirus 3′ UTR in a first S segment and the expression of a second of the two heterologous non-arenaviral polypeptides is under control of an arenavirus 5′ UTR in a second S segment. In certain embodiments, the expression of one heterologous non-arenaviral polypeptide is under control of an arenavirus 3′ UTR in one S segment and the expression of the other heterologous non-arenaviral polypeptide is under control of an arenavirus 5′ UTR in the other S segment. In certain embodiments, the expression of a first of the two heterologous non-arenaviral polypeptides is under control of an arenavirus 5′ UTR in a first S segment and the expression of a second of the two heterologous non-arenaviral polypeptides is under control of an arenavirus 3′ UTR in a second S segment. In certain embodiments, the expression of one heterologous non-arenaviral polypeptide is under control of an arenavirus 5′ UTR in one S segment and the expression of the other heterologous non-arenaviral polypeptide is under control of an arenavirus 3′ UTR in the other S segment. In certain embodiments, cells that are infected with the arenavirus particle of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) express (co-express) both heterologous non-arenaviral polypeptides. In certain embodiments, the combined expression level of the two heterologous non-arenaviral polypeptides (e.g., cells expressing both heterologous non-arenaviral polypeptides) is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide is under control of an arenavirus 5′ UTR of one S segment and expression of the other heterologous non-arenaviral polypeptide is under control of an arenavirus 5′ UTR of the other S segment). In certain embodiments, the combined expression level of the two heterologous non-arenaviral polypeptides (e.g., cells expressing both heterologous non-arenaviral polypeptides) is about 1%, 2%, 3%, 4%, 5%, 6%, 7%, 8%, 9%, 10, 1%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide under control of an arenavirus 5′ UTR of one S segment and expression of the other heterologous non-arenaviral polypeptide under control of an arenavirus 5′ UTR of the other S segment). In certain embodiments, the combined expression level of the two heterologous non-arenaviral polypeptides (e.g., cells expressing both heterologous non-arenaviral polypeptides) is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher than the combined expression level of the same two heterologous non-arenaviral polypeptides expressed under control of arenavirus 5′ UTRs (e.g., expression of one heterologous non-arenaviral polypeptide under control of an arenavirus 5′ UTR of one S segment and expression of the other heterologous non-arenaviral polypeptide under control of an arenavirus 5′ UTR of the other S segment). In certain embodiments, at least about, or about, 5%, 10%, 15%, 20%, 25%, 30%, 35%, 40%, 45%, 50%, 55%, 60%, 65%, 70%, 75%, 80%, 85%, 90%, 95%, 99%, or 100% of cells express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) the cells (e.g., to a population of cells). In certain embodiments, the proportion (or amount) of cells that express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells is higher as compared to the proportion (or amount) of cells that express (co-express) the two heterologous non-arenaviral polypeptides after an arenavirus expressing the same two heterologous non-arenaviral polypeptides under control of arenavirus 5′ UTRs (e.g., one heterologous non-arenaviral polypeptide expressed under control of an arenavirus 5′ UTR of one S segment and the other heterologous non-arenaviral polypeptide expressed under control of an arenavirus 5′ UTR of the other S segment) is introduced to (or infects) a population of cells. In certain embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% more cells express (co-express) the two heterologous non-arenaviral polypeptides after an arenavirus of the present disclosure (e.g., expressing one heterologous non-arenaviral polypeptide under control of an arenavirus 3′ UTR in one S segment and expressing another heterologous non-arenaviral polypeptide under control of an arenavirus 5′ UTR in another S segment) is introduced to (or infects) a population of cells as compared to the proportion (or amount) of cells that express (co-express) the two heterologous non-arenaviral polypeptides after an arenavirus expressing the same two heterologous non-arenaviral polypeptides under control of arenavirus 5′ UTRs (e.g., expressing one heterologous non-arenaviral polypeptide under control of an arenavirus 5′ UTR of one S segment and expressing the other heterologous non-arenaviral polypeptide under control of an arenavirus 5′ UTR of the other S segment) is introduced to (or infects) a comparable population of cells. In certain embodiments, the cells are from a biological sample from a subject. In certain embodiments, the biological sample is from an organ (e.g., spleen). In certain embodiments, the biological sample is a blood sample.

In certain embodiments, the arenavirus particle provided herein may show good growth in cell culture, enabling the arenavirus particle's production to high titers in industrial fermentation processes (production yields). In certain embodiments, the good growth in cell culture may be demonstrated using the assays described in this Section and in Section 6.

In certain embodiments, the titer of the arenavirus particle of the disclosure is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, the titer of the arenavirus particle of the disclosure is about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, the titer of the arenavirus particle of the disclosure is 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle. In certain embodiments, a persistent infection may be an infection that lasts at least 5 days, 10 days, 14 days, 20 days, 25 days, 30 days, 35 days, 40 days, 45 days, 50 days, 53 days, 55 days, 60 days, 65 days, 70 days, 75 days, 78 days, 80 days, 90 days, or 100 days. In certain embodiments, a persistent infection may be an infection that lasts 5 days, 10 days, 14 days, 20 days, 25 days, 30 days, 35 days, 40 days, 45 days, 50 days, 53 days, 55 days, 60 days, 65 days, 70 days, 75 days, 78 days, 80 days, 90 days, or 100 days.

### 5.14.1 Arenavirus Detection Assays

The skilled artesian could detect an arenavirus genomic or antigenomic segment or tri-segmented arenavirus particle, as described herein using techniques known in the art. For example, RT-PCR can be used with primers that are specific to a genetically engineered arenavirus segment to detect and quantify an arenavirus genomic or antigenomic segment as described herein in section 5.7. Western blot, ELISA, radioimmunoassay, immunoprecipitation, or immunocytochemistry in conjunction with flow cytometry can be used to quantify the gene products of the arenavirus genomic or antigenomic segment or tri-segmented arenavirus particle.

### 5.14.2 Assay to Measure Infectivity

Any assay known to the skilled artisan can be used for measuring the infectivity of an arenavirus vector preparation. For example, determination of the virus/vector titer can be done by a “focus forming unit assay” (FFU assay). In brief, complementing cells are plated and inoculated with different dilutions of a virus/vector sample. For determination of replication-deficient arenavirus vector titers, complementing (e.g. glycoprotein-expressing cells in the case of glycoprotein-deficient arenavirus vectors) BHK-21 of HEK293 cells are used. For replication-competent vectors e.g., MC57, 3T3 or VERO cells are common cell substrates. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against the respective arenavirus' NP or another protein expressed by the arenavirus particle or the tri-segmented arenavirus particle and a HRP-based color reaction. The titer of a virus/vector can be calculated in focus-forming units per milliliter (FFU/mL).

### 5.14.3 Growth of an Arenavirus Particle

Growth of an arenavirus particle described herein can be assessed by any method known in the art or described herein (e.g., cell culture). Viral growth may be performed by inoculating serial dilutions of an arenavirus particle described herein into cell cultures (e.g., Vero cells or BHK-21 cells or complementing versions thereof in case of replication-deficient vectors). After incubation of the virus for a specified time, the supernatant containing virus is collected and virions can be purified using standard methods.

### 5.14.4 Serum ELISA

Determination of the humoral immune response upon vaccination or immunotherapy of animals (e.g., mice, guinea pigs) can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### 5.14.5 Assay to Measure the Neutralizing Activity of Induced Antibodies

Determination of the neutralizing antibodies in sera can be performed by plaque reduction neutralization tests. Depending on the viral target, suitable cells are used, such as ARPE-19 cells from ATCC for testing cytomegalovirus-neutralizing antibodies and a GFP-tagged virus (for example, cytomegalovirus) as test article. In addition, supplemental guinea pig serum as a source of exogenous complement may be used. The assay is started with seeding of 6.5×10′ cells/well (50 μl/well) in a 384 well plate one or two days before using for neutralization. The neutralization is performed by mixing of a defined virus inoculum in 96-well sterile tissue culture plates without cells but with titrated concentrations of test serum for 1 hour at 37° C. After the neutralization incubation step the mixture is added to the cells and incubated for additional 4 days for GFP-detection with a plate reader. Alternatively, instead of GFP or similar reporters, a viral structural protein can be detected as described in section 5.14.2. A positive neutralizing human sera is used as assay positive control on each plate to check the reliability of all results. Titers (EC50) are determined using a 4 parameter logistic curve fitting. As additional testing the wells are checked with a fluorescence microscope.

### 5.14.6 LCMV Plaque Reduction Neutralization Test

In brief, plaque reduction (neutralization) assays for LCMV can be performed by use of a replication-competent or -deficient LCMV that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples.

### 5.14.7 Determination of LCMV Genome Copies

qPCR LCMV RNA genomes are isolated using QIAamp Viral RNA mini Kit (QIAGEN), according to the protocol provided by the manufacturer. LCMV RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the LCMV NP coding region or another genomic stretch of the arenavirus particle or the tri-segmented arenavirus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the LCMV NP coding sequence or another genomic stretch of the arenavirus particle or the tri-segmented arenavirus particle containing the primer and probe binding sites.

### 5.14.8 Western Blotting

Infected cells grown in tissue culture flasks or in suspension are lysed at specific time points post infection using RIPA buffer (Thermo Scientific) or used directly without cell-lysis. Samples are heated to 99° C. for 10 minutes with reducing agent and NuPage LDS Sample buffer (Novex, Thermo Fisher Scientific, USA) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with a primary antibody directed against proteins of interest and alkaline phosphatase conjugated secondary antibodies followed by staining with 1-Step NBT/BCIP solution (Invitrogen, USA).

### 5.14.9 MHC-Peptide Multimer Staining Assay for Detection of Antigen-Specific CD8+ T-Cells

Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for a defined antigen specificity and MHC haplotype of T-cells (typically fluorescently labeled). The tetramer is then detected by flow cytometry via the fluorescent label.

### 5.14.10 ELISPOT Assay for Detection of Antigen-Specific T-cells.

Any assay known to the skilled artisan can be used to test antigen-specific T-cell responses (CD4+ and CD8+). For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Briefly, the assay comprises the following steps: An immunospot plate is coated with an anti-cytokine antibody. Cells are incubated in the immunospot plate in the presence of specific stimulus e.g. peptides against which reactivity is to be tested. Cells secrete cytokines and are then washed off. Plates are then coated with a second biotyinlated-anticytokine antibody and cytokine secretion spots are visualized with an avidin-HRP system. 5.14.11 Intracellular Cytokine Assay for Detection of Functionality of CD8+ and CD4+ T-cell Responses.

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After a defined period of incubation, typically 5 hours, a washing steps follows, and antibodies to other cellular markers can be added to the cells. Cells are then fixed and permeabilized. The flurochrome-conjugated anti-cytokine antibodies are added and the cells can be analyzed by flow cytometry.

### 5.14.12 Assay for Confirming Replication-Deficiency of Viral Vectors

Any assay known to the skilled artisan that determines concentration of infectious and replication-competent virus particles can also be used as a to measure replication-deficient viral particles in a sample. For example, FFU assays with non-complementing cells can be used for this purpose.

Furthermore, plaque-based assays are a standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32:Immunology of Infection. Academic Press. ISBN 0-12-521532-0), and thereby exerts a cytopathic or lytic effect. Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient arenavirus particles or tri-segmented arenavirus particles.

5.14.13 Assay for Expression of Viral Antigens Including Transgenically Inserted Non-Arenaviral Polypeptides Expressed from Viral Vectors

Any assay known to the skilled artisan can be used for measuring expression of viral antigens and transgenically inserted non-arenaviral polypeptides expressed from viral vectors. For example, FFU assays can be performed. For detection, mono- or polyclonal antibody preparation(s) against the respective viral antigens are used (transgene-specific FFU). Methods to determine the binding of specific antibodies to proteins expressed by the replication-competent or -deficient virus (viral structural proteins, viral non-structural proteins and transgenically inserted non-arenaviral polypeptides) comprise flow cytometry, immunocytochemistry, Immunofluorescence, Western Blot or dot blot. Fluorescent reporter proteins can be visualized by direct fluorescence microscopy or flow cytometry.

### 5.14.14 Animal Models

To investigate recombination and infectivity of an arenavirus particle described herein in vivo animal models can be used. In certain embodiments, the animal models that can be used to investigate recombination and infectivity of a tri-segmented arenavirus particle include mouse, guinea pig, rabbit, and monkeys. In a preferred embodiment, the animal models that can be used to investigate recombination and infectivity of an arenavirus include mouse. In a more specific embodiment, the mice, which can be used to investigate recombination and infectivity of an arenavirus particle, are triple-deficient for type I interferon receptor, type II interferon receptor and recombination activating gene 1 (RAG1).

In certain embodiments, the animal models can be used to determine arenavirus infectivity and transgene stability. In some embodiments, viral RNA can be isolated from the serum of the animal. Techniques are readily known by those skilled in the art. The viral RNA can be reverse transcribed and the cDNA carrying the arenavirus ORFs can be PCR-amplified with gene-specific primers. Flow cytometry can also be used to investigate arenavirus infectivity and transgene expression.

### 6. EXAMPLES

### 6.1 the Hierarchy of Vector Fitness and Viability is Influenced by the Amino Acid Sequence Stretch Immediately Downstream of the Predicted Glycoprotein Signal Peptide Cleavage Site

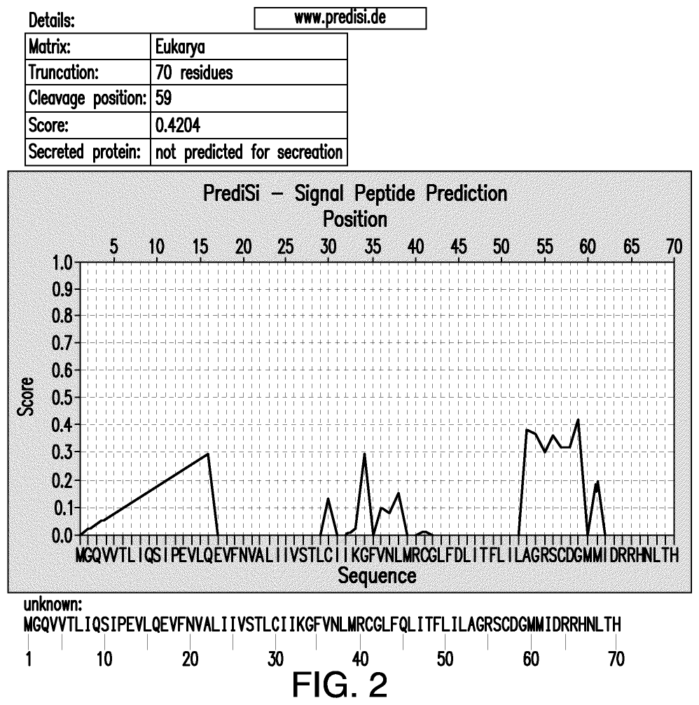

The arenavirus glycoprotein precursor GPC is inserted into the endoplasmid reticulum, followed by cleavage of its signal peptide, which remains non-covalently associated with the GP1 and GP2 subunits of GPC to form a tri-partite glycoprotein complex on infectious virions. The aforementioned arenavirus glycoprotein stable signal peptide (SSP) is commonly referred to as 58 amino acids (Aa) in length (Agnihothram, S S, et al. (2006) J Virol 80, 5189-5198; Eichler R, et al. (2003) EMBO Rep 4, 1084-1088; Schrempf S, et al. (2007) J Virol 81, 12515-12524). In this example, the Pichinde virus (PICV) glycoprotein sequence (PICV-GP) was submitted to the online signal peptide prediction algorithm PrediSi (PrediSi (Prediction of SIgnalpeptides) website (from the Technical University of Braunschweig)) and it was predicted that the signal peptide cleavage occurred between amino acids 59 and 60 (FIG. 2). This prediction resulted in a 59Aa-long signal peptide and the downstream GP1/GP2 sequences of PICV-GP from amino acids 60-508 of GPC.

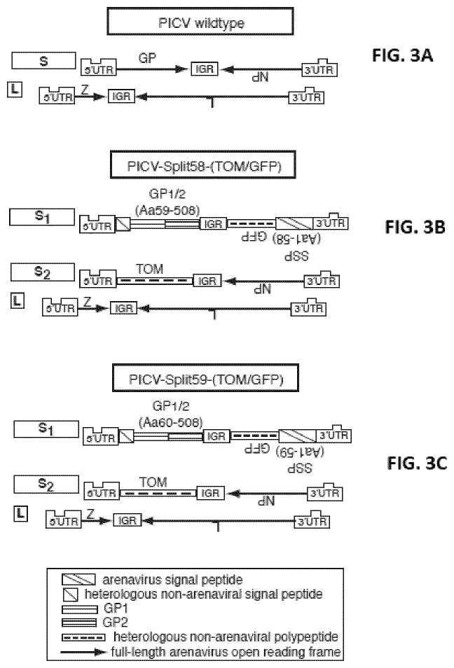

It was hypothesized that the wildtype PICV genome (FIG. 3A) could be converted into replication- and propagation-competent (i.e., “replication-competent”) tri-segmented PICV vectors. The genome would express the SSP and the GP1/GP2 portions of PICV-GP from separate transcriptional units and would, additionally, carry two non-arenaviral polypeptides. As described in FIGS. 3B and 3C, tri-segmented PICV vector genomes expressing as heterologous non-arenaviral polypeptides green fluorescent protein (GFP) and Tomato (TOM) were designed. GFP was expressed downstream and in-frame with the SSP under control of the genomic 3′ UTR promoter of the S segment 1 (S1), whereas the GP1/GP2 domains were expressed under control of the S1 segment's genomic 5′UTR promoter, preceded by an upstream in-frame heterologous non-arenaviral signal peptide, in this case the signal peptide of the vesicular stomatitis virus glycoprotein. The S segment 2 (S2) expressed the viral nucleoprotein and TOM under control of the genomic 3′ UTR and 5′ UTR promoters, respectively. Next, signal peptide lengths of 58Aa or 59Aa and GP1/GP2 domains corresponding to amino acids 59-508 or 60-508 of GPC, respectively, could generate replication-competent PICV vectors denominated PICV-Split58-(TOM/GFP) and PICV-Split59-(TOM/GFP), respectively (FIGS. 3A-3C).

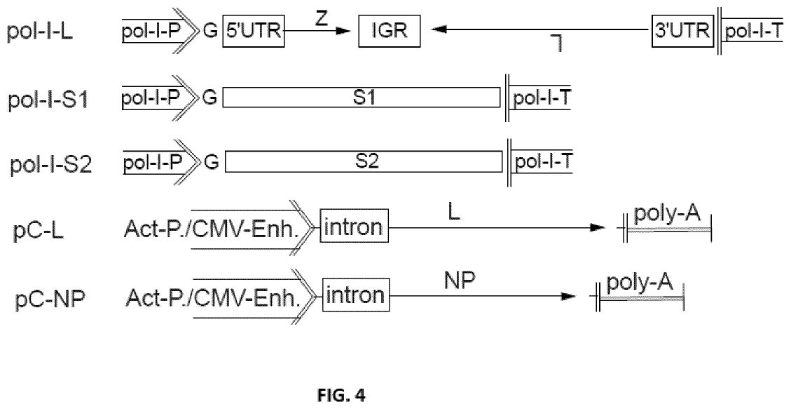

BHK-21 cells stably expressing the lymphocytic choriomeningitis virus (LCMV) glycoprotein (Flatz L, et al. Nat Med 2010; 16, 339-345) were co-transfected with plasmids:
i) polymerase-I-based expression plasmid encoding for a wildtype PICV L segment (pol-I-L),
ii) polymerase-I-based expression plasmids encoding for the respective PICV vectors' S1 and S2 segments (Pol-I-S1, Pol-I-S2), and iii) polymerase-II-driven expression plasmids for the PICV trans-acting factors L and NP (pC-L, pC-NP) (FIG. 4). Three days after transfection, the cells were passaged from M6 wells to T25 flasks and six days after transfection GFP and TOM expression in the cultures were determined by fluorescence microscopy (FIG. 5A). In addition to TOM-expressing cells, PICV-Split59-(TOM/GFP) rescue cultures exhibited bright and wide-spread GFP expression, suggesting the spread of a GFP-expressing, replication-competent vector. Infectious titers in the culture supernatants were determined seven days after transfection by immunofocus assays (FIG. 5B). 3T3 cells were used to selectively determine focus formation by replication-competent vector particles (FIG. 5B, “3T3 titer”). Further, 293T-GP cells, which express the LCMV glycoprotein and thus also allow for the formation of infectious foci by vector particles that lack a functional GP and/or are devoid of the S1 segment were used to determine the total infectivity of both replication-deficient and replication-competent particles (FIG. 5B, “293T-GP titer”). It was observed that both PICV-Split58-(TOM/GFP) and PICV-Split59-(TOM/GFP) reached comparable titers of replication-competent vectors (assessed on 3T3 cells, FIG. 5B); and the titers were similar based on the totality of replication-competent and replication-deficient vector particles as determined on 293T-GP cells (FIG. 5B). To determine potential fitness differences between PICV-Split58-(TOM/GFP) and PICV-Split59-(TOM/GFP), fresh BHK-21 cells were infected with vector at multiplicity of infection (MOI) of 0.01 and the replication-competent vector titers (3T3 titers) in supernatant were determined over time (FIG. 5C). While peak titers of the two vectors were in comparable ranges, PICV-Split59-(TOM/GFP) grew more rapidly, reaching peak titers earlier than PICV-Split58-(TOM/GFP), indicating that PICV-Split59-(TOM/GFP) was superior. These results suggested that a PICV signal peptide consisting of the 59 N-terminal amino acids of PICV GPC and a GP1/GP2 sequence corresponding to Aa60-508 of PICV GPC represented preferable definitions of functional domains that could be segregated in PICV-Split vectors.

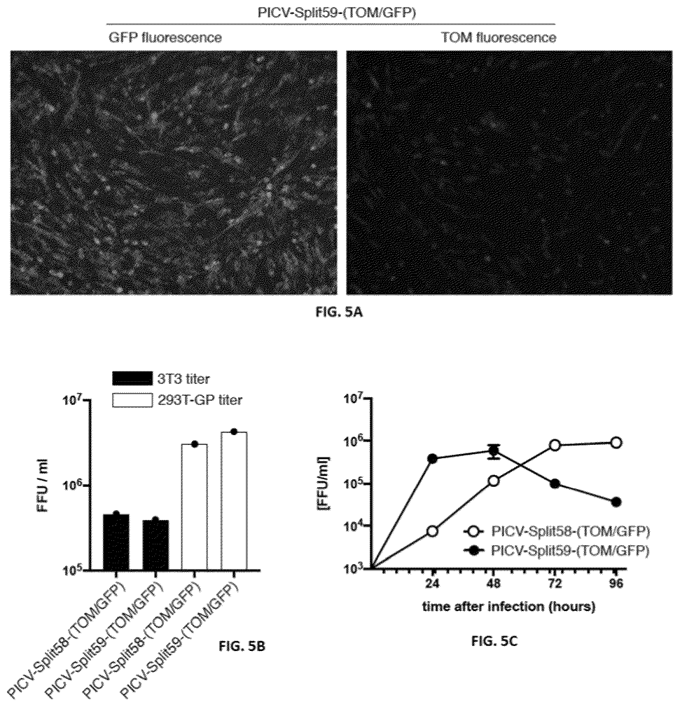

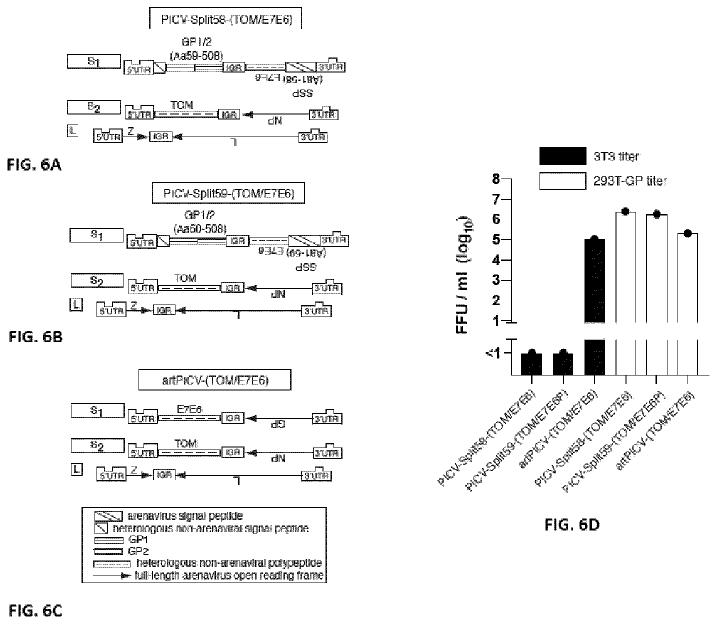

In order to generalize this concept, a non-oncogenic fusion antigen (E7E6) comprising the HPV16 E7 and E6 proteins (Cassetti et al. Vaccine 2004; 3-4, 520-527) was inserted downstream of SSP in the S1 segment. PICV-Split58-(TOM/E7E6) and PICV-Split59-(TOM/E7E6) vector genomes were used (FIGS. 6A-5B) and an artPICV vector expressing the same heterologous non-arenaviral polypeptides TOM and E7E6 (artPICV-(TOM/E7E6)) was used as a control (FIG. 6C). Transfections for the rescue of these vectors were performed as mentioned above, and infectious titers in culture supernatants were determined nine days after transfection. Intriguingly, neither the PICV-Split58-(TOM/E7E6) nor the PICV-Split59-(TOM/E7E6) vector yielded titers of replication-competent particles that were detectable on 3T3 cells, while artPICV-(TOM/E7E6) reached substantial titers. When vector titers were determined on LCMV GP-complementing 293T-GP cells, however, all three vector rescue cultures exhibited infectious non-replicating vector titers >10E5 focus-forming units (FFU) per milliliter of culture (FIG. 6D). This finding indicated that all transfections had been comparably efficient, thus yielding comparable quantities of replication-deficient vector particles. At the same time, it suggested that the GP-expressing S1 segments of PICV-Split(58)-(TOM/E7E6) and PICV-Split59-(TOM/E7E6), the only genetic difference between these two vectors and artPICV-(TOM/E7E6), failed to supply a functional glycoprotein complex, which would have allowed for the formation of replication-competent vector particles as observed for artPICV-(TOM/E7E6).

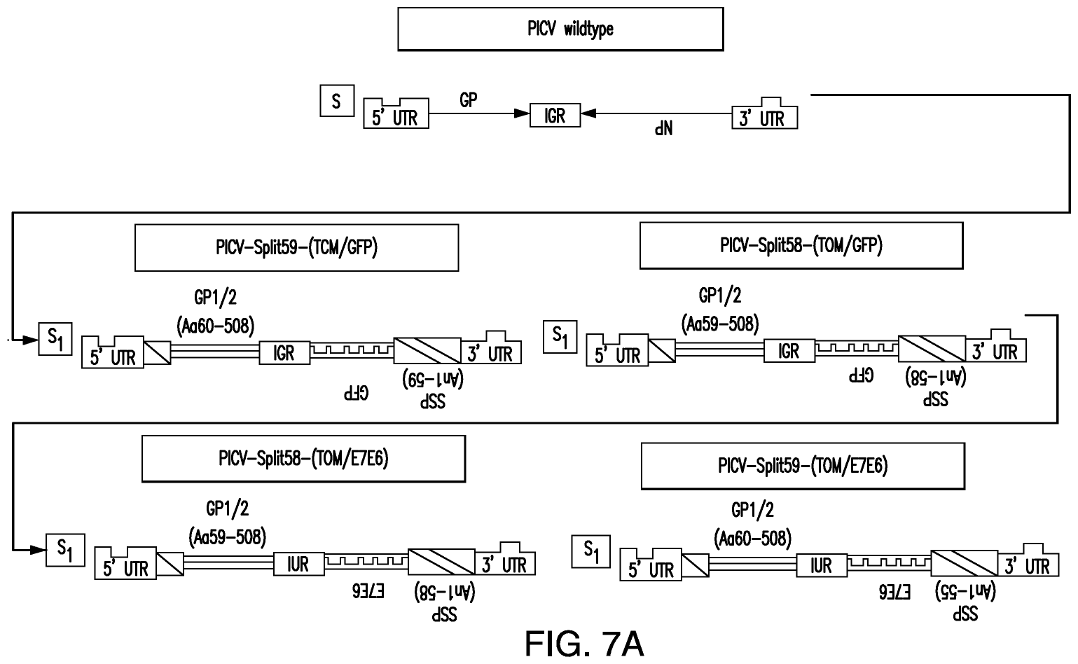

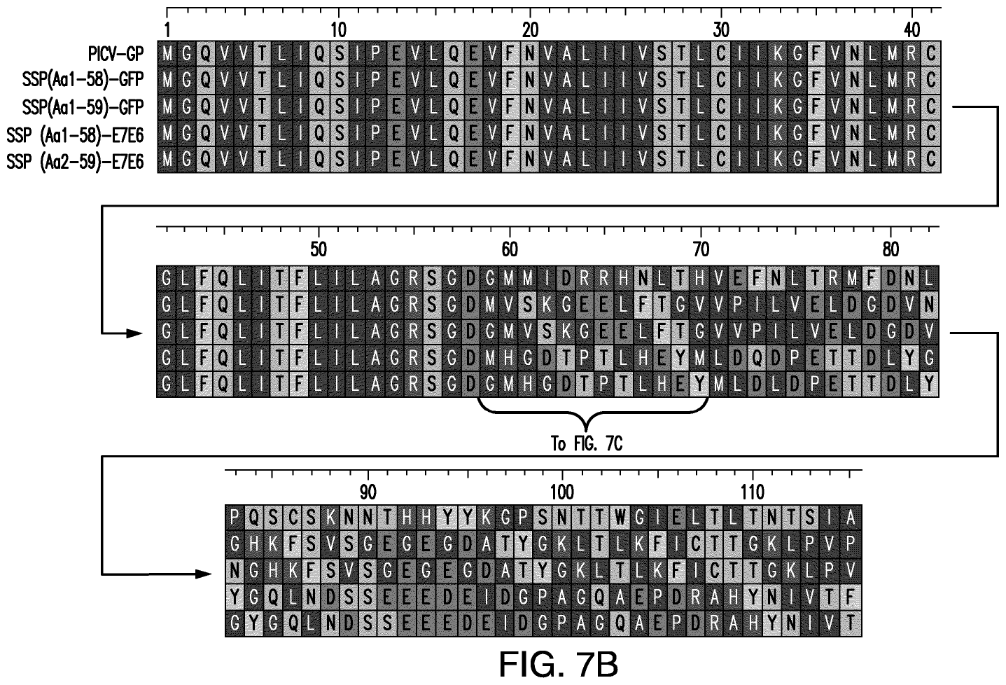

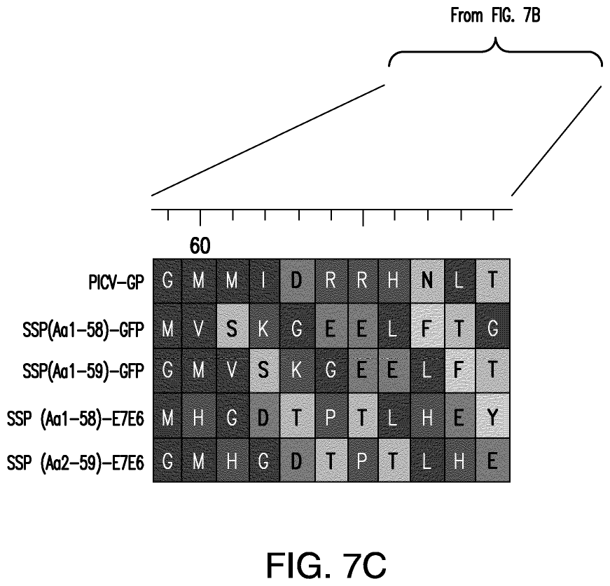

This experimental finding was intriguing and unexpected: the finding that PICV-Split(58)-(TOM/GFP) and PICV-Split59-(TOM/GFP) but neither PICV-Split(58)-(TOM/E7E6) nor PICV-Split59-(TOM/E7E6) formed replication-competent vectors was apparently related to these vectors differential S1 segment, since their S2 and L segments were identical. Moreover, the only difference between these vectors consisted in the transgene inserted downstream of the SSP (FIG. 7A), which was apparently key for the viability of these vectors. Both GFP and E7E6 are simple vector cargo and are not involved in the natural PICV viral life cycle, thus neither of them can be required for viral replication. Moreover E7E6 is well tolerated as a transgene in artPICV-(TOM/E7E6) arguing against the possibility that it was not tolerated as a transgene in the PICV vector context. As such, it was hypothesized that the transgene downstream of SSP impacted the function of the SSP itself. While it is known that signal peptide cleavage is a rate-limiting step of protein secretion (Geukens N, et al. (2004) Biochem. Biophys. Res. Commun. 314 (2), 459-467), impaired SSP cleavage and its limited availability for the formation of the tripartite SSP-GP1-GP2 mature glycoprotein complex would have required the transgene downstream of SSP to affect cleavage efficiency. Contrary to this hypothesis, eukaryotic signal peptide cleavage can be determined by amino acid residues inside the signal peptide itself, without clear impact of the amino acid sequence downstream of the cleavage site.

In contrast, signal peptidase cleavage in prokaryotes can be influenced by the sequence immediately downstream of the cleavage site (Allet B, et al. (1997) Protein Expr Purif, 9:61-68), the so-called “Pro-region”, which extends up to 30 amino acids. Negative or neutral charge is preferable in the Pro-region and the first six amino acids are particularly important (Owji H, et al. (2018) Eur J Cell Biol 97(6):422-441). In contrast, these amino acid positions are generally assumed to be of limited or no influence on eukaryotic signal peptide cleavage efficiency and resulting protein secretion (Owji H, et al. (2018) Eur J Cell Biol 97(6):422-441). The reason for this presumed difference between prokaryotes and eukaryotes is that in prokaryotes a negatively charged protein region downstream of the signal peptidase cleavage site is thought responsible for loop formation with the positively charged N-terminal region of the signal peptide, thereby compensating the electrochemical potential of the bacterial inner membrane. In contrast, positive charge is, supposedly, readily tolerated downstream of the cleavage site of eukaryotic signal peptides, owing to their lack of an electrochemical potential in the ER membrane (Low K. O., et al. (2013) Appl. Microbiol. Biotechnol. 97 (9), 3811).

Nevertheless, when aligning the PICV-GP with the open reading frames (ORFs) that contain the SSP in PICV-Split(58)-(TOM/GFP), PICV-Split59-(TOM/GFP), PICV-Split(58)-(TOM/E7E6) and PICV-Split59-(TOM/E7E6) (FIG. 7B), a tentative pattern was observed in the stretch of amino acids comprising position 59 and downstream (FIG. 7C), or the most N-terminal sequence where these ORFs deviate from each other. In PICV-GP, the natural SSP context, this sequence stretch started with four small and hydrophobic, thus neutral, amino acids (GMMI), followed by one acidic amino acid (D) prior to the first basic amino acid (R) at position 64. In PICV-Split59-(TOM/GFP), the vector amongst the four tested above that exhibited the highest fitness, this sequence stretch started with four small, hydrophobic or polar amino acids (GMVS), then followed by a basic amino acid (K at position 63). In the less fit but viable PICV-Split(58)-(TOM/GFP), the stretch of hydrophobic or small polar amino acids in this sequence stretch was shortened to three instead of four (MVS instead of GMVS), with the basic amino acid lysine (K) following already at position 62. Finally, the non-viable PICV-Split(58)-(TOM/E7E6) and PICV-Split59-(TOM/E7E6) had a basic amino acid (H) in position 60 and 61, respectively. Therefore, the hierarchy of vector fitness and viability correlated with the length of a sequence stretch immediately downstream of the signal peptide cleavage site that consisted of neutral, small apolar and acidic amino acids, distancing the first downstream basic amino acid from the cleavage site.

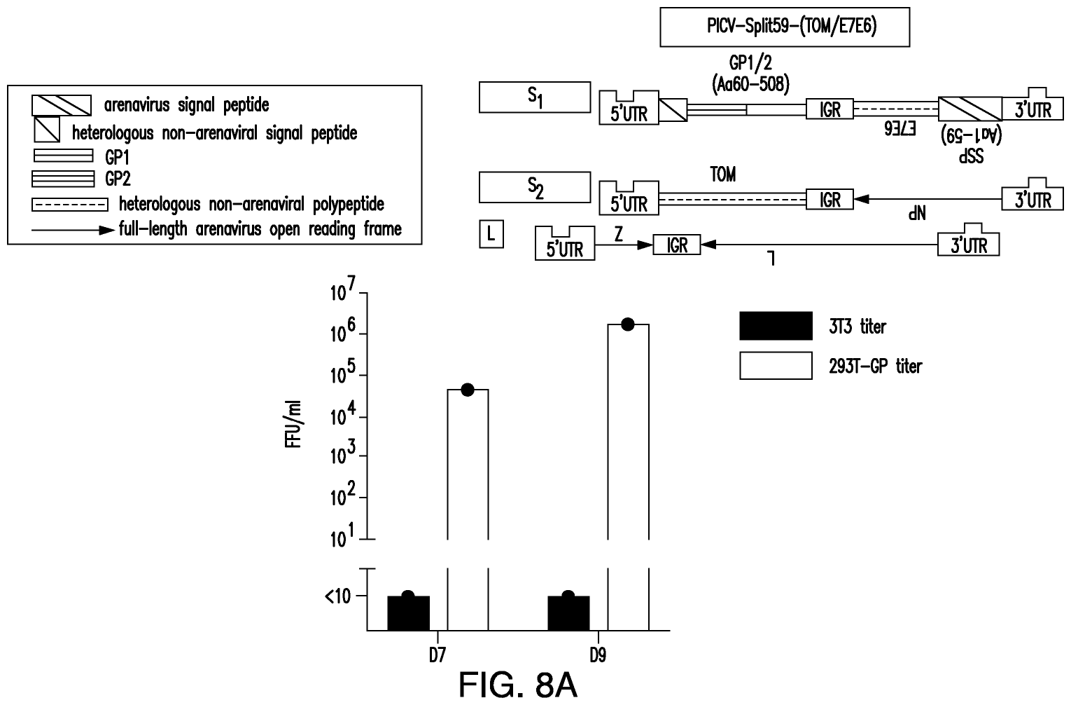

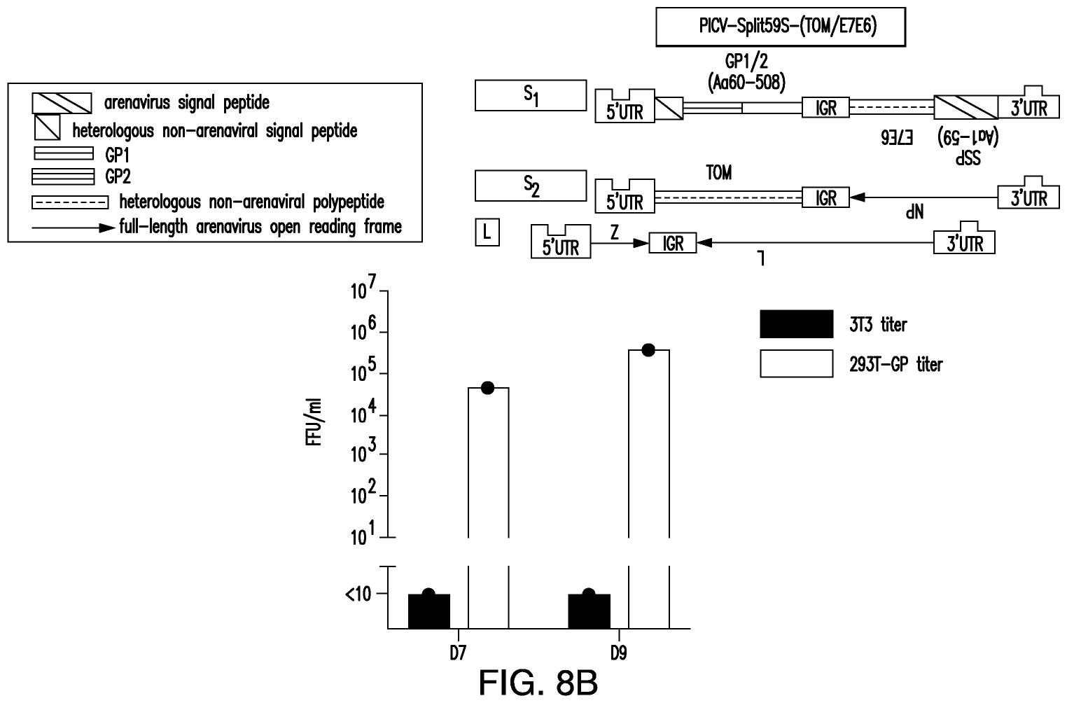

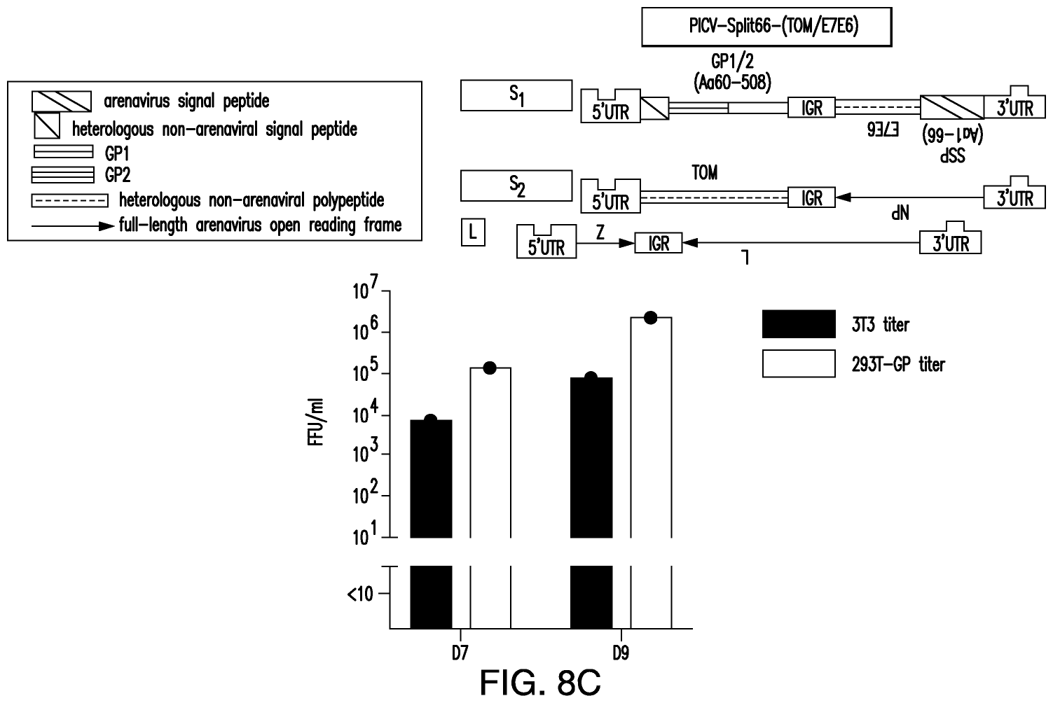

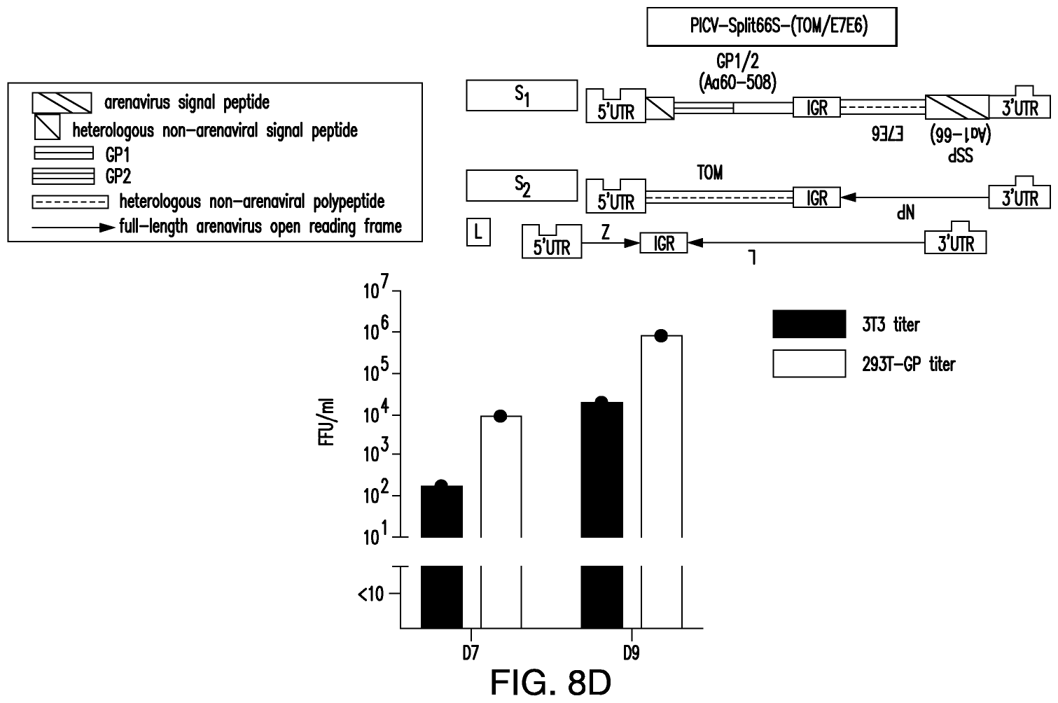

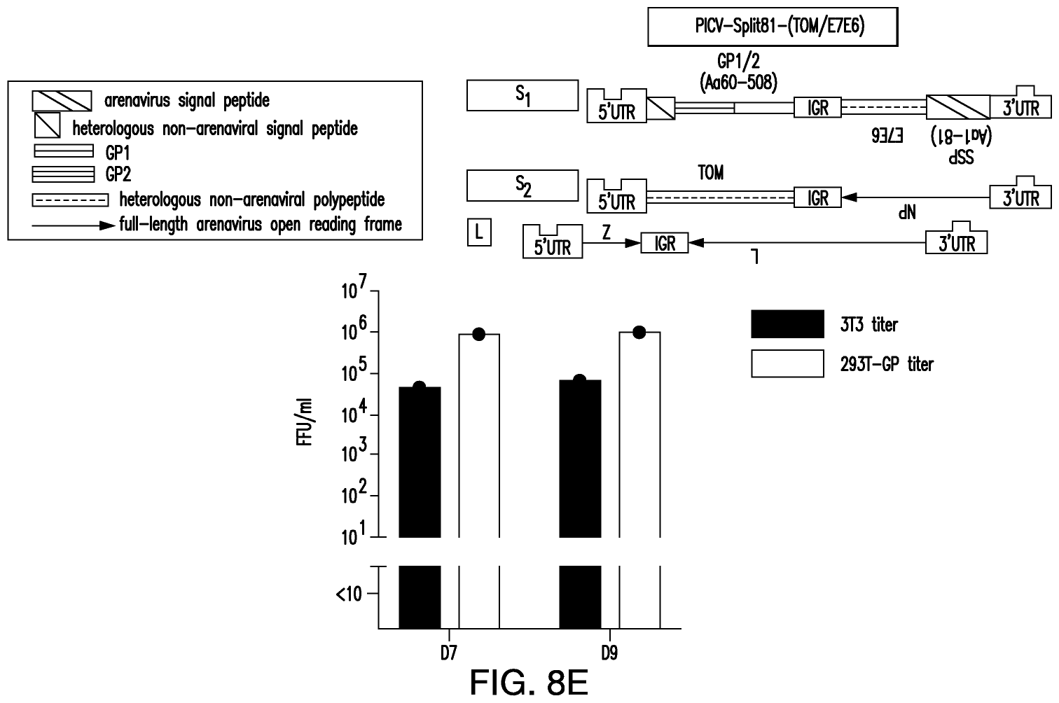

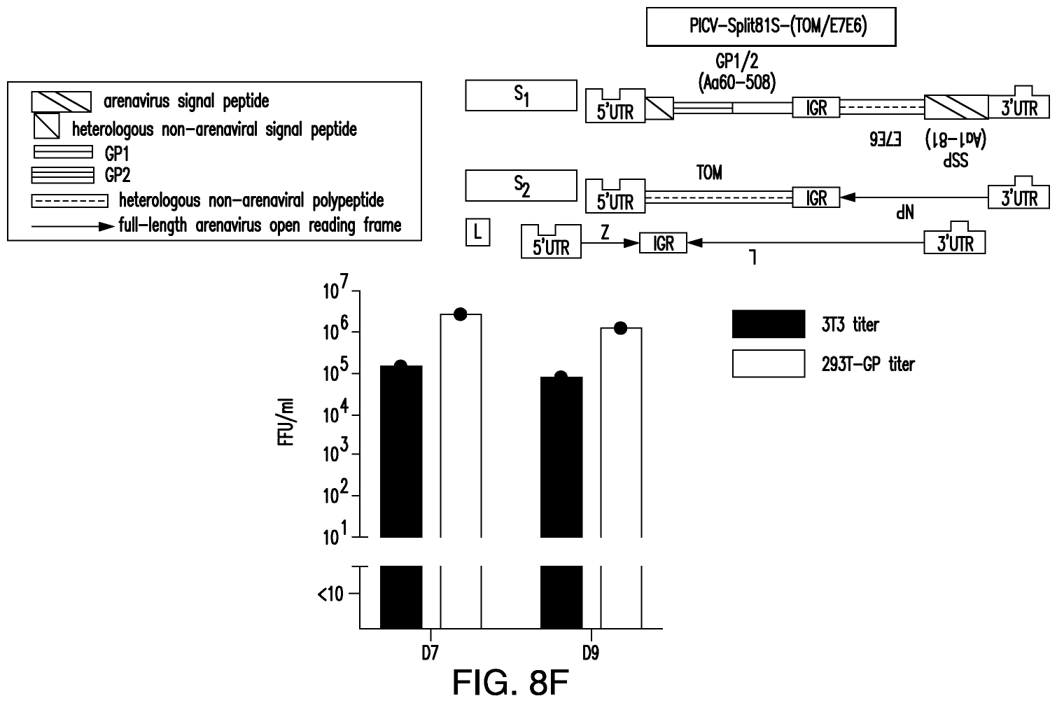

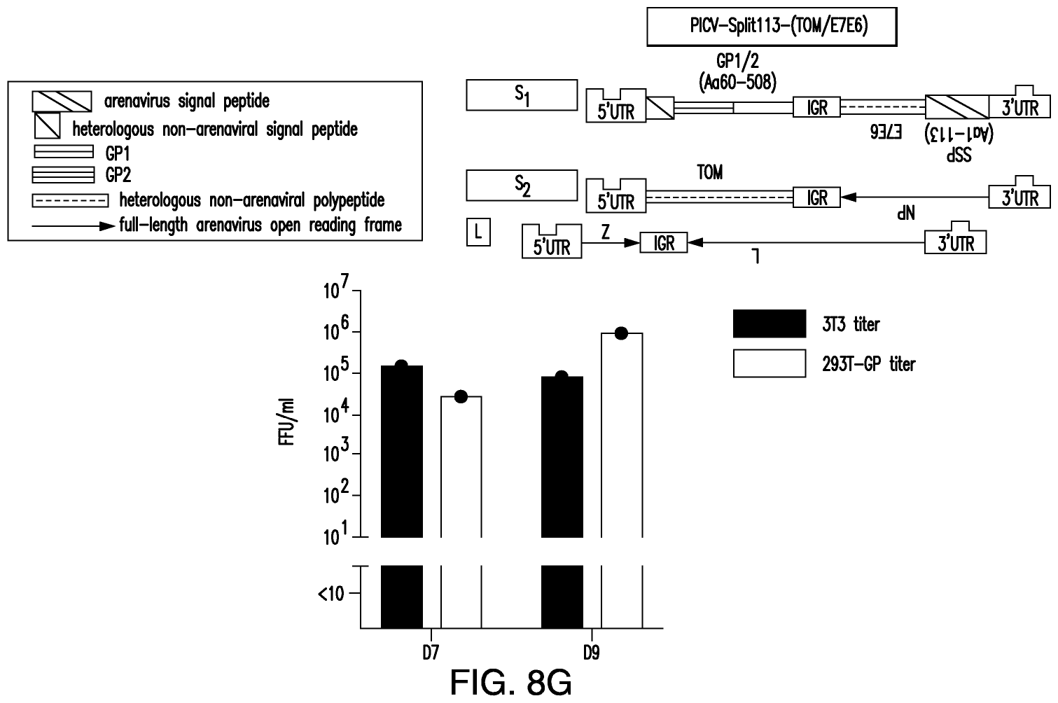

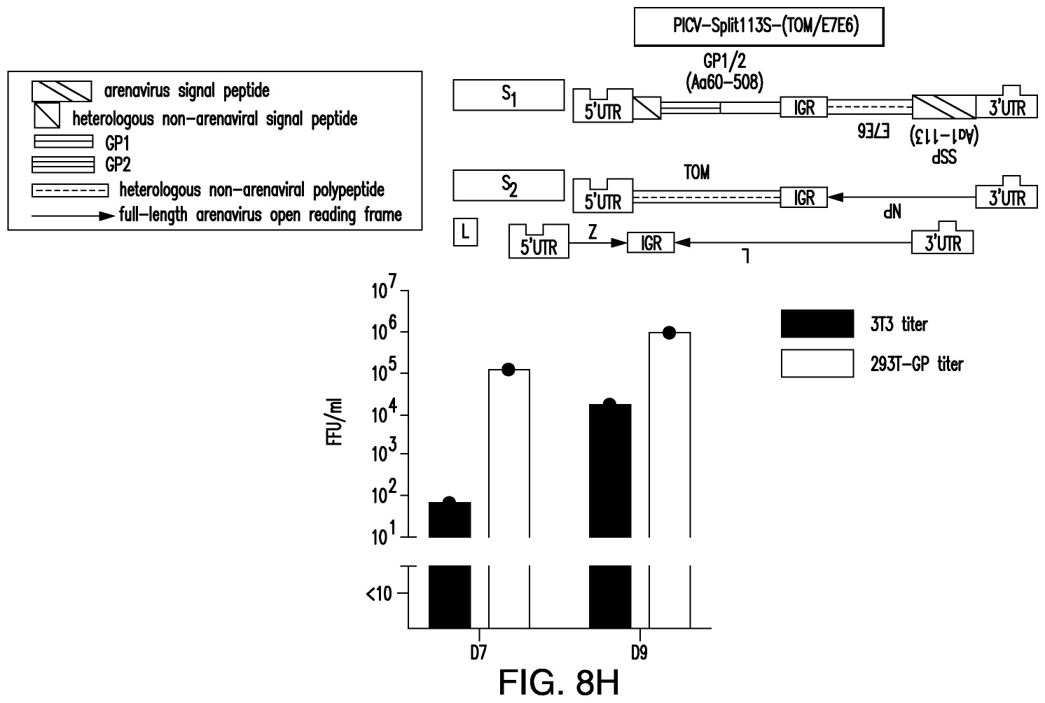

While this pattern of vector viability was reminiscent of the requirements for efficient prokaryotic signal peptide cleavage there was no direct evidence that these prokaryotic Pro-region characteristics indeed applied to signal peptidase cleavage downstream of the PICV SSP. It was not possible to know after which amino acid SSP cleavage occurred in these several constructs. Thus, potential downstream sequence requirements for efficient SSP cleavage were tested. In brief, additional E7E6-expressing vector constructs were generated, wherein additional PICV-GP-derived sequence, serving as a “buffer peptide,” were placed between the supposed 59Aa-long PICV signal peptide and the E7E6 transgene in PICV-Split59-(TOM/E7E6). Specifically, the fragment of PICV-GP N-terminal sequence that precedes E7E76 in the vector was extended from 59 in PICV-Split(59)-(TOM/E7E6) to either 66, 81 or 113 amino acids (buffer peptides of 7, 22 or 54 amino acids in length, respectively, when assuming a 59 amino acid-long signal peptide), yielding PICV-Split(66)-(TOM/E7E6), PICV-Split(81)-(TOM/E7E6) and PICV-Split(113)-(TOM/E7E6), respectively (FIGS. 8A, 8C, 8E, and 8G). Additionally, it was hypothesized that amino acid 59 of PICV-GP can be either G, as in the constructs tested above, or S (Zhang et al. (1999) Am J Trop Med Hyg.; 61(2):220-5.). Hence, additional vector constructs analogous to the above were generated, wherein the glycine at position 59 of SSP was substituted for a serine (PICV-Split(59S)-(TOM/E7E6), PICV-Split(66S)-(TOM/E7E6), PICV-Split(81S)-(TOM/E7E6) and PICV-Split(113S)-(TOM/E7E6) (refer to “SSP-59S variants”; FIGS. 8B, 8D, 8F, and 8H).

Transfection experiments to rescue the above vectors from cDNA were performed as outlined for the analogous experiment in FIGS. 5A, 5B and 6D. 3T3 titers and 293T-GP titers were determined seven and nine days after transfection. Similarly to the results shown in FIG. 6D, PICV-Split(59)-(TOM/E7E6) did not form replication-competent vector particles that formed infectious foci on 3T3 cells (FIG. 8A). Exchanging Aa59 of the SSP for a serine in construct PICV-Split(59S)-(TOM/E7E6) did not result in replication-competent vector formation either (FIG. 8B). Both transfections yielded, however, substantial titers of replication-deficient vector particles (293T-GP titer) attesting to the efficiency of the rescue procedure. In striking contrast, PICV-Split(66)-(TOM/E7E6), PICV-Split(81)-(TOM/E7E6) and PICV-Split(113)-(TOM/E7E6) as well as the corresponding SSP-59S variants consistently yielded replication-competent vector particles (FIGS. 8C-8H), with only minor differences in peak titers.

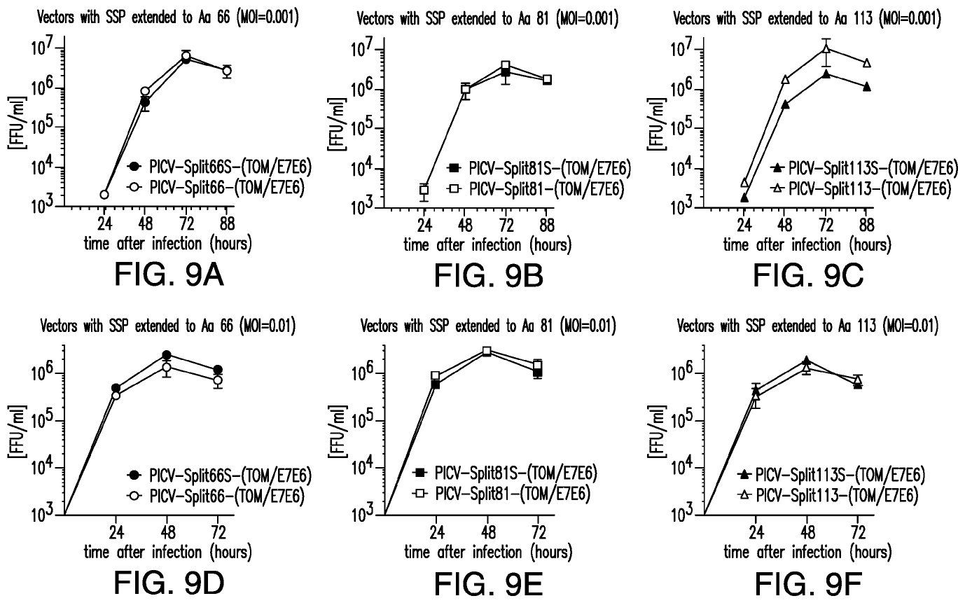

To better assess potential differences in the growth behavior of PICV-Split(66S)-(TOM/E7E6), PICV-Split(81S)-(TOM/E7E6) and PICV-Split(113S)-(TOM/E7E6) and their SSP-59S variants, BHK-21 cells were infected at MOI of 0.001 or 0.01 and 3T3 titers in supernatant were determined over time (FIGS. 9 and 10). Comparing PICV-Split(66)-(TOM/E7E6), PICV-Split(81)-(TOM/E7E6) and PICV-Split(113)-(TOM/E7E6) with their respective SSP-59S variant did not reveal a consistent advantage of vectors expressing either a serine of glycine at position 59 of the SSP (FIGS. 9A-9F). Neither did the direct comparison of vectors with 66, 81 or 113 Aa of N-terminal PICV-GP sequence preceding E7E6 evidence a clear hierarchy in vector growth, neither at MOI of 0.001 nor of 0.01, and independently of SSP position 59 being either serine or glycine (FIGS. 10A-10D). These findings suggested that the N-terminal 66 amino acids of PICV-GP contained the minimal sequence information that allowed efficient SSP functioning when fused downstream to an E7E6 transgene and expressed in the context of a PICV-Split vector.

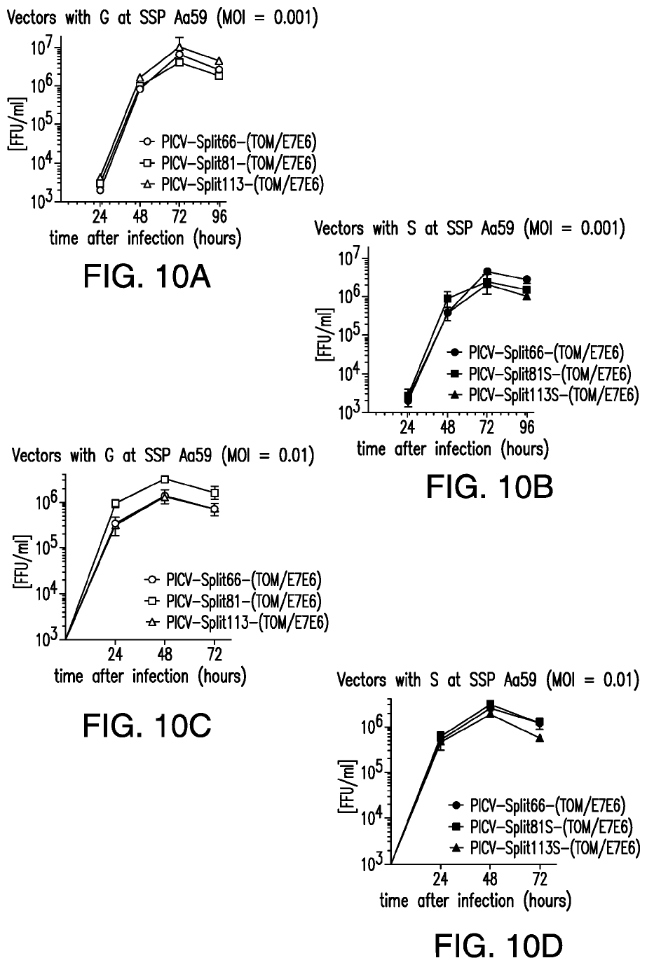

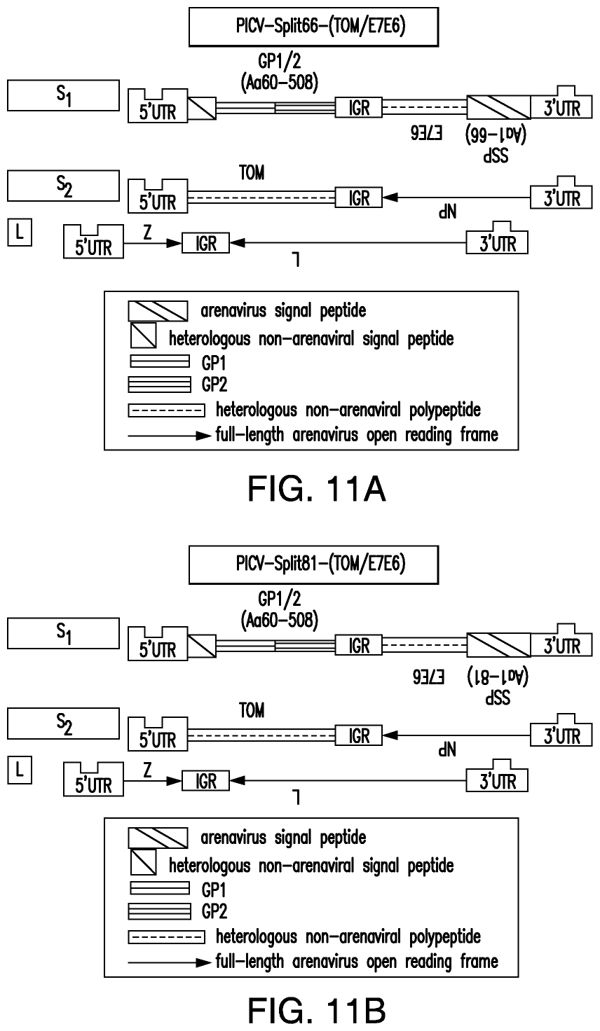

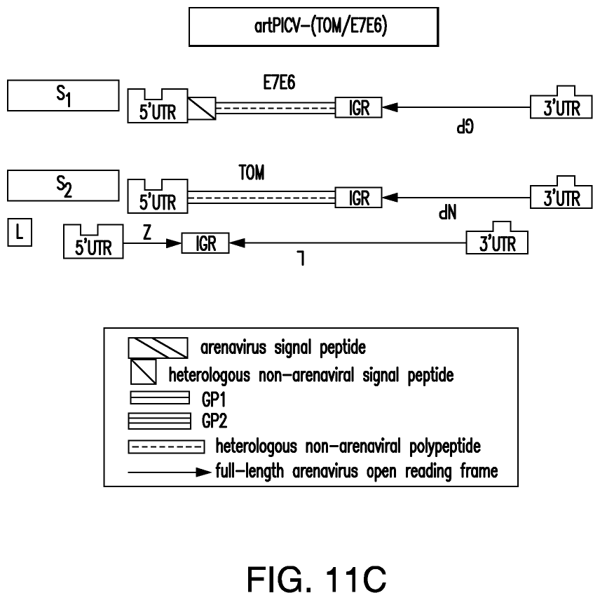

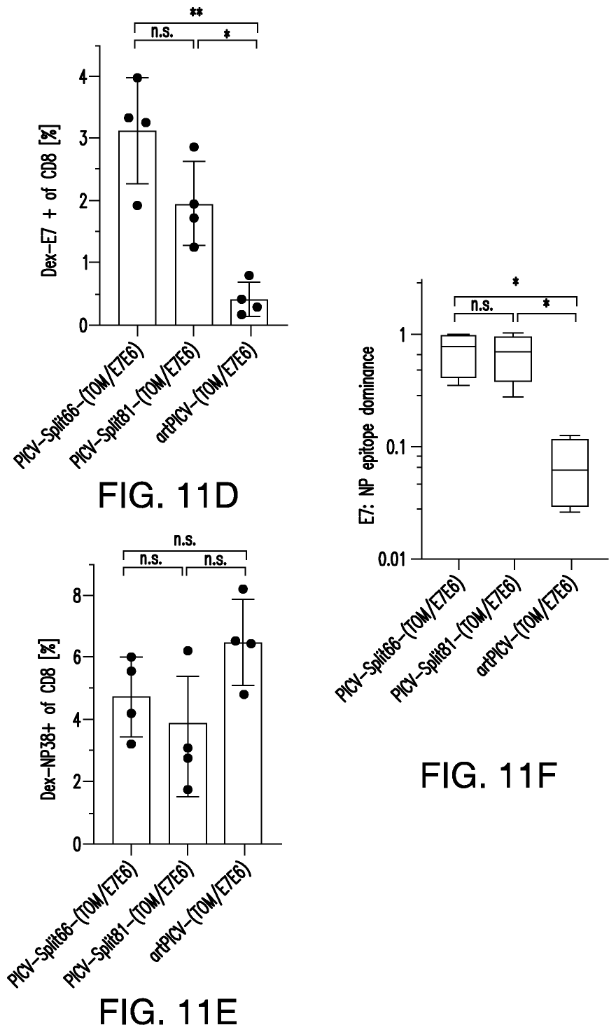

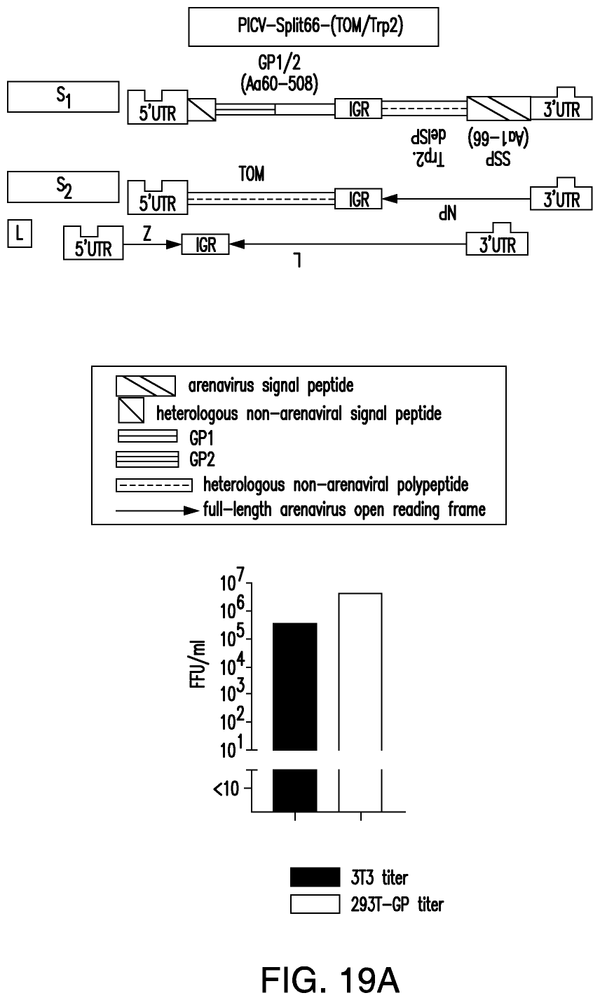

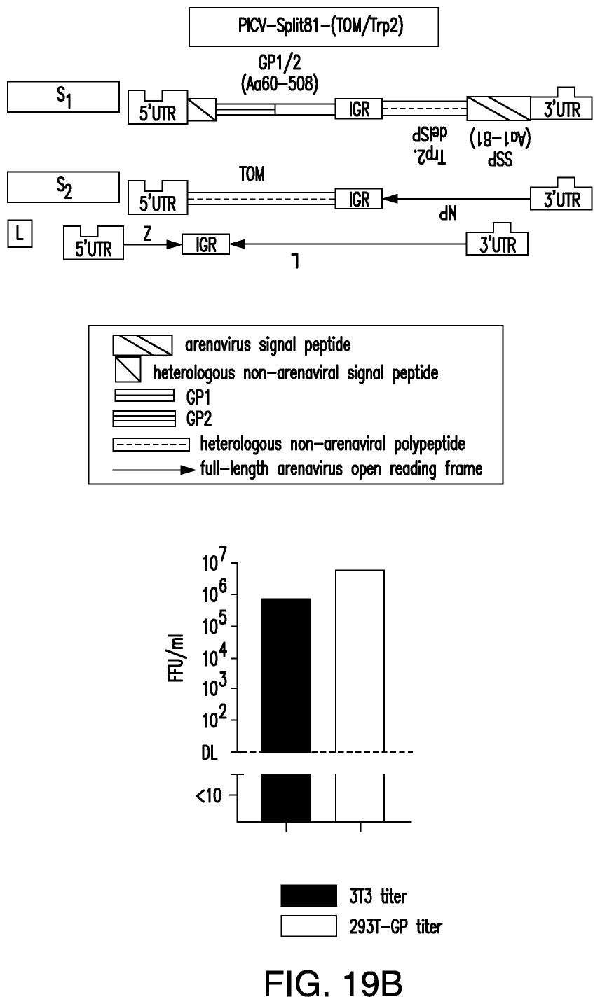

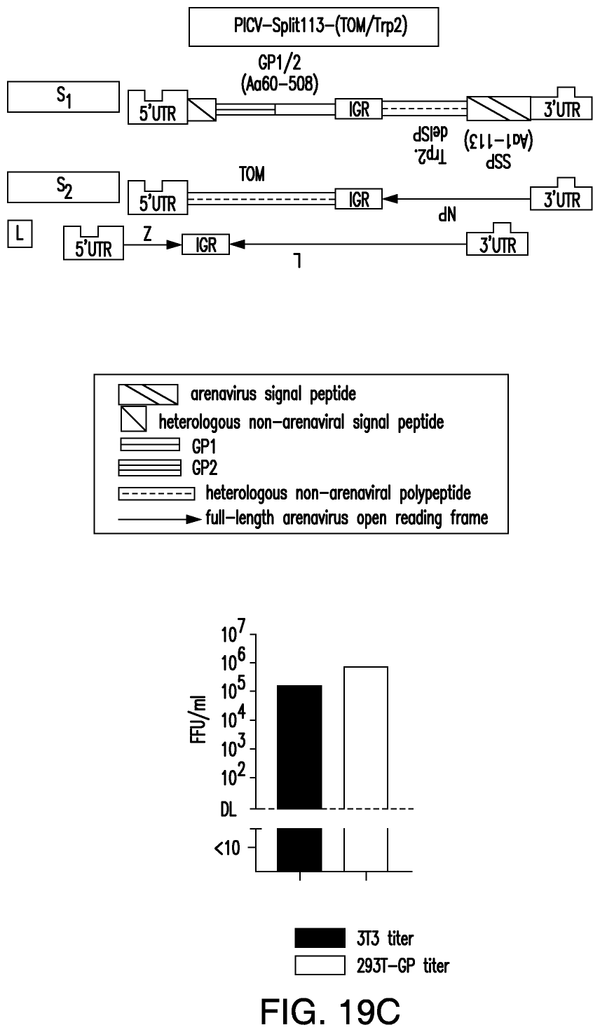

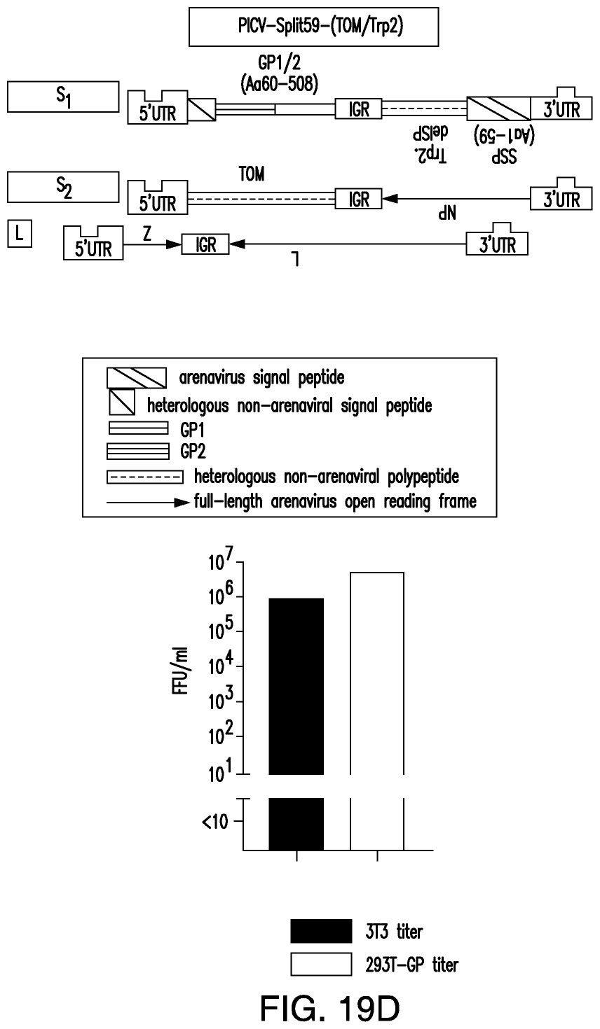

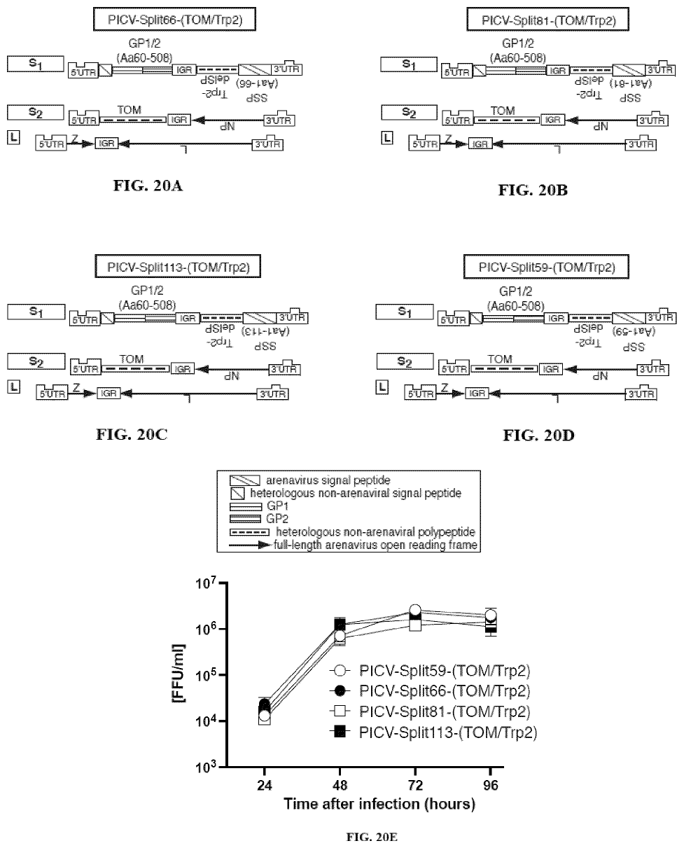

In order to extend these findings to other transgenes, PICV-Split vectors encoding the melanoma antigen Trp2 instead of E7E6 were generated (FIGS. 19A-19C). For this, it was taken into account that the Trp2 protein is naturally inserted into the endoplasmic reticulum (ER) of eukaryotic cells, thus entering the secretory pathway. In designing PICV-Split(66)-(TOM/Trp2), PICV-Split(81)-(TOM/Trp2) and PICV-Split(113)-(TOM/Trp2) vectors (FIGS. 19A-19C), the natural N-terminal signal sequence of Trp2 was substituted with the 59 Aa-long SSP of PICV. Additionally and analogous to the design of the above E7E6-expressing vectors (refer to FIGS. 8C-8H), the SSP and the Trp2 open reading frame (devoid of its own signal peptide; Trp2-delSP) were separated by in-frame “buffer peptide” coding sequence corresponding to either Aa 60-66, Aa 60-81 or Aa 60-113 of PICV-GP, resulting in one in-frame fusion protein (SSP-buffer peptide-Trp2-delSP). A construct devoid of an in-frame “buffer peptide”, PICV-Split(59)-(TOM/Trp2), was also generated (FIG. 19D). Transfection experiments were performed to rescue the above vectors from cDNA as outlined for the analogous experiment in FIGS. 5A-5B, FIG. 6D and FIGS. 8A-8H. 3T3 titers and 293T-GP titers were determined six days after transfection. All transfections yielded substantial titers of replication-deficient vector particles (293T-GP titer) attesting to the efficiency of the rescue procedure. In further analogy to their E7E6-expressing counterparts, PICV-Split(66)-(TOM/Trp2), PICV-Split(81)-(TOM/Trp2) and PICV-Split(113)-(TOM/Trp2) also consistently yielded replication-competent vector particles (FIGS. 11A-11C), with only minor differences in titers. In contrast to the E7E6-expressing set of vectors, however, PICV-Split(59)-(TOM/Trp2) also reached a substantial infectious titer despite its lack of a “buffer peptide” (FIG. 19D). Accordingly, PICV-Split(66)-(TOM/Trp2), PICV-Split(81)-(TOM/Trp2), PICV-Split(113)-(TOM/Trp2) and PICV-Split(59)-(TOM/Trp2) grew with similar kinetics and reached comparable peak titers when passaged on BHK-21 cells (MOI=0.01; FIGS. 20A-20E).

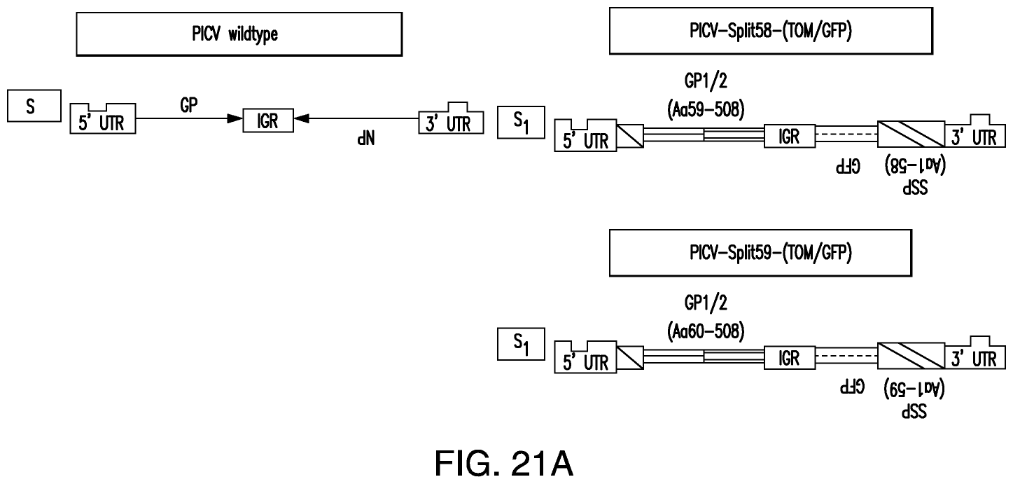

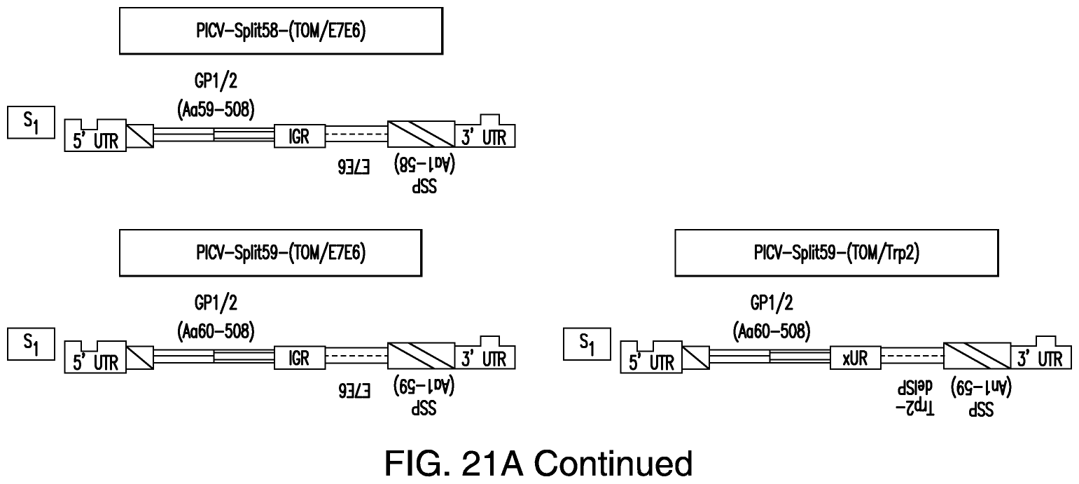

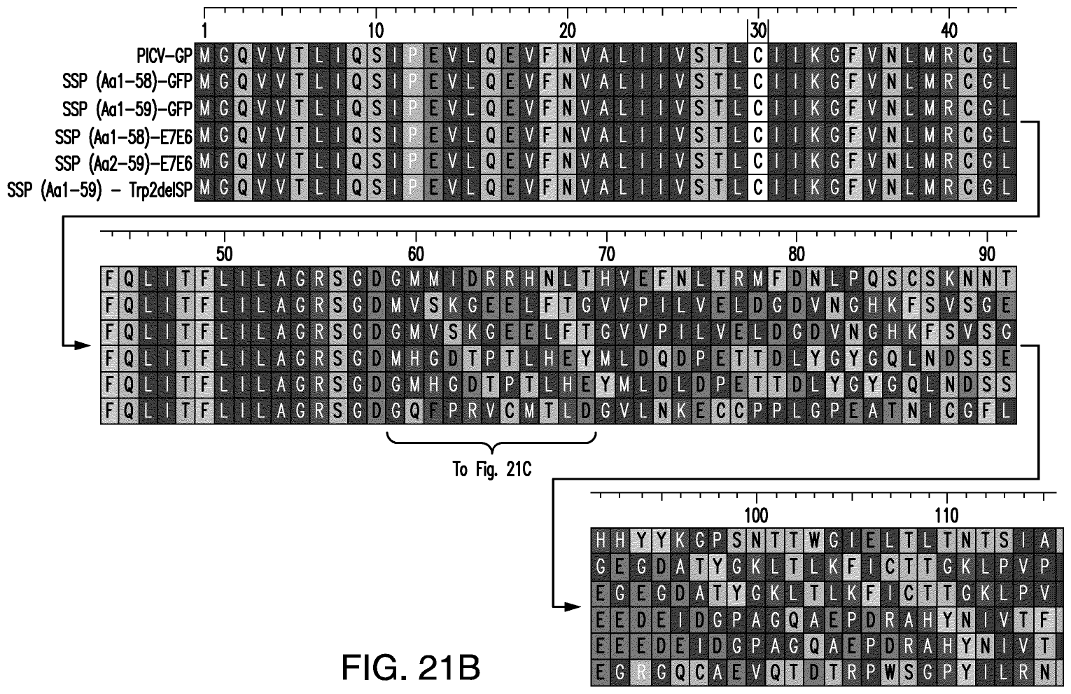

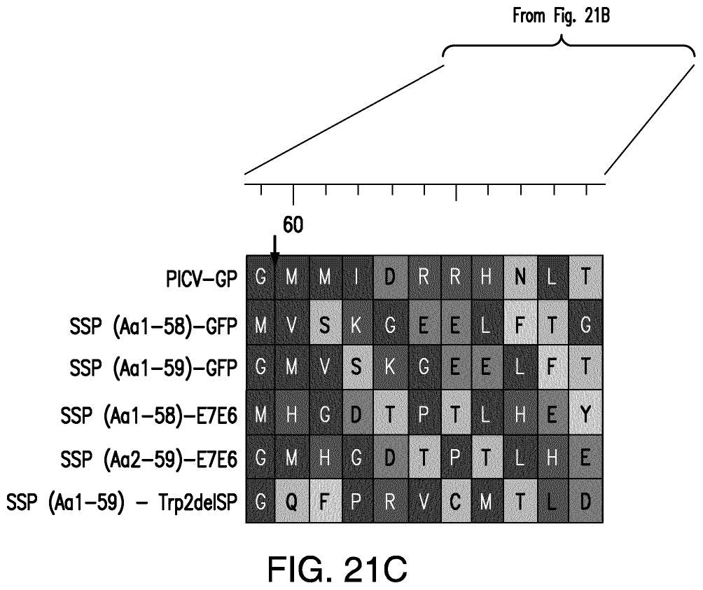

Consequently, the SSP(Aa1-59)-Trp2delSP sequence was included in the alignment as presented in FIG. 7 (FIGS. 21A-21C), to compare it to PICV-GP and to the open reading frames (ORFs) that contain the SSP in PICV-Split(58)-(TOM/GFP), PICV-Split59-(TOM/GFP), PICV-Split(58)-(TOM/E7E6) and PICV-Split59-(TOM/E7E6) (FIGS. 7B-7C). The SSP(Aa1-59)-Trp2delSP sequence resembled the one of PICV-Split59-(TOM/GFP), the vector amongst the other four recombinant constructs that exhibited the highest fitness: amino acids 60-62 of SSP(Aa1-59)-Trp2delSP, which follow immediately after the putative cleavage site at aa59, are devoid of any basic amino acid, with an arginine (R) following only at position 63. In PICV-GP the first basic amino acid, also an arginine, is found at position 64 and thus is only slightly further distanced from the putative cleavage site than in SSP(Aa1-59)-Trp2delSP of PICV-Split59-(TOM/E7E6) and in SSP(Aa1-59)-GFP expressed from PICV-Split59-(TOM/GFP). Therefore, the hierarchy of vector fitness and viability correlated with a minimal length of a sequence stretch immediately downstream of the signal peptide cleavage site that distanced the first downstream basic amino acid from the cleavage site.

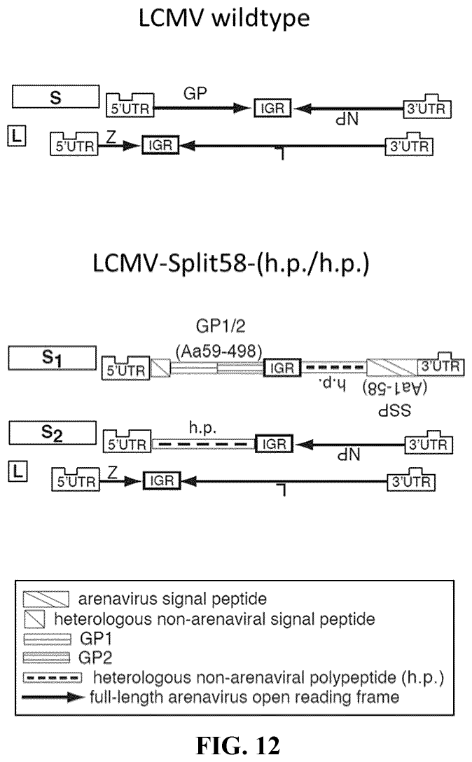

To directly test whether the sequence immediately downstream of the putative signal peptide cleavage site impacts vector growth properties, amino acid positions 60 and 61 of the SSP(Aa1-66)-E7E6 in PICV-Split66-(TOM/E7E6) were permutated, to either aspartate (DD variant), glutamate (EE variant), glycine (GG variant), leucine (LL variant), histidine (HH variant) or lysine (KK variant) (FIG. 22A). In the wildtype (WT) viral glycoprotein-derived “buffer peptide” sequence these amino acid residues consist of two methionines (MM, WT). The corresponding vectors were rescued from cDNA as described above (see also schematic overview in FIG. 12 and FIG. 4). Subsequently, BHK-21 cells were infected with either one of these vectors at MOI=0.01 and infectious replication-competent vector titers in the supernatant were assessed 24 hours later by immunofocus assay on 3T3 cells (FIG. 22B). Vector titers were highly consistent between the duplicate samples of each vector tested but differed between vector constructs by up to ˜10-fold. While several variants (DD, EE, GG, HH) reached titers that were comparable to or higher than the WT version, the LL variant exhibited a clear growth advantage over WT and the KK variant reached only comparable lower titers. Altogether these findings indicated that the amino acid sequence downstream of the signal peptide cleavage site, in the present example the sequence of the buffer peptide, is critical for vector growth.

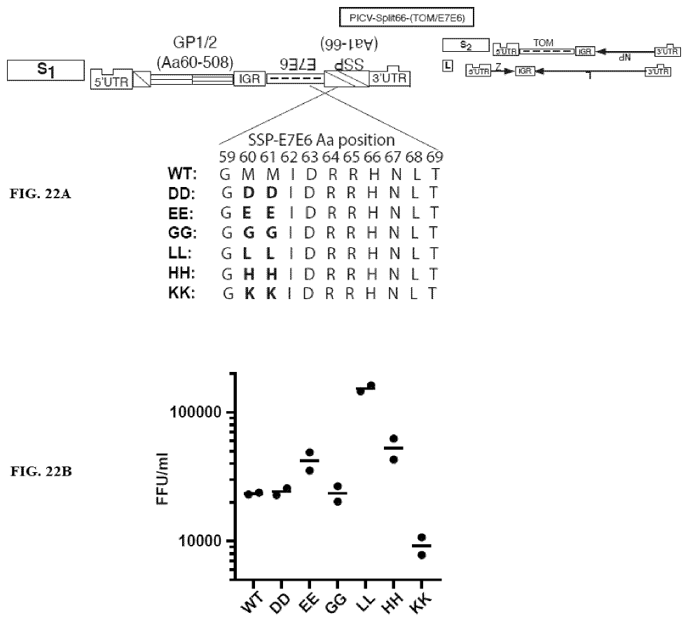

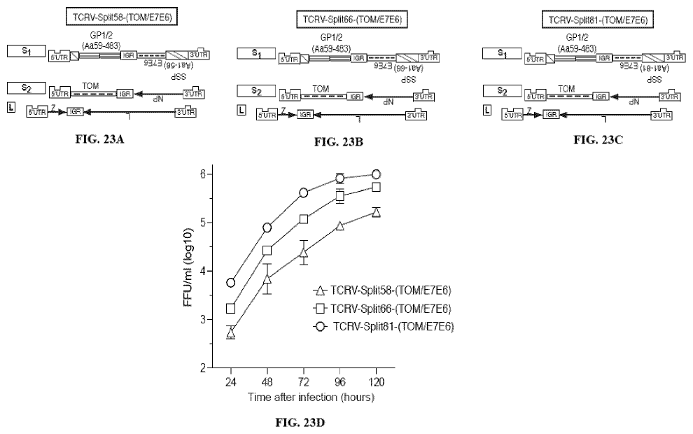

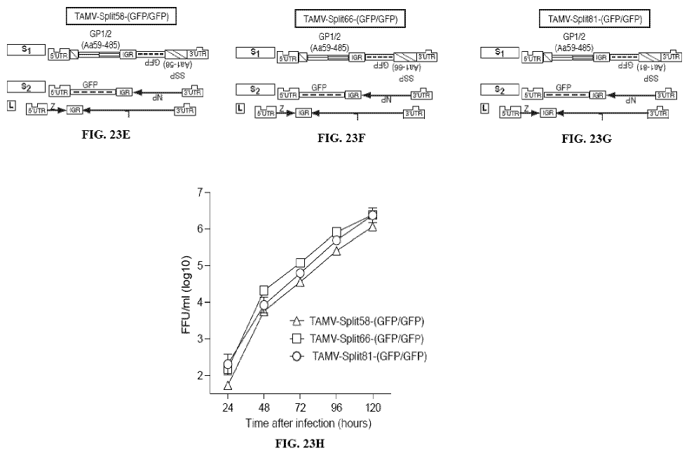

Next, experiments were performed to determine if the insertion of a buffer peptide may also improve the growth of Split vectors that are based on other New World arenaviruses than PICV. As such, Tacaribe virus- (TCRV-) based Split vectors were generated. The TCRV glycoprotein signal peptide length was predicted by the PrediSi (Prediction of SIgnalpeptides) website (from the Technical University of Braunschweig) to consist in the first 58 amino acids of the glycoprotein ORF. TCRV-Split58-(TOM/E7E6), TCRV-Split66-(TOM/E7E6) and TCRV-Split8l-(TOM/E7E6), carrying on their respective S1 segments an SSP-E7E6 ORF that either lacks a buffer peptide (TCRV-Split58-(TOM/E7E6)) or that contain buffer peptides of different lengths (TCRV-Split66-(TOM/E7E6) and TCRV-Split8l-(TOM/E7E6)) were generated (FIGS. 23A-23C). BHK-21 cells were infected with either one of these vectors at multiplicity of infection of 0.01, and infectious titers in the supernatant were determined over time. The buffer peptide-containing TCRV-Split8l-(TOM/E7E6) vector reached higher titers than TCRV-Split66-(TOM/E7E6), and the latter virus' titers over time were higher than those of TCRV-Split58-(TOM/E7E6) lacking a buffer peptide (FIG. 23D).

Similar studies were performed on Tamiami virus- (TAMV-) based Split vectors. The TAMV glycoprotein signal peptide length was predicted by the PrediSi (Prediction of SIgnalpeptides) website (from the Technical University of Braunschweig) to consist in the first 58 amino acids of the glycoprotein ORF. TAMV-Split58-(GFP/GFP), TAMV-Split66-(GFP/GFP) and TAMV-Split8l-(GFP/GFP), carrying on their respective S1 segments an SSP-E7E6 ORF that either lacks a buffer peptide (TAMV-Split58-(GFP/GFP)) or that contain buffer peptides of different lengths (TAMV-Split66-(GFP/GFP) and TAMV-Split8l-(GFP/GFP)) were generated (FIGS. 23E-23G). BHK-21 cells were infected with either one of these vectors at multiplicity of infection of 0.001 and infectious titers in the supernatant were determined over time. It was found that the buffer peptide-containing TAMV-Split8l-(GFP/GFP) and TAMV-Split66-(GFP/GFP) vectors reached higher peak (e.g., slightly higher) titers than TAMV-Split58-(GFP/GFP), which lacks a buffer peptide (FIG. 23H).

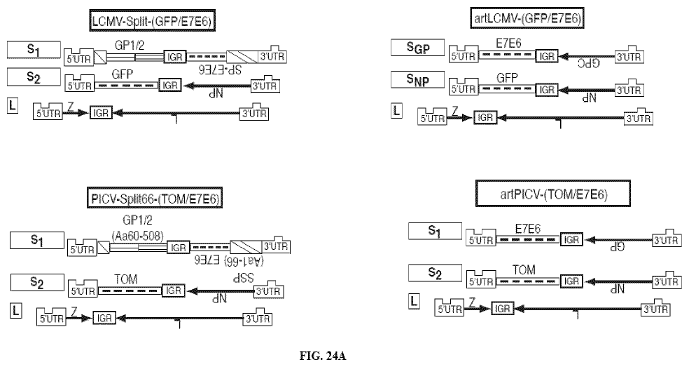

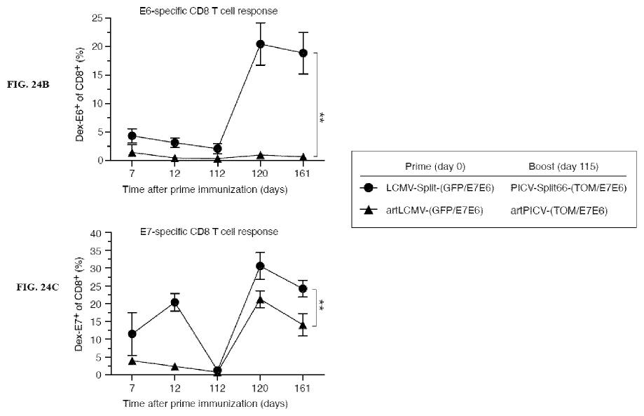

Next, PICV- and LCMV-based Split vectors were tested to determine if they can be efficiently combined in heterologous prime-boost immunization regimens. C57BL/6 mice were primed with LCMV-Split-(GFP/E7E6) or artLCMV-(GFP/E7E6) and 115 days later were boosted with PICV-Split66-(TOM/E7E6) or artPICV-(TOM/E7E6), respectively (FIGS. 24A-24C). E6 epitope- and E7 epitope-specific CD8 T cell responses in blood were followed over time (FIGS. 24B-24C). The animals immunized twice with Split-based vectors exhibited significantly higher E6- and E7-specific CD8 T cell frequencies than controls immunized with artARENA-based vectors. This difference was particularly pronounced for the subdominant E6 specificity.

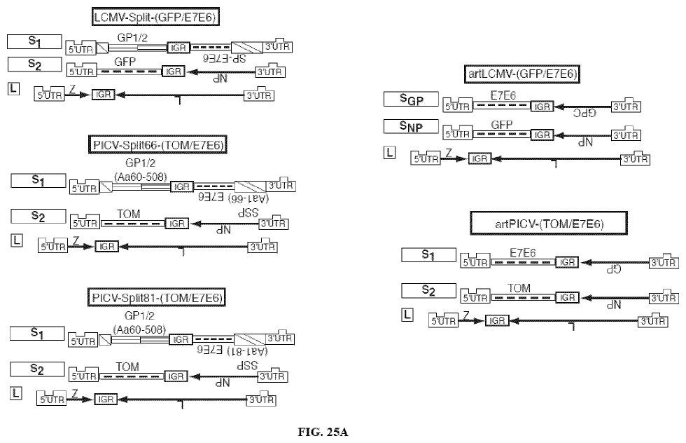

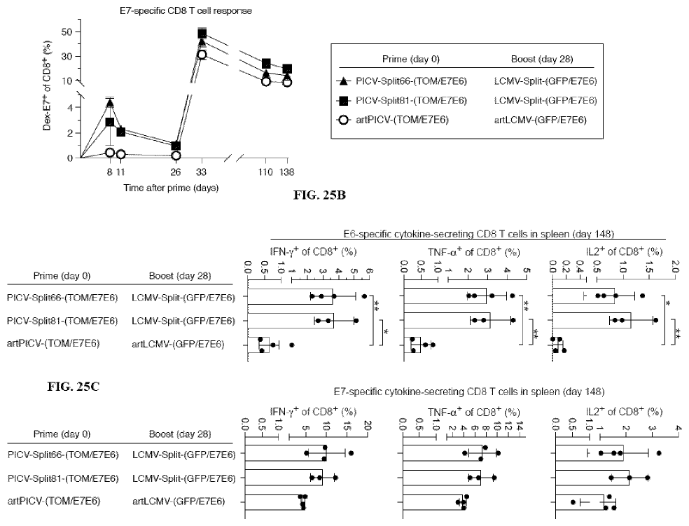

In a complementary experiment, animals were primed with PICV-based vectors followed by LCMV vector-based boost. PICV-Split66-(TOM/E7E6), PICV-Split8l-(TOM/E7E6) or artPICV-(TOM/E7E6) were used as prime on day 0 and LCMV-Split-(GFP/E7E6) or artLCMV-(GFP/E7E6) as a boost on day 28 (FIG. 25A) in combinations as outlined in FIGS. 25B-25C. E7-specific CD8 T cell frequencies in blood of Split-vaccinated mice were higher after prime than in artLCMV-vaccinated mice, but these differences became rather minor after heterologous boost and there was not appreciable difference in immunogenicity between PICV-Split66-(TOM/E7E6) and PICV-Split81-(TOM/E7E6). E6- and E7-specific CD8 T cell frequencies were analyzed in spleen on day 148 of the experiment (day 120 after boost) by intracellular cytokine staining after restimulation with overlapping peptide sets spanning the entire E6 and E7 proteins, respectively. Animals undergoing Split-based vaccination haboured ˜15-20-fold higher E6-specific IFN-g-, TNF-α- and IL-2-secreting CD8 T cell frequencies. In contrast, E7-specific responses were comparable between the different immunization regimens, and there was no clear difference between the responses elicited by PICV-Split66-(TOM/E7E6) and PICV-Split8l-(TOM/E7E6).

Next, the Pichinde virus glycoprotein signal peptide length was experimentally determined to further consolidate the buffer peptide concept for Split-PICV vectors. ArtPICV virions were purified and concentrated by ultracentrifugation. The virions were then lysed in a buffer containing 100 mM Tris pH=8.5/1% Sodium Deoxycholate/10 mM TCEP by heating to 950 C for 10 min and by twenty cycles of sonication (30 seconds on, 30 seconds off per cycle) on a Bioruptor system (Dianode). Proteins were alkylated using 15 mM chloroacetamide at 370 C for 30 min and further digested using sequencing-grade chymotrypsin (1/50 w/w, Roche) at 37° C. for 12 hours. To enhance digestion efficiency, 10 mM CaCl₂) was added during digestion. After digestion, the samples were acidified using TFA (final 1%). Peptide desalting was performed using iST cartridges (PreOmics, Germany) following the manufacture's instructions. After drying the samples under vacuum, peptides were stored at −20° C. until further use.

Peptides were fractionated by high-pH reversed phase separation using a XBridge Peptide BEH C18 column (3.5 μm, 130 Å, 1 mm×150 mm, Waters) on an Agilent 1260 Infinity HPLC system. Peptides (100 pg) were loaded in buffer A (20 mM ammonium formate in water, pH 10) and eluted using a two-step linear gradient from 2% to 10% in 5 minutes and then to 50% buffer B (20 mM ammonium formate in 90% acetonitrile, pH 10) over 55 minutes at a flow rate of 42 μl/min. Elution of peptides was monitored with a UV detector (215 nm, 254 nm). A total of 36 fractions were collected and pooled into 12 fractions using a post-concatenation strategy as previously described (Wang et al. Proteomics 2011 May; 11(10):2019-26). Peptides were dried under vacuum until further use. For mass spectrometry analysis, dried peptides were resuspended in 0.1% aqueous formic acid and subjected to LC-MS/MS analysis using an Orbitrap Eclipse Mass Spectrometer fitted with an Ultimate 3000 nano-LC (both Thermo Fisher Scientific) and a custom-made column heater set to 60° C. Peptides were resolved using a RP-HPLC column (75 m×30 cm) packed in-house with C18 resin (ReproSil-Pur C18-AQ, 1.9 m resin; Dr. Maisch GmbH) at a flow rate of 0.3 Lmin-1. The following gradient was used for peptide separation: from 2% B to 12% B over 5 min to 35% B over 45 min to 50% B over 10 min to 95% B over 2 min followed by 18 min at 95% B. Buffer A was composed of 0.1% formic acid in water and buffer B was composed of 80% acetonitrile, 0.1% formic acid in water.

The mass spectrometer was operated in data-independent acquisition (DIA) mode with a total cycle time of approximately 3 see (1 MS1 scan followed by 70 DIA scans). For MS1, 1.2E6 ions were accumulated in the Orbitrap over a maximum time of 45 ms and scanned at a resolution of 120,000 FWHM (at 200 m/z) over a range of 350-1400 m/z. DIA scans were acquired at a target setting of 1.2E6 ions, maximum injection time of 22 ms and a resolution of 15,000 FWHM (at 200 m/z) over a range of 386-1016 m/z. The normalized collision energy was set to 28%, the isolation window was set to 9 m/z and window overlap was set to 1 m/z.

For protein identification the acquired raw-files were analysed by directDIA using the PULSAR search engine (SpectroNaut version 15.7.220308.50606, Biognosys AG) against the UniProt Human and Pichinde Virus database containing commonly observed contaminants. Search criteria included: semi-specific chymotryptic cleavage, permission for 2 missed cleavages, carbamidomethylation (C) was set as fixed modification and oxidation (M) and Deamidation (N) were set as a variable modifications. A Protein Qvalue Cutoff of 0.01 was used to filter the protein identification results.

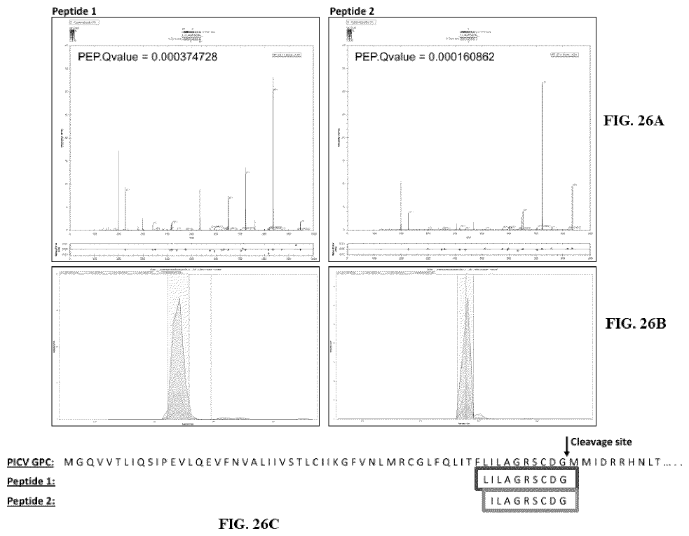

As a result of this workstream, two semi-specific chymotryptic peptides corresponding to the C-terminus of the PICV-GP signal peptide were identified (FIGS. 26A-26B): F.LILAGRSC[Carbamidomethyl]DG.M and L.ILAGRSC[Carbamidomethyl]DG.M (LILAGRSC[Carbamidomethyl]DG and ILAGRSC[Carbamidomethyl]DG with flanking aa residues from PICV GPC separated by a dot). Chymotrypsin cleaves after large hydrophobic amino acids (tyrosine, tryptophane and phenylalanine; single letter codes Y, W, F, respectively) and after leucine (single letter code L) but virtually never after glycine (single letter code G). Accordingly, only the N-terminus of the identified peptides can be explained by chymotrypsin cleavage after F and L, respectively, but the same cannot apply for their C-terminus consisting of a G residue. The identical C-terminal glycine residue of these two independently identified peptides (G59 of the PICV GPC sequence, FIG. 26C) must, therefore, correspond to the naturally processed C-terminus of the PICV glycoprotein signal peptide as it is found in the infectious virion.

Next, PICV-Split(66S)-(TOM/E7E6) and/or PICV-Split(81S)-(TOM/E7E6) were tested to determine if they could be used to induce an immune response against the E7E6 transgene. The immune response was compared to the responses to those elicited by artPICV-(TOM/E7E6) (FIGS. 11A-C). It was observed that mice immunized with PICV-Split(66S)-(TOM/E7E6) and PICV-Split(81S)-(TOM/E7E6) exhibited substantial E7-dextramer-binding CD8+ T cell frequencies in peripheral blood, eight days after intravenous immunization at a dose of 10E5 replication-competent focus forming units (FFU) (FIG. 11D). These responses exceeded those of artPICV-(TOM/E7E6)-immunized mice ˜7-fold and ˜5-fold, respectively. The responses to the nucleoprotein backbone-derived epitope NP38 were, however, comparable between the three groups of mice (FIG. 11E). The relative immunodominance of E7-directed responses was calculated by dividing the frequency of E7-specific responses by the frequency of NP38-specific responses. It was shown that E7-specific responses in PICV-Split(66S)-(TOM/E7E6) and PICV-Split(81S)-(TOM/E7E6) immunized mice were approximately 10-fold more immunodominant than in artPICV-(TOM/E7E6)-immunized mice.

### 6.2 SplitC-LCMV

Many transgenes that are being or should be encoded into the SplitC platform harbor an internal signal peptide sequence that can potentially interfere with the cleavage efficiency of the arenavirus signal peptide (SSP) when encoded on the respective S1 segment. To investigate a potential interference, various transgenes that carry a signal peptide at their N-terminus were selected and either the full-length version or a version lacking the respective native signal peptide (_delSP) was encoded on the S1 segment. LCMV-Split58-vectors encoding TRP2, PSA and GP70 were generated as representative examples.

To generate respective vectors, BHK-21 cells stably expressing the lymphocytic choriomeningitis virus (LCMV) glycoprotein (BHK-23 cells; Flatz L, et al. Nat Med 2010; 16, 339-345) were co-transfected with plasmids (Schematic overview FIG. 12 and FIG. 4): i) a polymerase-I-based expression plasmid encoding for a wildtype LCMV L segment (pol-I-L), ii) polymerase-I-based expression plasmids encoding for the respective LCMV vectors' S1 and S2 segments (Pol-I-S1, Pol-I-S2) including the respective transgenes (i.e., heterologous non-arenaviral polypeptides) and, iii) polymerase-II-driven expression plasmids for the LCMV trans-acting factors L and NP (pC-L, pC-NP). Three days after transfection, cells were propagated from 6-well plates into T75 cell culture flask. The vector containing supernatant was harvested 6 days post transfection and infectious titers were determined by Focus Forming Unit assay (FFU determined on 293T-GP cells; and RCV FFU determined on adherent HEK293 cells).

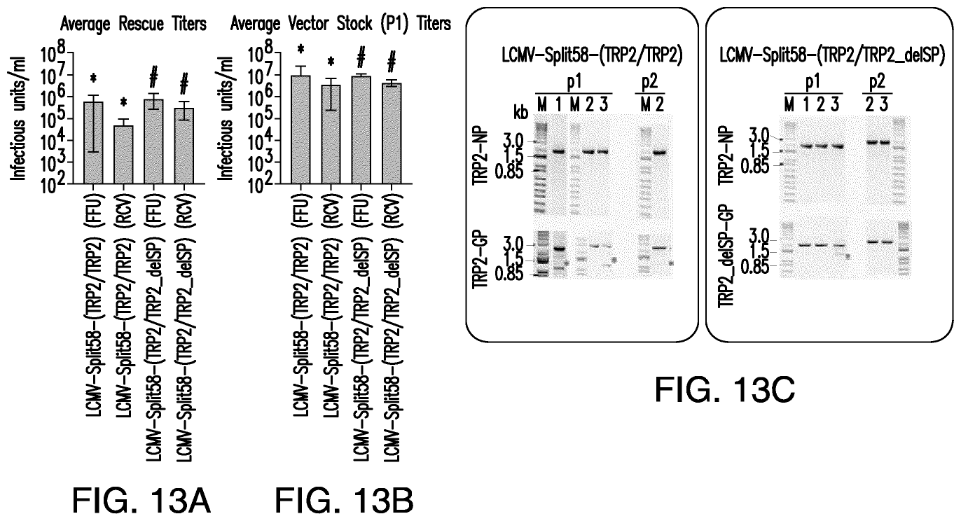

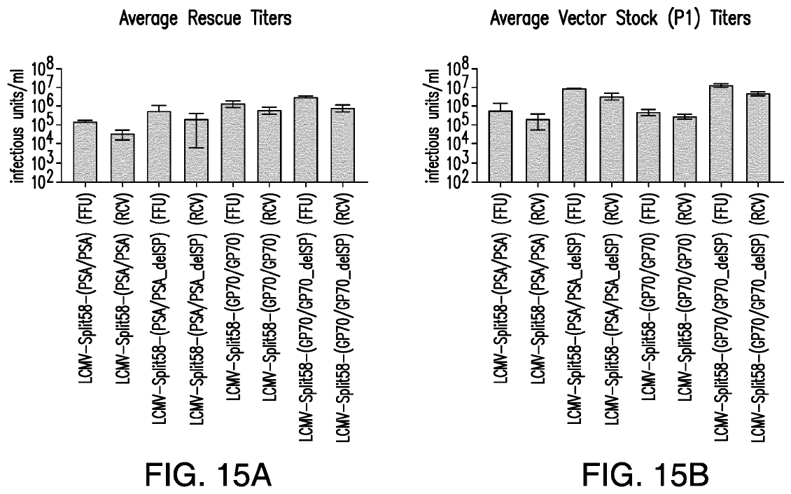

It was observed that the rescue efficiency was very similar for all constructs (100%). However, there was a trend for higher vector titers when the native signal peptide of the transgene encoded on the S1 segment of the respective vector was deleted (i.e., LCMV-Split58-(TRP2/TRP2_delSP), LCMV-Split58-(PSA/PSA_delSP), LCMV-Split58-(GP70/GP70_delSP)) compared to vectors that encoded the native version of the respective heterologous non-arenaviral polypeptides on both S segments (i.e., LCMV-Split58-(TRP2/TRP2), LCMV-Split58-(PSA/PSA), LCMV-Split58-(GP70/GP70)) (FIGS. 13A-13B and FIGS. 15A-15B).

The rescued vector material was further passaged on HEK293 suspension cells, using a MOI of 0.01 for infection. Vector containing cell culture supernatant was harvested 4 days post infection and subjected to titration by FFU and RCV FFU assays. Interestingly, a trend was also observed towards higher titers for vector constructs encoding transgenes devoid of their native signal peptide on the S1 segment (FIG. 13B, FIG. 15B).

Next, the integrity of the encoded TRP2 transgene was investigated by RT-PCR analysis. Vector samples of passages 1 and 2 were subjected to RNA extraction, reverse transcription, and subsequent PCR analysis. PCR primer were selected to flank the transgenes, such that the size of the transgene and potential deletions therein could be analyzed by gel electrophoresis. Interestingly, it was observed that the TRP2 transgene encoded on the S1 segment (Pol-I-S1) revealed significant transgene instability (FIG. 13C) from passage 1 onwards (evident in accessory “TRP2-GP” amplicons of shorter than expected length, FIG. 13C), whereas 2 out of 3 replicates of the vector construct encoding TRP2_delSP (i.e., the TRP2 variant in which the TRP2 signal peptide has been removed, “TRP2_delSP-GP” amplicons in FIG. 13C) on the S1 segment showed transgene stability until passage 2 (FIG. 13C).

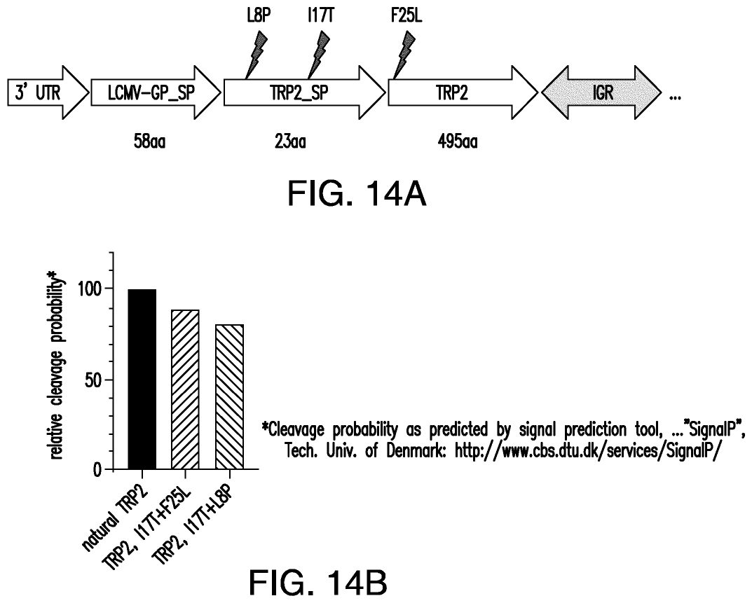

To investigate the influence of the transgene internal signal sequence on the vector rescue performance and stability of the encoded TRP2 transgene, sequence analyses of the PCR products from two different passage 1 vector samples were performed using Sanger consensus sequencing (FIGS. 14A-14B). Three mutations were identified, which were located within the TRP2-internal signal peptide or in close proximity to the TRP2-internal signal peptide cleavage site at amino acid position 23/24 of TRP2 (FIG. 14A). Of note, one mutation (I17T) was detected in both independently rescued vector samples analyzed. Upon analysis of these mutated sequences using the signal peptide analysis tool SignalP we found that the combination of two of the above mutations (I17T plus F25L or I17T plus L8P) is predicted to result in a reduced cleavage probability of 74% and 67%, respectively (FIG. 14B).

The results from this example point to an increased rescue probability/growth potential of vectors encoding transgenes that have a lower cleavage probability for their internal signal peptide, possibly by favoring cleavage of the arenavirus signal peptide, and thus promoting correct assembly of all three parts of the arenaviral glycoprotein (GP1, GP2 and SSP). If this hypothesis is true, the identification of individual signal peptide mutations could potentially enable modification of transgenes of interest (that encode for an internal signal peptide) in a targeted way; thus, potentially obviating the necessity to delete the whole signal peptide from the antigen.

### 6.3 SplitC-PICV

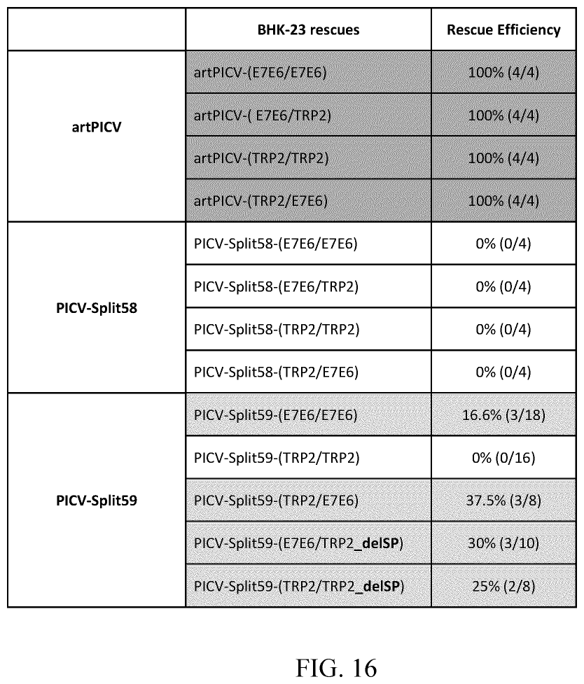

When the Split-C vector design was applied to PICV, analogous to what is described above for LCMV-Split58 (refer to example in Section 6.2), it was observed that vectors expressing E7E6 and Trp2 as transgenes on their S1 and S2 segments, respectively, could not be rescued by co-transfection of BHK-21 cells stably expressing the LCMV glycoprotein (BHK-23 cells) (FIG. 16). While bi-segmented, non-replicating vector particles were detectable, the generation of replication competent vectors was not possible (FIG. 16). Thus, it was hypothesized that the length of the PICV glycoprotein signal peptide may not be 58 amino acids as reported for LCMV, Lassa virus and Junin virus (Agnihothram, S S, et al. (2006) J Virol 80, 5189-5198; Eichler R, et al. (2003) EMBO Rep 4, 1084-1088; Schrempf S, et al. (2007) J Virol 81, 12515-12524). As such, plasmids were re-designed to encode a signal peptide of either 58 Aa or 59 Aa in length, the latter as predicted by the PrediSi (Prediction of SIgnalpeptides) website (from the Technical University of Braunschweig) (FIG. 2). For this example, the following vectors were generated: (i) PICV-Split vectors with a 58Aa-long PICV signal peptide (PICV-Split58 vectors), (ii) PICV-Split vectors with a 59Aa-long PICV signal peptide (PICV-Split59 vectors), and/or (iii) replication-competent, tri-segmented PICV vectors where the PICV-GP was expressed as one open reading frame (ORF) under control of the genomic 3′ UTR promoter of the S1 segment (artPICV vectors). The respective vector constructs encoded the following heterologous non-arenaviral polypeptides on their respective S1 and/or S2 segments: (a) E7E6, (b) TRP2, or (c) TRP2 devoid of its native N-terminal signal peptide (TRP2_delSP). Upon co-transfection of BHK-23 cells with the respective plasmids it was found that replication competent PICV-Split59 but no PICV-Split58 vectors could be rescued. artPICV vectors encoding the above transgenes in all possible combinations on their S1 and S2 segments were recovered in all four independent transfection cultures (100%). In contrast, the recovery rate of replication-competent PICV-Split59 vectors ranged from 16.6% 37.5% only. Furthermore, it was observed that only PICV-Split59 vectors encoding TRP2_delSP (but not full-length TRP2) on their S1 segment yielded replication competent vectors (FIG. 16, compare PICV-Split59-(TRP2/TRP2_delSP) and PICV-Split59-(TRP2/TRP2)).

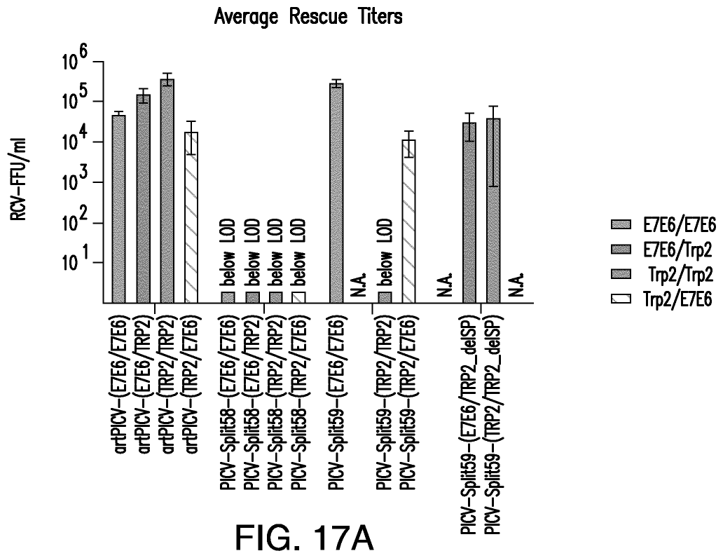

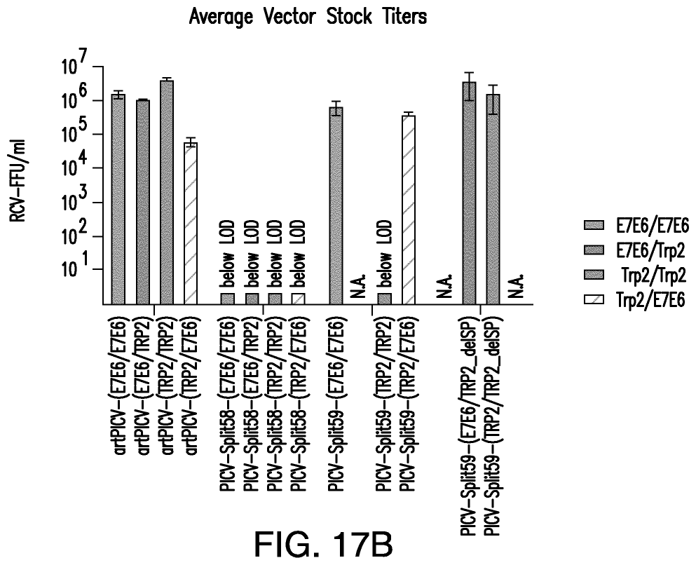

All successful vector rescues in the above experiment yielded substantial replication-competent titers (FIG. 17A). This finding was corroborated and extended when the viable vectors were passaged in HEK293 suspension cells (FIG. 17B).

### 7. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

TABLE 5
Assession number for specific arenaviruses

| | | GenBank Accession | Ref. Seq. |
|---|---|---|---|
| Virus name | Isolate | number | number |
| Allpahuayo virus | CHLP-2472 | L: AY216502; | L: NC_010249; |
| | | S: AY012687 | S: NC_010253 |
| Alxa virus | RtDs-AreV-IM2014 | L: KY432892; | |
| | | S: KY432893 | |
| Junín virus | XJ13 | L: AY358022; | L: NC_005080; |
| | | S: AY358023 | S: NC_005081 |
| Bear Canyon virus | AV A0070039 | L: AY924390; | L: NC_010255; |
| | | S: AY924391 | S: NC_010256 |
| Sabiá virus | SPH114202 | L: AY358026; | L: NC_006313; |
| | | S: U41071 | S: NC_006317 |
| Pichindé virus | AN3739 | L: AF427517; | L: NC_006439; |
| | | S: K02734 | S: NC_006447 |
| Chapare virus | 810419 | L: EU260464; | L: NC_010563; |
| | | S: EU260463 | S: NC_010562 |
| Lìjiāng virus | KS4 | L: MF414201; | |
| | | S: MF414202 | |
| Cupixi virus | BeAn 119303 | L: AY216519; | L: NC_010252; |
| | | S: AF512832 | S: NC_010254 |
| Flexal virus | BeAn 293022 | L: EU627611; | L: NC_010759; |
| | | S: AF512831 | S: NC_010757 |
| Gairo virus | TZ-27421 | L: KJ855307; | L: NC_026247; |
| | | S: KJ855308 | S: NC_026246 |
| Guanarito virus | INH-95551 | L: AY358024; | L: NC_005082; |
| | | S: AY129247 | S: NC_005077 |
| Ippy virus | Dak An B 188d | L: DQ328878; | L: NC_007906; |
| | | S: DQ328877 | S: NC_007905 |
| Lassa virus | Josiah | L: U73034; | L: NC_004297; |
| | | S: J04324 | S: NC_004296 |
| Latino virus | MARU 10924 | L: EU627612; | L: NC_010760; |
| | | S: AF512830 | S: NC_010758 |
| Loei River virus | R5074 | L: KC669693; | L: NC_038365; |
| | | S: KC669698 | S: NC_038364 |
| Lujo virus | R4356 | L: FJ952385; | L: NC_012777; |
| | | S: FJ952384 | S: NC_012776 |
| Luna virus | LSK-1 | L: AB586645; | L: NC_016153; |
| | | S: AB586644 | S: NC_016152 |
| Luli virus | SLW-1 | L: AB972431; | |
| | | S: AB972430 | |
| Lunk virus | NKS-1 | L: AB693151; | L: NC_018711; |
| | | S: AB693150 | S: NC_018710 |
| lymphocytic | Armstrong 53b | L: AY847351; | L: NC_004291; |
| choriomeningitis | | S: AY847350 | S: NC_004294 |
| virus | | | |
| Machupo virus | Carvallo | L: AY358021; | L: NC_005079; |
| | | S: AY129248 | S: NC_005078 |
| Mariental virus | N27 MrMi.n9 | L: KP867641; | L: NC_027136; |
| | | S: KM272987 | S: NC_027134 |
| Merino Walk virus | Merino Walk | L: GU078661; | L: NC_023763; |
| | | S: GU078660 | S: NC_023764 |
| mobala virus | Acar 3080 | L: DQ328876; | L: NC_007904; |
| | | S: AY342390 | S: NC_007903 |
| Mopeia virus | AN20410 | L: AY772169; | L: NC_006574; |
| | | S: AY772170 | S: NC_006575 |
| Morogoro virus | 3017/2004 | L: EU914104; | |
| | | S: EU914103 | |
| Okahandja virus | N73 OkhMi.n4 | L: KP867642; | L: NC_027137; |
| | | S: KM272988 | S: NC_027135 |
| Oliveros virus | 3229 | L: AY216514; | L: NC_010250; |
| | | S: U34248 | S: NC_010248 |
| Paraná virus | 12056 | L: EU627613; | L: NC_010761; |
| | | S: AF485261 | S: NC_010756 |
| Pirital virus | VAV-488 | L: AY494081; | L: NC_005897; |
| | | S: AF485262 | S: NC_005894 |
| Aporé virus | LBCE 12071 | L: MF317491; | L: NC_040763; |
| | | S: MF317490 | S: NC_040762 |
| Ryukyu virus | YN2013 | L: KM020190; | L: NC_039010; |
| | | S: KM020191 | S: NC_039009 |
| Amaparí virus | BeAn 70563 | L: AY216517; | L: NC_010251; |
| | | S: AF485256 | S: NC_010247 |
| Solwezi virus | 13ZR68 | L: AB972429; | L: NC_038366; |
| | | S: AB972428 | S: NC_038367 |
| souris virus | PREDICT-05775- | L: KP050226; | L: NC_039011; |
| | 5302-5304 | S: KP050227 | S: NC_039012 |
| Tacaribe virus | T.RVL.II 573 | L: J04340; | L: NC_004292; |
| | | S: M20304 | S: NC_004293 |
| Tamiami virus | W10777 | L: AY924393; | L: NC_010702; |
| | | S: AF485263 | S: NC_010701 |
| Wēnzhōu virus | Rn-242 | L: KJ909795; | L: NC_026019; |
| | | S: KJ909794 | S: NC_026018 |
| Whitewater Arroyo | AV 9310135 | L: AY924395; | L: NC_010703; |
| virus | | S: AF228063 | S: NC_010700 |
| Big Brushy Tank | AV D0390174 | L: EU938665; | |
| virus | | S: EF619035 | |
| Catarina virus | AV N0010001 | S: JX237768 | |
| Skinner Tank virus | AV D1000090 | L: EU938659; | |
| | | S: EU123328 | |
| Tonto Creek virus | AV D0390060 | L: EU938663; | |
| | | S: EF619034 | |
| Xapuri virus | LBCE 19881 | S: MG976578; | |
| | | L: MG976577 | |

S indicates the GenBank accession number or the sequence number of the S segment;
L indicates GenBank accession number or the sequence number of the L segment

What is claimed:

1. An engineered arenavirus particle, wherein the genome of the arenavirus particle comprises:
a. an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a heterologous non-arenaviral signal peptide, arenavirus GP1 and arenavirus GP2 under the control of an arenavirus genomic 5′ UTR;
b. an S segment that encodes NP under the control of an arenavirus genomic 3′ UTR and a second heterologous non-arenaviral polypeptide or no polypeptide under the control of an arenavirus genomic 5′ UTR; and
c. an L segment that encodes the L protein under the control of an arenavirus genomic 3′ UTR and the Z protein under the control of an arenavirus genomic 5′ UTR; or
wherein the genome of the arenavirus particle comprises:
d. an S segment that encodes (i) an arenavirus GP signal peptide, a buffer peptide, and a first heterologous non-arenaviral polypeptide or (ii) the arenavirus GP signal peptide and the buffer peptide under the control of an arenavirus genomic 3′ UTR and a second heterologous non-arenaviral polypeptide or no polypeptide under the control of an arenavirus genomic 5′ UTR;
e. an S segment that encodes NP under the control of an arenavirus genomic 3′ UTR and a heterologous non-arenaviral signal peptide, arenavirus GP1 and arenavirus GP2 under the control of an arenavirus genomic 5′ UTR; and
f. an L segment that encodes the L protein under the control of an arenavirus genomic 3′ UTR and the Z protein under the control of an arenavirus genomic 5′ UTR, wherein the first heterologous non-arenaviral polypeptide and the second heterologous non-arenaviral polypeptide are the same or different from each other.

2. The engineered arenavirus particle of claim 1, wherein the buffer peptide comprises or consists of a fragment of the arenavirus GP1.

3. The engineered arenavirus particle of claim 2, wherein the fragment of the arenavirus GP1 is shorter than the arenavirus GP1.

4. The engineered arenavirus particle of claim 3, wherein the fragment of the arenavirus GP1 comprises about, at most about, or at least about 1, 2, 3, 4, 5, 10, 15, 20, 25, 30, 35, 40, 45, 50, 55, 60, or more than 60 amino acids of the arenavirus GP1.

5. The engineered arenavirus particle of any one of claims 2 to 4, wherein the fragment of the arenavirus GP1 is the N-terminal fragment of the arenavirus GP1.

6. The engineered arenavirus particle of claim 1, wherein the buffer peptide comprises about or at most about 1%, 2%, 5%, 10%, 15%, 20%, 25%, 30% basic amino acids, or wherein the first 8 N-terminal amino acids of the buffer peptide comprise at most 1, 2, 3, 4, or 5 basic amino acids.

7. The engineered arenavirus particle of any one of claims 1-6, wherein the first 5 N-terminal amino acids of the buffer peptide comprise 1, 2, 3, 4, or 5 acidic and/or neutral and/or nonpolar aliphatic amino acids; wherein the first 6 N-terminal amino acids of the buffer peptide comprise 1, 2, 3, 4, 5, or 6 acidic and/or neutral and/or nonpolar aliphatic amino acids; wherein the first 7 N-terminal amino acids of the buffer peptide comprise 1, 2, 3, 4, 5, 6, or 7 acidic and/or neutral and/or nonpolar aliphatic amino acids; or wherein the first 8 N-terminal amino acids of the buffer peptide comprise 1, 2, 3, 4, 5, 6, 7, or 8 acidic and/or neutral and/or nonpolar aliphatic amino acids.

8. The engineered arenavirus particle of claim 7, wherein the last C-terminal amino acid of the buffer peptide is a basic amino acid; wherein the last 2 C-terminal amino acids of the buffer peptide comprise 1 or 2 basic amino acids; wherein the last 3 C-terminal amino acids of the buffer peptide comprise 1, 2, or 3 basic amino acids; wherein the last 4 C-terminal amino acids of the buffer peptide comprise 1, 2, 3, or 4 basic amino acids; or wherein the last 5 C-terminal amino acids of the buffer peptide comprise 1, 2, 3, 4, or 5 basic amino acids.

9. The engineered arenavirus particle of claim 7, wherein the 5 amino acids C-terminal to the first 5, 6, 7, or 8 N-terminal amino acids of the buffer peptide comprise about or at most about 1, 2, or 3 basic amino acids.

10. The engineered arenavirus particle of any one of claims 1-7, wherein the buffer peptide does not comprise basic amino acids.

11. The engineered arenavirus particle of any one of claims 1-10, wherein the first 3, 4, 5, 6, 7, or 8 N-terminal amino acids of the buffer peptide do not comprise basic amino acids.

12. The engineered arenavirus particle of any one of claims 1-11, wherein the buffer peptide comprises about or at least about 50%, 60%, 70%, 80%, 90%, 95%, 96%, 97%, 98%, 99%, or 100% acidic amino acids and/or neutral and/or nonpolar aliphatic amino acids.

13. The engineered arenavirus particle of any one of claims 1-12, wherein the first 3, 4, 5, 6, 7, or 8 N-terminal amino acids of the buffer peptide comprise about or at least about 75%, 80%, 85%, 90%, 95%, 96%, 97%, 98%, 99%, or 100% acidic amino acids and/or neutral and/or nonpolar aliphatic amino acids.

14. The engineered arenavirus particle of any one of claims 1-13, wherein the buffer peptide is about, at least about, or at most about, 1, 2, 3, 4, 5, 10, 15, 20, 25, 30, 35, 40, 45, 50, 55, 60, 65, 70, or more than 70 amino acids.

15. The engineered arenavirus particle of any one of claims 1-14, wherein any native signal peptide of the first heterologous non-arenaviral polypeptide has been truncated or deleted.

16. The engineered arenavirus particle of any one of claims 1-15, wherein the arenavirus GP signal peptide can be cleaved from the buffer peptide or a portion thereof by a signal peptide peptidase.

17. The engineered arenavirus particle of claim 16, wherein the signal peptide peptidase cleaves immediately N-terminal to the buffer peptide, after the first N-terminal amino acid of the buffer peptide, after the second N-terminal amino acid of the buffer peptide, after the third N-terminal amino acid of the buffer peptide, after the fourth N-terminal amino acid of the buffer peptide, or after the fifth N-terminal amino acid of the buffer peptide.

18. The engineered arenavirus particle of any one of claims 1-17, wherein any native signal peptide of the second heterologous non-arenaviral polypeptide is retained.

19. The engineered arenavirus particle of any one of claims 1-18, wherein the arenavirus GP signal peptide is cleaved at exactly the position where the buffer peptide starts, at amino acid 1, 2, 3, 4, 5, 6, 7, 8, 9, 10, or more than 10 amino acids N-terminal from where the buffer peptide starts, or at amino acid 1, 2, 3, 4, 5, 6, 7, 8, 9, 10, or more than 10 amino acids C-terminal from where the buffer peptide starts.

20. The engineered arenavirus particle of any one of claims 1-19, wherein the buffer peptide is about, at least about, or at most about, 1, 2, 3, 4, 5, 10, 15, 20, 25, 30, 35, 40, 45, 50, 55, 60, 65, 70, or more than 70 amino acids.

21. The engineered arenavirus particle of any one of claims 1-20, wherein the arenavirus particle is derived from an arenavirus of Clade A.

22. The engineered arenavirus particle of claim 21, wherein the arenavirus of Clade A is Pichinde virus (PICV).

23. The engineered arenavirus particle of any one of claims 1-22, wherein the sequence of the arenavirus GP signal peptide, the buffer peptide, and the first heterologous non-arenaviral polypeptide is from N-terminus to C-terminus.

24. The engineered arenavirus particle of any one of claims 1-23, wherein the arenavirus GP signal peptide, the buffer peptide, and the first heterologous non-arenaviral polypeptide form a fusion protein.

25. The engineered arenavirus particle of any one of claims 1-24, wherein the heterologous non-arenaviral signal peptide is the signal peptide of vesicular stomatitis virus glycoprotein.

26. The engineered arenavirus particle of any one of claims 1-25, wherein inter-segmental recombination of the two S segments, uniting two arenavirus ORFs on only one instead of two separate S segments, results in a bi-segmented arenavirus that is not viable or that in cell culture reaches peak titers that are lower than or equivalent to the tri-segmented arenavirus from which the bi-segmented arenavirus was recombined.

27. The engineered arenavirus particle of any one of claims 1-26, wherein the arenavirus GP1 and arenavirus GP2 comprises an amino acid sequence starting at about amino acid 59 to about 508 of SEQ ID NO:3, or a biologically active fragment thereof.

28. The engineered arenavirus particle of any one of claims 1-27, wherein the GP signal peptide fused to the buffer peptide comprises or consists of an amino acid sequence that is about or at least about 95%, 96%, 97%, 98%, 99%, or 100% identical to the sequence of amino acid 1 to amino acid 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:3.

29. The engineered arenavirus particle of claim 28, wherein the GP signal peptide fused to the buffer peptide comprises or consists of an amino acid sequence that is the sequence of amino acid 1 to amino acid 66 of SEQ ID NO:3.

30. The engineered arenavirus particle of claim 28, wherein the GP signal peptide fused to the buffer peptide comprises or consists of an amino acid sequence that is the sequence of amino acid 1 to amino acid 81 of SEQ ID NO:3.

31. The engineered arenavirus particle of claim 28, wherein the GP signal peptide fused to the buffer peptide comprises or consists of an amino acid sequence that is the sequence of amino acid 1 to amino acid 113 of SEQ ID NO:3.

32. The engineered arenavirus particle of claim 1, wherein the coding sequence of the first heterologous non-arenaviral polypeptide starts at about amino acid 60 or more than about 60 amino acids downstream of the methionine start codon of the GP signal peptide.

33. The engineered arenavirus particle of any one of claims 1 to 32, wherein the first heterologous non-arenaviral polypeptide, the second heterologous non-arenaviral polypeptide, or both heterologous non-arenaviral polypeptides are each an antigen derived from an infectious organism, tumor, or allergen.

34. The engineered arenavirus particle of claim 33, wherein the antigen is selected from the group consisting of
a. viral antigens, wherein the viral antigen is from a virus family selected from the group consisting of adenoviridae, herpesviridae, leviviridae, orthomyxoviridae, parvoviridae, filoviridae, hantaviridae, poxviridae, papillomaviridae, polyomaviridae, paramyxoviridae, pneumoviridae, picornaviridae, reoviridae, retroviridae, flaviviridae, hepadnaviridae, togaviridae, rhabdoviridae, arenaviridae, and coronaviridae;
b. bacterial antigens, wherein the bacterial antigen is from a bacteria family selected from the group consisting of Aquaspirillum family, Azospirillum family, Azotobacteraceae family, Bacteroidaceae family, Bartonellaceae family, Bdellovibrio family, Campylobacteraceae family, Chlamydiaceae family, Clostridiaceae family, Enterobacteriaceae family, Gardinella family, Pasteurellaceae family, Halobacteriaceae family, Helicobacter family, Legionallaceae family, Listeriaceae family, Methylococcaceae family, mycobacteriaceae, Neisseriaceae family, Oceanospirillum family, Pasteurellaceae family, Streptococcaceae family, Pseudomonadaceae family, Rhizobiaceae family, Spirillum family, Spirosomaceae family, Staphylococcaceae family, Helicobacter family, Yersinia family, Bacillus antracis and Vampirovibrio family, and
c. tumor neoantigens or neo-epitopes and tumor associated antigens; and wherein the tumor associated antigen is selected from the group consisting of artificial fusion protein of HPV 16 E7 and E6 proteins, oncogenic viral antigens, cancer-testis antigens, oncofetal antigens, tissue differentiation antigens, mutant protein antigens, Adipophilin, AIM-2, ALDHIAI, BCLX (L), BING-4, CALCA, CD45, CPSF, cyclin DI, DKKI, ENAH (hMcna), Ga733 (EpCAM), EphA3, EZH2, FGF5, glypican-3, G250/MN/CAIX, HER-2/neu, IDOI, IGF2B3, IL13Ralpha2, Intestinal carboxyl esterase, alphafoetoprotein, Kallikrein 4, KIF20A, Lengsin, M-CSF, MCSP, mdm-2, Meloe, MMP-2, MMP-7, MUCI, MUC5AC, p53 (non-mutant), PAX5, PBF, PRAME, PSMA, RAGE, RAGE-I, RGS5, RhoC, RNF43, RU2AS, secernin 1, SOX10, STEAPI (six-transmembrane epithelial antigen of the prostate 1), survivin, Telomerase, VEGF, WT1, EGF-R, CEA, CD20, CD33, CD52, MELANA/MART1, MART2, NY-ESO-1, p53, MAGE A1, MAGE A3, MAGE-4, MAGE-5, MAGE-6, CDK4, alpha-actinin-4, ARTC1, BCR-ABL, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML, ETV6-AML1 fusion protein, FLT3-ITD, FNl, GPNMB, LDLR-fucosyltransferase fusion protein, NFYC, OGT, OS-9, pml-RARalpha fusion protein, PRDX5, PTPRK, H-ras, K-ras (V-Ki-ras2 Kirsten rat sarcoma viral oncogene), N-ras, RBAF600, SIRT2, SNRPD1, SSX, SSX2, SYT-SSXl or -SSX2 fusion protein, TGFbetaRII, Triosephosphate isomerase, ormdm-2, LMP2, HPV E6, HPV E7, EGFRvIII (epidermal growth factor variant III), Idiotype, GD2, ganglioside G2), Ras-mutant, p53 (mutant), Proteinase3 (PRI), Tyrosinase, PSA, hTERT, Sarcoma translocation breakpoints, EphA2, prostatic acid phosphatase PAP, neo-PAP, ML-IAP, AFP, ERG (TMPRSS2 ETS Fusion gene), NAI 7, PAX3, ALK, Androgen Receptor, Cyclin B1, Polysialic acid, MYCN, TRP2, TRP2-Int2, GD3, Fucosyl GMI, Mesothelin, PSCA, sLe(a), cyp1B1, PLACI, GM3, BORIS, Tn, GLoboH, NY-BR-I, SART3, STn, Carbonic Anhydrase IX, OY-TESI, Sperm protein 17, LCK, high molecular weight melanoma-associated antigen (HMWMAA), AKAP-4, SSX2, XAGE 1, B7H3, Legumain, Tie 2, Page4, VEGFR2, MAD-CT-I, FAP, PDGFR-beta, MADCT-2, For-related antigen 1, TRPI, GP100, CA-125, CA19-9, Calretinin, Epithelial membrane antigen (EMA), Epithelial tumor antigen (ETA), CD19, CD34, CD99, CDI 17, Chromogranin, Cytokeratin, Desmin, Glial fibrillary acidic protein (GFAP), gross cystic disease fluid protein (GCDFP-15), HMB-45 antigen, Myo-D1, muscle-specific actin (MSA), neurofilament, neuronspecific enolase (NSE), placental alkaline phosphatase, synaptophysis, thyroglobulin, thyroid transcription factor-1, dimeric form of the pyruvate kinase isoenzyme type M2 (tumor M2-PK), BAGE BAGE-1, CAGE, CTAGE, FATE, GAGE, GAGE-I, GAGE-2, GAGE-3, GAGE-4, GAGE-5, GAGE-6, GAGE-7, HCA661, HOM-TES-85, MAGEA, MAGEB, MAGEC, NA88, NY-SAR-35, SPANXBI, SPAI 7, SSX, SYCP1, TPTE, Carbohydrate/ganglioside GM2 (oncofetal antigen-immunogenic-1 OFA-I-1), GM3, CA 15-3 (CA 27.29BCAA), CA 195, CA 242, CA 50, CAM 43, CEA, EBNA, EF2, Epstein-Barr virus antigen, HLA-A2, HLA-Al 1, HSP70-2, KIAAO205, MUM-I, MUM-2, MUM-3, Myosin class I, GnTV, Herv-K-mel, LAGE-I, LAGE-2, (sperm protein) SPI 7, SCP-I, P15(58), Hom/Mel-40, E2A-PRL, H4-RET, IGH-IGK, MYL-RAR, TSP-180, P185erbB2, p180erbB-3, c-met, nm-23H1, TAG-72, TAG-72-4, CA-72-4, CAM 17.1, NuMa, 13-catenin, P16, TAGE, CT7, 43-9F, 5T4, 791Tgp72, 13HCG, BCA225, BTAA, CD68KP1, CO-029, HTgp-175, M344, MG7-Ag, MOV18, NB70K, NYCO-1, RCAS1, SDCCAG16, TA-90, TAAL6, TLP, TPS, CD22, CD27, CD30, CD70, prostein, TARP (T cell receptor gamma alternate reading frame protein), Trp-p8, integrin αvβ3 (CD61), galactin, or Ral-B, CD123, CLL-1, CD38, CS-1, CD138, and ROR1.

35. The engineered arenavirus particle of any one of claims 1 to 34, wherein the genome of the arenavirus particle encodes the first heterologous non-arenaviral polypeptide and/or the second heterologous non-arenaviral polypeptide; wherein the growth or infectivity of the arenavirus particle is not inferior to a second arenavirus particle; wherein the genome of the second arenavirus particle encodes the same first heterologous non-arenaviral polypeptide and/or the same second heterologous non-arenaviral polypeptide; and wherein all arenaviral GP, NP, Z and L in the second arenavirus particle are each expressed as one ORF.

36. The engineered arenavirus particle of any one of claims 1 to 35, wherein the titer of the arenavirus particle is at least about 1%, 2%, 3%, 4%, 5%, 6%, 7%, 8%, 9%, 10%, 15%, 20%, 30%, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold lower during a persistent infection in AGRAG mice than the titer of the respective wild-type parental arenavirus particle.

37. The engineered arenavirus particle of any one of claims 1 to 36, wherein the arenavirus particle expresses a heterologous non-arenaviral polypeptide under control of an arenavirus genomic 3′ UTR; wherein the arenavirus particle induces at least about 1%, 2%, 3%, 4%, 5%, 6%, 7%, 8%, 9%, 10%, 15%, 20%, ³⁰, 40%, 50%, 60%, 70%, 80%, 90%, 100%, 1.5-fold, 2-fold, 3-fold, 4-fold, 5-fold, 6-fold, 7-fold, 8-fold, 9-fold, 10-fold, 12-fold, 15-fold, 18-fold, 20-fold, 25-fold, or 30-fold higher immune responses against the heterologous non-arenaviral polypeptide in a subject after the arenavirus particle is administered to the subject as compared to after another arenavirus particle expressing the same heterologous non-arenaviral polypeptide under control of an arenavirus genomic 5′ UTR is administered to the subject or to a comparable subject.

38. A set of cDNAs from which the genomic segments of the engineered arenavirus particle of any one of claims 1 to 37 can be transcribed.

39. A set of DNA expression vectors comprising the cDNAs of claim 38.

40. A host cell comprising the set of cDNAs of claim 38 or the set of DNA expression vectors of claim 39.

41. A method of manufacturing the engineered arenavirus particle of any one of claims 1 to 37, comprising maintaining the host cell of claim 40 under conditions suitable for virus formation and harvesting the engineered arenavirus particle.

42. A pharmaceutical composition comprising the engineered arenavirus particle of any one of claims 1 to 37 and a pharmaceutically acceptable carrier.

43. Use of the engineered arenavirus particle of any one of claims 1 to 37 for treating or preventing disease in a human patient.

44. A nucleotide sequence comprising an open reading frame encoding a polypeptide consisting of an amino acid sequence that is about or at least about 95%, 96%, 97%, 98%, 99%, or 100% identical to the sequence of amino acid 1 to amino acid 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than 113 of SEQ ID NO:3.

45. The nucleotide sequence of claim 44, wherein the nucleotide sequence comprises an open reading frame encoding a polypeptide consisting of amino acid 1 to 66 of SEQ ID NO:3.

46. The nucleotide sequence of claim 44, wherein the nucleotide sequence comprises an open reading frame encoding a polypeptide consisting of amino acid 1 to 81 of SEQ ID NO:3.

47. The nucleotide sequence of claim 44, wherein the nucleotide sequence comprises an open reading frame encoding a polypeptide consisting of amino acid 1 to 113 of SEQ ID NO:3.

48. The engineered arenavirus particle of any one of claims 1-27, wherein the GP signal peptide fused to the buffer peptide comprises or consists of an amino acid sequence that is about or at least about 95%, 96%, 97%, 98%, 99%, or 100% identical to the sequence of amino acid 1 to amino acid: 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:6.

49. The engineered arenavirus particle of claim 48, wherein the GP signal peptide fused to the buffer peptide comprises or consists of an amino acid sequence that is the sequence of amino acid 1 to amino acid 66 of SEQ ID NO:6.

50. The engineered arenavirus particle of claim 48, wherein the GP signal peptide fused to the buffer peptide comprises or consists of an amino acid sequence that is the sequence of amino acid 1 to amino acid 81 of SEQ ID NO:6.

51. The engineered arenavirus particle of any one of claims 1-27, wherein the GP signal peptide fused to the buffer peptide comprises or consists of an amino acid sequence that is about or at least about 95%, 96%, 97%, 98%, 99%, or 100% identical to the sequence of amino acid 1 to amino acid: 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, or longer than amino acid 113 of SEQ ID NO:120.

52. The engineered arenavirus particle of claim 51, wherein the GP signal peptide fused to the buffer peptide comprises or consists of an amino acid sequence that is the sequence of amino acid 1 to amino acid 66 of SEQ ID NO:120.

53. The engineered arenavirus particle of claim 51, wherein the GP signal peptide fused to the buffer peptide comprises or consists of an amino acid sequence that is the sequence of amino acid 1 to amino acid 81 of SEQ ID NO:120.

54. A method of immunizing a subject suspected of having, diagnosed with, or at risk of developing a disease or disorder, comprising:
a. administering a first engineered arenavirus particle to the subject, wherein the first engineered arenavirus particle is the engineered arenavirus particle of any one of claims 1-37 and 48-53; and
b. administering a second engineered arenavirus particle to the subject, wherein the second engineered arenavirus particle is the engineered arenavirus particle of any one of claims 1-37 and 48-53, wherein the first engineered arenavirus particle is derived from a first arenavirus species and the second engineered arenavirus particle is derived from a second arenavirus species.

55. The method of claim 54, wherein the first engineered arenavirus particle and the second engineered arenavirus particle are derived from the same arenavirus species.

56. The method of claim 54, wherein the first engineered arenavirus particle and the second engineered arenavirus particle are derived from different arenavirus species.

57. The method of claim 55, wherein the same arenavirus species is PICV.

58. The method of claim 55, wherein the same arenavirus species is LCMV.

59. The method of claim 55, wherein the same arenavirus species is Tamiami virus (TAMV).

60. The method of claim 55, wherein the same arenavirus species is Tacaribe virus (TCRV).

61. The method of claim 56, wherein the first arenavirus species is LCMV and the second arenavirus species is PICV.

62. The method of claim 56, wherein the first arenavirus species is PICV and the second arenavirus species is LCMV.

63. The method of claim 56, wherein the first arenavirus species is LCMV and the second arenavirus species is TAMV.

64. The method of claim 56, wherein the first arenavirus species is TAMV and the second arenavirus species is LCMV.

65. The method of claim 56, wherein the first arenavirus species is LCMV and the second arenavirus species is TCRV.

66. The method of claim 56, wherein the first arenavirus species is TCRV and the second arenavirus species is LCMV.

67. The method of claim 56, wherein the first arenavirus species is TAMV and the second arenavirus species is PICV.

68. The method of claim 56, wherein the first arenavirus species is PICV and the second arenavirus species is TAMV.

69. The method of claim 56, wherein the first arenavirus species is TCRV and the second arenavirus species is PICV.

70. The method of claim 56, wherein the first arenavirus species is PICV and the second arenavirus species is TCRV.

71. The method of claim 56, wherein the first arenavirus species is TCRV and the second arenavirus species is TAMV.

72. The method of claim 56, wherein the first arenavirus species is TAMV and the second arenavirus species is TCRV.

73. The method of any one of claims 54-72, wherein the disease or disorder is an infection.

74. The method of any one of claims 54-72, wherein the disease or disorder is a cancer.
